(19) United States
(12) Patent Application Publication
(10) Pub. No.: US 2023/0279399 A1
(43) Pub. Date: Sep. 7, 2023

(54) RNAI CONSTRUCTS FOR INHIBITING HSD17B13 EXPRESSION AND METHODS OF USE THEREOF

(71) Applicant: Amgen Inc., Thousand Oaks, CA (US)

(72) Inventors: Daniel C. H. LIN, Redwood City, CA (US); Michael OLLMANN, San Carlos, CA (US); Justin K. MURRAY, Moorpark, CA (US); Bradley J. HERBERICH, Newbury Park, CA (US); Amrita DAS, San Ramon, CA (US); Patrick COLLINS, Millbrae, CA (US); Oliver HOMANN, Berkeley, CA (US)

(73) Assignee: Amgen Inc., Thousand Oaks, CA (US)

(21) Appl. No.: 18/007,560

(22) PCT Filed: Jun. 3, 2021
(86) PCT No.: PCT/US2021/035730

§ 371 Date: Dec. 1, 2022

Related U.S. Application Data
(60) Provisional application No. 63/033,128, filed on Jun. 1, 2020.

(51) Int. Cl. C12N 15/113

(52) U.S. Cl. CPC C12N 15/1137; C12N 2310/321; C12N 2310/322; C12N 2310/315; C12N 2310/11

(57) ABSTRACT

The present invention relates to RNAi constructs for reducing expression of the HSD17B13 gene. Methods of using such RNAi constructs to treat or prevent liver disease, nonalcoholic fatty liver disease (NAFLD) are also described.

### FIELD OF THE INVENTION

The present invention relates to compositions and methods for modulating liver expression of 17β-Hydroxysteroid dehydrogenase type 13 (HSD17B13), In particular, the present invention relates to nucleic acid-based therapeutics for reducing HSD17B13 expression via RNA interference and methods of using such nucleic acid-based therapeutics to treat or prevent liver disease, such as nonalcoholic fatty liver disease (NAFLD).

### BACKGROUND OF THE INVENTION

Comprising a spectrum of hepatic pathologies, nonalcoholic fatty liver disease (NAFLD) is the most common chronic liver disease in the world, the prevalence of which doubled in the last 20 years and now is estimated to affect approximately 20% of the world population (Sattar et al. (2014) BMJ 349:g4596; Loomba and Sanyal (2013) Nature Reviews Gastroenterology & hepatology 10(11):686-690; Kim and Kim (2017) Clin Gastroenterol Hepatol 15(4):474-485; Petta et al. (2016) Dig Liver Dis 48(3):333-342). NAFLD begins with the accumulation of triglyceride in the liver and is defined by the presence of cytoplasmic lipid droplets in more than 5% of hepatocytes in an individual 1) without a history of significant alcohol consumption and 2) in which the diagnosis of other types of liver disease have been excluded (Zhu et al (2016) World J Gastroenterol 22(36):8226-33; Rinella (2015) JAMA 313(22):2263-73; Yki-Jarvinen (2016) Diabetologia 59(6):1104-11). In some individuals the accumulation of ectopic fat in the liver, called steatosis, triggers inflammation and hepatocellular injury leading to a more advanced stage of disease called, nonalcoholic steatohepatitis (NASH) (Rinella, supra). As of 2015, 75-100 million Americans are predicted to have NAFLD; NASH accounted for approximately 10-30% of NAFLD diagnoses (Rinella, supra; Younossi et al (2016) Hepatology 64(5):1577-1586).

17β-Hydroxysteroid dehydrogenase type 13 (HSD17B13), also known as 17β-HSD type 13, is a member of the 17β-Hydroxysteroid dehydrogenase (HSD17B) family that comprise a family of enzymes catalyzing the conversion between 17-keto- and 17-hydroxysteroids (Su et al. (2019) Molecular and Cellular Endocrinology; 489:119-125). HSD17B13, originally named SCDR9, was first cloned from a human liver cDNA library in 2007 (Liu et al. (2007) Acta Biochim 54:213-218). In 2008, Horiguchi identified HSD17B13 as a new LD-associated protein with expression mainly restricted to the liver (Horiguchi et al. (2008) Biochem Biophys Res Commun 370:235-238). Hepatic overexpression of HSD17B13 promotes lipid accumulation in the liver. HSD17B13 expression is markedly unregulated in patients and mice with non-alcoholic fatty liver disease (NAFLD) (Su et al. (2014) PNAS 111:11437-11442). Accordingly, novel therapeutics targeting HSD17B13 represents a novel approach to reducing HSD17B13 levels and treating hepatologic diseases, such as nonalcoholic fatty liver disease.

### SUMMARY OF THE INVENTION

The present invention is based, in part, on the design and generation of RNAi constructs that target the HSD17B13 mRNA and reduce expression of HSD17B13 in liver cells. The sequence specific inhibition of HSD17B13 expression is useful for treating or preventing conditions associated with HSD17B13 expression, such as liver-related diseases, such as, for example, simple fatty liver (steatosis), nonalcoholic steatohepatitis (NASH), cirrhosis (irreversible, advanced scarring of the liver), or HSD17B13 related obesity. Accordingly, in one embodiment, the present invention provides an RNAi construct comprising a sense strand and an antisense strand, wherein the antisense strand comprises a region having a sequence that is complementary to a HSD17B13 mRNA sequence. In certain embodiments, the antisense strand comprises a region having at least 15 contiguous nucleotides from an antisense sequence listed in Table 1 or Table 2.

In some embodiments, the sense strand of the RNAi constructs described herein comprises a sequence that is sufficiently complementary to the sequence of the antisense strand to form a duplex region of about 15 to about 30 base pairs in length. In these and other embodiments, the sense and antisense strands each are about 15 to about 30 nucleotides in length. In some embodiments, the RNAi constructs comprise at least one blunt end. In other embodiments, the RNAi constructs comprise at least one nucleotide overhang. Such nucleotide overhangs may comprise at least 1 to 6 unpaired nucleotides and can be located at the 3′ end of the sense strand, the 3′ end of the antisense strand, or the 3′ end of both the sense and antisense strand. In certain embodiments, the RNAi constructs comprise an overhang of two unpaired nucleotides at the 3′ end of the sense strand and the 3′ end of the antisense strand. In other embodiments, the RNAi constructs comprise an overhang of two unpaired nucleotides at the 3′ end of the antisense strand and a blunt end of the 3′ end of the sense strand/5′ end of the antisense strand.

The RNAi constructs of the invention may comprise one or more modified nucleotides, including nucleotides having modifications to the ribose ring, nucleobase, or phosphodiester backbone. In some embodiments, the RNAi constructs comprise one or more 2′-modified nucleotides. Such 2′-modified nucleotides can include 2′-fluoro modified nucleotides, 2′-O-methyl modified nucleotides, 2′-deoxy modified nucleotides, 2′-O-methoxyethyl modified nucleotides, 2′-O-allyl modified nucleotides, bicyclic nucleic acids (BNA), glycol nucleic acids (GNAs), inverted bases (e.g. inverted adenosine) or combinations thereof. In one particular embodiment, the RNAi constructs comprise one or more 2′-fluoro modified nucleotides, 2′-O-methyl modified nucleotides, or combinations thereof. In some embodiments, all of the nucleotides in the sense and antisense strand of the RNAi construct are modified nucleotides.

In some embodiments, the RNAi constructs comprise at least one backbone modification, such as a modified internucleotide or internucleoside linkage. In certain embodiments, the RNAi constructs described herein comprise at least one phosphorothioate internucleotide linkage. In particular embodiments, the phosphorothioate internucleotide linkages may be positioned at the 3′ or 5′ ends of the sense and/or antisense strands.

In some embodiments, the antisense strand and/or the sense strand of the RNAi constructs of the invention may comprise or consist of a sequence from the antisense and sense sequences listed in Tables 1 or 2. In certain embodiments, the RNAi construct may be any one of the duplex compounds listed in any one of Tables 1 to 2.

### DETAILED DESCRIPTION OF THE INVENTION

The present invention is directed to compositions and methods for regulating the expression of the 17β-Hydroxysteroid dehydrogenase type 13 (HSD17B13) gene. In some embodiments, the gene may be within a cell or subject, such as a mammal (e.g. a human). In some embodiments, compositions of the invention comprise RNAi constructs that target a HSD17B13 mRNA and reduce HSD17B13 expression in a cell or mammal. Such RNAi constructs are useful for treating or preventing various forms of liver-related diseases, such as, for example, simple fatty liver (steatosis), nonalcoholic steatohepatitis (NASH), cirrhosis (irreversible, advanced scarring of the liver), or HSD17B13 related obesity.

RNA interference (RNAi) is the process of introducing exogeneous RNA into a cell leading to specific degradation of the mRNA encoding the targeted protein with a resultant decrease in protein expression. Advances in both the RNAi technology and hepatic delivery and growing positive outcomes with other RNAi-based therapies, suggest RNAi as a compelling means to therapeutically treat NAFLD by directly targeting HSD17B13.

As used herein, the term “RNAi construct” refers to an agent comprising a RNA molecule that is capable of downregulating expression of a target gene (e.g. HSD17B13) via a RNA interference mechanism when introduced into a cell. RNA interference is the process by which a nucleic acid molecule induces the cleavage and degradation of a target RNA molecule (e.g. messenger RNA or mRNA molecule) in a sequence-specific manner, e.g. through a RNA induced silencing complex (RISC) pathway. In some embodiments, the RNAi construct comprises a double-stranded RNA molecule comprising two antiparallel strands of contiguous nucleotides that are sufficiently complementary to each other to hybridize to form a duplex region. “Hybridize” or “hybridization” refers to the pairing of complementary polynucleotides, typically via hydrogen bonding (e.g. Watson-Crick, Hoogsteen or reversed Hoogsteen hydrogen bonding) between complementary bases in the two polynucleotides. The strand comprising a region having a sequence that is substantially complementary to a target sequence (e.g. target mRNA) is referred to as the “antisense strand.” The “sense strand” refers to the strand that includes a region that is substantially complementary to a region of the antisense strand. In some embodiments, the sense strand may comprise a region that has a sequence that is substantially identical to the target sequence.

In some embodiments, the invention is an RNAi construct directed to HSD17B13. In some embodiments, the invention includes an RNAi construct that contains any of the sequences found in Table 1 or 2.

A double-stranded RNA molecule may include chemical modifications to ribonucleotides, including modifications to the ribose sugar, base, or backbone components of the ribonucleotides, such as those described herein or known in the art. Any such modifications, as used in a double-stranded RNA molecule (e.g. siRNA, shRNA, or the like), are encompassed by the term “double-stranded RNA” for the purposes of this disclosure.

As used herein, a first sequence is “complementary” to a second sequence if a polynucleotide comprising the first sequence can hybridize to a polynucleotide comprising the second sequence to form a duplex region under certain conditions, such as physiological conditions. Other such conditions can include moderate or stringent hybridization conditions, which are known to those of skill in the art. A first sequence is considered to be fully complementary (100% complementary) to a second sequence if a polynucleotide comprising the first sequence base pairs with a polynucleotide comprising the second sequence over the entire length of one or both nucleotide sequences without any mismatches. A sequence is “substantially complementary” to a target sequence if the sequence is at least about 80%, 85%, 90%, 95%, 96%, 97%, 98%, 99% or 100% complementary to a target sequence. Percent complementarity can be calculated by dividing the number of bases in a first sequence that are complementary to bases at corresponding positions in a second or target sequence by the total length of the first sequence. A sequence may also be said to be substantially complementary to another sequence if there are no more than 5, 4, 3, 2, or 1 mismatches over a 30 base pair duplex region when the two sequences are hybridized. Generally, if any nucleotide overhangs, as defined herein, are present, the sequence of such overhangs is not considered in determining the degree of complementarity between two sequences. By way of example, a sense strand of 21 nucleotides in length and an antisense strand of 21 nucleotides in length that hybridize to form a 19 base pair duplex region with a 2 nucleotide overhang at the 3′ end of each strand would be considered to be fully complementary as the term is used herein.

In some embodiments, a region of the antisense strand comprises a sequence that is fully complementary to a region of the target RNA sequence (e.g. HSD17B13 mRNA). In such embodiments, the sense strand may comprise a sequence that is fully complementary to the sequence of the antisense strand. In other such embodiments, the sense strand may comprise a sequence that is substantially complementary to the sequence of the antisense strand, e.g. having 1, 2, 3, 4, or 5 mismatches in the duplex region formed by the sense and antisense strands. In certain embodiments, it is preferred that any mismatches occur within the terminal regions (e.g. within 6, 5, 4, 3, 2, or 1 nucleotides of the 5′ and/or 3′ ends of the strands). In one embodiment, any mismatches in the duplex region formed from the sense and antisense strands occur within 6, 5, 4, 3, 2, or 1 nucleotides of the 5′ end of the antisense strand.

In certain embodiments, the sense strand and antisense strand of the double-stranded RNA may be two separate molecules that hybridize to form a duplex region, but are otherwise unconnected. Such double-stranded RNA molecules formed from two separate strands are referred to as “small interfering RNAs” or “short interfering RNAs” (siRNAs). Thus, in some embodiments, the RNAi constructs of the invention comprise a siRNA.

Where the two substantially complementary strands of a dsRNA are comprised by separate RNA molecules, those molecules need not, but can be covalently connected. Where the two strands are connected covalently by means other than an uninterrupted chain of nucleotides between the 3 ‘-end of one strand and the 5’-end of the respective other strand forming the duplex structure, the connecting structure is referred to as a “linker.” The RNA strands may have the same or a different number of nucleotides. The maximum number of base pairs in the duplex is the number of nucleotides in the shortest strand of the dsRNA minus any overhangs that are present in the duplex. In addition to the duplex structure, an RNAi construct may comprise one or more nucleotide overhangs.

In other embodiments, the sense strand and the antisense strand that hybridize to form a duplex region may be part of a single RNA molecule, i.e. the sense and antisense strands are part of a self-complementary region of a single RNA molecule. In such cases, a single RNA molecule comprises a duplex region (also referred to as a stem region) and a loop region. The 3′ end of the sense strand is connected to the 5′ end of the antisense strand by a contiguous sequence of unpaired nucleotides, which will form the loop region. The loop region is typically of a sufficient length to allow the RNA molecule to fold back on itself such that the antisense strand can base pair with the sense strand to form the duplex or stem region. The loop region can comprise from about 3 to about 25, from about 5 to about 15, or from about 8 to about 12 unpaired nucleotides. Such RNA molecules with at least partially self-complementary regions are referred to as “short hairpin RNAs” (shRNAs). In some embodiments, the loop region can comprise at least 1, 2, 3, 4, 5, 10, 20, or 25 unpaired nucleotides. In some embodiments, the loop region can have 10, 9, 8, 7, 6, 5, 4, 3, 2, or fewer unpaired nucleotides. In certain embodiments, the RNAi constructs of the invention comprise a shRNA. The length of a single, at least partially self-complementary RNA molecule can be from about 35 nucleotides to about 100 nucleotides, from about 45 nucleotides to about 85 nucleotides, or from about 50 to about 60 nucleotides and comprise a duplex region and loop region each having the lengths recited herein.

In some embodiments, the RNAi constructs of the invention comprise a sense strand and an antisense strand, wherein the antisense strand comprises a region having a sequence that is substantially or fully complementary to a HSD17B13 messenger RNA (mRNA) sequence. As used herein, a “HSD17B13 mRNA sequence” refers to any messenger RNA sequence, including splice variants, encoding a HSD17B13 protein, including HSD17B13 protein variants or isoforms from any species (e.g. mouse, rat, non-human primate, human).

An HSD17B13 mRNA sequence also includes the transcript sequence expressed as its complementary DNA (cDNA) sequence. A cDNA sequence refers to the sequence of an mRNA transcript expressed as DNA bases (e.g. guanine, adenine, thymine, and cytosine) rather than RNA bases (e.g. guanine, adenine, uracil, and cytosine). Thus, the antisense strand of the RNAi constructs of the invention may comprise a region having a sequence that is substantially or fully complementary to a target HSD17B13 mRNA sequence or HSD17B13 cDNA sequence. A HSD17B13 mRNA or cDNA sequence can include, but is not limited to, any HSD17B13 mRNA or cDNA sequence such as can be derived from the NCBI Reference sequence NM_178135.4 or NM_001136230.2.

A region of the antisense strand can be substantially complementary or fully complementary to at least 15 consecutive nucleotides of the HSD17B13 mRNA sequence. In some embodiments, the target region of the HSD17B13 mRNA sequence to which the antisense strand comprises a region of complementarity can range from about 15 to about 30 consecutive nucleotides, from about 16 to about 28 consecutive nucleotides, from about 18 to about 26 consecutive nucleotides, from about 17 to about 24 consecutive nucleotides, from about 19 to about 25 consecutive nucleotides, from about 19 to about 23 consecutive nucleotides, or from about 19 to about 21 consecutive nucleotides. In certain embodiments, the region of the antisense strand comprising a sequence that is substantially or fully complementary to a HSD17B13 mRNA sequence may, in some embodiments, comprise at least 15 contiguous nucleotides from an antisense sequence listed in Table 1 or Table 2. In other embodiments, the antisense sequence comprises at least 16, at least 17, at least 18, or at least 19 contiguous nucleotides from an antisense sequence listed in Table 1 or Table 2. In some embodiments, the sense and/or antisense sequence comprises at least 15 nucleotides from a sequence listed in Table 1 or 2 with no more than 1, 2, or 3 nucleotide mismatches.

The sense strand of the RNAi construct typically comprises a sequence that is sufficiently complementary to the sequence of the antisense strand such that the two strands hybridize under physiological conditions to form a duplex region. A “duplex region” refers to the region in two complementary or substantially complementary polynucleotides that form base pairs with one another, either by Watson-Crick base pairing or other hydrogen bonding interaction, to create a duplex between the two polynucleotides. The duplex region of the RNAi construct should be of sufficient length to allow the RNAi construct to enter the RNA interference pathway, e.g. by engaging the Dicer enzyme and/or the RISC complex. For instance, in some embodiments, the duplex region is about 15 to about 30 base pairs in length. Other lengths for the duplex region within this range are also suitable, such as about 15 to about 28 base pairs, about 15 to about 26 base pairs, about 15 to about 24 base pairs, about 15 to about 22 base pairs, about 17 to about 28 base pairs, about 17 to about 26 base pairs, about 17 to about 24 base pairs, about 17 to about 23 base pairs, about 17 to about 21 base pairs, about 19 to about 25 base pairs, about 19 to about 23 base pairs, or about 19 to about 21 base pairs. In one embodiment, the duplex region is about 17 to about 24 base pairs in length. In another embodiment, the duplex region is about 19 to about 21 base pairs in length.

In some embodiments, an RNAi construct of the invention contains a duplex region of about 24 to about 30 nucleotides that interacts with a target RNA sequence, e.g., an HSD17B13 target mRNA sequence, to direct the cleavage of the target RNA. Without wishing to be bound by theory, long double stranded RNA introduced into cells can be broken down into siRNA by a Type III endonuclease known as Dicer (Sharp et al. (2001) Genes Dev. 15:485). Dicer, a ribonuclease-III-like enzyme, processes the dsRNA into 19-23 base pair short interfering RNAs with characteristic two base 3′ overhangs (Bernstein, et al., (2001) Nature 409:363). The siRNAs are then incorporated into an RNA-induced silencing complex (RISC) where one or more helicases unwind the siRNA duplex, enabling the complementary antisense strand to guide target recognition (Nykanen, et al., (2001) Cell 107:309). Upon binding to the appropriate target mRNA, one or more endonucleases within the RISC cleave the target to induce silencing (Elbashir, et al., (2001) Genes Dev. 15: 188).

For embodiments in which the sense strand and antisense strand are two separate molecules (e.g. RNAi construct comprises a siRNA), the sense strand and antisense strand need not be the same length as the length of the duplex region. For instance, one or both strands may be longer than the duplex region and have one or more unpaired nucleotides or mismatches flanking the duplex region. Thus, in some embodiments, the RNAi construct comprises at least one nucleotide overhang. As used herein, a “nucleotide overhang” refers to the unpaired nucleotide or nucleotides that extend beyond the duplex region at the terminal ends of the strands. Nucleotide overhangs are typically created when the 3′ end of one strand extends beyond the 5′ end of the other strand or when the 5′ end of one strand extends beyond the 3′ end of the other strand. The length of a nucleotide overhang is generally between 1 and 6 nucleotides, 1 and 5 nucleotides, 1 and 4 nucleotides, 1 and 3 nucleotides, 2 and 6 nucleotides, 2 and 5 nucleotides, or 2 and 4 nucleotides. In some embodiments, the nucleotide overhang comprises 1, 2, 3, 4, 5, or 6 nucleotides. In one particular embodiment, the nucleotide overhang comprises 1 to 4 nucleotides. In certain embodiments, the nucleotide overhang comprises 2 nucleotides. The nucleotides in the overhang can be ribonucleotides, deoxyribonucleotides, or modified nucleotides as described herein. In some embodiments, the overhang comprises a 5′-uridine-uridine-3′ (5′-UU-3′) dinucleotide. In such embodiments, the UU dinucleotide may comprise ribonucleotides or modified nucleotides, e.g. 2′-modified nucleotides. In other embodiments, the overhang comprises a 5′-deoxythymidine-deoxythymidine-3′ (5′-dTdT-3′) dinucleotide.

The nucleotide overhang can be at the 5′ end or 3′ end of one or both strands. For example, in one embodiment, the RNAi construct comprises a nucleotide overhang at the 5′ end and the 3′ end of the antisense strand. In another embodiment, the RNAi construct comprises a nucleotide overhang at the 5′ end and the 3′ end of the sense strand. In some embodiments, the RNAi construct comprises a nucleotide overhang at the 5′ end of the sense strand and the 5′ end of the antisense strand. In other embodiments, the RNAi construct comprises a nucleotide overhang at the 3′ end of the sense strand and the 3′ end of the antisense strand.

The RNAi constructs may comprise a single nucleotide overhang at one end of the double-stranded RNA molecule and a blunt end at the other. A “blunt end” means that the sense strand and antisense strand are fully base-paired at the end of the molecule and there are no unpaired nucleotides that extend beyond the duplex region. In some embodiments, the RNAi construct comprises a nucleotide overhang at the 3′ end of the sense strand and a blunt end at the 5′ end of the sense strand and 3′ end of the antisense strand. In other embodiments, the RNAi construct comprises a nucleotide overhang at the 3′ end of the antisense strand and a blunt end at the 5′ end of the antisense strand and the 3′ end of the sense strand. In certain embodiments, the RNAi construct comprises a blunt end at both ends of the double-stranded RNA molecule. In such embodiments, the sense strand and antisense strand have the same length and the duplex region is the same length as the sense and antisense strands (i.e. the molecule is double-stranded over its entire length).

The sense strand and antisense strand can each independently be about 15 to about 30 nucleotides in length, about 18 to about 28 nucleotides in length, about 19 to about 27 nucleotides in length, about 19 to about 25 nucleotides in length, about 19 to about 23 nucleotides in length, about 21 to about 25 nucleotides in length, or about 21 to about 23 nucleotides in length. In certain embodiments, the sense strand and antisense strand are each about 18, about 19, about 20, about 21, about 22, about 23, about 24, or about 25 nucleotides in length. In some embodiments, the sense strand and antisense strand have the same length but form a duplex region that is shorter than the strands such that the RNAi construct has two nucleotide overhangs. For instance, in one embodiment, the RNAi construct comprises (i) a sense strand and an antisense strand that are each 21 nucleotides in length, (ii) a duplex region that is 19 base pairs in length, and (iii) nucleotide overhangs of 2 unpaired nucleotides at both the 3′ end of the sense strand and the 3′ end of the antisense strand. In another embodiment, the RNAi construct comprises (i) a sense strand and an antisense strand that are each 23 nucleotides in length, (ii) a duplex region that is 21 base pairs in length, and (iii) nucleotide overhangs of 2 unpaired nucleotides at both the 3′ end of the sense strand and the 3′ end of the antisense strand. In other embodiments, the sense strand and antisense strand have the same length and form a duplex region over their entire length such that there are no nucleotide overhangs on either end of the double-stranded molecule. In one such embodiment, the RNAi construct is blunt ended and comprises (i) a sense strand and an antisense strand, each of which is 21 nucleotides in length, and (ii) a duplex region that is 21 base pairs in length. In another such embodiment, the RNAi construct is blunt ended and comprises (i) a sense strand and an antisense strand, each of which is 23 nucleotides in length, and (ii) a duplex region that is 23 base pairs in length.

In other embodiments, the sense strand or the antisense strand is longer than the other strand and the two strands form a duplex region having a length equal to that of the shorter strand such that the RNAi construct comprises at least one nucleotide overhang. For example, in one embodiment, the RNAi construct comprises (i) a sense strand that is 19 nucleotides in length, (ii) an antisense strand that is 21 nucleotides in length, (iii) a duplex region of 19 base pairs in length, and (iv) a single nucleotide overhang of 2 unpaired nucleotides at the 3′ end of the antisense strand. In another embodiment, the RNAi construct comprises (i) a sense strand that is 21 nucleotides in length, (ii) an antisense strand that is 23 nucleotides in length, (iii) a duplex region of 21 base pairs in length, and (iv) a single nucleotide overhang of 2 unpaired nucleotides at the 3′ end of the antisense strand.

The antisense strand of the RNAi constructs of the invention can comprise the sequence of any one of the antisense sequences listed in Table 1 or Table 2 or the sequence of nucleotides 1-19 of any of these antisense sequences. Each of the antisense sequences listed in Tables 1 and 6 comprises a sequence of 19 consecutive nucleotides (first 19 nucleotides counting from the 5′ end) that is complementary to a HSD17B13 mRNA sequence plus a two nucleotide overhang sequence. Thus, in some embodiments, the antisense strand comprises a sequence of nucleotides 1-19 of any one of SEQ ID NOs: 1-646 or 648-1292.

### Modified Nucleotides

The RNAi constructs of the invention may comprise one or more modified nucleotides. A “modified nucleotide” refers to a nucleotide that has one or more chemical modifications to the nucleoside, nucleobase, pentose ring, or phosphate group. As used herein, modified nucleotides do not encompass ribonucleotides containing adenosine monophosphate, guanosine monophosphate, uridine monophosphate, and cytidine monophosphate, and deoxyribonucleotides containing deoxyadenosine monophosphate, deoxyguanosine monophosphate, deoxythymidine monophosphate, and deoxycytidine monophosphate. However, the RNAi constructs may comprise combinations of modified nucleotides, ribonucleotides, and deoxyribonucleotides. Incorporation of modified nucleotides into one or both strands of double-stranded RNA molecules can improve the in vivo stability of the RNA molecules, e.g., by reducing the molecules' susceptibility to nucleases and other degradation processes. The potency of RNAi constructs for reducing expression of the target gene can also be enhanced by incorporation of modified nucleotides.

In certain embodiments, the modified nucleotides have a modification of the ribose sugar. These sugar modifications can include modifications at the 2′ and/or 5′ position of the pentose ring as well as bicyclic sugar modifications. A 2′-modified nucleotide refers to a nucleotide having a pentose ring with a substituent at the 2′ position other than H or OH. Such 2′ modifications include, but are not limited to, 2′-O-alkyl (e.g. O—C1-C10 or O—C1-C10 substituted alkyl), 2′-O-allyl (O—CH2CH═CH2), 2′-C-allyl, 2′-fluoro, 2′-O-methyl (OCH3), 2′-O-methoxyethyl (O—(CH2)2OCH3), 2′-OCF3, 2′-O(CH2)2SCH3, 2′-O-aminoalkyl, 2′-amino (e.g. NH2), 2′-O-ethylamine, and 2′-azido. Modifications at the 5′ position of the pentose ring include, but are not limited to, 5′-methyl (R or S); 5′-vinyl, and 5′-methoxy.

A “bicyclic sugar modification” refers to a modification of the pentose ring where a bridge connects two atoms of the ring to form a second ring resulting in a bicyclic sugar structure. In some embodiments the bicyclic sugar modification comprises a bridge between the 4′ and 2′ carbons of the pentose ring. Nucleotides comprising a sugar moiety with a bicyclic sugar modification are referred to herein as bicyclic nucleic acids or BNAs. Exemplary bicyclic sugar modifications include, but are not limited to, α-L-Methyleneoxy (4′-CH2-O-2′) bicyclicnucleic acid (BNA); P-D-Methyleneoxy (4′-CH2-O-2′) BNA (also referred to as a locked nucleic acid or LNA); Ethyleneoxy (4′-(CH2)2-O-2′) BNA; Aminooxy (4′-CH2-O—N(R)-2′) BNA; Oxyamino (4′-CH2-N(R)—O-2′) BNA; Methyl(methyleneoxy) (4′-CH(CH3)-O-2′) BNA (also referred to as constrained ethyl or cEt); methylene-thio (4′-CH2-S-2′) BNA; methylene-amino (4′-CH2-N(R)-2′) BNA; methyl carbocyclic (4′-CH2-CH(CH3)-2′) BNA; propylene carbocyclic (4′-(CH2)3-2′) BNA; and Methoxy(ethyleneoxy) (4′-CH(CH2OMe)-O-2′) BNA (also referred to as constrained MOE or cMOE). These and other sugar-modified nucleotides that can be incorporated into the RNAi constructs of the invention are described in U.S. Pat. No. 9,181,551, U.S. Patent Publication No. 2016/0122761, and Deleavey and Damha, Chemistry and Biology, Vol. 19: 937-954, 2012, all of which are hereby incorporated by reference in their entireties.

In some embodiments, the RNAi constructs comprise one or more 2′-fluoro modified nucleotides, 2′-O-methyl modified nucleotides, 2′-O-methoxyethyl modified nucleotides, 2′-O-allyl modified nucleotides, bicyclic nucleic acids (BNAs), glycol nucleic acids, or combinations thereof. In certain embodiments, the RNAi constructs comprise one or more 2′-fluoro modified nucleotides, 2′-O-methyl modified nucleotides, 2′-O-methoxyethyl modified nucleotides, or combinations thereof. In one particular embodiment, the RNAi constructs comprise one or more 2′-fluoro modified nucleotides, 2′-O-methyl modified nucleotides or combinations thereof.

Both the sense and antisense strands of the RNAi constructs can comprise one or multiple modified nucleotides. For instance, in some embodiments, the sense strand comprises 1, 2, 3, 4, 5, 6, 7, 8, 9, 10 or more modified nucleotides. In certain embodiments, all nucleotides in the sense strand are modified nucleotides. In some embodiments, the antisense strand comprises 1, 2, 3, 4, 5, 6, 7, 8, 9, 10 or more modified nucleotides. In other embodiments, all nucleotides in the antisense strand are modified nucleotides. In certain other embodiments, all nucleotides in the sense strand and all nucleotides in the antisense strand are modified nucleotides. In these and other embodiments, the modified nucleotides can be 2′-fluoro modified nucleotides, 2′-O-methyl modified nucleotides, or combinations thereof.

In some embodiments, all pyrimidine nucleotides preceding an adenosine nucleotide in the sense strand, antisense strand, or both strands are modified nucleotides. For example, where the sequence 5′-CA-3′ or 5′-UA-3′ appears in either strand, the cytidine and uridine nucleotides are modified nucleotides, preferably 2′-O-methyl modified nucleotides. In certain embodiments, all pyrimidine nucleotides in the sense strand are modified nucleotides (e.g. 2′-O-methyl modified nucleotides), and the 5′ nucleotide in all occurrences of the sequence 5′-CA-3′ or 5′-UA-3′ in the antisense strand are modified nucleotides (e.g. 2′-O-methyl modified nucleotides). In other embodiments, all nucleotides in the duplex region are modified nucleotides. In such embodiments, the modified nucleotides are preferably 2′-O-methyl modified nucleotides, 2′-fluoro modified nucleotides or combinations thereof.

In embodiments in which the RNAi construct comprises a nucleotide overhang, the nucleotides in the overhang can be ribonucleotides, deoxyribonucleotides, or modified nucleotides. In one embodiment, the nucleotides in the overhang are deoxyribonucleotides, e.g., deoxythymidine. In another embodiment, the nucleotides in the overhang are modified nucleotides. For instance, in some embodiments, the nucleotides in the overhang are 2′-O-methyl modified nucleotides, 2′-fluoro modified nucleotides, 2′-methoxyethyl modified nucleotides, or combinations thereof.

The RNAi constructs of the invention may also comprise one or more modified internucleotide linkages. As used herein, the term “modified internucleotide linkage” refers to an internucleotide linkage other than the natural 3′ to 5′ phosphodiester linkage. In some embodiments, the modified internucleotide linkage is a phosphorous-containing internucleotide linkage, such as a phosphotriester, aminoalkyl phosphotriester, an alkylphosphonate (e.g. methylphosphonate, 3′-alkylene phosphonate), a phosphinate, a phosphoramidate (e.g. 3′-aminophosphoramidate and aminoalkylphosphoramidate), a phosphorothioate (P═S), a chiralphosphorothioate, a phosphorodithioate, a thionophosphoramidate, a thionoalkylphosphonate, athionoalkylphosphotriester, and a boranophosphate. In one embodiment, a modified internucleotide linkage is a 2′ to 5′ phosphodiester linkage. In other embodiments, the modified internucleotide linkage is a non-phosphorous-containing internucleotide linkage and thus can be referred to as a modified internucleoside linkage. Such non-phosphorous-containing linkages include, but are not limited to, morpholino linkages (formed in part from the sugar portion of a nucleoside); siloxane linkages (—O—Si(H)2-O—); sulfide, sulfoxide and sulfone linkages; formacetyl and thioformacetyl linkages; alkene containing backbones; sulfamate backbones; methylenemethylimino (—CH2-N(CH3)-O—CH2-) and methylenehydrazino linkages; sulfonate and sulfonamide linkages; amide linkages; and others having mixed N, O, S and CH2 component parts. In one embodiment, the modified internucleoside linkage is a peptide-based linkage (e.g. aminoethylglycine) to create a peptide nucleic acid or PNA, such as those described in U.S. Pat. Nos. 5,539,082; 5,714,331; and 5,719,262. Other suitable modified internucleotide and internucleoside linkages that may be employed in the RNAi constructs of the invention are described in U.S. Pat. Nos. 6,693,187, 9,181,551, U.S. Patent Publication No. 2016/0122761, and Deleavey and Damha, Chemistry and Biology, Vol. 19: 937-954, 2012, all of which are hereby incorporated by reference in their entireties.

In certain embodiments, the RNAi constructs comprise one or more phosphorothioate internucleotide linkages. The phosphorothioate internucleotide linkages may be present in the sense strand, antisense strand, or both strands of the RNAi constructs. For instance, in some embodiments, the sense strand comprises 1, 2, 3, 4, 5, 6, 7, 8, or more phosphorothioate internucleotide linkages. In other embodiments, the antisense strand comprises 1, 2, 3, 4, 5, 6, 7, 8, or more phosphorothioate internucleotide linkages. In still other embodiments, both strands comprise 1, 2, 3, 4, 5, 6, 7, 8, or more phosphorothioate internucleotide linkages. The RNAi constructs can comprise one or more phosphorothioate internucleotide linkages at the 3′-end, the 5′-end, or both the 3′- and 5′-ends of the sense strand, the antisense strand, or both strands. For instance, in certain embodiments, the RNAi construct comprises about 1 to about 6 or more (e.g., about 1, 2, 3, 4, 5, 6 or more) consecutive phosphorothioate internucleotide linkages at the 3′-end of the sense strand, the antisense strand, or both strands. In other embodiments, the RNAi construct comprises about 1 to about 6 or more (e.g., about 1, 2, 3, 4, 5, 6 or more) consecutive phosphorothioate internucleotide linkages at the 5′-end of the sense strand, the antisense strand, or both strands. In one embodiment, the RNAi construct comprises a single phosphorothioate internucleotide linkage at the 3′ end of the sense strand and a single phosphorothioate internucleotide linkage at the 3′ end of the antisense strand. In another embodiment, the RNAi construct comprises two consecutive phosphorothioate internucleotide linkages at the 3′ end of the antisense strand (i.e. a phosphorothioate internucleotide linkage at the first and second internucleotide linkages at the 3′ end of the antisense strand). In another embodiment, the RNAi construct comprises two consecutive phosphorothioate internucleotide linkages at both the 3′ and 5′ ends of the antisense strand. In yet another embodiment, the RNAi construct comprises two consecutive phosphorothioate internucleotide linkages at both the 3′ and 5′ ends of the antisense strand and two consecutive phosphorothioate internucleotide linkages at the 5′ end of the sense strand. In still another embodiment, the RNAi construct comprises two consecutive phosphorothioate internucleotide linkages at both the 3′ and 5′ ends of the antisense strand and two consecutive phosphorothioate internucleotide linkages at both the 3′ and 5′ ends of the sense strand (i.e. a phosphorothioate internucleotide linkage at the first and second internucleotide linkages at both the 5′ and 3′ ends of the antisense strand and a phosphorothioate internucleotide linkage at the first and second internucleotide linkages at both the 5′ and 3′ ends of the sense strand). In any of the embodiments in which one or both strands comprises one or more phosphorothioate internucleotide linkages, the remaining internucleotide linkages within the strands can be the natural 3′ to 5′ phosphodiester linkages. For instance, in some embodiments, each internucleotide linkage of the sense and antisense strands is selected from phosphodiester and phosphorothioate, wherein at least one internucleotide linkage is a phosphorothioate.

In embodiments in which the RNAi construct comprises a nucleotide overhang, two or more of the unpaired nucleotides in the overhang can be connected by a phosphorothioate internucleotide linkage. In certain embodiments, all the unpaired nucleotides in a nucleotide overhang at the 3′ end of the antisense strand and/or the sense strand are connected by phosphorothioate internucleotide linkages. In other embodiments, all the unpaired nucleotides in a nucleotide overhang at the 5′ end of the antisense strand and/or the sense strand are connected by phosphorothioate internucleotide linkages. In still other embodiments, all the unpaired nucleotides in any nucleotide overhang are connected by phosphorothioate internucleotide linkages.

In certain embodiments, the modified nucleotides incorporated into one or both of the strands of the RNAi constructs of the invention have a modification of the nucleobase (also referred to herein as “base”). A “modified nucleobase” or “modified base” refers to a base other than the naturally occurring purine bases adenine (A) and guanine (G) and pyrimidine bases thymine (T), cytosine (C), and uracil (U). Modified nucleobases can be synthetic or naturally occurring modifications and include, but are not limited to, universal bases, 5-methylcytosine (5-me-C), 5-hydroxymethyl cytosine, xanthine (X), hypoxanthine (I), 2-aminoadenine, 6-methyladenine, 6-methylguanine, and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine, and abasic residues (apurinic/apyrimidinic residues which lack the purine or pyrimidine base, lacking a nucleobase at position 1 of the ribose sugar), and inverted nucleotides (nucleotides having 3′-3′ linkage, and can be inverted nucleotides of any of the above, including inverted abasic nucleotides and inverted deoxynucleotides).

In some embodiments, the modified base is a universal base. A “universal base” refers to a base analog that indiscriminately forms base pairs with all of the natural bases in RNA and DNA without altering the double helical structure of the resulting duplex region. Universal bases are known to those of skill in the art and include, but are not limited to, inosine, C-phenyl, C-naphthyl and other aromatic derivatives, azole carboxamides, and nitroazole derivatives, such as 3-nitropyrrole, 4-nitroindole, 5-nitroindole, and 6-nitroindole.

Other suitable modified bases that can be incorporated into the RNAi constructs of the invention include those described in Herdewijn, Antisense Nucleic Acid Drug Dev., Vol. 10:297-310, 2000 and Peacock et al., J. Org. Chem., Vol. 76: 7295-7300, 2011, both of which are hereby incorporated by reference in their entireties. The skilled person is well aware that guanine, cytosine, adenine, thymine, and uracil may be replaced by other nucleobases, such as the modified nucleobases described above, without substantially altering the base pairing properties of a polynucleotide comprising a nucleotide bearing such replacement nucleobase.

In some embodiments of the RNAi constructs of the invention, the 5′ end of the sense strand, antisense strand, or both the antisense and sense strands comprises a phosphate moiety. As used herein, the term “phosphate moiety” refers to a terminal phosphate group that includes unmodified phosphates (—O—P═O)(OH)OH) as well as modified phosphates. Modified phosphates include phosphates in which one or more of the O and OH groups is replaced with H, O, S, N(R) or alkyl where R is H, an amino protecting group or unsubstituted or substituted alkyl. Exemplary phosphate moieties include, but are not limited to, 5′-monophosphate; 5′diphosphate; 5′-triphosphate; 5′-guanosine cap (7-methylated or non-methylated); 5′-adenosinecap or any other modified or unmodified nucleotide cap structure; 5′-monothiophosphate (phosphorothioate); 5′-monodithiophosphate (phosphorodithioate); 5′-alpha-thiotriphosphate; 5′-gamma-thiotriphosphate, 5′-phosphoramidates; 5′-vinylphosphates; 5′-alkylphosphonates (e.g., alkyl=methyl, ethyl, isopropyl, propyl, etc.); and 5′-alkyletherphosphonates (e.g., alkylether=methoxymethyl, ethoxymethyl, etc.).

The modified nucleotides that can be incorporated into the RNAi constructs of the invention may have more than one chemical modification described herein. For instance, the modified nucleotide may have a modification to the ribose sugar as well as a modification to the nucleobase. By way of example, a modified nucleotide may comprise a 2′ sugar modification (e.g. 2′-fluoro or 2′-methyl) and comprise a modified base (e.g. 5-methyl cytosine or pseudouracil). In other embodiments, the modified nucleotide may comprise a sugar modification in combination with a modification to the 5′ phosphate that would create a modified internucleotide or internucleotide linkage when the modified nucleotide was incorporated into a polynucleotide. For instance, in some embodiments, the modified nucleotide may comprise a sugar modification, such as a 2′-fluoro modification, a 2′-O-methyl modification, or a bicyclic sugar modification, as well as a 5′ phosphorothioate group. Accordingly, in some embodiments, one or both strands of the RNAi constructs of the invention comprise a combination of 2′ modified nucleotides or BNAs and phosphorothioate internucleotide linkages. In certain embodiments, both the sense and antisense strands of the RNAi constructs of the invention comprise a combination of 2′-fluoro modified nucleotides, 2′-O-methyl modified nucleotides, and phosphorothioate internucleotide linkages. Exemplary RNAi constructs comprising modified nucleotides and internucleotide linkages are shown in Table 2.

### Function of RNAi Constructs

Preferably, the RNAi constructs of the invention reduce or inhibit the expression of HSD17B13 in cells, particularly liver cells. Accordingly, in one embodiment, the present invention provides a method of reducing HSD17B13 expression in a cell by contacting the cell with any RNAi construct described herein. The cell may be in vitro or in vivo. HSD17B13 expression can be assessed by measuring the amount or level of HSD17B13 mRNA, HSD17B13 protein, or another biomarker linked to HSD17B13 expression. The reduction of HSD17B13 expression in cells or animals treated with an RNAi construct of the invention can be determined relative to the HSD17B13 expression in cells or animals not treated with the RNAi construct or treated with a control RNAi construct. For instance, in some embodiments, reduction of HSD17B13 expression is assessed by (a) measuring the amount or level of HSD17B13 mRNA in liver cells treated with a RNAi construct of the invention, (b) measuring the amount or level of HSD17B13 mRNA in liver cells treated with a control RNAi construct (e.g., RNAi construct directed to a RNA molecule not expressed in liver cells or a RNAi construct having a nonsense or scrambled sequence) or no construct, and (c) comparing the measured HSD17B13 mRNA levels from treated cells in (a) to the measured HSD17B13 mRNA levels from control cells in (b). The HSD17B13 mRNA levels in the treated cells and controls cells can be normalized to RNA levels for a control gene (e.g. 18S ribosomal RNA) prior to comparison. HSD17B13 mRNA levels can be measured by a variety of methods, including Northern blot analysis, nuclease protection assays, fluorescence in situ hybridization (FISH), reverse-transcriptase (RT)-PCR, real-time RT-PCR, quantitative PCR, and the like.

In other embodiments, reduction of HSD17B13 expression is assessed by (a) measuring the amount or level of HSD17B13 protein in liver cells treated with a RNAi construct of the invention, (b) measuring the amount or level of HSD17B13 protein in liver cells treated with a control RNAi construct (e.g. RNAi construct directed to a RNA molecule not expressed in liver cells or a RNAi construct having a nonsense or scrambled sequence) or no construct, and (c) comparing the measured HSD17B13 protein levels from treated cells in (a) to the measured HSD17B13 protein levels from control cells in (b). Methods of measuring HSD17B13 protein levels are known to those of skill in the art, and include Western Blots, immunoassays (e.g. ELISA), and flow cytometry. An exemplary droplet digital PCR method for assessing HSD17B13 expression is described in Example 2. Any method capable of measuring HSD17B13 mRNA or protein can be used to assess the efficacy of the RNAi constructs of the invention.

In some embodiments, the methods to assess HSD17B13 expression levels are performed in vitro in cells that natively express HSD17B13 (e.g. liver cells) or cells that have been engineered to express HSD17B13. In certain embodiments, the methods are performed in vitro in liver cells. Suitable liver cells include, but are not limited to, primary hepatocytes (e.g. human, non-human primate, or rodent hepatocytes), HepAD38 cells, HuH-6 cells, HuH-7 cells, HuH-5-2 cells, BNLCL2 cells, Hep3B cells, or HepG2 cells.

In other embodiments, the methods to assess HSD17B13 expression levels are performed in vivo. The RNAi constructs and any control RNAi constructs can be administered to an animal (e.g. rodent or non-human primate) and HSD17B13 mRNA or protein levels assessed in liver tissue harvested from the animal following treatment. Alternatively or additionally, a biomarker or functional phenotype associated with HSD17B13 expression can be assessed in the treated animals.

In certain embodiments, expression of HSD17B13 is reduced in liver cells by at least 10%, at least 15%, at least 20%, at least 25%, at least 30%, at least 35%, at least 40%, at least 45%, or at least 50% by an RNAi construct of the invention. In some embodiments, expression of HSD17B13 is reduced in liver cells by at least 60%, at least 65%, at least 70%, at least 75%, at least 80%, or at least 85% by an RNAi construct of the invention. In other embodiments, the expression of HSD17B13 is reduced in liver cells by about 90% or more, e.g., 91%, 92%, 93%, 94%, 95%, 96%, 97%, 98%, 99%, or more by an RNAi construct of the invention. The percent reduction of HSD17B13 expression can be measured by any of the methods described herein as well as others known in the art. For instance, in certain embodiments, the RNAi constructs of the invention inhibit at least 70% of HSD17B13 expression at 5 nM in primary hepatic cells (expresses wild type HSD17B13) in vitro. In related embodiments, the RNAi constructs of the invention inhibit at least 50%, at least 55%, at least 60%, at least 65%, at least 70%, or at least 75% of HSD17B13 expression at 5 nM in vitro. In other embodiments, the RNAi constructs of the invention inhibit at least 80%, at least 85%, at least 90%, at least 92%, at least 94%, at least 96%, or at least 98% of HSD17B13 expression at 5 nM in primary hepatocytes in vitro. Reduction of HSD17B13 can be measured using a variety of techniques including RNA FISH or droplet digital PCR, as described in Example 2.

In some embodiments, an IC50 value is calculated to assess the potency of an RNAi construct of the invention for inhibiting HSD17B13 expression in liver cells. An “IC50 value” is the dose/concentration required to achieve 50% inhibition of a biological or biochemical function or level. The IC50 value of any particular substance or antagonist can be determined by constructing a dose-response curve and examining the effect of different concentrations of the substance or antagonist on expression levels or functional activity in any assay. IC50 values can be calculated for a given antagonist or substance by determining the concentration needed to inhibit half of the maximum biological response or native expression levels. Thus, the IC50 value for any RNAi construct can be calculated by determining the concentration of the RNAi construct needed to inhibit half of the native HSD17B13 expression level in liver cells (e.g. HSD17B13 expression level in control liver cells) in any assay, such as the immunoassay or RNA FISH assay or droplet digital PCR assays, as described in the Examples. The RNAi constructs of the invention may inhibit HSD17B13 expression in liver cells (e.g. primary hepatocytes) with an IC50 of less than about 40 nM. For example, the RNAi constructs inhibit HSD17B13 expression in liver cells with an IC50 of about 0.001 nM to about 40 nM, about 0.001 nM to about 30 nM, about 0.001 nM to about 20 nM, about 0.001 nM to about 15 nM, about 0.1 nM to about 10 nM, about 0.1 nM to about 5 nM, or about 0.1 nM to about 1 nM.

The RNAi constructs of the invention can readily be made using techniques known in the art, for example, using conventional nucleic acid solid phase synthesis. The polynucleotides of the RNAi constructs can be assembled on a suitable nucleic acid synthesizer utilizing standard nucleotide or nucleoside precursors (e.g. phosphoramidites). Automated nucleic acid synthesizers are sold commercially by several vendors, including DNA/RNA synthesizers from Applied Biosystems (Foster City, Calif.), MerMade synthesizers from BioAutomation (Irving, Tex.), and OligoPilot synthesizers from GE Healthcare Life Sciences (Pittsburgh, Pa.).

The 2′ silyl protecting group can be used in conjunction with acid labile dimethoxytrityl (DMT) at the 5′ position of ribonucleosides to synthesize oligonucleotides via phosphoramidite chemistry. Final deprotection conditions are known not to significantly degrade RNA products. All syntheses can be conducted in any automated or manual synthesizer on large, medium, or small scale. The syntheses may also be carried out in multiple well plates, columns, or glass slides.

The 2′-O-silyl group can be removed via exposure to fluoride ions, which can include any source of fluoride ion, e.g., those salts containing fluoride ion paired with inorganic counterions, e.g., cesium fluoride and potassium fluoride or those salts containing fluoride ion paired with an organic counterion, e.g., a tetraalkylammonium fluoride. A crown ether catalyst can be utilized in combination with the inorganic fluoride in the deprotection reaction. Preferred fluoride ion source are tetrabutylammonium fluoride or aminohydrofluorides (e.g., combining aqueous HF with triethylamine in a dipolar aprotic solvent, e.g., dimethylformamide).

The choice of protecting groups for use on the phosphite triesters and phosphotriesters can alter the stability of the triesters towards fluoride. Methyl protection of the phosphotriester or phosphitetriester can stabilize the linkage against fluoride ions and improve process yields.

Since ribonucleosides have a reactive 2′ hydroxyl substituent, it can be desirable to protect the reactive 2′ position in RNA with a protecting group that is orthogonal to a 5′-O-dimethoxytrityl protecting group, e.g., one stable to treatment with acid. Silyl protecting groups meet this criterion and can be readily removed in a final fluoride deprotection step that can result in minimal RNA degradation.

Tetrazole catalysts can be used in the standard phosphoramidite coupling reaction. Preferred catalysts include, e.g., tetrazole, S-ethyl-tetrazole, benzylthiotetrazole, pnitrophenyltetrazole.

As can be appreciated by the skilled artisan, further methods of synthesizing the RNAi constructs described herein will be evident to those of ordinary skill in the art. Additionally, the various synthetic steps may be performed in an alternate sequence or order to give the desired compounds. Other synthetic chemistry transformations, protecting groups (e.g., for hydroxyl, amino, etc. present on the bases) and protecting group methodologies (protection and deprotection) useful in synthesizing the RNAi constructs described herein are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof Custom synthesis of RNAi constructs is also available from several commercial vendors, including Dharmacon, Inc. (Lafayette, Colo.), AxoLabs GmbH (Kulmbach, Germany), and Ambion, Inc. (Foster City, Calif.).

The RNAi constructs of the invention may comprise a ligand. As used herein, a “ligand” refers to any compound or molecule that is capable of interacting with another compound or molecule, directly or indirectly. The interaction of a ligand with another compound or molecule may elicit a biological response (e.g. initiate a signal transduction cascade, induce receptor mediated endocytosis) or may just be a physical association. The ligand can modify one or more properties of the double-stranded RNA molecule to which is attached, such as the pharmacodynamic, pharmacokinetic, binding, absorption, cellular distribution, cellular uptake, charge and/or clearance properties of the RNA molecule.

The ligand may comprise a serum protein (e.g., human serum albumin, low-density lipoprotein, globulin), a cholesterol moiety, a vitamin (biotin, vitamin E, vitamin B12), a folate moiety, a steroid, a bile acid (e.g. cholic acid), a fatty acid (e.g., palmitic acid, myristic acid), a carbohydrate (e.g., a dextran, pullulan, chitin, chitosan, inulin, cyclodextrin or hyaluronic acid), a glycoside, a phospholipid, or antibody or binding fragment thereof (e.g. antibody or binding fragment that targets the RNAi construct to a specific cell type, such as liver). Other examples of ligands include dyes, intercalating agents (e.g. acridines), cross-linkers (e.g. psoralene, mitomycin C), porphyrins (TPPC4, texaphyrin, Sapphyrin), polycyclic aromatic hydrocarbons (e.g., phenazine, dihydrophenazine), artificial endonucleases (e.g. EDTA), lipophilic molecules, e.g, adamantane acetic acid, 1-pyrene butyric acid, dihydrotestosterone, 1,3-BisO(hexadecyl)glycerol, geranyloxyhexyl group, hexadecylglycerol, borneol, menthol, 1,3-propanediol, heptadecyl group, 03-(oleoyl)lithocholic acid, 03-(oleoyl)cholenic acid, dimethoxytrityl, or phenoxazine), peptides (e.g., antennapedia peptide, Tat peptide, RGD peptides), alkylating agents, polymers, such as polyethylene glycol (PEG)(e.g., PEG-40K), poly amino acids, and polyamines (e.g. spermine, spermidine).

In certain embodiments, the ligands have endosomolytic properties. The endosomolytic ligands promote the lysis of the endosome and/or transport of the RNAi construct of the invention, or its components, from the endosome to the cytoplasm of the cell. The endosomolytic ligand may be a polycationic peptide or peptidomimetic which shows pH dependent membrane activity and fusogenicity. In one embodiment, the endosomolytic ligand assumes its active conformation at endosomal pH. The “active” conformation is that conformation in which the endosomolytic ligand promotes lysis of the endosome and/or transport of the RNAi construct of the invention, or its components, from the endosome to the cytoplasm of the cell. Exemplary endosomolytic ligands include the GALA peptide (Subbarao et al., Biochemistry, Vol. 26: 2964-2972, 1987), the EALA peptide (Vogel et al., J. Am. Chem. Soc., Vol. 118: 1581-1586, 1996), and their derivatives (Turk et al., Biochem. Biophys. Acta, Vol. 1559: 56-68, 2002). In one embodiment, the endosomolytic component may contain a chemical group (e.g., an amino acid) which will undergo a change in charge or protonation in response to a change in pH. The endosomolytic component may be linear or branched.

In some embodiments, the ligand comprises a lipid or other hydrophobic molecule. In one embodiment, the ligand comprises a cholesterol moiety or other steroid. Cholesterol conjugated oligonucleotides have been reported to be more active than their unconjugated counterparts (Manoharan, Antisense Nucleic Acid Drug Development, Vol. 12: 103-228, 2002). Ligands comprising cholesterol moieties and other lipids for conjugation to nucleic acid molecules have also been described in U.S. Pat. Nos. 7,851,615; 7,745,608; and 7,833,992, all of which are hereby incorporated by reference in their entireties. In another embodiment, the ligand comprises a folate moiety. Polynucleotides conjugated to folate moieties can be taken up by cells via a receptor-mediated endocytosis pathway. Such folate-polynucleotide conjugates are described in U.S. Pat. No. 8,188,247, which is hereby incorporated by reference in its entirety.

Given that HSD17B13 is expressed in liver cells (e.g. hepatocytes), in certain embodiments, it is desirable to specifically deliver the RNAi construct to those liver cells. In some embodiments, RNAi constructs can be specifically targeted to the liver by employing ligands that bind to or interact with proteins expressed on the surface of liver cells. For example, in certain embodiments, the ligands may comprise antigen binding proteins (e.g. antibodies or binding fragments thereof (e.g. Fab, scFv)) that specifically bind to a receptor expressed on hepatocytes, such as for example, ASGR1.

In certain embodiments, the ligand comprises a carbohydrate. A “carbohydrate” refers to a compound made up of one or more monosaccharide units having at least 6 carbon atoms (which can be linear, branched or cyclic) with an oxygen, nitrogen or sulfur atom bonded to each carbon atom. Carbohydrates include, but are not limited to, the sugars (e.g., monosaccharides, disaccharides, trisaccharides, tetrasaccharides, and oligosaccharides containing from about 4, 5, 6, 7, 8, or 9 monosaccharide units), and polysaccharides, such as starches, glycogen, cellulose and polysaccharide gums. In some embodiments, the carbohydrate incorporated into the ligand is a monosaccharide selected from a pentose, hexose, or heptose and di- and tri-saccharides including such monosaccharide units. In other embodiments, the carbohydrate incorporated into the ligand is an amino sugar, such as galactosamine, glucosamine, N-acetylgalactosamine, and N-acetylglucosamine.

In some embodiments, the ligand comprises a hexose or hexosamine. The hexose may be selected from glucose, galactose, mannose, fucose, or fructose. The hexosamine may be selected from fructosamine, galactosamine, glucosamine, or mannosamine. In certain embodiments, the ligand comprises glucose, galactose, galactosamine, or glucosamine. In one embodiment, the ligand comprises glucose, glucosamine, or N-acetylglucosamine. In another embodiment, the ligand comprises galactose, galactosamine, or N-acetyl-galactosamine. In particular embodiments, the ligand comprises N-acetyl-galactosamine. Ligands comprising glucose, galactose, and N-acetyl-galactosamine (GalNAc) are particularly effective in targeting compounds to liver cells. See, e.g., D'Souza and Devarajan, J. Control Release, Vol. 203: 126-139, 2015. Examples of GalNAc- or galactose-containing ligands that can be incorporated into the RNAi constructs of the invention are described in U.S. Pat. Nos. 7,491,805; 8,106,022; and 8,877,917; U.S. Patent Publication No. 20030130186; and WIPO Publication No. WO2013166155, all of which are hereby incorporated by reference in their entireties.

In certain embodiments, the ligand comprises a multivalent carbohydrate moiety. As used herein, a “multivalent carbohydrate moiety” refers to a moiety comprising two or more carbohydrate units capable of independently binding or interacting with other molecules. For example, a multivalent carbohydrate moiety comprises two or more binding domains comprised of carbohydrates that can bind to two or more different molecules or two or more different sites on the same molecule. The valency of the carbohydrate moiety denotes the number of individual binding domains within the carbohydrate moiety. For instance, the terms “monovalent,” “bivalent,” “trivalent,” and “tetravalent” with reference to the carbohydrate moiety refer to carbohydrate moieties with one, two, three, and four binding domains, respectively. The multivalent carbohydrate moiety may comprise a multivalent lactose moiety, a multivalent galactose moiety, a multivalent glucose moiety, a multivalent N-acetyl-galactosamine moiety, a multivalent N-acetyl-glucosamine moiety, a multivalent mannose moiety, or a multivalent fucose moiety. In some embodiments, the ligand comprises a multivalent galactose moiety. In other embodiments, the ligand comprises a multivalent N-acetyl-galactosamine moiety. In these and other embodiments, the multivalent carbohydrate moiety is bivalent, trivalent, or tetravalent. In such embodiments, the multivalent carbohydrate moiety can be bi-antennary or tri-antennary. In one particular embodiment, the multivalent N-acetyl-galactosamine moiety is trivalent or tetravalent. In another particular embodiment, the multivalent galactose moiety is trivalent or tetravalent. Exemplary trivalent and tetravalent GalNAc-containing ligands for incorporation into the RNAi constructs of the invention are described in detail below.

The ligand can be attached or conjugated to the RNA molecule of the RNAi construct directly or indirectly. For instance, in some embodiments, the ligand is covalently attached directly to the sense or antisense strand of the RNAi construct. In other embodiments, the ligand is covalently attached via a linker to the sense or antisense strand of the RNAi construct. The ligand can be attached to nucleobases, sugar moieties, or internucleotide linkages of polynucleotides (e.g. sense strand or antisense strand) of the RNAi constructs of the invention. Conjugation or attachment to purine nucleobases or derivatives thereof can occur at any position including, endocyclic and exocyclic atoms. In certain embodiments, the 2-, 6-, 7-, or 8-positions of a purine nucleobase are attached to a ligand. Conjugation or attachment to pyrimidine nucleobases or derivatives thereof can also occur at any position. In some embodiments, the 2-, 5-, and 6-positions of a pyrimidine nucleobase can be attached to a ligand. Conjugation or attachment to sugar moieties of nucleotides can occur at any carbon atom. Example carbon atoms of a sugar moiety that can be attached to a ligand include the 2′, 3′, and 5′ carbon atoms. The 1′ position can also be attached to a ligand, such as in an a basic residue. Internucleotide linkages can also support ligand attachments. For phosphorus-containing linkages (e.g., phosphodiester, phosphorothioate, phosphorodithiotate, phosphoroamidate, and the like), the ligand can be attached directly to the phosphorus atom or to an O, N, or S atom bound to the phosphorus atom. For amine- or amide-containing internucleoside linkages (e.g., PNA), the ligand can be attached to the nitrogen atom of the amine or amide or to an adjacent carbon atom.

In certain embodiments, the ligand may be attached to the 3′ or 5′ end of either the sense or antisense strand. In certain embodiments, the ligand is covalently attached to the 5′ end of the sense strand. In other embodiments, the ligand is covalently attached to the 3′ end of the sense strand. For example, in some embodiments, the ligand is attached to the 3′-terminal nucleotide of the sense strand. In certain such embodiments, the ligand is attached at the 3′-position of the 3′-terminal nucleotide of the sense strand. In alternative embodiments, the ligand is attached near the 3′ end of the sense strand, but before one or more terminal nucleotides (i.e. before 1, 2, 3, or 4 terminal nucleotides). In some embodiments, the ligand is attached at the 2′-position of the sugar of the 3′-terminal nucleotide of the sense strand.

In certain embodiments, the ligand is attached to the sense or antisense strand via a linker. A “linker” is an atom or group of atoms that covalently joins a ligand to a polynucleotide component of the RNAi construct. The linker may be from about 1 to about 30 atoms in length, from about 2 to about 28 atoms in length, from about 3 to about 26 atoms in length, from about 4 to about 24 atoms in length, from about 6 to about 20 atoms in length, from about 7 to about 20 atoms in length, from about 8 to about 20 atoms in length, from about 8 to about 18 atoms in length, from about 10 to about 18 atoms in length, and from about 12 to about 18 atoms in length. In some embodiments, the linker may comprise a bifunctional linking moiety, which generally comprises an alkyl moiety with two functional groups. One of the functional groups is selected to bind to the compound of interest (e.g. sense or antisense strand of the RNAi construct) and the other is selected to bind essentially any selected group, such as a ligand as described herein. In certain embodiments, the linker comprises a chain structure or an oligomer of repeating units, such as ethylene glycol or amino acid units. Examples of functional groups that are typically employed in a bifunctional linking moiety include, but are not limited to, electrophiles for reacting with nucleophilic groups and nucleophiles for reacting with electrophilic groups. In some embodiments, bifunctional linking moieties include amino, hydroxyl, carboxylic acid, thiol, unsaturated bonds (e.g., double or triple bonds), and the like.

Linkers that may be used to attach a ligand to the sense or antisense strand in the RNAi constructs of the invention include, but are not limited to, pyrrolidine, 8-amino-3,6-di oxaoctanoic acid, succinimidyl 4-(N-maleimidomethyl) cyclohexane-1-carboxylate, 6-aminohexanoic acid, substituted C1-C10 alkyl, substituted or unsubstituted C2-C10 alkenyl or substituted or unsubstituted C2-C10 alkynyl. Preferred substituent groups for such linkers include, but are not limited to, hydroxyl, amino, alkoxy, carboxy, benzyl, phenyl, nitro, thiol, thioalkoxy, halogen, alkyl, aryl, alkenyl and alkynyl.

In certain embodiments, the linkers are cleavable. A cleavable linker is one which is sufficiently stable outside the cell, but which upon entry into a target cell is cleaved to release the two parts the linker is holding together. In some embodiments, the cleavable linker is cleaved at least 10 times, 20 times, 30 times, 40 times, 50 times, 60 times, 70 times, 80 times, 90 times, or more, or at least 100 times faster in the target cell or under a first reference condition (which can, e.g., be selected to mimic or represent intracellular conditions) than in the blood of a subject, or under a second reference condition (which can, e.g., be selected to mimic or represent conditions found in the blood or serum).

Cleavable linkers are susceptible to cleavage agents, e.g., pH, redox potential or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable linker by reduction; esterases; endosomes or agents that can create an acidic environment, e.g., those that result in a pH of five or lower; enzymes that can hydrolyze or degrade an acid cleavable linker by acting as a general acid, peptidases (which can be substrate specific), and phosphatases.

A cleavable linker may comprise a moiety that is susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some linkers will have a cleavable group that is cleaved at a preferred pH, thereby releasing the RNA molecule from the ligand inside the cell, or into the desired compartment of the cell.

A linker can include a cleavable group that is cleavable by a particular enzyme. The type of cleavable group incorporated into a linker can depend on the cell to be targeted. For example, liver-targeting ligands can be linked to RNA molecules through a linker that includes an ester group. Liver cells are rich in esterases, and therefore the linker will be cleaved more efficiently in liver cells than in cell types that are not esterase-rich. Other types of cells rich in esterases include cells of the lung, renal cortex, and testis. Linkers that contain peptide bonds can be used when targeting cells rich in peptidases, such as liver cells and synoviocytes.

In general, the suitability of a candidate cleavable linker can be evaluated by testing the ability of a degradative agent (or condition) to cleave the candidate linker. It will also be desirable to also test the candidate cleavable linker for the ability to resist cleavage in the blood or when in contact with other non-target tissue. Thus, one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell free systems, in cells, in cell culture, in organ or tissue culture, or in whole animals. It may be useful to make initial evaluations in cell-free or culture conditions and to confirm by further evaluations in whole animals. In some embodiments, useful candidate linkers are cleaved at least 2, 4, 10, 20, 50, 70, or 100 times faster in the target cell (or under in vitro conditions selected to mimic intracellular conditions) as compared to blood or serum (or under in vitro conditions selected to mimic extracellular conditions).

In other embodiments, redox cleavable linkers are utilized. Redox cleavable linkers are cleaved upon reduction or oxidation. An example of reductively cleavable group is a disulfide linking group (—S—S—). To determine if a candidate cleavable linker is a suitable “reductively cleavable linker,” or for example is suitable for use with a particular RNAi construct and particular ligand, one can use one or more methods described herein. For example, a candidate linker can be evaluated by incubation with dithiothreitol (DTT), or other reducing agent known in the art, which mimics the rate of cleavage that would be observed in a cell, e.g., a target cell. The candidate linkers can also be evaluated under conditions which are selected to mimic blood or serum conditions. In a specific embodiment, candidate linkers are cleaved by at most 10% in the blood. In other embodiments, useful candidate linkers are degraded at least 2, 4, 10, 20, 50, 70, or 100 times faster in the cell (or under in vitro conditions selected to mimic intracellular conditions) as compared to blood (or under in vitro conditions selected to mimic extracellular conditions).

In yet other embodiments, phosphate-based cleavable linkers are cleaved by agents that degrade or hydrolyze the phosphate group. An example of an agent that hydrolyzes phosphate groups in cells are enzymes, such as phosphatases in cells. Examples of phosphate-based cleavable groups are —O—P(O)(ORk)-O—, —O—P(S)(ORk)-O—, —O—P(S)(SRk)-O—, —S—P(O)(ORk)-O—, —O—P(O)(ORk)-S—, —S—P(O)(ORk)-S—, —O—P(S)(ORk)-S—, —S—P(S)(ORk)-O—, —O—P(O)(Rk)-O—, —O—P(S)(Rk)-O—, —S—P(O)(Rk)-O—, —S—P(S)(Rk)-O—, —S—P(O)(Rk)-S—, —O—P(S)(Rk)-S—. Specific embodiments include —O—P(O)(OH)—O—, —O—P(S)(OH)—O—, —O—P(S)(SH)—O—, —S—P(O)(OH)—O—, —O—P(O)(OH)—S—, —S—P(O)(OH)—S—, —O—P(S)(OH)—S—, —SP(S)(OH)—O—, —O—P(O)(H)—O—, —O—P(S)(H)—O—, —S—P(O)(H)—O—, —S—P(S)(H)—O—, —S—P(O)(H)—S—, —O—P(S)(H)—S—. Another specific embodiment is —O—P(O)(OH)—O—. These candidate linkers can be evaluated using methods analogous to those described above.

In other embodiments, the linkers may comprise acid cleavable groups, which are groups that are cleaved under acidic conditions. In some embodiments, acid cleavable groups are cleaved in an acidic environment with a pH of about 6.5 or lower (e.g., about 6.0, 5.5, 5.0, or lower), or by agents, such as enzymes that can act as a general acid. In a cell, specific low pH organelles, such as endosomes and lysosomes, can provide a cleaving environment for acid cleavable groups. Examples of acid cleavable linking groups include, but are not limited to, hydrazones, esters, and esters of amino acids. Acid cleavable groups can have the general formula —C═NN—, C(O)O, or —OC(O). A specific embodiment is when the carbon attached to the oxygen of the ester (the alkoxy group) is an aryl group, substituted alkyl group, or tertiaryalkyl group such as dimethyl, pentyl or t-butyl. These candidates can be evaluated using methods analogous to those described above.

In other embodiments, the linkers may comprise ester-based cleavable groups, which are cleaved by enzymes, such as esterases and amidases in cells. Examples of ester-based cleavable groups include, but are not limited to, esters of alkylene, alkenylene and alkynylene groups. Ester cleavable groups have the general formula —C(O)O—, or —OC(O)—. These candidate linkers can be evaluated using methods analogous to those described above.

In further embodiments, the linkers may comprise peptide-based cleavable groups, which are cleaved by enzymes, such as peptidases and proteases in cells. Peptide-based cleavable groups are peptide bonds formed between amino acids to yield oligopeptides (e.g., dipeptides, tripeptides etc.) and polypeptides. Peptide-based cleavable groups do not include the amide group (—C(O)NH—). The amide group can be formed between any alkylene, alkenylene or alkynelene. A peptide bond is a special type of amide bond formed between amino acids to yield peptides and proteins. The peptide-based cleavage group is generally limited to the peptide bond (i.e., the amide bond) formed between amino acids yielding peptides and proteins and does not include the entire amide functional group. Peptide-based cleavable linking groups have the general formula —NHCHRAC(O)NHCHRBC(O)—, where RA and RB are the R groups of the two adjacent amino acids. These candidates can be evaluated using methods analogous to those described above.

Other types of linkers suitable for attaching ligands to the sense or antisense strands in the RNAi constructs of the invention are known in the art and can include the linkers described in U.S. Pat. Nos. 7,723,509; 8,017,762; 8,828,956; 8,877,917; and 9,181,551, all of which are hereby incorporated by reference in their entireties.

In certain embodiments, the ligand covalently attached to the sense or antisense strand of the RNAi constructs of the invention comprises a GalNAc moiety, e.g, a multivalent GalNAc moiety. In some embodiments, the multivalent GalNAc moiety is a trivalent GalNAc moiety and is attached to the 3′ end of the sense strand. In other embodiments, the multivalent GalNAc moiety is a trivalent GalNAc moiety and is attached to the 5′ end of the sense strand. In yet other embodiments, the multivalent GalNAc moiety is a tetravalent GalNAc moiety and is attached to the 3′ end of the sense strand. In still other embodiments, the multivalent GalNAc moiety is a tetravalent GalNAc moiety and is attached to the 5′ end of the sense strand. In yet other embodiments, the multivalent GalNAc moiety is a tetravalent GalNAc moiety and is attached to the 3′ end of the sense strand. In still other embodiments, the multivalent GalNAc moiety is a tetravalent GalNAc moiety and is attached to the 5′ end of the sense strand. In some embodiments, a GalNAc moiety is attached to the 5′ end of the sense strand of the odd numbered sequences of SEQ ID NOs: 1-645 or 647-1291.

In some embodiments, the RNAi constructs of the invention may be delivered to a cell or tissue of interest by administering a vector that encodes and controls the intracellular expression of the RNAi construct. A “vector” (also referred to herein as an “expression vector) is a composition of matter which can be used to deliver a nucleic acid of interest to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. Examples of viral vectors include, but are not limited to, adenoviral vectors, adeno-associated viral vectors, retroviral vectors, and the like. A vector can be replicated in a living cell, or it can be made synthetically.

Generally, a vector for expressing an RNAi construct of the invention will comprise one or more promoters operably linked to sequences encoding the RNAi construct. The phrase “operably linked” or “under transcriptional control” as used herein means that the promoter is in the correct location and orientation in relation to a polynucleotide sequence to control the initiation of transcription by RNA polymerase and expression of the polynucleotide sequence. A “promoter” refers to a sequence recognized by the synthetic machinery of the cell, or introduced synthetic machinery, required to initiate the specific transcription of a gene sequence. Suitable promoters include, but are not limited to, RNA pol I, pol II, HI or U6 RNA pol III, and viral promoters (e.g. human cytomegalovirus (CMV) immediate early gene promoter, the SV40 early promoter, and the Rous sarcoma virus long terminal repeat). In some embodiments, a HI or U6 RNA pol III promoter is preferred. The promoter can be a tissue-specific or inducible promoter. Of particular interest are liver-specific promoters, such as promoter sequences from human alpha1-antitrypsin gene, albumin gene, hemopexin gene, and hepatic lipase gene. Inducible promoters include promoters regulated by ecdysone, estrogen, progesterone, tetracycline, and isopropyl-PD1-thiogalactopyranoside (IPTG).

In some embodiments in which the RNAi construct comprises a siRNA, the two separate strands (sense and antisense strand) can be expressed from a single vector or two separate vectors. For example, in one embodiment, the sequence encoding the sense strand is operably linked to a promoter on a first vector and the sequence encoding the antisense strand is operably linked to a promoter on a second vector. In such an embodiment, the first and second vectors are co-introduced, e.g., by infection or transfection, into a target cell, such that the sense and antisense strands, once transcribed, will hybridize intracellularly to form the siRNA molecule. In another embodiment, the sense and antisense strands are transcribed from two separate promoters located in a single vector. In some such embodiments, the sequence encoding the sense strand is operably linked to a first promoter and the sequence encoding the antisense strand is operably linked to a second promoter, wherein the first and second promoters are located in a single vector. In one embodiment, the vector comprises a first promoter operably linked to a sequence encoding the siRNA molecule, and a second promoter operably linked to the same sequence in the opposite direction, such that transcription of the sequence from the first promoter results in the synthesis of the sense strand of the siRNA molecule and transcription of the sequence from the second promoter results in synthesis of the antisense strand of the siRNA molecule.

In other embodiments in which the RNAi construct comprises a shRNA, a sequence encoding the single, at least partially self-complementary RNA molecule is operably linked to a promoter to produce a single transcript. In some embodiments, the sequence encoding the shRNA comprises an inverted repeat joined by a linker polynucleotide sequence to produce the stem and loop structure of the shRNA following transcription.

In some embodiments, the vector encoding an RNAi construct of the invention is a viral vector. Various viral vector systems that are suitable to express the RNAi constructs described herein include, but are not limited to, adenoviral vectors, retroviral vectors (e.g., lentiviral vectors, maloney murine leukemia virus), adeno-associated viral vectors; herpes simplex viral vectors; SV 40 vectors; polyoma viral vectors; papilloma viral vectors; picornaviral vectors; and pox viral vectors (e.g. vaccinia virus). In certain embodiments, the viral vector is a retroviral vector (e.g. lentiviral vector).

Various vectors suitable for use in the invention, methods for inserting nucleic acid sequences encoding siRNA or shRNA molecules into vectors, and methods of delivering the vectors to the cells of interest are within the skill of those in the art. See, e.g., Dornburg, Gene Therap., Vol. 2: 301-310, 1995; Eglitis, Biotechniques, Vol. 6: 608-614, 1988; Miller, HumGene Therap., Vol. 1: 5-14, 1990; Anderson, Nature, Vol. 392: 25-30, 1998; Rubinson D A et al., Nat. Genet., Vol. 33: 401-406, 2003; Brummelkamp et al., Science, Vol. 296: 550-553, 2002; Brummelkamp et al., Cancer Cell, Vol. 2: 243-247, 2002; Lee et al., Nat Biotechnol, Vol. 20:500-505, 2002; Miyagishi et al., Nat Biotechnol, Vol. 20: 497-500, 2002; Paddison et al., Genes Dev, Vol. 16: 948-958, 2002; Paul et al., Nat Biotechnol, Vol. 20: 505-508, 2002; Sui et al., Proc Natl Acad Sci USA, Vol. 99: 5515-5520, 2002; and Yu et al., Proc Natl Acad Sci USA, Vol. 99:6047-6052, 2002, all of which are hereby incorporated by reference in their entireties.

The present invention also includes pharmaceutical compositions and formulations comprising the RNAi constructs described herein and pharmaceutically acceptable carriers, excipients, or diluents. Such compositions and formulations are useful for reducing expression of HSD17B13 in a subject in need thereof. Where clinical applications are contemplated, pharmaceutical compositions and formulations will be prepared in a form appropriate for the intended application. Generally, this will entail preparing compositions that are essentially free of pyrogens, as well as other impurities that could be harmful to humans or animals.

The phrases “pharmaceutically acceptable” or “pharmacologically acceptable” refer to molecular entities and compositions that do not produce adverse, allergic, or other untoward reactions when administered to an animal or a human. As used herein, “pharmaceutically acceptable carrier, excipient, or diluent” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Except insofar as any conventional media or agent is incompatible with the RNAi constructs of the present invention, its use in therapeutic compositions is contemplated. Supplementary active ingredients also can be incorporated into the compositions, provided they do not inactivate the vectors or RNAi constructs of the compositions.

Compositions and methods for the formulation of pharmaceutical compositions depend on a number of criteria, including, but not limited to, route of administration, type and extent of disease or disorder to be treated, or dose to be administered. In some embodiments, the pharmaceutical compositions are formulated based on the intended route of delivery. For instance, in certain embodiments, the pharmaceutical compositions are formulated for parenteral delivery. Parenteral forms of delivery include intravenous, intraarterial, subcutaneous, intrathecal, intraperitoneal or intramuscular injection or infusion. In one embodiment, the pharmaceutical composition is formulated for intravenous delivery. In such an embodiment, the pharmaceutical composition may include a lipid-based delivery vehicle. In another embodiment, the pharmaceutical composition is formulated for subcutaneous delivery. In such an embodiment, the pharmaceutical composition may include a targeting ligand (e.g. GalNAc containing ligands described herein).

In some embodiments, the pharmaceutical compositions comprise an effective amount of an RNAi construct described herein. An “effective amount” is an amount sufficient to produce a beneficial or desired clinical result. In some embodiments, an effective amount is an amount sufficient to reduce HSD17B13 expression in hepatocytes of a subject. In some embodiments, an effective amount may be an amount sufficient to only partially reduce HSD17B13 expression, for example, to a level comparable to expression of the wild-type HSD17B13 allele in human heterozygotes.

An effective amount of an RNAi construct of the invention may be from about 0.01 mg/kg body weight to about 100 mg/kg body weight, about 0.05 mg/kg body weight to about 75 mg/kg body weight, about 0.1 mg/kg body weight to about 50 mg/kg body weight, about 1 mg/kg to about 30 mg/kg body weight, about 2.5 mg/kg of body weight to about 20 mg/kg bodyweight, or about 5 mg/kg body weight to about 15 mg/kg body weight. In certain embodiments, a single effective dose of an RNAi construct of the invention may be about 0.1 mg/kg, about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, or about 10 mg/kg. The pharmaceutical composition comprising an effective amount of RNAi construct can be administered weekly, biweekly, monthly, quarterly, or biannually. The precise determination of what would be considered an effective amount and frequency of administration may be based on several factors, including a patient's size, age, and general condition, type of disorder to be treated (e.g. myocardial infarction, heart failure, coronary artery disease, hypercholesterolemia), particular RNAi construct employed, and route of administration. Estimates of effective dosages and in vivo half-lives for any particular RNAi construct of the invention can be ascertained using conventional methods and/or testing in appropriate animal models.

Administration of the pharmaceutical compositions of the present invention may be via any common route so long as the target tissue is available via that route. Such routes include, but are not limited to, parenteral (e.g., subcutaneous, intramuscular, intraperitoneal or intravenous), oral, nasal, buccal, intradermal, transdermal, and sublingual routes, or by direct injection into liver tissue or delivery through the hepatic portal vein. In some embodiments, the pharmaceutical composition is administered parenterally. For instance, in certain embodiments, the pharmaceutical composition is administered intravenously. In other embodiments, the pharmaceutical composition is administered subcutaneously.

Colloidal dispersion systems, such as macromolecule complexes, nanocapsules, microspheres, beads, and lipid-based systems, including oil-in-water emulsions, micelles, mixed micelles, and liposomes, may be used as delivery vehicles for the RNAi constructs of the invention or vectors encoding such constructs. Commercially available fat emulsions that are suitable for delivering the nucleic acids of the invention include Intralipid®, Liposyn®, Liposyn®II, Liposyn®III, Nutrilipid, and other similar lipid emulsions. A preferred colloidal system for use as a delivery vehicle in vivo is a liposome (i.e., an artificial membrane vesicle). The RNAi constructs of the invention may be encapsulated within liposomes or may form complexes thereto, in particular to cationic liposomes. Alternatively, RNAi constructs of the invention may be complexed to lipids, in particular to cationic lipids. Suitable lipids and liposomes include neutral (e.g., dioleoylphosphatidyl ethanolamine (DOPE), dimyristoylphosphatidyl choline (DMPC), and dipalmitoyl phosphatidylcholine (DPPC)), distearolyphosphatidyl choline), negative (e.g., dimyristoylphosphatidyl glycerol (DMPG)), and cationic (e.g., dioleoyltetramethylaminopropyl (DOTAP) and dioleoylphosphatidyl ethanolamine (DOTMA)). The preparation and use of such colloidal dispersion systems is well known in the art. Exemplary formulations are also disclosed in U.S. Pat. Nos. 5,981,505; 6,217,900; 6,383,512; 5,783,565; 7,202,227; 6,379,965; 6,127,170; 5,837,533; 6,747,014; and WO03/093449.

In some embodiments, the RNAi constructs of the invention are fully encapsulated in a lipid formulation, e.g., to form a SPLP, pSPLP, SNALP, or other nucleic acid-lipid particle. As used herein, the term “SNALP” refers to a stable nucleic acid-lipid particle, including SPLP. As used herein, the term “SPLP” refers to a nucleic acid-lipid particle comprising plasmid DNA encapsulated within a lipid vesicle. SNALPs and SPLPs typically contain a cationic lipid, a noncationic lipid, and a lipid that prevents aggregation of the particle (e.g., a PEG-lipid conjugate). SNALPs and SPLPs are exceptionally useful for systemic applications, as they exhibit extended circulation lifetimes following intravenous injection and accumulate at distal sites (e.g., sites physically separated from the administration site). SPLPs include “pSPLP,” which include an encapsulated condensing agent-nucleic acid complex as set forth in PCT Publication No. WO00/03683. The nucleic acid-lipid particles typically have a mean diameter of about 50 nm to about 150 nm, about 60 nm to about 130 nm, about 70 nm to about 110 nm, or about 70 nm to about 90 nm, and are substantially nontoxic. In addition, the nucleic acids when present in the nucleic acid-lipid particles are resistant in aqueous solution to degradation with a nuclease. Nucleic acid-lipid particles and their method of preparation are disclosed in, e.g., U.S. Pat. Nos. 5,976,567; 5,981,501; 6,534,484; 6,586,410; 6,815,432; and PCT Publication No. WO96/40964.

The pharmaceutical compositions suitable for injectable use include, for example, sterile aqueous solutions or dispersions and sterile powders for the extemporaneous preparation of sterile injectable solutions or dispersions. Generally, these preparations are sterile and fluid to the extent that easy injectability exists. Preparations should be stable under the conditions of manufacture and storage and should be preserved against the contaminating action of microorganisms, such as bacteria and fungi. Appropriate solvents or dispersion media may contain, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions may be prepared by incorporating the active compounds in an appropriate amount into a solvent along with any other ingredients (for example as enumerated above) as desired, followed by filtered sterilization. Generally, dispersions are prepared by incorporating the various sterilized active ingredients into a sterile vehicle which contains the basic dispersion medium and the desired other ingredients, e.g., as enumerated above. In the case of sterile powders for the preparation of sterile injectable solutions, the preferred methods of preparation include vacuum-drying and freeze-drying techniques which yield a powder of the active ingredient(s) plus any additional desired ingredient from a previously sterile-filtered solution thereof.

The compositions of the present invention generally may be formulated in a neutral or salt form. Pharmaceutically-acceptable salts include, for example, acid addition salts (formed with free amino groups) derived from inorganic acids (e.g., hydrochloric or phosphoric acids), or from organic acids (e.g., acetic, oxalic, tartaric, mandelic, and the like). Salts formed with the free carboxyl groups can also be derived from inorganic bases (e.g., sodium, potassium, ammonium, calcium, or ferric hydroxides) or from organic bases (e.g., isopropylamine, trimethylamine, histidine, procaine and the like).

For parenteral administration in an aqueous solution, for example, the solution generally is suitably buffered and the liquid diluent first rendered isotonic for example with sufficient saline or glucose. Such aqueous solutions may be used, for example, for intravenous, intramuscular, subcutaneous and intraperitoneal administration. Preferably, sterile aqueous media are employed as is known to those of skill in the art, particularly in light of the present disclosure. By way of illustration, a single dose may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). For human administration, preparations should meet sterility, pyrogenicity, general safety and purity standards as required by FDA standards. In certain embodiments, a pharmaceutical composition of the invention comprises or consists of a sterile saline solution and an RNAi construct described herein. In other embodiments, a pharmaceutical composition of the invention comprises or consists of an RNAi construct described herein and sterile water (e.g. water for injection, WFI). In still other embodiments, a pharmaceutical composition of the invention comprises or consists of an RNAi construct described herein and phosphate-buffered saline (PBS).

In some embodiments, the pharmaceutical compositions of the invention are packaged with or stored within a device for administration. Devices for injectable formulations include, but are not limited to, injection ports, pre-filled syringes, auto injectors, injection pumps, on-body injectors, and injection pens. Devices for aerosolized or powder formulations include, but are not limited to, inhalers, insufflators, aspirators, and the like. Thus, the present invention includes administration devices comprising a pharmaceutical composition of the invention for treating or preventing one or more of the disorders described herein.

### Methods for Inhibiting HSD17B13 Expression

The present invention also provides methods of inhibiting expression of a HSD17B13 gene in a cell. The methods include contacting a cell with an RNAi construct, e.g., double stranded RNAi construct, in an amount effective to inhibit expression of HSD17B13 in the cell, thereby inhibiting expression of HSD17B13 in the cell. Contacting of a cell with an RNAi construct, e.g., a double stranded RNAi construct, may be done in vitro or in vivo. Contacting a cell in vivo with the RNAi construct includes contacting a cell or group of cells within a subject, e.g., a human subject, with the RNAi construct. Combinations of in vitro and in vivo methods of contacting a cell are also possible.

The present invention provides methods for reducing or inhibiting expression of HSD17B13 in a subject in need thereof as well as methods of treating or preventing conditions, diseases, or disorders associated with HSD17B13 expression or activity. A “condition, disease, or disorder associated with HSD17B13 expression” refers to conditions, diseases, or disorders in which HSD17B13 expression levels are altered or where elevated expression levels of HSD17B13 are associated with an increased risk of developing the condition, disease or disorder.

Contacting a cell may be direct or indirect, as discussed above. Furthermore, contacting a cell may be accomplished via a targeting ligand, including any ligand described herein or known in the art. In preferred embodiments, the targeting ligand is a carbohydrate moiety, e.g., a GalNAc ligand, or a trivalent GalNAc moiety, or any other ligand that directs the RNAi construct to a site of interest.

In one embodiment, contacting a cell with an RNAi construct includes “introducing” or “delivering the RNAi construct into the cell” by facilitating or effecting uptake or absorption into the cell. Absorption or uptake of an RNAi construct can occur through unaided diffusive or active cellular processes, or by auxiliary agents or devices. Introducing an RNAi construct into a cell may be in vitro and/or in vivo. For example, for in vivo introduction, RNAi constructs can be injected into a tissue site or administered systemically. In vitro introduction into a cell includes methods known in the art such as electroporation and lipofection. Further approaches are described herein below and/or are known in the art.

The term “inhibiting,” as used herein, is used interchangeably with “reducing,” “silencing,” “downregulating”, “suppressing”, and other similar terms, and includes any level of inhibition.

The phrase “inhibiting expression of a HSD17B13” is intended to refer to inhibition of expression of any HSD17B13 gene (such as, e.g., a mouse HSD17B13 gene, a rat HSD17B13 gene, a monkey HSD17B13 gene, or a human HSD17B13 gene) as well as variants or mutants of a HSD17B13 gene. Thus, the HSD17B13 gene may be a wild-type HSD17B13 gene, a mutant HSD17B13 gene, or a transgenic HSD17B13 gene in the context of a genetically manipulated cell, group of cells, or organism.

“Inhibiting expression of a HSD17B13 gene” includes any level of inhibition of a HSD17B13 gene, e.g., at least partial suppression of the expression of a HSD17B13 gene. The expression of the HSD17B13 gene may be assessed based on the level, or the change in the level, of any variable associated with HSD17B13 gene expression, e.g., HSD17B13 mRNA level, HSD17B13 protein level, or the number or extent of amyloid deposits. This level may be assessed in an individual cell or in a group of cells, including, for example, a sample derived from a subject.

Inhibition may be assessed by a decrease in an absolute or relative level of one or more variables that are associated with HSD17B13 expression compared with a control level. The control level may be any type of control level that is utilized in the art, e.g., a pre-dose baseline level, or a level determined from a similar subject, cell, or sample that is untreated or treated with a control (such as, e.g., buffer only control or inactive agent control). In some embodiments of the methods of the invention, expression of a HSD17B13 gene is inhibited by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%. at least about 95%, at least about 96%, at least about 97%, at least about 98%, or at least about 99%.

Inhibition of the expression of a HSD17B13 gene may be manifested by a reduction of the amount of mRNA expressed by a first cell or group of cells (such cells may be present, for example, in a sample derived from a subject) in which a HSD17B13 gene is transcribed and which has or have been treated (e.g., by contacting the cell or cells with an RNAi construct of the invention, or by administering an RNAi construct of the invention to a subject in which the cells are or were present) such that the expression of a HSD17B13 gene is inhibited, as compared to a second cell or group of cells substantially identical to the first cell or group of cells but which has not or have not been so treated (control cell(s)). In preferred embodiments, the inhibition is assessed by expressing the level of mRNA in treated cells as a percentage of the level of mRNA in control cells, using the following formula:

$$\frac{( mRNA in control cells ) - ( mRNA in treated cells )}{( mRNA in control cells )} \cdot 100 \%$$

Alternatively, inhibition of the expression of a HSD17B13 gene may be assessed in terms of a reduction of a parameter that is functionally linked to HSD17B13 gene expression. HSD17B13 gene silencing may be determined in any cell expressing HSD17B13, either constitutively or by genomic engineering, and by any assay known in the art.

Inhibition of the expression of a HSD17B13 protein may be manifested by a reduction in the level of the HSD17B13 protein that is expressed by a cell or group of cells (e.g., the level of protein expressed in a sample derived from a subject). As explained above, for the assessment of mRNA suppression, the inhibition of protein expression levels in a treated cell or group of cells may similarly be expressed as a percentage of the level of protein in a control cell or group of cells.

A control cell or group of cells that may be used to assess the inhibition of the expression of a HSD17B13 gene includes a cell or group of cells that has not yet been contacted with an RNAi construct of the invention. For example, the control cell or group of cells may be derived from an individual subject (e.g., a human or animal subject) prior to treatment of the subject with an RNAi construct.

The level of HSD17B13 mRNA that is expressed by a cell or group of cells, or the level of circulating HSD17B13 mRNA, may be determined using any method known in the art for assessing mRNA expression. In one embodiment, the level of expression of HSD17B13 in a sample is determined by detecting a transcribed polynucleotide, or portion thereof, e.g., mRNA of the HSD17B13 gene. RNA may be extracted from cells using RNA extraction techniques including, for example, using acid phenol/guanidine isothiocyanate extraction (RNAzol B; Biogenesis), RNeasy RNA preparation kits (Qiagen) or PAXgene (PreAnalytix, Switzerland). Typical assay formats utilizing ribonucleic acid hybridization include nuclear run-on assays, RT-PCR, RNase protection assays (Melton et al., Nuc. Acids Res. 12:7035), Northern blotting, in situ hybridization, and microarray analysis. Circulating mRNA may be detected using methods the described in PCT/US2012/043584, the entire contents of which are hereby incorporated herein by reference.

In one embodiment, the level of expression of HSD17B13 is determined using a nucleic acid probe. The term “probe”, as used herein, refers to any molecule that is capable of selectively binding to a specific HSD17B13. Probes can be synthesized by one of skill in the art, or derived from appropriate biological preparations. Probes may be specifically designed to be labeled. Examples of molecules that can be utilized as probes include, but are not limited to, RNA, DNA, proteins, antibodies, and organic molecules.

Isolated mRNA can be used in hybridization or amplification assays that include, but are not limited to, Southern or Northern analyses, polymerase chain reaction (PCR) analyses and probe arrays. One method for the determination of mRNA levels involves contacting the isolated mRNA with a nucleic acid molecule (probe) that can hybridize to HSD17B13 mRNA. In one embodiment, the mRNA is immobilized on a solid surface and contacted with a probe, for example by running the isolated mRNA on an agarose gel and transferring the mRNA from the gel to a membrane, such as nitrocellulose. In an alternative embodiment, the probe(s) are immobilized on a solid surface and the mRNA is contacted with the probe(s), for example, in an Affymetrix gene chip array. A skilled artisan can readily adapt known mRNA detection methods for use in determining the level of HSD17B13 mRNA.

An alternative method for determining the level of expression of HSD17B13 in a sample involves the process of nucleic acid amplification and/or reverse transcriptase (to prepare cDNA) of for example mRNA in the sample, e.g., by RT-PCR (the experimental embodiment set forth in Mullis, 1987, U.S. Pat. No. 4,683,202), ligase chain reaction (Barany (1991) Proc. Natl. Acad. Sci. USA 88: 189-193), self-sustained sequence replication (Guatelli et al. (1990) Proc. Natl. Acad. Sci. USA 87: 1874-1878), transcriptional amplification system (Kwoh et al. (1989) Proc. Natl. Acad. Sci. USA 86: 1173-1177), Q-Beta Replicase (Lizardi et al. (1988) Bio/Technology 6: 1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. In particular aspects of the invention, the level of expression of HSD17B13 is determined by quantitative fluorogenic RT-PCR (i.e., the TaqMan™ System). The expression levels of HSD17B13 mRNA may be monitored using a membrane blot (such as used in hybridization analysis such as Northern, Southern, dot, and the like), or microwells, sample tubes, gels, beads or fibers (or any solid support comprising bound nucleic acids). See U.S. Pat. Nos. 5,770,722, 5,874,219, 5,744,305, 5,677,195 and 5,445,934, which are incorporated herein by reference. The determination of HSD17B13 expression level may also comprise using nucleic acid probes in solution.

In preferred embodiments, the level of mRNA expression is assessed using, for example, branched DNA (bDNA) assays, real time PCR (qPCR), or quantitative FISH assays. The use of these methods is described and exemplified in the Examples presented herein.

The level of HSD17B13 protein expression may be determined using any method known in the art for the measurement of protein levels. Such methods include, for example, electrophoresis, capillary electrophoresis, high performance liquid chromatography (HPLC), thin layer chromatography (TLC), hyperdiffusion chromatography, fluid or gel precipitin reactions, absorption spectroscopy, a colorimetric assays, spectrophotometric assays, flow cytometry, immunodiffusion (single or double), Immunoelectrophoresis, Western blotting, radioimmunoassay (RIA), enzyme-linked immunosorbent assays (ELISAs), immunofluorescent assays, electrochemiluminescence assays, and the like.

In some embodiments, the efficacy of the methods of the invention can be monitored by detecting or monitoring a reduction in a symptom of a HSD17B13 disease, such as biomarkers of liver disease, such as AST and ALT. These symptoms may be assessed in vitro or in vivo using any method known in the art.

In some embodiments of the methods of the invention, the RNAi construct is administered to a subject such that the RNAi construct is delivered to a specific site within the subject. The inhibition of expression of HSD17B13 may be assessed using measurements of the level or change in the level of HSD17B13 mRNA or HSD17B13 protein in a sample derived from fluid or tissue from the specific site within the subject. In preferred embodiments, the site is selected from the group consisting of liver, choroid plexus, retina, and pancreas. The site may also be a subsection or subgroup of cells from any one of the aforementioned sites. The site may also include cells that express a particular type of receptor.

### Methods of Treating or Preventing HSD17B13-Associated Diseases

The present invention provides therapeutic and prophylactic methods which include administering to a subject with a HSD17B13-associated disease, disorder, and/or condition, or prone to developing, a HSD17B13-associated disease, disorder, and/or condition, compositions comprising an RNAi construct, or pharmaceutical compositions comprising an RNAi construct, or vectors comprising an RNAi construct of the invention. Non-limiting examples of HSD17B13-associated diseases include, for example, fatty liver (steatosis), nonalcoholic steatohepatitis (NASH), cirrhosis of the liver, accumulation of fat in the liver, inflammation of the liver, hepatocellular necrosis, liver fibrosis, obesity, or nonalcoholic fatty liver disease (NAFLD). In one embodiment, the HSD17B13-associated disease is NAFLD. In another embodiment, the HSD17B13-associated disease is NASH. In another embodiment, the HSD17B13-associated disease is fatty liver (steatosis). In another embodiment, the HSD17B13-associated disease is insulin resistance. In another embodiment, the HSD17B13-associated disease is not insulin resistance.

In certain embodiments, the present invention provides a method for reducing the expression of HSD17B13 in a patient in need thereof comprising administering to the patient any of the RNAi constructs described herein. The term “patient,” as used herein, refers to a mammal, including humans, and can be used interchangeably with the term “subject.” Preferably, the expression level of HSD17B13 in hepatocytes in the patient is reduced following administration of the RNAi construct as compared to the HSD17B13 expression level in a patient not receiving the RNAi construct.

The methods of the invention are useful for treating a subject having a HSD17B13-associated disease, e.g., a subject that would benefit from reduction in HSD17B13 gene expression and/or HSD17B13 protein production. In one aspect, the present invention provides methods of reducing the level of 17β-Hydroxysteroid dehydrogenase type 13 (HSD17B13) gene expression in a subject having nonalcoholic fatty liver disease (NAFLD). In another aspect, the present invention provides methods of reducing the level of HSD17B13 protein in a subject with NAFLD.

In another aspect, the present invention provides methods of treating a subject having an NAFLD. In one aspect, the present invention provides methods of treating a subject having an HSD17B13-associated disease, e.g., fatty liver (steatosis), nonalcoholic steatohepatitis (NASH), cirrhosis of the liver, accumulation of fat in the liver, inflammation of the liver, hepatocellular necrosis, liver fibrosis, obesity, or nonalcoholic fatty liver disease (NAFLD). The treatment methods (and uses) of the invention include administering to the subject, e.g., a human, a therapeutically effective amount of an RNAi construct of the invention targeting a HSD17B13 gene or a pharmaceutical composition comprising an RNAi construct of the invention targeting a HSD17B13 gene or a vector of the invention comprising an RNAi construct targeting an HSD17B13 gene.

In one aspect, the invention provides methods of preventing at least one symptom in a subject having NAFLD, e.g., the presence of elevated hedgehog signaling pathways, fatigue, weakness, weight loss, loss of apetite, nausea, abdominal pain, spider-like blood vessels, yellowing of the skin and eyes (jaundice), itching, fluid build up and swelling of the legs (edema), abdomen swelling (ascites), and mental confusion. The methods include administering to the subject a therapeutically effective amount of the RNAi construct, e.g. dsRNA, pharmaceutical compositions, or vectors of the invention, thereby preventing at least one symptom in the subject having a disorder that would benefit from reduction in HSD17B13 gene expression.

In another aspect, the present invention provides uses of a therapeutically effective amount of an RNAi construct of the invention for treating a subject, e.g., a subject that would benefit from a reduction and/or inhibition of HSD17B13 gene expression. In a further aspect, the present invention provides uses of an RNAi construct, e.g., a dsRNA, of the invention targeting an HSD17B13 gene or pharmaceutical composition comprising an RNAi construct targeting an HSD17B13 gene in the manufacture of a medicament for treating a subject, e.g., a subject that would benefit from a reduction and/or inhibition of HSD17B13 gene expression and/or HSD17B13 protein production, such as a subject having a disorder that would benefit from reduction in HSD17B13 gene expression, e.g., a HSD17B13-associated disease.

In another aspect, the invention provides uses of an RNAi, e.g., a dsRNA, of the invention for preventing at least one symptom in a subject suffering from a disorder that would benefit from a reduction and/or inhibition of HSD17B13 gene expression and/or HSD17B13 protein production.

In a further aspect, the present invention provides uses of an RNAi construct of the invention in the manufacture of a medicament for preventing at least one symptom in a subject suffering from a disorder that would benefit from a reduction and/or inhibition of HSD17B13 gene expression and/or HSD17B13 protein production, such as a HSD17B13-associated disease.

In one embodiment, an RNAi construct targeting HSD17B13 is administered to a subject having a HSD17B13-associated disease, e.g., nonalcoholic fatty liver disease (NAFLD), such that the expression of a HSD17B13 gene, e.g., in a cell, tissue, blood or other tissue or fluid of the subject are reduced by at least about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 62%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least about 99% or more when the dsRNA agent is administered to the subject.

The methods and uses of the invention include administering a composition described herein such that expression of the target HSD17B13 gene is decreased, such as for about 1, 2, 3, 4 5, 6, 7, 8, 12, 16, 18, 24, 28, 32, 36, 40, 44, 48, 52, 56, 60, 64, 68, 72, 76, or about 80 hours. In one embodiment, expression of the target HSD17B13 gene is decreased for an extended duration, e.g., at least about two, three, four, five, six, seven days or more, e.g., about one week, two weeks, three weeks, or about four weeks or longer.

Administration of the dsRNA according to the methods and uses of the invention may result in a reduction of the severity, signs, symptoms, and/or markers of such diseases or disorders in a patient with a HSD17B13-associated disease, e.g., nonalcoholic fatty liver disease (NAFLD). By “reduction” in this context is meant a statistically significant decrease in such level. The reduction can be, for example, at least about 5%, 10%, 15%, 20%, 25%, 30%, 35%, 40%, 45%, 50%, 55%, 60%, 65%, 70%, 75%, 80%, 85%, 90%, 95%, or about 100%. Efficacy of treatment or prevention of disease can be assessed, for example by measuring disease progression, disease remission, symptom severity, reduction in pain, quality of life, dose of a medication required to sustain a treatment effect, level of a disease marker or any other measurable parameter appropriate for a given disease being treated or targeted for prevention. It is well within the ability of one skilled in the art to monitor efficacy of treatment or prevention by measuring any one of such parameters, or any combination of parameters. For example, efficacy of treatment of NAFLD may be assessed, for example, by periodic monitoring of NAFLD symptoms, liver fat levels, or expression of downstream genes. Comparison of the later readings with the initial readings provide a physician an indication of whether the treatment is effective. It is well within the ability of one skilled in the art to monitor efficacy of treatment or prevention by measuring any one of such parameters, or any combination of parameters. In connection with the administration of an RNAi construct targeting HSD17B13 or pharmaceutical composition thereof, “effective against” an HSD17B13-associated disease indicates that administration in a clinically appropriate manner results in a beneficial effect for at least a statistically significant fraction of patients, such as improvement of symptoms, a cure, a reduction in disease, extension of life, improvement in quality of life, or other effect generally recognized as positive by medical doctors familiar with treating NAFLD and/or an HSD17B13-associated disease and the related causes.

A treatment or preventive effect is evident when there is a statistically significant improvement in one or more parameters of disease status, or by a failure to worsen or to develop symptoms where they would otherwise be anticipated. As an example, a favorable change of at least 10% in a measurable parameter of disease, and preferably at least 20%, 30%, 40%, 50% or more can be indicative of effective treatment. Efficacy for a given RNAi drug or formulation of that drug can also be judged using an experimental animal model for the given disease as known in the art. When using an experimental animal model, efficacy of treatment is evidenced when a statistically significant reduction in a marker or symptom is observed.

Administration of the RNAi construct can reduce the presence of HSD17B13 protein levels, e.g., in a cell, tissue, blood, urine or other compartment of the patient by at least about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least about 99% or more.

Before administration of a full dose of the RNAi construct, patients can be administered a smaller dose, such as a 5% infusion, and monitored for adverse effects, such as an allergic reaction. In another example, the patient can be monitored for unwanted immunostimulatory effects, such as increased cytokine (e.g., TNF-alpha or INF-alpha) levels.

Owing to the inhibitory effects on HSD17B13 expression, a composition according to the invention or a pharmaceutical composition prepared therefrom can enhance the quality of life.

An RNAi construct of the invention may be administered in “naked” form, where the modified or unmodified RNAi construct is directly suspended in aqueous or suitable buffer solvent, as a “free RNAi.” A free RNAi is administered in the absence of a pharmaceutical composition.

Alternatively, an RNAi of the invention may be administered as a pharmaceutical composition, such as a dsRNA liposomal formulation.

Subjects that would benefit from a reduction and/or inhibition of HSD17B13 gene expression are those having nonalcoholic fatty liver disease (NAFLD) and/or an HSD17B13-associated disease or disorder as described herein.

Treatment of a subject that would benefit from a reduction and/or inhibition of HSD17B13 gene expression includes therapeutic and prophylactic treatment.

The invention further provides methods and uses of an RNAi construct or a pharmaceutical composition thereof for treating a subject that would benefit from reduction and/or inhibition of HSD17B13 gene expression, e.g., a subject having a HSD17B13-associated disease, in combination with other pharmaceuticals and/or other therapeutic methods, e.g., with known pharmaceuticals and/or known therapeutic methods, such as, for example, those which are currently employed for treating these disorders.

For example, in certain embodiments, an RNAi construct targeting a HSD17B13 gene is administered in combination with, e.g., an agent useful in treating an HSD17B13-associated disease as described elsewhere herein. For example, additional therapeutics and therapeutic methods suitable for treating a subject that would benefit from reduction in HSD17B13 expression, e.g., a subject having a HSD17B13-associated disease, include an RNAi construct targeting a different portion of the HSD17B13 gene, a therapeutic agent, and/or procedures for treating a HSD17B13-associated disease or a combination of any of the foregoing.

In certain embodiments, a first RNAi construct targeting a HSD17B13 gene is administered in combination with a second RNAi construct targeting a different portion of the HSD17B13 gene. For example, the first RNAi construct comprises a first sense strand and a first antisense strand forming a double stranded region, wherein substantially all of the nucleotides of said first sense strand and substantially all of the nucleotides of the first antisense strand are modified nucleotides, wherein said first sense strand is conjugated to a ligand attached at the 3′-terminus, and wherein the ligand is one or more GalNAc derivatives attached through a bivalent or trivalent branched linker; and the second RNAi construct comprises a second sense strand and a second antisense strand forming a double stranded region, wherein substantially all of the nucleotides of the second sense strand and substantially all of the nucleotides of the second antisense strand are modified nucleotides, wherein the second sense strand is conjugated to a ligand attached at the 3′-terminus, and wherein the ligand is one or more GalNAc derivatives attached through a bivalent or trivalent branched linker.

In one embodiment, all of the nucleotides of the first and second sense strand and/or all of the nucleotides of the first and second antisense strand comprise a modification.

In one embodiment, the at least one of the modified nucleotides is selected from the group consisting of a 3′-terminal deoxy-thymine (dT) nucleotide, a 2′-O-methyl modified nucleotide, a 2′-fluoro modified nucleotide, a locked nucleotide, an unlocked nucleotide, a conformationally restricted nucleotide, a constrained ethyl nucleotide, an abasic nucleotide, a 2′-amino-modified nucleotide, a 2′-O-allyl-modified nucleotide, 2′-C-alkyl-modified nucleotide, 2′-hydroxly-modified nucleotide, a 2′-methoxyethyl modified nucleotide, a 2′-O-alkyl-modified nucleotide, a morpholino nucleotide, a phosphoramidate, a non-natural base comprising nucleotide, a tetrahydropyran modified nucleotide, a 1,5-anhydrohexitol modified nucleotide, a cyclohexenyl modified nucleotide, a nucleotide comprising a phosphorothioate group, a nucleotide comprising a methylphosphonate group, a nucleotide comprising a 5 ‘-phosphate, and a nucleotide comprising a 5’-phosphate mimic.

In certain embodiments, a first RNAi construct targeting a HSD17B13 gene is administered in combination with a second RNAi construct targeting a gene that is different from the HSD17B13 gene. For example, the RNAi construct targeting the HSD17B13 gene may be administered in combination with an RNAi construct targeting the SCAP gene. The first RNAi construct targeting a HSD17B13 gene and the second RNAi construct targeting a gene different from the HSD17B13 gene, e.g., the SCAP gene, may be administered as parts of the same pharmaceutical composition. Alternatively, the first RNAi construct targeting a HSD17B13 gene and the second RNAi construct targeting a gene different from the HSD17B13 gene, e.g., the SCAP gene, may be administered as parts of different pharmaceutical compositions.

The RNAi construct and an additional therapeutic agent and/or treatment may be administered at the same time and/or in the same combination, e.g., parenterally, or the additional therapeutic agent can be administered as part of a separate composition or at separate times and/or by another method known in the art or described herein.

The present invention also provides methods of using an RNAi construct of the invention and/or a composition containing an RNAi construct of the invention to reduce and/or inhibit HSD17B13 expression in a cell. In other aspects, the present invention provides an RNAi construct of the invention and/or a composition comprising an RNAi construct of the invention for use in reducing and/or inhibiting HSD17B13 gene expression in a cell. In yet other aspects, use of an RNAi of the invention and/or a composition comprising an RNAi of the invention for the manufacture of a medicament for reducing and/or inhibiting HSD17B13 gene expression in a cell are provided. In still other aspects, the the present invention provides an RNAi of the invention and/or a composition comprising an RNAi of the invention for use in reducing and/or inhibiting HSD17B13 protein production in a cell. In yet other aspects, use of an RNAi of the invention and/or a composition comprising an RNAi of the invention for the manufacture of a medicament for reducing and/or inhibiting HSD17B13 protein production in a cell are provided. The methods and uses include contacting the cell with an RNAi construct, e.g., a dsRNA, of the invention and maintaining the cell for a time sufficient to obtain degradation of the mRNA transcript of an HSD17B13 gene, thereby inhibiting expression of the HSD17B13 gene or inhibiting HSD17B13 protein production in the cell.

Reduction in gene expression can be assessed by any methods known in the art. For example, a reduction in the expression of HSD17B13 may be determined by determining the mRNA expression level of HSD17B13 using methods routine to one of ordinary skill in the art, e.g., Northern blotting, qRT-PCR, by determining the protein level of HSD17B13 using methods routine to one of ordinary skill in the art, such as Western blotting, immunological techniques, flow cytometry methods, ELISA, and/or by determining a biological activity of HSD17B13.

In the methods and uses of the invention the cells may be contacted in vitro or in vivo, i.e., the cell may be within a subject.

A cell suitable for treatment using the methods of the invention may be any cell that expresses the HSD17B13 gene, e.g., a cell from a subject having NAFLD or a cell comprising an expression vector comprising a HSD17B13 gene or portion of a HSD17B13 gene. A cell suitable for use in the methods and uses of the invention may be a mammalian cell, e.g., a primate cell (such as a human cell or a non-human primate cell, e.g., a monkey cell or a chimpanzee cell), a non-primate cell (such as a cow cell, a pig cell, a camel cell, a llama cell, a horse cell, a goat cell, a rabbit cell, a sheep cell, a hamster, a guinea pig cell, a cat cell, a dog cell, a rat cell, a mouse cell, a lion cell, a tiger cell, a bear cell, or a buffalo cell), a bird cell (e.g., a duck cell or a goose cell), or a whale cell. In one embodiment, the cell is a human cell.

HSD17B13 gene expression may be inhibited in the cell by at least about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or about 100%.

HSD17B13 protein production may be inhibited in the cell by at least about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or about 100%.

The in vivo methods and uses of the invention may include administering to a subject a composition containing an RNAi construct, where the RNAi construct includes a nucleotide sequence that is complementary to at least a part of an RNA transcript of the HSD17B13 gene of the mammal to be treated. When the organism to be treated is a human, the composition can be administered by any means known in the art including, but not limited to subcutaneous, intravenous, oral, intraperitoneal, or parenteral routes, including intracranial (e.g., intraventricular, intraparenchymal and intrathecal), intramuscular, transdermal, airway (aerosol), nasal, rectal, and topical (including buccal and sublingual) administration. In certain embodiments, the compositions are administered by subcutaneous or intravenous infusion or injection. In one embodiment, the compositions are administered by subcutaneous injection.

In some embodiments, the administration is via a depot injection. A depot injection may release the RNAi in a consistent way over a prolonged time period. Thus, a depot injection may reduce the frequency of dosing needed to obtain a desired effect, e.g., a desired inhibition of HSD17B13, or a therapeutic or prophylactic effect. A depot injection may also provide more consistent serum concentrations. Depot injections may include subcutaneous injections or intramuscular injections. In preferred embodiments, the depot injection is a subcutaneous injection.

In some embodiments, the administration is via a pump. The pump may be an external pump or a surgically implanted pump. In certain embodiments, the pump is a subcutaneously implanted osmotic pump. In other embodiments, the pump is an infusion pump. An infusion pump may be used for intravenous, subcutaneous, arterial, or epidural infusions. In preferred embodiments, the infusion pump is a subcutaneous infusion pump. In other embodiments, the pump is a surgically implanted pump that delivers the RNAi construct to the subject.

The mode of administration may be chosen based upon whether local or systemic treatment is desired and based upon the area to be treated. The route and site of administration may be chosen to enhance targeting.

In one aspect, the present invention also provides methods for inhibiting the expression of an HSD17B13 gene in a mammal, e.g., a human. The present invention also provides a composition comprising an RNAi construct, e.g., a dsRNA, that targets an HSD17B13 gene in a cell of a mammal for use in inhibiting expression of the HSD17B13 gene in the mammal. In another aspect, the present invention provides use of an RNAi, e.g., a dsRNA, that targets an HSD17B13 gene in a cell of a mammal in the manufacture of a medicament for inhibiting expression of the HSD17B13 gene in the mammal.

The methods and uses include administering to the mammal, e.g., a human, a composition comprising an RNAi, e.g., a dsRNA, that targets an HSD17B13 gene in a cell of the mammal and maintaining the mammal for a time sufficient to obtain degradation of the mRNA transcript of the HSD17B13 gene, thereby inhibiting expression of the HSD17B13 gene in the mammal.

Reduction in gene expression can be assessed in peripheral blood sample of the RNAi-administered subject by any methods known it the art, e.g. qRT-PCR, described herein. Reduction in protein production can be assessed by any methods known it the art and by methods, e.g., ELISA or Western blotting, described herein. In one embodiment, a tissue sample serves as the tissue material for monitoring the reduction in HSD17B13 gene and/or protein expression. In another embodiment, a blood sample serves as the tissue material for monitoring the reduction in HSD17B13 gene and/or protein expression.

In one embodiment, verification of RISC medicated cleavage of target in vivo following administration of RNAi construct is done by performing 5′-RACE or modifications of the protocol as known in the art (Lasham A et al., (2010) Nucleic Acid Res., 38 (3) p-e19) (Zimmermann et al. (2006) Nature 441: 111-4).

It is understood that all ribonucleic acid sequences disclosed herein can be converted to deoxyribonucleic acid sequences by substituting a thymine base for a uracil base in the sequence. Likewise, all deoxyribonucleic acid sequences disclosed herein can be converted to ribonucleic acid sequences by substituting a uracil base for a thymine base in the sequence. Deoxyribonucleic acid sequences, ribonucleic acid sequences, and sequences containing mixtures of deoxyribonucleotides and ribonucleotides of all sequences disclosed herein are included in the invention.

Additionally, any nucleic acid sequences disclosed herein may be modified with any combination of chemical modifications. One of skill in the art will readily appreciate that such designation as “RNA” or “DNA” to describe modified polynucleotides is, in certain instances, arbitrary. For example, a polynucleotide comprising a nucleotide having a 2′-OH substituent on the ribose sugar and a thymine base could be described as a DNA molecule having a modified sugar (2′-OH for the natural 2′-H of DNA) or as an RNA molecule having a modified base (thymine (methylated uracil) for natural uracil of RNA).

Accordingly, nucleic acid sequences provided herein, including, but not limited to those in the sequence listing, are intended to encompass nucleic acids containing any combination of natural or modified RNA and/or DNA, including, but not limited to such nucleic acids having modified nucleobases. By way of a further example and without limitation, a polynucleotide having the sequence “ATCGATCG” encompasses any polynucleotides having such a sequence, whether modified or unmodified, including, but not limited to, such compounds comprising RNA bases, such as those having sequence “AUCGAUCG” and those having some DNA bases and some RNA bases such as “AUCGATCG” and polynucleotides having other modified bases, such as “ATmeCGAUCG,” wherein meC indicates a cytosine base comprising a methyl group at the 5-position.

The following examples, including the experiments conducted and the results achieved, are provided for illustrative purposes only and are not to be construed as limiting the scope of the appended claims.

### INCORPORATION BY REFERENCE

All publications, patents, and patent applications mentioned in this specification are herein incorporated by reference to the same extent as if each individual publication, patent, or patent application was specifically and individually indicated to be incorporated by reference. However, the citation of a reference herein should not be construed as an acknowledgement that such reference is prior art to the present invention. To the extent that any of the definitions or terms provided in the references incorporated by reference differ from the terms and discussion provided herein, the present terms and definitions control.

### EQUIVALENTS

The foregoing written specification is considered to be sufficient to enable one skilled in the art to practice the invention. The foregoing description and examples detail certain preferred embodiments of the invention and describe the best mode contemplated by the inventors. It will be appreciated, however, that no matter how detailed the foregoing may appear in text, the invention may be practiced in many ways and the invention should be construed in accordance with the appended claims and any equivalents thereof.

The following examples, including the experiments conducted and results achieved, are provided for illustrative purposes only and are not to be construed as limiting the present invention.

All animal experiments described herein were approved by the Institutional Animal Care and Use Committee (IACUC) of Amgen and cared for in accordance to the Guide for the Care and Use of Laboratory Animals, 8th Edition (National Research Council (U.S.). Committee for the Update of the Guide for the Care and Use of Laboratory Animals., Institute for Laboratory Animal Research (U.S.), and National Academies Press (U.S.) (2011) Guide for the care and use of laboratory animals. 8th Ed., National Academies Press, Washington, D.C. Mice were single-housed in an air-conditioned room at 22±2° C. with a twelve-hour light; twelve-hour darkness cycle (0600-1800 hours). Animals had ad libitum access to a regular chow diet (Envigo, 2920X, or a diet as stated otherwise) and to water (reverse osmosis-purified) via automatic watering system, unless otherwise indicated. At termination, blood was collected by cardiac puncture under deep anesthesia, and then, following Association for Assessment and Accreditation of Laboratory Animal Care (AAALAC) guidelines, euthanized by a secondary physical method.

### Example 1: Selection, Design and Synthesis of Modified HSD17B13 siRNA Molecules

The identification and selection of optimal sequences for therapeutic siRNA molecules targeting 17β-Hydroxysteroid dehydrogenase type 13 (HSD17B13) were identified using bioinformatics analysis of a human HSD17B13 transcript (NM_178135.4 or NM_001136230.2). Table 1 shows sequences identified as having therapeutic properties. Throughout the various sequences, {INVAB} is an inverted abasic, {INVDA} is an inverted deoxyadenosine, GNA is a glycol nucleic acid, dT is deoxythymidine and dC is deoxycytosine.

TABLE 1
siRNA sequences directed to HSD17B13

| | | SEQ | | SEQ ID |
|---|---|---|---|---|
| | | ID | | NO: |
| DUPLEX | | NO: | | (ANTI |
| NO. | SENSE SEQUENCE (5′-3′) | (SENSE) | ANTISENSE SEQUENCE (5′-3′) | SENSE) |
| D-1000 | UUCUGCUUCUGAUCACCAUC{INVAB} | 1 | UGAUGGUGAUCAGAAGCAGAAUU | 2 |
| D-1001 | UCUGCUUCUGAUCACCAUCA{INVAB} | 3 | AUGAUGGUGAUCAGAAGCAGAUU | 4 |
| D-1002 | CUUCUGAUCACCAUCAUCUA{INVAB} | 5 | AUAGAUGAUGGUGAUCAGAAGUU | 6 |
| D-1003 | UCUCAUUACUGGAGCUGGGC{INVAB} | 7 | UGCCCAGCUCCAGUAAUGAGAUU | 8 |
| D-1004 | GUGAAUAAUGCUGGGACAGU{INVAB} | 9 | UACUGUCCCAGCAUUAUUCACUU | 10 |
| D-1005 | UAAUGCUGGGACAGUAUAUC{INVAB} | 11 | AGAUAUACUGUCCCAGCAUUAUU | 12 |
| D-1006 | AAUGCUGGGACAGUAUAUCC{INVAB} | 13 | UGGAUAUACUGUCCCAGCAUUUU | 14 |
| D-1007 | GGGACAGUAUAUCCAGCCGA{INVAB} | 15 | AUCGGCUGGAUAUACUGUCCCUU | 16 |
| D-1008 | GGACAGUAUAUCCAGCCGAU{INVAB} | 17 | AAUCGGCUGGAUAUACUGUCCUU | 18 |
| D-1009 | GACAGUAUAUCCAGCCGAUC{INVAB} | 19 | AGAUCGGCUGGAUAUACUGUCUU | 20 |
| D-1010 | ACAGUAUAUCCAGCCGAUCU{INVAB} | 21 | AAGAUCGGCUGGAUAUACUGUUU | 22 |
| D-1011 | CAGUAUAUCCAGCCGAUCUU{INVAB} | 23 | AAAGAUCGGCUGGAUAUACUGUU | 24 |
| D-1012 | GACAUUUGAGGUCAACAUCC{INVAB} | 25 | AGGAUGUUGACCUCAAAUGUCUU | 26 |
| D-1013 | UGAGGUCAACAUCCUAGGAC{INVAB} | 27 | UGUCCUAGGAUGUUGACCUCAUU | 28 |
| D-1014 | AGGUCAACAUCCUAGGACAU{INVAB} | 29 | AAUGUCCUAGGAUGUUGACCUUU | 30 |
| D-1015 | GGUCAACAUCCUAGGACAUU{INVAB} | 31 | AAAUGUCCUAGGAUGUUGACCUU | 32 |
| D-1016 | GUCAACAUCCUAGGACAUUU{INVAB} | 33 | AAAAUGUCCUAGGAUGUUGACUU | 34 |
| D-1017 | UCAACAUCCUAGGACAUUUU{INVAB} | 35 | AAAAAUGUCCUAGGAUGUUGAUU | 36 |
| D-1018 | CAACAUCCUAGGACAUUUUU{INVAB} | 37 | AAAAAAUGUCCUAGGAUGUUGUU | 38 |
| D-1019 | CAAAAGCACUUCUUCCAUCG{INVAB} | 39 | UCGAUGGAAGAAGUGCUUUUGUU | 40 |
| D-1020 | AAAAGCACUUCUUCCAUCGA{INVAB} | 41 | AUCGAUGGAAGAAGUGCUUUUUU | 42 |
| D-1021 | AAAGCACUUCUUCCAUCGAU{INVAB} | 43 | AAUCGAUGGAAGAAGUGCUUUUU | 44 |
| D-1022 | AAGCACUUCUUCCAUCGAUG{INVAB} | 45 | UCAUCGAUGGAAGAAGUGCUUUU | 46 |
| D-1023 | AGCACUUCUUCCAUCGAUGA{INVAB} | 47 | AUCAUCGAUGGAAGAAGUGCUUU | 48 |
| D-1024 | UUCCUUACCUCAUCCCAUAU{INVAB} | 49 | AAUAUGGGAUGAGGUAAGGAAUU | 50 |
| D-1025 | CCUUACCUCAUCCCAUAUUG{INVAB} | 51 | ACAAUAUGGGAUGAGGUAAGGUU | 52 |
| D-1026 | ACCUCAUCCCAUAUUGUUCC{INVAB} | 53 | UGGAACAAUAUGGGAUGAGGUUU | 54 |
| D-1027 | CCUCAUCCCAUAUUGUUCCA{INVAB} | 55 | AUGGAACAAUAUGGGAUGAGGUU | 56 |
| D-1028 | UCCCAUAUUGUUCCAGCAAA{INVAB} | 57 | AUUUGCUGGAACAAUAUGGGAUU | 58 |
| D-1029 | GGCUUUCACAGAGGUCUGAC{INVAB} | 59 | UGUCAGACCUCUGUGAAAGCCUU | 60 |
| D-1030 | UUUGUGAAUACUGGGUUCAC{INVAB} | 61 | AGUGAACCCAGUAUUCACAAAUU | 62 |
| D-1031 | UUGUGAAUACUGGGUUCACC{INVAB} | 63 | UGGUGAACCCAGUAUUCACAAUU | 64 |
| D-1032 | GAAUACUGGGUUCACCAAAA{INVAB} | 65 | UUUUUGGUGAACCCAGUAUUCUU | 66 |
| D-1033 | AUACUGGGUUCACCAAAAAU{INVAB} | 67 | AAUUUUUGGUGAACCCAGUAUUU | 68 |
| D-1034 | UACUGGGUUCACCAAAAAUC{INVAB} | 69 | AGAUUUUUGGUGAACCCAGUAUU | 70 |
| D-1035 | UUUUAAAUCGUAUGCAGAAU{INVAB} | 71 | UAUUCUGCAUACGAUUUAAAAUU | 72 |
| D-1036 | UUUAAAUCGUAUGCAGAAUA{INVAB} | 73 | AUAUUCUGCAUACGAUUUAAAUU | 74 |
| D-1037 | UAAAUCGUAUGCAGAAUAUU{INVAB} | 75 | AAAUAUUCUGCAUACGAUUUAUU | 76 |
| D-1038 | AAAUCGUAUGCAGAAUAUUC{INVAB} | 77 | UGAAUAUUCUGCAUACGAUUUUU | 78 |
| D-1039 | AAUCGUAUGCAGAAUAUUCA{INVAB} | 79 | UUGAAUAUUCUGCAUACGAUUUU | 80 |
| D-1040 | UCGUAUGCAGAAUAUUCAAU{INVAB} | 81 | AAUUGAAUAUUCUGCAUACGAUU | 82 |
| D-1041 | CGUAUGCAGAAUAUUCAAUU{INVAB} | 83 | AAAUUGAAUAUUCUGCAUACGUU | 84 |
| D-1042 | UAUGCAGAAUAUUCAAUUUG{INVAB} | 85 | UCAAAUUGAAUAUUCUGCAUAUU | 86 |
| D-1043 | AAUAUUCAAUUUGAAGCAGU{INVAB} | 87 | AACUGCUUCAAAUUGAAUAUUUU | 88 |
| D-1044 | AAAUGAAAUGAAUAAAUAAG{INVAB} | 89 | ACUUAUUUAUUCAUUUCAUUUUU | 90 |
| D-1045 | AAUCAAUGCUGCAAAGCUUU{INVAB} | 91 | UAAAGCUUUGCAGCAUUGAUUUU | 92 |
| D-1046 | UGCUGCAAAGCUUUAUUUCA{INVAB} | 93 | AUGAAAUAAAGCUUUGCAGCAUU | 94 |
| D-1047 | GCUGCAAAGCUUUAUUUCAC{INVAB} | 95 | UGUGAAAUAAAGCUUUGCAGCUU | 96 |
| D-1048 | UUAAAAACAUUGGUUUGGCA{INVAB} | 97 | AUGCCAAACCAAUGUUUUUAAUU | 98 |
| D-1049 | AAAAACAUUGGUUUGGCACU{INVAB} | 99 | UAGUGCCAAACCAAUGUUUUUUU | 100 |
| D-1050 | AACAAGAUUAAUUACCUGUC{INVAB} | 101 | AGACAGGUAAUUAAUCUUGUUUU | 102 |
| D-1051 | CAAGAUUAAUUACCUGUCUU{INVAB} | 103 | AAAGACAGGUAAUUAAUCUUGUU | 104 |
| D-1052 | UAAUUACCUGUCUUCCUGUU{INVAB} | 105 | AAACAGGAAGACAGGUAAUUAUU | 106 |
| D-1053 | CCUGUCUUCCUGUUUCUCAA{INVAB} | 107 | AUUGAGAAACAGGAAGACAGGUU | 108 |
| D-1054 | UUUCCUUUCAUGCCUCUUAA{INVAB} | 109 | UUUAAGAGGCAUGAAAGGAAAUU | 110 |
| D-1055 | UUCCUUUCAUGCCUCUUAAA{INVAB} | 111 | UUUUAAGAGGCAUGAAAGGAAUU | 112 |
| D-1056 | UUUUCCAUUUAAAGGUGGAC{INVAB} | 113 | UGUCCACCUUUAAAUGGAAAAUU | 114 |
| D-1057 | UUUCCAUUUAAAGGUGGACA{INVAB} | 115 | UUGUCCACCUUUAAAUGGAAAUU | 116 |
| D-1058 | UUCCAUUUAAAGGUGGACAA{INVAB} | 117 | UUUGUCCACCUUUAAAUGGAAUU | 118 |
| D-1059 | UCCAUUUAAAGGUGGACAAA{INVAB} | 119 | UUUUGUCCACCUUUAAAUGGAUU | 120 |
| D-1060 | GAACUUAUUUACACAGGGAA{INVAB} | 121 | AUUCCCUGUGUAAAUAAGUUCUU | 122 |
| D-1061 | CUUAUUUACACAGGGAAGGU{INVAB} | 123 | AACCUUCCCUGUGUAAAUAAGUU | 124 |
| D-1062 | AUUUACACAGGGAAGGUUUA{INVAB} | 125 | UUAAACCUUCCCUGUGUAAAUUU | 126 |
| D-1063 | UUUACACAGGGAAGGUUUAA{INVAB} | 127 | AUUAAACCUUCCCUGUGUAAAUU | 128 |
| D-1064 | CAGGGAAGGUUUAAGACUGU{INVAB} | 129 | AACAGUCUUAAACCUUCCCUGUU | 130 |
| D-1065 | GGAAGGUUUAAGACUGUUCA{INVAB} | 131 | UUGAACAGUCUUAAACCUUCCUU | 132 |
| D-1066 | AGGUUUAAGACUGUUCAAGU{INVAB} | 133 | UACUUGAACAGUCUUAAACCUUU | 134 |
| D-1067 | GGUUUAAGACUGUUCAAGUA{INVAB} | 135 | AUACUUGAACAGUCUUAAACCUU | 136 |
| D-1068 | AGACUGUUCAAGUAGCAUUC{INVAB} | 137 | AGAAUGCUACUUGAACAGUCUUU | 138 |
| D-1069 | GACUGUUCAAGUAGCAUUCC{INVAB} | 139 | UGGAAUGCUACUUGAACAGUCUU | 140 |
| D-1070 | ACUGUUCAAGUAGCAUUCCA{INVAB} | 141 | UUGGAAUGCUACUUGAACAGUUU | 142 |
| D-1071 | CUGUUCAAGUAGCAUUCCAA{INVAB} | 143 | AUUGGAAUGCUACUUGAACAGUU | 144 |
| D-1072 | UGUUCAAGUAGCAUUCCAAU{INVAB} | 145 | AAUUGGAAUGCUACUUGAACAUU | 146 |
| D-1073 | CAAGAACACAGAAUGAGUGC{INVAB} | 147 | UGCACUCAUUCUGUGUUCUUGUU | 148 |
| D-1074 | ACAGAAUGAGUGCACAGCUA{INVAB} | 149 | UUAGCUGUGCACUCAUUCUGUUU | 150 |
| D-1075 | AGGCAGCUUUAUCUCAACCU{INVAB} | 151 | AAGGUUGAGAUAAAGCUGCCUUU | 152 |
| D-1076 | UUUUAAGAUUCAGCAUUUGA{INVAB} | 153 | UUCAAAUGCUGAAUCUUAAAAUU | 154 |
| D-1077 | AGAUUCAGCAUUUGAAAGAU{INVAB} | 155 | AAUCUUUCAAAUGCUGAAUCUUU | 156 |
| D-1078 | AUUUGAAAGAUUUCCCUAGC{INVAB} | 157 | AGCUAGGGAAAUCUUUCAAAUUU | 158 |
| D-1079 | UUCCCUAGCCUCUUCCUUUU{INVAB} | 159 | AAAAAGGAAGAGGCUAGGGAAUU | 160 |
| D-1080 | CUAUUCUGGACUUUAUUACU{INVAB} | 161 | AAGUAAUAAAGUCCAGAAUAGUU | 162 |
| D-1081 | AGUCCACCAAAAGUGGACCC{INVAB} | 163 | AGGGUCCACUUUUGGUGGACUUU | 164 |
| D-1082 | CACCAAAAGUGGACCCUCUA{INVAB} | 165 | AUAGAGGGUCCACUUUUGGUGUU | 166 |
| D-1083 | ACCAAAAGUGGACCCUCUAU{INVAB} | 167 | UAUAGAGGGUCCACUUUUGGUUU | 168 |
| D-1084 | CAAAAGUGGACCCUCUAUAU{INVAB} | 169 | AAUAUAGAGGGUCCACUUUUGUU | 170 |
| D-1085 | AAAAGUGGACCCUCUAUAUU{INVAB} | 171 | AAAUAUAGAGGGUCCACUUUUUU | 172 |
| D-1086 | AAAGUGGACCCUCUAUAUUU{INVAB} | 173 | AAAAUAUAGAGGGUCCACUUUUU | 174 |
| D-1087 | AAGUGGACCCUCUAUAUUUC{INVAB} | 175 | AGAAAUAUAGAGGGUCCACUUUU | 176 |
| D-1088 | UUCAUAUAUCCUUGGUCCCA{INVAB} | 177 | AUGGGACCAAGGAUAUAUGAAUU | 178 |
| D-1089 | GAUGUUUAGACAAUUUUAGG{INVAB} | 179 | ACCUAAAAUUGUCUAAACAUCUU | 180 |
| D-1090 | AUGUUUAGACAAUUUUAGGC{INVAB} | 181 | AGCCUAAAAUUGUCUAAACAUUU | 182 |
| D-1091 | UGUUUAGACAAUUUUAGGCU{INVAB} | 183 | AAGCCUAAAAUUGUCUAAACAUU | 184 |
| D-1092 | GUUUAGACAAUUUUAGGCUC{INVAB} | 185 | UGAGCCUAAAAUUGUCUAAACUU | 186 |
| D-1093 | UUUAGACAAUUUUAGGCUCA{INVAB} | 187 | UUGAGCCUAAAAUUGUCUAAAUU | 188 |
| D-1094 | UUAGACAAUUUUAGGCUCAA{INVAB} | 189 | UUUGAGCCUAAAAUUGUCUAAUU | 190 |
| D-1095 | AGACAAUUUUAGGCUCAAAA{INVAB} | 191 | UUUUUGAGCCUAAAAUUGUCUUU | 192 |
| D-1096 | AUUUUAGGCUCAAAAAUUAA{INVAB} | 193 | UUUAAUUUUUGAGCCUAAAAUUU | 194 |
| D-1097 | UUUAGGCUCAAAAAUUAAAG{INVAB} | 195 | ACUUUAAUUUUUGAGCCUAAAUU | 196 |
| D-1098 | UUAGGCUCAAAAAUUAAAGC{INVAB} | 197 | AGCUUUAAUUUUUGAGCCUAAUU | 198 |
| D-1099 | AAAAAUUAAAGCUAACACAG{INVAB} | 199 | ACUGUGUUAGCUUUAAUUUUUUU | 200 |
| D-1100 | AAAAUUAAAGCUAACACAGG{INVAB} | 201 | UCCUGUGUUAGCUUUAAUUUUUU | 202 |
| D-1101 | AAAGCUAACACAGGAAAAGG{INVAB} | 203 | UCCUUUUCCUGUGUUAGCUUUUU | 204 |
| D-1102 | AAGCUAACACAGGAAAAGGA{INVAB} | 205 | UUCCUUUUCCUGUGUUAGCUUUU | 206 |
| D-1103 | GGAAAAGGAACUGUACUGGC{INVAB} | 207 | AGCCAGUACAGUUCCUUUUCCUU | 208 |
| D-1104 | AGGAACUGUACUGGCUAUUA{INVAB} | 209 | AUAAUAGCCAGUACAGUUCCUUU | 210 |
| D-1105 | CCGACUCCCACUACAUCAAG{INVAB} | 211 | UCUUGAUGUAGUGGGAGUCGGUU | 212 |
| D-1106 | GACUCCCACUACAUCAAGAC{INVAB} | 213 | AGUCUUGAUGUAGUGGGAGUCUU | 214 |
| D-1107 | UCCCACUACAUCAAGACUAA{INVAB} | 215 | AUUAGUCUUGAUGUAGUGGGAUU | 216 |
| D-1108 | CCCACUACAUCAAGACUAAU{INVAB} | 217 | AAUUAGUCUUGAUGUAGUGGGUU | 218 |
| D-1109 | CACUACAUCAAGACUAAUCU{INVAB} | 219 | AAGAUUAGUCUUGAUGUAGUGUU | 220 |
| D-1110 | ACUACAUCAAGACUAAUCUU{INVAB} | 221 | AAAGAUUAGUCUUGAUGUAGUUU | 222 |
| D-1111 | CUACAUCAAGACUAAUCUUG{INVAB} | 223 | ACAAGAUUAGUCUUGAUGUAGUU | 224 |
| D-1112 | GUUUUUCACAUGUAUUAUAG{INVAB} | 225 | UCUAUAAUACAUGUGAAAAACUU | 226 |
| D-1113 | UCACAUGUAUUAUAGAAUGC{INVAB} | 227 | AGCAUUCUAUAAUACAUGUGAUU | 228 |
| D-1114 | ACAUGUAUUAUAGAAUGCUU{INVAB} | 229 | AAAGCAUUCUAUAAUACAUGUUU | 230 |
| D-1115 | UGUAUUAUAGAAUGCUUUUG{INVAB} | 231 | ACAAAAGCAUUCUAUAAUACAUU | 232 |
| D-1116 | GAAUGCUUUUGCAUGGACUA{INVAB} | 233 | AUAGUCCAUGCAAAAGCAUUCUU | 234 |
| D-1117 | AAUGCUUUUGCAUGGACUAU{INVAB} | 235 | AAUAGUCCAUGCAAAAGCAUUUU | 236 |
| D-1118 | GCUUUUGCAUGGACUAUCCU{INVAB} | 237 | AAGGAUAGUCCAUGCAAAAGCUU | 238 |
| D-1119 | UUUGCAUGGACUAUCCUCUU{INVAB} | 239 | AAAGAGGAUAGUCCAUGCAAAUU | 240 |
| D-1120 | UUGCAUGGACUAUCCUCUUG{INVAB} | 241 | ACAAGAGGAUAGUCCAUGCAAUU | 242 |
| D-1121 | UGCAUGGACUAUCCUCUUGU{INVAB} | 243 | AACAAGAGGAUAGUCCAUGCAUU | 244 |
| D-1122 | GCAUGGACUAUCCUCUUGUU{INVAB} | 245 | AAACAAGAGGAUAGUCCAUGCUU | 246 |
| D-1123 | AUGGACUAUCCUCUUGUUUU{INVAB} | 247 | AAAAACAAGAGGAUAGUCCAUUU | 248 |
| D-1124 | GGACUAUCCUCUUGUUUUUA{INVAB} | 249 | AUAAAAACAAGAGGAUAGUCCUU | 250 |
| D-1125 | AAUAACCUCUUGUAGUUAUA{INVAB} | 251 | UUAUAACUACAAGAGGUUAUUUU | 252 |
| D-1126 | AUAACCUCUUGUAGUUAUAA{INVAB} | 253 | UUUAUAACUACAAGAGGUUAUUU | 254 |
| D-1127 | ACCUCUUGUAGUUAUAAAAU{INVAB} | 255 | UAUUUUAUAACUACAAGAGGUUU | 256 |
| D-1128 | GGUCAACAUCCUAGGACAUU{INVAB} | 257 | AAAUGUCCUAGGAUGUUGACCUU | 258 |
| D-1129 | GUCAACAUCCUAGGACAUUU{INVAB} | 259 | AAAAUGUCCUAGGAUGUUGACUU | 260 |
| D-1130 | UCAACAUCCUAGGACAUUUU{INVAB} | 261 | AAAUGUCCUAGGAUGUUGAUU | 262 |
| D-1131 | CAACAUCCUAGGACAUUUUU{INVAB} | 263 | AAAAUGUCCUAGGAUGUUGUU | 264 |
| D-1132 | GGUCAACAUCCUAGGACAUU{INVAB} | 265 | AAAUGUCCUAGGAUGUUGACCUU | 266 |
| D-1133 | GUCAACAUCCUAGGACAUUU{INVAB} | 267 | AAAAUGUCCUAGGAUGUUGACUU | 268 |
| D-1134 | GGUCAACAUCCUAGGACAUU{INVAB} | 269 | AAAUGUCCUAGGAUGUUGACC | 270 |
| D-1135 | GUCAACAUCCUAGGACAUUU{INVAB} | 271 | AAAAUGUCCUAGGAUGUUGAC | 272 |
| D-1136 | GGUCAACAUCCUAGGACAUU{INVAB} | 273 | AAAUGUCCUAGGAUGUUGACC | 274 |
| D-1137 | GUCAACAUCCUAGGACAUUU{INVAB} | 275 | AAAAUGUCCUAGGAUGUUGAC | 276 |
| D-1138 | GUCAACAUCCUAGGACAUUU{INVAB} | 277 | AAAAUGUCCUAGGAUGUUGAC | 278 |
| D-1139 | GGUCAACAUCCUAGGACAUU{INVAB} | 279 | AAAUGUCCUAGGAUGUUGACC | 280 |
| D-1140 | [INVAB]UCAACAUCCUAGGACAUUU | 281 | AAAUGUCCUAGGAUGUUGAUU | 282 |
| D-1141 | [INVAB]CAACAUCCUAGGACAUUUU | 283 | AAAAUGUCCUAGGAUGUUGUU | 284 |
| D-1142 | UCAACAUCCUAGGACAUU{INVAB} | 285 | AAAUGUCCUAGGAUGUUGAUU | 286 |
| D-1143 | CAACAUCCUAGGACAUUU{INVAB} | 287 | AAAAUGUCCUAGGAUGUUGUU | 288 |
| D-1144 | GGUCAACAUCGAUGGUCAUU{INVAB} | 289 | AAAUGACCAUCGAUGUUGACCUU | 290 |
| D-1145 | GUCAACAUCCAUCGAGAUUU{INVAB} | 291 | AAAAUCUCGAUGGAUGUUGACUU | 292 |
| D-1146 | GUCAACAUCCUAGGACAUUU{INVAB} | 293 | AAUGUCCUAGGAUGUUGACUU | 294 |
| D-1147 | CUCCCACUACAUCAAGACUU{INVAB} | 295 | AGUCUUGAUGUAGUGGGAGUU | 296 |
| D-1148 | CCACUACAUCAAGACUAAUU{INVAB} | 297 | AUUAGUCUUGAUGUAGUGGUU | 298 |
| D-1149 | UUCCUUAUCUCAUCCCUU{INVAB} | 299 | UAAGGGAUGAGAUAAGGAAUU | 300 |
| D-1150 | UUCCUUAUGAGAUCCCUU{INVAB} | 301 | UAAGGGAUCUCAUAAGGAAUU | 302 |
| D-1151 | UCAACAUCGAUGGUCAUU{INVAB} | 303 | AAAUGACCAUCGAUGUUGAUU | 304 |
| D-1152 | UACAUCAAGACUAAUCUUGUU | 305 | AACAAG[GNA-A]UUAGUCUUGAUGUAGU | 306 |
| D-1153 | AUGCUUUUGCAUGGACUAUC{INVAB} | 307 | AGAUAG[GNA-T]CCAUGCAAAAGCAUUC | 308 |
| D-1154 | CGUAUGCAGAAUAUUCAAUUU | 309 | AAAUUG[GNA-A]AUAUUCUGCAUACGAU | 310 |
| D-1155 | CGUAUGCAGAAUAUUCAAUUU | 311 | AAAUUG[GNA-A]AUAUUCUGCAUACGAU | 312 |
| D-1156 | CUACAUCAAGACUAAUCUUGU | 313 | ACAAGA[GNA-T]UAGUCUUGAUGUAGUG | 314 |
| D-1157 | AAGCCAUGAACAUCAUCCUA{INVAB} | 315 | AUAGGAUGAUGUUCAUGGCUUUU | 316 |
| D-1158 | AGCCAUGAACAUCAUCCUAG{INVAB} | 317 | UCUAGGAUGAUGUUCAUGGCUUU | 318 |
| D-1159 | CCAUGAACAUCAUCCUAGAA{INVAB} | 319 | UUUCUAGGAUGAUGUUCAUGGUU | 320 |
| D-1160 | UGAACAUCAUCCUAGAAAUC{INVAB} | 321 | AGAUUUCUAGGAUGAUGUUCAUU | 322 |
| D-1161 | GAAGUUUUUCAUUCCUCAGA{INVAB} | 323 | AUCUGAGGAAUGAAAAACUUCUU | 324 |
| D-1162 | GGAGAAAAUCUGUGGCUGGG{INVAB} | 325 | ACCCAGCCACAGAUUUUCUCCUU | 326 |
| D-1163 | GUGGCUGGGGAGAUUGUUCU{INVAB} | 327 | AAGAACAAUCUCCCCAGCCACUU | 328 |
| D-1164 | GGAAUAGGCAGGCAGACUAC{INVAB} | 329 | AGUAGUCUGCCUGCCUAUUCCUU | 330 |
| D-1165 | GAAUAGGCAGGCAGACUACU{INVAB] | 331 | AAGUAGUCUGCCUGCCUAUUCUU | 332 |
| D-1166 | UUUGCAAAACGACAGAGCAU{INVAB} | 333 | UAUGCUCUGUCGUUUUGCAAAUU | 334 |
| D-1167 | GCAAAACGACAGAGCAUAUU{INVAB} | 335 | AAAUAUGCUCUGUCGUUUUGCUU | 336 |
| D-1168 | ACGACAGAGCAUAUUGGUUC{INVAB} | 337 | AGAACCAAUAUGCUCUGUCGUUU | 338 |
| D-1169 | CGACAGAGCAUAUUGGUUCU{INVAB} | 339 | AAGAACCAAUAUGCUCUGUCGUU | 340 |
| D-1170 | GACAGAGCAUAUUGGUUCUG{INVAB} | 341 | ACAGAACCAAUAUGCUCUGUCUU | 342 |
| D-1171 | ACAGAGCAUAUUGGUUCUGU{INVAB} | 343 | AACAGAACCAAUAUGCUCUGUUU | 344 |
| D-1172 | CAGAGCAUAUUGGUUCUGUG{INVAB} | 345 | ACACAGAACCAAUAUGCUCUGUU | 346 |
| D-1173 | AGAGCAUAUUGGUUCUGUGG{INVAB} | 347 | ACCACAGAACCAAUAUGCUCUUU | 348 |
| D-1174 | GAGCAUAUUGGUUCUGUGGG{INVAB} | 349 | UCCCACAGAACCAAUAUGCUCUU | 350 |
| D-1175 | CUGUGGGAUAUUAAUAAGCG{INVAB} | 351 | ACGCUUAUUAAUAUCCCACAGUU | 352 |
| D-1176 | UUAAUAAGCGCGGUGUGGAG{INVAB} | 353 | ACUCCACACCGCGCUUAUUAAUU | 354 |
| D-1177 | UAAUAAGCGCGGUGUGGAGG{INVAB} | 355 | UCCUCCACACCGCGCUUAUUAUU | 356 |
| D-1178 | AUAAGCGCGGUGUGGAGGAA{INVAB} | 357 | UUUCCUCCACACCGCGCUUAUUU | 358 |
| D-1179 | UAAGCGCGGUGUGGAGGAAA{INVAB} | 359 | AUUUCCUCCACACCGCGCUUAUU | 360 |
| D-1180 | GGCGUCACUGCGCAUGCGUA{INVAB} | 361 | AUACGCAUGCGCAGUGACGCCUU | 362 |
| D-1181 | GCGUCACUGCGCAUGCGUAU{INVAB} | 363 | AAUACGCAUGCGCAGUGACGCUU | 364 |
| D-1182 | CGUCACUGCGCAUGCGUAUG{INVAB} | 365 | ACAUACGCAUGCGCAGUGACGUU | 366 |
| D-1183 | AGCAACAGAGAAGAGAUCUA{INVAB} | 367 | AUAGAUCUCUUCUCUGUUGCUUU | 368 |
| D-1184 | CAACAGAGAAGAGAUCUAUC{INVAB} | 369 | AGAUAGAUCUCUUCUCUGUUGUU | 370 |
| D-1185 | AACAGAGAAGAGAUCUAUCG{INVAB} | 371 | ACGAUAGAUCUCUUCUCUGUUUU | 372 |
| D-1186 | CAGAGAAGAGAUCUAUCGCU{INVAB} | 373 | AAGCGAUAGAUCUCUUCUCUGUU | 374 |
| D-1187 | AGAGAAGAGAUCUAUCGCUC{INVAB} | 375 | AGAGCGAUAGAUCUCUUCUCUUU | 376 |
| D-1188 | GAGAAGAGAUCUAUCGCUCU{INVAB} | 377 | AAGAGCGAUAGAUCUCUUCUCUU | 378 |
| D-1189 | AAGAGAUCUAUCGCUCUCUA{INVAB} | 379 | UUAGAGAGCGAUAGAUCUCUUUU | 380 |
| D-1190 | GAGAUCUAUCGCUCUCUAAA{INVAB} | 381 | AUUUAGAGAGCGAUAGAUCUCUU | 382 |
| D-1191 | GAUCUAUCGCUCUCUAAAUC{INVAB} | 383 | UGAUUUAGAGAGCGAUAGAUCUU | 384 |
| D-1192 | AUCUAUCGCUCUCUAAAUCA{INVAB} | 385 | AUGAUUUAGAGAGCGAUAGAUUU | 386 |
| D-1193 | CGCUCUCUAAAUCAGGUGAA{INVAB} | 387 | AUUCACCUGAUUUAGAGAGCGUU | 388 |
| D-1194 | CUCUCUAAAUCAGGUGAAGA{INVAB} | 389 | UUCUUCACCUGAUUUAGAGAGUU | 390 |
| D-1195 | AAAGAAGUGGGUGAUGUAAC{INVAB} | 391 | UGUUACAUCACCCACUUCUUUUU | 392 |
| D-1196 | AAGAAGUGGGUGAUGUAACA{INVAB} | 393 | UUGUUACAUCACCCACUUCUUUU | 394 |
| D-1197 | AGAAGUGGGUGAUGUAACAA{INVAB} | 395 | AUUGUUACAUCACCCACUUCUUU | 396 |
| D-1198 | GAAGUGGGUGAUGUAACAAU{INVAB} | 397 | AAUUGUUACAUCACCCACUUCUU | 398 |
| D-1199 | GAUGUAACAAUCGUGGUGAA{INVAB} | 399 | AUUCACCACGAUUGUUACAUCUU | 400 |
| D-1200 | AUGUAACAAUCGUGGUGAAU{INVAB} | 401 | UAUUCACCACGAUUGUUACAUUU | 402 |
| D-1201 | UGUAACAAUCGUGGUGAAUA{INVAB} | 403 | UUAUUCACCACGAUUGUUACAUU | 404 |
| D-1202 | GUAACAAUCGUGGUGAAUAA{INVAB} | 405 | AUUAUUCACCACGAUUGUUACUU | 406 |
| D-1203 | UAACAAUCGUGGUGAAUAAU{INVAB} | 407 | AAUUAUUCACCACGAUUGUUAUU | 408 |
| D-1204 | GGUGAAUAAUGCUGGGACAG{INVAB} | 409 | ACUGUCCCAGCAUUAUUCACCUU | 410 |
| D-1205 | GUGAAUAAUGCUGGGACAGU{INVAB} | 411 | UACUGUCCCAGCAUUAUUCACUU | 412 |
| D-1206 | UAAUGCUGGGACAGUAUAUC{INVAB} | 413 | AGAUAUACUGUCCCAGCAUUAUU | 414 |
| D-1207 | AAUGCUGGGACAGUAUAUCC{INVAB} | 415 | UGGAUAUACUGUCCCAGCAUUUU | 416 |
| D-1208 | AAGAGAUUACCAAGACAUUU{INVAB} | 417 | AAAAUGUCUUGGUAAUCUCUUUU | 418 |
| D-1209 | AGAGAUUACCAAGACAUUUG{INVAB} | 419 | UCAAAUGUCUUGGUAAUCUCUUU | 420 |
| D-1210 | GAGAUUACCAAGACAUUUGA{INVAB} | 421 | AUCAAAUGUCUUGGUAAUCUCUU | 422 |
| D-1211 | UGAGGUCAACAUCCUAGGAC{INVAB} | 423 | UGUCCUAGGAUGUUGACCUCAUU | 424 |
| D-1212 | AGGUCAACAUCCUAGGACAU{INVAB} | 425 | AAUGUCCUAGGAUGUUGACCUUU | 426 |
| D-1213 | GGUCAACAUCCUAGGACAUU{INVAB} | 427 | AAAUGUCCUAGGAUGUUGACCUU | 428 |
| D-1214 | GUCAACAUCCUAGGACAUUU{INVAB} | 429 | AAAAUGUCCUAGGAUGUUGACUU | 430 |
| D-1215 | UCAACAUCCUAGGACAUUUU{INVAB} | 431 | AAAAAUGUCCUAGGAUGUUGAUU | 432 |
| D-1216 | CAACAUCCUAGGACAUUUUU{INVAB} | 433 | AAAAAAUGUCCUAGGAUGUUGUU | 434 |
| D-1217 | CAAAAGCACUUCUUCCAUCG{INVAB} | 435 | UCGAUGGAAGAAGUGCUUUUGUU | 436 |
| D-1218 | AAAAGCACUUCUUCCAUCGA{INVAB} | 437 | AUCGAUGGAAGAAGUGCUUUUUU | 438 |
| D-1219 | AAAGCACUUCUUCCAUCGAU{INVAB} | 439 | AAUCGAUGGAAGAAGUGCUUUUU | 440 |
| D-1220 | AAGCACUUCUUCCAUCGAUG{INVAB} | 441 | UCAUCGAUGGAAGAAGUGCUUUU | 442 |
| D-1221 | AGCACUUCUUCCAUCGAUGA{INVAB} | 443 | AUCAUCGAUGGAAGAAGUGCUUU | 444 |
| D-1222 | CACUUCUUCCAUCGAUGAUG{INVAB} | 445 | ACAUCAUCGAUGGAAGAAGUGUU | 446 |
| D-1223 | ACUUCUUCCAUCGAUGAUGG{INVAB} | 447 | UCCAUCAUCGAUGGAAGAAGUUU | 448 |
| D-1224 | UUCCAUCGAUGAUGGAGAGA{INVAB} | 449 | UUCUCUCCAUCAUCGAUGGAAUU | 450 |
| D-1225 | CCAUCGAUGAUGGAGAGAAA{INVAB} | 451 | AUUUCUCUCCAUCAUCGAUGGUU | 452 |
| D-1226 | GAUGGAGAGAAAUCAUGGCC{INVAB} | 453 | UGGCCAUGAUUUCUCUCCAUCUU | 454 |
| D-1227 | GUGGCUUCAGUGUGCGGCCA{INVAB} | 455 | AUGGCCGCACACUGAAGCCACUU | 456 |
| D-1228 | UGGCUUCAGUGUGCGGCCAC{INVAB} | 457 | AGUGGCCGCACACUGAAGCCAUU | 458 |
| D-1229 | GCUUCAGUGUGCGGCCACGA{INVAB} | 459 | UUCGUGGCCGCACACUGAAGCUU | 460 |
| D-1230 | UUCAGUGUGCGGCCACGAAG{INVAB} | 461 | ACUUCGUGGCCGCACACUGAAUU | 462 |
| D-1231 | UUGUGAAUACUGGGUUCACC{INVAB} | 463 | UGGUGAACCCAGUAUUCACAAUU | 464 |
| D-1232 | GAAUACUGGGUUCACCAAAA{INVAB} | 465 | UUUUUGGUGAACCCAGUAUUCUU | 466 |
| D-1233 | AUACUGGGUUCACCAAAAAU{INVAB} | 467 | AAUUUUUGGUGAACCCAGUAUUU | 468 |
| D-1234 | AGCACAAGAUUAUGGCCUGU{INVAB} | 469 | UACAGGCCAUAAUCUUGUGCUUU | 470 |
| D-1235 | CACAAGAUUAUGGCCUGUAU{INVAB} | 471 | AAUACAGGCCAUAAUCUUGUGUU | 472 |
| D-1236 | ACAAGAUUAUGGCCUGUAUU{INVAB} | 473 | AAAUACAGGCCAUAAUCUUGUUU | 474 |
| D-1237 | CAAGAUUAUGGCCUGUAUUG{INVAB} | 475 | ACAAUACAGGCCAUAAUCUUGUU | 476 |
| D-1238 | AGAUUAUGGCCUGUAUUGGA{INVAB} | 477 | AUCCAAUACAGGCCAUAAUCUUU | 478 |
| D-1239 | GAUUAUGGCCUGUAUUGGAG{INVAB} | 479 | UCUCCAAUACAGGCCAUAAUCUU | 480 |
| D-1240 | GAAGUCUGAUAGAUGGAAUA{INVAB} | 481 | AUAUUCCAUCUAUCAGACUUCUU | 482 |
| D-1241 | AAGUCUGAUAGAUGGAAUAC{INVAB} | 483 | AGUAUUCCAUCUAUCAGACUUUU | 484 |
| D-1242 | AGUCUGAUAGAUGGAAUACU{INVAB} | 485 | AAGUAUUCCAUCUAUCAGACUUU | 486 |
| D-1243 | GUCUGAUAGAUGGAAUACUU{INVAB} | 487 | UAAGUAUUCCAUCUAUCAGACUU | 488 |
| D-1244 | UCUGAUAGAUGGAAUACUUA{INVAB} | 489 | AUAAGUAUUCCAUCUAUCAGAUU | 490 |
| D-1245 | AUAGAUGGAAUACUUACCAA{INVAB} | 491 | AUUGGUAAGUAUUCCAUCUAUUU | 492 |
| D-1246 | UAGAUGGAAUACUUACCAAU{INVAB} | 493 | UAUUGGUAAGUAUUCCAUCUAUU | 494 |
| D-1247 | AGAUGGAAUACUUACCAAUA{INVAB} | 495 | UUAUUGGUAAGUAUUCCAUCUUU | 496 |
| D-1248 | GAUGGAAUACUUACCAAUAA{INVAB} | 497 | AUUAUUGGUAAGUAUUCCAUCUU | 498 |
| D-1249 | AUGGAAUACUUACCAAUAAG{INVAB} | 499 | UCUUAUUGGUAAGUAUUCCAUUU | 500 |
| D-1250 | UGGAAUACUUACCAAUAAGA{INVAB} | 501 | UUCUUAUUGGUAAGUAUUCCAUU | 502 |
| D-1251 | AUAUCAAUAUCUUUCUGAGA{INVAB} | 503 | AUCUCAGAAAGAUAUUGAUAUUU | 504 |
| D-1252 | CUUUCUGAGACUACAGAAGU{INVAB} | 505 | AACUUCUGUAGUCUCAGAAAGUU | 506 |
| D-1253 | UUUCUGAGACUACAGAAGUU{INVAB} | 507 | AAACUUCUGUAGUCUCAGAAAUU | 508 |
| D-1254 | UUCUGAGACUACAGAAGUUU{INVAB} | 509 | AAAACUUCUGUAGUCUCAGAAUU | 510 |
| D-1255 | UCUGAGACUACAGAAGUUUC{INVAB} | 511 | AGAAACUUCUGUAGUCUCAGAUU | 512 |
| D-1256 | UGGUUGGCCACAAAAUCAAA{INVAB} | 513 | UUUUGAUUUUGUGGCCAACCAUU | 514 |
| D-1257 | AAAUGAAAUGAAUAAAUAAG{INVAB} | 515 | ACUUAUUUAUUCAUUUCAUUUUU | 516 |
| D-1258 | UUCACAUUUUUUCAGUCCUG{INVAB} | 517 | UCAGGACUGAAAAAAUGUGAAUU | 518 |
| D-1259 | GUUUGGCACUAGCAGCAGUC{INVAB} | 519 | UGACUGCUGCUAGUGCCAAACUU | 520 |
| D-1260 | UUUGGCACUAGCAGCAGUCA{INVAB} | 521 | UUGACUGCUGCUAGUGCCAAAUU | 522 |
| D-1261 | UUGGCACUAGCAGCAGUCAA{INVAB} | 523 | UUUGACUGCUGCUAGUGCCAAUU | 524 |
| D-1262 | UGGCACUAGCAGCAGUCAAA{INVAB} | 525 | AUUUGACUGCUGCUAGUGCCAUU | 526 |
| D-1263 | GGCACUAGCAGCAGUCAAAC{INVAB} | 527 | AGUUUGACUGCUGCUAGUGCCUU | 528 |
| D-1264 | AUUUACGUAGUUUUUCAUAG{INVAB} | 529 | ACUAUGAAAAACUACGUAAAUUU | 530 |
| D-1265 | UUACGUAGUUUUUCAUAGGU{INVAB} | 531 | AACCUAUGAAAAACUACGUAAUU | 532 |
| D-1266 | UACGUAGUUUUUCAUAGGUC{INVAB} | 533 | AGACCUAUGAAAAACUACGUAUU | 534 |
| D-1267 | UUACAUAAACAUACUUAAAA{INVAB} | 535 | AUUUUAAGUAUGUUUAUGUAAUU | 536 |
| D-1268 | UUAAAGGUGGACAAAAGCUA{INVAB} | 537 | AUAGCUUUUGUCCACCUUUAAUU | 538 |
| D-1269 | UAAAGGUGGACAAAAGCUAC{INVAB} | 539 | AGUAGCUUUUGUCCACCUUUAUU | 540 |
| D-1270 | AAGGUGGACAAAAGCUACCU{INVAB} | 541 | AAGGUAGCUUUUGUCCACCUUUU | 542 |
| D-1271 | GGUGGACAAAAGCUACCUCC{INVAB} | 543 | AGGAGGUAGCUUUUGUCCACCUU | 544 |
| D-1272 | ACAGCUAAGAGAUCAAGUUU{INVAB} | 545 | AAAACUUGAUCUCUUAGCUGUUU | 546 |
| D-1273 | CAGCUAAGAGAUCAAGUUUC{INVAB} | 547 | UGAAACUUGAUCUCUUAGCUGUU | 548 |
| D-1274 | AGCUAAGAGAUCAAGUUUCA{INVAB} | 549 | AUGAAACUUGAUCUCUUAGCUUU | 550 |
| D-1275 | CCUGGACAUAUUUUAAGAUU{INVAB} | 551 | AAAUCUUAAAAUAUGUCCAGGUU | 552 |
| D-1276 | CUGGACAUAUUUUAAGAUUC{INVAB} | 553 | UGAAUCUUAAAAUAUGUCCAGUU | 554 |
| D-1277 | CUUCCUUUUUCAUUAGCCCA{INVAB} | 555 | UUGGGCUAAUGAAAAAGGAAGUU | 556 |
| D-1278 | UUCCUUUUUCAUUAGCCCAA{INVAB} | 557 | UUUGGGCUAAUGAAAAAGGAAUU | 558 |
| D-1279 | CCCUCUAUAUUUCCUCCCUU{INVAB} | 559 | AAAGGGAGGAAAUAUAGAGGGUU | 560 |
| D-1280 | UAUUUCCUCCCUUUUUAUAG{INVAB} | 561 | ACUAUAAAAAGGGAGGAAAUAUU | 562 |
| D-1281 | UUCCUCCCUUUUUAUAGUCU{INVAB} | 563 | AAGACUAUAAAAAGGGAGGAAUU | 564 |
| D-1282 | UCCUCCCUUUUUAUAGUCUU{INVAB} | 565 | UAAGACUAUAAAAAGGGAGGAUU | 566 |
| D-1283 | CCUUUUUAUAGUCUUAUAAG{INVAB} | 567 | UCUUAUAAGACUAUAAAAAGGUU | 568 |
| D-1284 | CUUUUUAUAGUCUUAUAAGA{INVAB} | 569 | AUCUUAUAAGACUAUAAAAAGUU | 570 |
| D-1285 | UUUUUAUAGUCUUAUAAGAU{INVAB} | 571 | UAUCUUAUAAGACUAUAAAAAUU | 572 |
| D-1286 | UUUUAUAGUCUUAUAAGAUA{INVAB} | 573 | AUAUCUUAUAAGACUAUAAAAUU | 574 |
| D-1287 | UUUAUAGUCUUAUAAGAUAC{INVAB} | 575 | UGUAUCUUAUAAGACUAUAAAUU | 576 |
| D-1288 | UUAUAGUCUUAUAAGAUACA{INVAB} | 577 | AUGUAUCUUAUAAGACUAUAAUU | 578 |
| D-1289 | UAUAGUCUUAUAAGAUACAU{INVAB} | 579 | AAUGUAUCUUAUAAGACUAUAUU | 580 |
| D-1290 | UCUUAUAAGAUACAUUAUGA{INVAB} | 581 | UUCAUAAUGUAUCUUAUAAGAUU | 582 |
| D-1291 | UUUUAAGUUCUAGCCCCAUG{INVAB} | 583 | UCAUGGGGCUAGAACUUAAAAUU | 584 |
| D-1292 | UUUAAGUUCUAGCCCCAUGA{INVAB} | 585 | AUCAUGGGGCUAGAACUUAAAUU | 586 |
| D-1293 | UAAGUUCUAGCCCCAUGAUA{INVAB} | 587 | UUAUCAUGGGGCUAGAACUUAUU | 588 |
| D-1294 | AAGUUCUAGCCCCAUGAUAA{INVAB} | 589 | AUUAUCAUGGGGCUAGAACUUUU | 590 |
| D-1295 | AGUUCUAGCCCCAUGAUAAC{INVAB} | 591 | AGUUAUCAUGGGGCUAGAACUUU | 592 |
| D-1296 | GUUCUAGCCCCAUGAUAACC{INVAB} | 593 | AGGUUAUCAUGGGGCUAGAACUU | 594 |
| D-1297 | CUAGCCCCAUGAUAACCUUU{INVAB} | 595 | AAAAGGUUAUCAUGGGGCUAGUU | 596 |
| D-1298 | AGCCCCAUGAUAACCUUUUU{INVAB} | 597 | AAAAAAGGUUAUCAUGGGGCUUU | 598 |
| D-1299 | GCCCCAUGAUAACCUUUUUC{INVAB} | 599 | AGAAAAAGGUUAUCAUGGGGCUU | 600 |
| D-1300 | CCCAUGAUAACCUUUUUCUU{INVAB} | 601 | AAAGAAAAAGGUUAUCAUGGGUU | 602 |
| D-1301 | CCAUGAUAACCUUUUUCUUU{INVAB} | 603 | AAAAGAAAAAGGUUAUCAUGGUU | 604 |
| D-1302 | CAUGAUAACCUUUUUCUUUG{INVAB} | 605 | ACAAAGAAAAAGGUUAUCAUGUU | 606 |
| D-1303 | AUAACCUUUUUCUUUGUAAU{INVAB} | 607 | AAUUACAAAGAAAAAGGUUAUUU | 608 |
| D-1304 | UUUUUCUUUGUAAUUUAUGC{INVAB} | 609 | AGCAUAAAUUACAAAGAAAAAUU | 610 |
| D-1305 | UUUUCUUUGUAAUUUAUGCU{INVAB} | 611 | AAGCAUAAAUUACAAAGAAAAUU | 612 |
| D-1306 | GGCUAUUACAUAAGAAACAA{INVAB} | 613 | AUUGUUUCUUAUGUAAUAGCCUU | 614 |
| D-1307 | CUAUUACAUAAGAAACAAUG{INVAB} | 615 | ACAUUGUUUCUUAUGUAAUAGUU | 616 |
| D-1308 | UUACAUAAGAAACAAUGGAC{INVAB} | 617 | AGUCCAUUGUUUCUUAUGUAAUU | 618 |
| D-1309 | UACAUAAGAAACAAUGGACC{INVAB} | 619 | AGGUCCAUUGUUUCUUAUGUAUU | 620 |
| D-1310 | ACAUAAGAAACAAUGGACCC{INVAB} | 621 | UGGGUCCAUUGUUUCUUAUGUUU | 622 |
| D-1311 | AAGAAACAAUGGACCCAAGA{INVAB} | 623 | AUCUUGGGUCCAUUGUUUCUUUU | 624 |
| D-1312 | AGAAACAAUGGACCCAAGAG{INVAB} | 625 | UCUCUUGGGUCCAUUGUUUCUUU | 626 |
| D-1313 | GAAACAAUGGACCCAAGAGA{INVAB} | 627 | UUCUCUUGGGUCCAUUGUUUCUU | 628 |
| D-1314 | AAUAGAAAAAAUAAUCCGAC{INVAB} | 629 | AGUCGGAUUAUUUUUUCUAUUUU | 630 |
| D-1315 | AUAGAAAAAAUAAUCCGACU{INVAB} | 631 | AAGUCGGAUUAUUUUUUCUAUUU | 632 |
| D-1316 | AAAACAAUUCACUAAAAAUA{INVAB} | 633 | UUAUUUUUAGUGAAUUGUUUUUU | 634 |
| D-1317 | UGUAGUUAUAAAAUAAAACG{INVAB} | 635 | ACGUUUUAUUUUAUAACUACAUU | 636 |
| D-1318 | AAUAAAACGUUUGACUUCUA{INVAB} | 637 | UUAGAAGUCAAACGUUUUAUUUU | 638 |
| D-1319 | AUAAAACGUUUGACUUCUAA{INVAB} | 639 | UUUAGAAGUCAAACGUUUUAUUU | 640 |
| D-1320 | UAAAACGUUUGACUUCUAAA{INVAB} | 641 | AUUUAGAAGUCAAACGUUUUAUU | 642 |
| D-1321 | AAAACGUUUGACUUCUAAAC{INVAB} | 643 | AGUUUAGAAGUCAAACGUUUUUU | 644 |
| D-1322 | AAACGUUUGACUUCUAAACU{INVAB} | 645 | AAGUUUAGAAGUCAAACGUUUUU | 646 |

To improve the potency and in vivo stability of HSD17B13 siRNA sequences, chemical modifications were incorporated into HSD17B13 siRNA molecules. Specifically, 2′-O-methyl and 2′-fluoro modifications of the ribose sugar were incorporated at specific positions within the HSD17B13 siRNAs. Phosphorothioate internucleotide linkages were also incorporated at the terminal ends of the antisense and/or sense sequences. Table 2 below depicts the modifications in the sense and antisense sequences for each of the modified HSD17B13 siRNAs. The nucleotide sequences in Table 2 and other parts of the application are listed according to the following notations: A, U, G, and C=corresponding ribonucleotide; dT=deoxythymidine; dA=deoxyadenosine; dC=deoxycytidine; dG=deoxyguanosine; invDT=inverted deoxythymidine; invDA=inverted deoxyadenosine; invDC=inverted deoxycytidine; invDG=inverted deoxyguanosine; a, u, g, and c=corresponding 2′-O-methyl ribonucleotide; Af, Uf, Gf, and Cf=corresponding 2′-deoxy-2′-fluoro (“2′-fluoro”) ribonucleotide; Ab=Abasic; MeO—I=2′-methoxy inosine; GNA=glycol nucleic acid; sGNA=glycol nucleic acid with 3′ phosphorothioate; LNA=locked nucleic acid. Insertion of an “s” in the sequence indicates that the two adjacent nucleotides are connected by a phosphorothiodiester group (e.g. a phosphorothioate internucleotide linkage). Unless indicated otherwise, all other nucleotides are connected by 3′-5′ phosphodiester groups. Each of the siRNA compounds in Table 2 comprises a 21 base pair duplex region with either a 2 nucleotide overhang at the 3′ end of both strands or bluntmer at one or both ends. The 5′ end of the sense strand in each of the siRNA compounds has been linked to the GalNAc structure of Formula I below via a phosphorothioate or phosphodiester linkage:

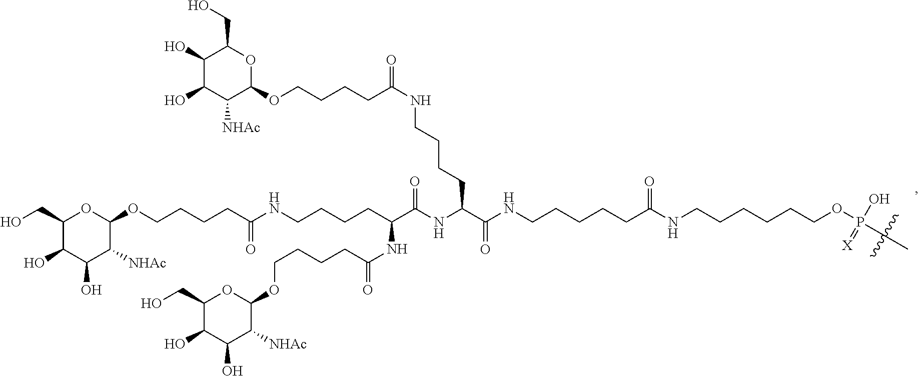

wherein X=O or S.

TABLE 2
siRNA sequences directed to HSD17B13 with modifications

| | | SEQ | | SEQ ID |
|---|---|---|---|---|
| | | ID | | NO: |
| Duplex | | NO: | | (anti- |
| No. | Sense sequence (5′-3′) | (sense) | Antisense sequence (5′-3′) | sense) |
| D-2000 | {sGalNAc3K2AhxC6}uucugcuuCfuGfAfUfCf | 647 | usGfsauggUfgaucAfgAfagcagaasusu | 648 |
| | accaucs{invAb} | | | |
| D-2001 | {sGalNAc3K2AhxC6}ucugcuucUfgAfUfCfAf | 649 | asUfsgaugGfugauCfaGfaagcagasusu | 650 |
| | ccaucas{invAb} | | | |
| D-2002 | {sGalNAc3K2AhxC6}cuucugauCfaCfCfAfUfc | 651 | asUfsagauGfauggUfgAfucagaagsusu | 652 |
| | aucuas{invAb} | | | |
| D-2003 | {sGalNAc3K2AhxC6}ucucauuaCfuGfGfAfGf | 653 | usGfscccaGfcuccAfgUfaaugagasusu | 654 |
| | cugggcs{invAb} | | | |
| D-2004 | {sGalNAc3K2AhxC6}gugaauaaUfgCfUfGfGf | 655 | usAfscuguCfccagCfaUfuauucacsusu | 656 |
| | gacagus{invAb} | | | |
| D-2005 | {sGalNAc3K2AhxC6}uaaugcugGfgAfCfAfGf | 657 | asGfsauauAfcuguCfcCfagcauuasusu | 658 |
| | uauaucs{invAb} | | | |
| D-2006 | {sGalNAc3K2AhxC6}aaugcuggGfaCfAfGfUf | 659 | usGfsgauaUfacugUfcCfcagcauususu | 660 |
| | auauccs{invAb} | | | |
| D-2007 | {sGalNAc3K2AhxC6}gggacaguAfuAfUfCfCf | 661 | asUfscggcUfggauAfuAfcugucccsusu | 662 |
| | agccgas{invAb} | | | |
| D-2008 | {sGalNAc3K2AhxC6}ggacaguaUfaUfCfCfAf | 663 | asAfsucggCfuggaUfaUfacuguccsusu | 664 |
| | gccgaus{invAb] | | | |
| D-2009 | {sGalNAc3K2AhxC6}gacaguauAfuCfCfAfGf | 665 | asGfsaucgGfcuggAfuAfuacugucsusu | 666 |
| | ccgaucs{invAb] | | | |
| D-2010 | {sGalNAc3K2AhxC6}acaguauaUfcCfAfGfCfc | 667 | asAfsgaucGfgcugGfaUfauacugususu | 668 |
| | gaucus{invAb} | | | |
| D-2011 | {sGalNAc3K2AhxC6}caguauauCfcAfGfCfCfg | 669 | asAfsagauCfggcuGfgAfuauacugsusu | 670 |
| | aucuus{invAb} | | | |
| D-2012 | {sGalNAc3K2AhxC6}gacauuugAfgGfUfCfAf | 671 | asGfsgaugUfugacCfuCfaaaugucsusu | 672 |
| | acauccs{invAb} | | | |
| D-2013 | {sGalNAc3K2AhxC6}ugaggucaAfcAfUfCfCf | 673 | usGfsuccuAfggauGfuUfgaccucasusu | 674 |
| | uaggacs{invAb} | | | |
| D-2014 | {sGalNAc3K2AhxC6}aggucaacAfuCfCfUfAfg | 675 | asAfsugucCfuaggAfuGfuugaccususu | 676 |
| | gacaus{invAb} | | | |
| D-2015 | {sGalNAc3K2AhxC6}ggucaacaUfcCfUfAfGf | 677 | asAfsauguCfcuagGfaUfguugaccsusu | 678 |
| | gacauus{invAb} | | | |
| D-2016 | {sGalNAc3K2AhxC6}gucaacauCfcUfAfGfGf | 679 | asAfsaaugUfccuaGfgAfuguugacsusu | 680 |
| | acauuus{invAb} | | | |
| D-2017 | {sGalNAc3K2AhxC6}ucaacaucCfuAfGfGfAf | 681 | asAfsaaauGfuccuAfgGfauguugasusu | 682 |
| | cauuuus{invAb} | | | |
| D-2018 | {sGalNAc3K2AhxC6}caacauccUfaGfGfAfCfa | 683 | asAfsaaaaUfguccUfaGfgauguugsusu | 684 |
| | uuuuus{invAb} | | | |
| D-2019 | {sGalNAc3K2AhxC6}caaaagcaCfuUfCfUfUf | 685 | usCfsgaugGfaagaAfgUfgcuuuugsusu | 686 |
| | ccaucgs{invAb} | | | |
| D-2020 | {sGalNAc3K2AhxC6}aaaagcacUfuCfUfUfCf | 687 | asUfscgauGfgaagAfaGfugcuuuususu | 688 |
| | caucgas{invAb} | | | |
| D-2021 | {sGalNAc3K2AhxC6}aaagcacuUfcUfUfCfCf | 689 | asAfsucgaUfggaaGfaAfgugcuuususu | 690 |
| | aucgaus{invAb} | | | |
| D-2022 | {sGalNAc3K2AhxC6}aagcacuuCfuUfCfCfAf | 691 | usCfsaucgAfuggaAfgAfagugcuususu | 692 |
| | ucgaugs{invAb} | | | |
| D-2023 | {sGalNAc3K2AhxC6}agcacuucUfuCfCfAfUfc | 693 | asUfscaucGfauggAfaGfaagugcususu | 694 |
| | gaugas{invAb} | | | |
| D-2024 | {sGalNAc3K2AhxC6}uuccuuacCfuCfAfUfCfc | 695 | asAfsuaugGfgaugAfgGfuaaggaasusu | 696 |
| | cauaus{invAb} | | | |
| D-2025 | {sGalNAc3K2AhxC6}ccuuaccuCfaUfCfCfCfa | 697 | asCfsaauaUfgggaUfgAfgguaaggsusu | 698 |
| | uauugs{invAb} | | | |
| D-2026 | {sGalNAc3K2AhxC6}accucaucCfcAfUfAfUfu | 699 | usGfsgaacAfauauGfgGfaugaggususu | 700 |
| | guuccs{invAb} | | | |
| D-2027 | {sGalNAc3K2AhxC6}ccucauccCfaUfAfUfUf | 701 | asUfsggaaCfaauaUfgGfgaugaggsusu | 702 |
| | guuccas{invAb} | | | |
| D-2028 | {sGalNAc3K2AhxC6}ucccauauUfgUfUfCfCf | 703 | asUfsuugcUfggaaCfaAfuaugggasusu | 704 |
| | agcaaas{invAb} | | | |
| D-2029 | {sGalNAc3K2AhxC6}ggcuuucaCfaGfAfGfGf | 705 | usGfsucagAfccucUfgUfgaaagccsusu | 706 |
| | ucugacs{invAb} | | | |
| D-2030 | {sGalNAc3K2AhxC6}uuugugaaUfaCfUfGfGf | 707 | asGfsugaaCfccagUfaUfucacaaasusu | 708 |
| | guucacs{invAb} | | | |
| D-2031 | {sGalNAc3K2AhxC6}uugugaauAfcUfGfGfGf | 709 | usGfsgugaAfcccaGfuAfuucacaasusu | 710 |
| | uucaccs{invAb} | | | |
| D-2032 | {sGalNAc3K2AhxC6}gaauacugGfgUfUfCfAf | 711 | usUfsuuugGfugaaCfcCfaguauucsusu | 712 |
| | ccaaaas{invAb} | | | |
| D-2033 | {sGalNAc3K2AhxC6}auacugggUfuCfAfCfCf | 713 | asAfsuuuuUfggugAfaCfccaguaususu | 714 |
| | aaaaaus{invAb} | | | |
| D-2034 | {sGalNAc3K2AhxC6}uacuggguUfcAfCfCfAf | 715 | asGfsauuuUfugguGfaAfcccaguasusu | 716 |
| | aaaaucs{invAb} | | | |
| D-2035 | {sGaINAc3K2AhxC6}uuuuaaauCfgUfAfUfGf | 717 | usAfsuucuGfcauaCfgAfuuuaaaasusu | 718 |
| | cagaaus{invAb} | | | |
| D-2036 | {sGalNAc3K2AhxC6}uuuaaaucGfuAfUfGfCf | 719 | asUfsauucUfgcauAfcGfauuuaaasusu | 720 |
| | agaauas{invAb} | | | |
| D-2037 | {sGalNAc3K2AhxC6}uaaaucguAfuGfCfAfGf | 721 | asAfsauauUfcugcAfuAfcgauuuasusu | 722 |
| | aauauus{invAb} | | | |
| D-2038 | {sGalNAc3K2AhxC6}aaaucguaUfgCfAfGfAf | 723 | usGfsaauaUfucugCfaUfacgauuususu | 724 |
| | auauucs{invAb} | | | |
| D-2039 | {sGalNAc3K2AhxC6}aaucguauGfcAfGfAfAf | 725 | usUfsgaauAfuucuGfcAfuacgauususu | 726 |
| | uauucas{invAb} | | | |
| D-2040 | {sGalNAc3K2AhxC6}ucguaugcAfgAfAfUfAf | 727 | asAfsuugaAfuauuCfuGfcauacgasusu | 728 |
| | uucaaus{invAb] | | | |
| D-2041 | {sGalNAc3K2AhxC6}cguaugcaGfaAfUfAfUf | 729 | asAfsauugAfauauUfcUfgcauacgsusu | 730 |
| | ucaauus{invAb} | | | |
| D-2042 | {sGalNAc3K2AhxC6}uaugcagaAfuAfUfUfCf | 731 | usCfsaaauUfgaauAfuUfcugcauasusu | 732 |
| | aauuugs{invAb} | | | |
| D-2043 | {sGalNAc3K2AhxC6}aauauucaAfuUfUfGfAf | 733 | asAfscugcUfucaaAfuUfgaauauususu | 734 |
| | agcagus{invAb} | | | |
| D-2044 | {sGalNAc3K2AhxC6}aaaugaaaUfgAfAfUfAf | 735 | asCfsuuau UfuauuCfaUfuucauuususu | 736 |
| | aauaags{invAb} | | | |
| D-2045 | {sGalNAc3K2AhxC6}aaucaaugCfuGfCfAfAf | 737 | usAfsaagcUfuugcAfgCfauugauususu | 738 |
| | agcuuus{invAb} | | | |
| D-2046 | {sGalNAc3K2AhxC6}ugcugcaaAfgCfUfUfUf | 739 | asUfsgaaaUfaaagCfuUfugcagcasusu | 740 |
| | auuucas{invAb} | | | |
| D-2047 | {sGalNAc3K2AhxC6}gcugcaaaGfcUfUfUfAf | 741 | usGfsugaaAfuaaaGfcUfuugcagcsusu | 742 |
| | uuucacs{invAb} | | | |
| D-2048 | {sGalNAc3K2AhxC6}uuaaaaacAfuUfGfGfUf | 743 | asUfsgccaAfaccaAfuGfuuuuuaasusu | 744 |
| | uuggcas{invAb} | | | |
| D-2049 | {sGalNAc3K2AhxC6}aaaaacauUfgGfUfUfUf | 745 | usAfsgugcCfaaacCfaAfuguuuuususu | 746 |
| | ggcacus{invAb} | | | |
| D-2050 | {sGalNAc3K2AhxC6}aacaagauUfaAfUfUfAf | 747 | asGfsacagGfuaauUfaAfucuuguususu | 748 |
| | ccugucs{invAb} | | | |
| D-2051 | {sGalNAc3K2AhxC6}caagauuaAfuUfAfCfCf | 749 | asAfsagacAfgguaAfuUfaaucuugsusu | 750 |
| | ugucuus{invAb} | | | |
| D-2052 | {sGalNAc3K2AhxC6}uaauuaccUfgUfCfUfUf | 751 | asAfsacagGfaagaCfaGfguaauuasusu | 752 |
| | ccuguus{invAb} | | | |
| D-2053 | {sGalNAc3K2AhxC6}ccugucuuCfcUfGfUfUf | 753 | asUfsugagAfaacaGfgAfagacaggsusu | 754 |
| | ucucaas{invAb} | | | |
| D-2054 | {sGalNAc3K2AhxC6}uuuccuuuCfaUfGfCfCf | 755 | usUfsuaagAfggcaUfgAfaaggaaasusu | 756 |
| | ucuuaas{invAb} | | | |
| D-2055 | {sGalNAc3K2AhxC6}uuccuuucAfuGfCfCfUf | 757 | usUfsuuaaGfaggcAfuGfaaaggaasusu | 758 |
| | cuuaaas{invAb} | | | |
| D-2056 | {sGalNAc3K2AhxC6}uuuuccauUfuAfAfAfGf | 759 | usGfsuccaCfcuuuAfaAfuggaaaasusu | 760 |
| | guggacs{invAb} | | | |
| D-2057 | {sGalNAc3K2AhxC6}uuuccauuUfaAfAfGfGf | 761 | usUfsguccAfccuu UfaAfauggaaasusu | 762 |
| | uggacas{invAb} | | | |
| D-2058 | {sGalNAc3K2AhxC6}uuccauuuAfaAfGfGfUf | 763 | usUfsugucCfaccuUfuAfaauggaasusu | 764 |
| | ggacaas{invAb} | | | |
| D-2059 | {sGalNAc3K2AhxC6}uccauuuaAfaGfGfUfGf | 765 | usUfsuuguCfcaccUfuUfaaauggasusu | 766 |
| | gacaaas{invAb} | | | |
| D-2060 | {sGalNAc3K2AhxC6}gaacuuauUfuAfCfAfCf | 767 | asUfsucccUfguguAfaAfuaaguucsusu | 768 |
| | agggaas{invAb} | | | |
| D-2061 | {sGalNAc3K2AhxC6}cuuauuuaCfaCfAfGfGf | 769 | asAfsccuuCfccugUfgUfaaauaagsusu | 770 |
| | gaaggus{invAb} | | | |
| D-2062 | {sGalNAc3K2AhxC6}auuuacacAfgGfGfAfAf | 771 | usUfsaaacCfuuccCfuGfuguaaaususu | 772 |
| | gguuuas{invAb} | | | |
| D-2063 | {sGalNAc3K2AhxC6}uuuacacaGfgGfAfAfGf | 773 | asUfsuaaaCfcuucCfcUfguguaaasusu | 774 |
| | guuuaas{invAb] | | | |
| D-2064 | {sGalNAc3K2AhxC6}cagggaagGfuUfUfAfAf | 775 | asAfscaguCfuuaaAfcCfuucccugsusu | 776 |
| | gacugus{invAb} | | | |
| D-2065 | {sGalNAc3K2AhxC6}ggaagguuUfaAfGfAfCf | 777 | usUfsgaacAfgucuUfaAfaccuuccsusu | 778 |
| | uguucas{invAb] | | | |
| D-2066 | {sGalNAc3K2AhxC6}agguuuaaGfaCfUfGfUf | 779 | usAfscuugAfacagUfcUfuaaaccususu | 780 |
| | ucaagus{invAb} | | | |
| D-2067 | {sGalNAc3K2AhxC6}gguuuaagAfcUfGfUfUf | 781 | asUfsacuuGfaacaGfuCfuuaaaccsusu | 782 |
| | caaguas{invAb} | | | |
| D-2068 | {sGalNAc3K2AhxC6}agacuguuCfaAfGfUfAf | 783 | asGfsaaugCfuacuUfgAfacagucususu | 784 |
| | gcauucs{invAb} | | | |
| D-2069 | {sGalNAc3K2AhxC6}gacuguucAfaGfUfAfGf | 785 | usGfsgaauGfcuacUfuGfaacagucsusu | 786 |
| | cauuccs{invAb} | | | |
| D-2070 | {sGalNAc3K2AhxC6}acuguucaAfgUfAfGfCf | 787 | usUfsggaaUfgcuaCfuUfgaacagususu | 788 |
| | auuccas{invAb} | | | |
| D-2071 | {sGalNAc3K2AhxC6}cuguucaaGfuAfGfCfAf | 789 | asUfsuggaAfugcuAfcUfugaacagsusu | 790 |
| | uuccaas{invAb} | | | |
| D-2072 | {sGalNAc3K2AhxC6}uguucaagUfaGfCfAfUf | 791 | asAfsuuggAfaugcUfaCfuugaacasusu | 792 |
| | uccaaus{invAb} | | | |
| D-2073 | {sGalNAc3K2AhxC6}caagaacaCfaGfAfAfUfg | 793 | usGfscacuCfauucUfgUfguucuugsusu | 794 |
| | agugcs{invAb} | | | |
| D-2074 | {sGalNAc3K2AhxC6}acagaaugAfgUfGfCfAf | 795 | usUfsagcuGfugcaCfuCfauucugususu | 796 |
| | cagcuas{invAb} | | | |
| D-2075 | {sGalNAc3K2AhxC6}aggcagcuUfuAfUfCfUf | 797 | asAfsgguuGfagauAfaAfgcugccususu | 798 |
| | caaccus{invAb} | | | |
| D-2076 | {sGalNAc3K2AhxC6}uuuuaagaUfuCfAfGfCf | 799 | usUfscaaaUfgcugAfaUfcuuaaaasusu | 800 |
| | auuugas{invAb} | | | |
| D-2077 | {sGalNAc3K2AhxC6}agauucagCfaUfUfUfGf | 801 | asAfsucuuUfcaaaUfgCfugaaucususu | 802 |
| | aaagaus{invAb} | | | |
| D-2078 | {sGalNAc3K2AhxC6}auuugaaaGfaUfUfUfCf | 803 | asGfscuagGfgaaaUfcUfuucaaaususu | 804 |
| | ccuagcs{invAb] | | | |
| D-2079 | {sGalNAc3K2AhxC6}uucccuagCfcUfCfUfUf | 805 | asAfsaaagGfaagaGfgCfuagggaasusu | 806 |
| | ccuuuus{invAb} | | | |
| D-2080 | {sGalNAc3K2AhxC6}cuauucugGfaCfUfUfUf | 807 | asAfsguaaUfaaagUfcCfagaauagsusu | 808 |
| | auuacus{invAb} | | | |
| D-2081 | {sGalNAc3K2AhxC6}aguccaccAfaAfAfGfUfg | 809 | asGfsggucCfacuu UfuGfguggacususu | 810 |
| | gacccs{invAb} | | | |
| D-2082 | {sGalNAc3K2AhxC6}caccaaaaGfuGfGfAfCfc | 811 | asUfsagagGfguccAfcUfuuuggugsusu | 812 |
| | cucuas{invAb} | | | |
| D-2083 | {sGalNAc3K2AhxC6}accaaaagUfgGfAfCfCfc | 813 | usAfsuagaGfggucCfaCfuuuuggususu | 814 |
| | ucuaus{invAb} | | | |
| D-2084 | {sGalNAc3K2AhxC6}caaaagugGfaCfCfCfUfc | 815 | asAfsuauaGfagggUfcCfacuuuugsusu | 816 |
| | uauaus{invAb} | | | |
| D-2085 | {sGalNAc3K2AhxC6}aaaaguggAfcCfCfUfCfu | 817 | asAfsauauAfgaggGfuCfcacuuuususu | 818 |
| | auauus{invAb} | | | |
| D-2086 | {sGalNAc3K2AhxC6}aaaguggaCfcCfUfCfUf | 819 | asAfsaauaUfagagGfgUfccacuuususu | 820 |
| | auauuus{invAb} | | | |
| D-2087 | {sGalNAc3K2AhxC6}aaguggacCfcUfCfUfAf | 821 | asGfsaaauAfuagaGfgGfuccacuususu | 822 |
| | uauuucs{invAb} | | | |
| D-2088 | {sGalNAc3K2AhxC6}uucauauaUfcCfUfUfGf | 823 | asUfsgggaCfcaagGfaUfauaugaasusu | 824 |
| | gucccas{invAb} | | | |
| D-2089 | {sGalNAc3K2AhxC6}gauguuuaGfaCfAfAfUf | 825 | asCfscuaaAfauugUfcUfaaacaucsusu | 826 |
| | uuuaggs{invAb} | | | |
| D-2090 | {sGalNAc3K2AhxC6}auguuuagAfcAfAfUfUf | 827 | asGfsccuaAfaauuGfuCfuaaacaususu | 828 |
| | uuaggcs{invAb} | | | |
| D-2091 | {sGalNAc3K2AhxC6}uguuuagaCfaAfUfUfUf | 829 | asAfsgccuAfaaauUfgUfcuaaacasusu | 830 |
| | uaggcus{invAb} | | | |
| D-2092 | {sGalNAc3K2AhxC6}guuuagacAfaUfUfUfUf | 831 | usGfsagccUfaaaaUfuGfucuaaacsusu | 832 |
| | aggcucs{invAb} | | | |
| D-2093 | {sGalNAc3K2AhxC6}uuuagacaAfuUfUfUfAf | 833 | usUfsgagcCfuaaaAfuUfgucuaaasusu | 834 |
| | ggcucas{invAb} | | | |
| D-2094 | {sGalNAc3K2AhxC6}uuagacaaUfuUfUfAfGf | 835 | usUfsugagCfcuaaAfaUfugucuaasusu | 836 |
| | gcucaas{invAb} | | | |
| D-2095 | {sGalNAc3K2AhxC6}agacaauuUfuAfGfGfCf | 837 | usUfsuuugAfgccuAfaAfauugucususu | 838 |
| | ucaaaas{invAb} | | | |
| D-2096 | {sGalNAc3K2AhxC6}auuuuaggCfuCfAfAfAf | 839 | usUfsuaauUfuuugAfgCfcuaaaaususu | 840 |
| | aauuaas{invAb} | | | |
| D-2097 | {sGalNAc3K2AhxC6}uuuaggcuCfaAfAfAfAf | 841 | asCfsuuuaAfuuuuUfgAfgccuaaasusu | 842 |
| | uuaaags{invAb} | | | |
| D-2098 | {sGalNAc3K2AhxC6}uuaggcucAfaAfAfAfUf | 843 | asGfscuuuAfauuuUfuGfagccuaasusu | 844 |
| | uaaagcs{invAb} | | | |
| D-2099 | {sGalNAc3K2AhxC6}aaaaauuaAfaGfCfUfAf | 845 | asCfsugugUfuagcUfuUfaauuuuususu | 846 |
| | acacags{invAb} | | | |
| D-2100 | {sGalNAc3K2AhxC6}aaaauuaaAfgCfUfAfAf | 847 | usCfscuguGfuuagCfuUfuaauuuususu | 848 |
| | cacaggs{invAb} | | | |
| D-2101 | {sGalNAc3K2AhxC6}aaagcuaaCfaCfAfGfGf | 849 | usCfscuuuUfccugUfgUfuagcuuususu | 850 |
| | aaaaggs{invAb} | | | |
| D-2102 | {sGalNAc3K2AhxC6}aagcuaacAfcAfGfGfAf | 851 | usUfsccuuUfuccuGfuGfuuagcuususu | 852 |
| | aaaggas{invAb} | | | |
| D-2103 | {sGalNAc3K2AhxC6}ggaaaaggAfaCfUfGfUf | 853 | asGfsccagUfacagUfuCfcuuuuccsusu | 854 |
| | acuggcs{invAb} | | | |
| D-2104 | {sGalNAc3K2AhxC6}aggaacugUfaCfUfGfGf | 855 | asUfsaauaGfccagUfaCfaguuccususu | 856 |
| | cuauuas{invAb} | | | |
| D-2105 | {sGalNAc3K2AhxC6}ccgacuccCfaCfUfAfCfa | 857 | usCfsuugaUfguagUfgGfgagucggsusu | 858 |
| | ucaags{invAb} | | | |
| D-2106 | {sGalNAc3K2AhxC6}gacucccaCfuAfCfAfUfc | 859 | asGfsucuuGfauguAfgUfgggagucsusu | 860 |
| | aagacs{invAb} | | | |
| D-2107 | {sGalNAc3K2AhxC6}ucccacuaCfaUfCfAfAfg | 861 | asUfsuaguCfuugaUfgUfagugggasusu | 862 |
| | acuaas{invAb} | | | |
| D-2108 | {sGalNAc3K2AhxC6}cccacuacAfuCfAfAfGfa | 863 | asAfsuuagUfcuugAfuGfuagugggsusu | 864 |
| | cuaaus{invAb} | | | |
| D-2109 | {sGalNAc3K2AhxC6}cacuacauCfaAfGfAfCfu | 865 | asAfsgauuAfgucuUfgAfuguagugsusu | 866 |
| | aaucus{invAb} | | | |
| D-2110 | {sGalNAc3K2AhxC6}acuacaucAfaGfAfCfUf | 867 | asAfsagauUfagucUfuGfauguagususu | 868 |
| | aaucuus{invAb} | | | |
| D-2111 | {sGalNAc3K2AhxC6}cuacaucaAfgAfCfUfAfa | 869 | asCfsaagaUfuaguCfuUfgauguagsusu | 870 |
| | ucuugs{invAb} | | | |
| D-2112 | {sGalNAc3K2AhxC6}guuuuucaCfaUfGfUfAf | 871 | usCfsuauaAfuacaUfgUfgaaaaacsusu | 872 |
| | uuauags{invAb} | | | |
| D-2113 | {sGalNAc3K2AhxC6}ucacauguAfuUfAfUfAf | 873 | asGfscauuCfuauaAfuAfcaugugasusu | 874 |
| | gaaugcs{invAb} | | | |
| D-2114 | {sGalNAc3K2AhxC6}acauguauUfaUfAfGfAf | 875 | asAfsagcaUfucuaUfaAfuacaugususu | 876 |
| | augcuus{invAb} | | | |
| D-2115 | {sGalNAc3K2AhxC6}uguauuauAfgAfAfUfGf | 877 | asCfsaaaaGfcauuCfuAfuaauacasusu | 878 |
| | cuuuugs{invAb} | | | |
| D-2116 | {sGalNAc3K2AhxC6}gaaugcuuUfuGfCfAfUf | 879 | asUfsagucCfaugcAfaAfagcauucsusu | 880 |
| | ggacuas{invAb} | | | |
| D-2117 | {sGalNAc3K2AhxC6}aaugcuuuUfgCfAfUfGf | 881 | asAfsuaguCfcaugCfaAfaagcauususu | 882 |
| | gacuaus{invAb} | | | |
| D-2118 | {sGalNAc3K2AhxC6}gcuuuugcAfuGfGfAfCf | 883 | asAfsggauAfguccAfuGfcaaaagcsusu | 884 |
| | uauccus{invAb} | | | |
| D-2119 | {sGalNAc3K2AhxC6}uuugcaugGfaCfUfAfUf | 885 | asAfsagagGfauagUfcCfaugcaaasusu | 886 |
| | ccucuus{invAb} | | | |
| D-2120 | {sGalNAc3K2AhxC6}uugcauggAfcUfAfUfCf | 887 | asCfsaagaGfgauaGfuCfcaugcaasusu | 888 |
| | cucuugs{invAb} | | | |
| D-2121 | {sGalNAc3K2AhxC6}ugcauggaCfuAfUfCfCf | 889 | asAfscaagAfggauAfgUfccaugcasusu | 890 |
| | ucuugus{invAb} | | | |
| D-2122 | {sGalNAc3K2AhxC6}gcauggacUfaUfCfCfUf | 891 | asAfsacaaGfaggaUfaGfuccaugcsusu | 892 |
| | cuuguus{invAb} | | | |
| D-2123 | {sGalNAc3K2AhxC6}auggacuaUfcCfUfCfUf | 893 | asAfsaaacAfagagGfaUfaguccaususu | 894 |
| | uguuuus{invAb} | | | |
| D-2124 | {sGalNAc3K2AhxC6}ggacuaucCfuCfUfUfGf | 895 | asUfsaaaaAfcaagAfgGfauaguccsusu | 896 |
| | uuuuuas{invAb} | | | |
| D-2125 | {sGalNAc3K2AhxC6}aauaaccuCfuUfGfUfAf | 897 | usUfsauaaCfuacaAfgAfgguuauususu | 898 |
| | guuauas{invAb} | | | |
| D-2126 | {sGalNAc3K2AhxC6}auaaccucUfuGfUfAfGf | 899 | usUfsuauaAfcuacAfaGfagguuaususu | 900 |
| | uuauaas{invAb} | | | |
| D-2127 | {sGalNAc3K2AhxC6}accucuugUfaGfUfUfAf | 901 | usAfsuuuuAfuaacUfaCfaagaggususu | 902 |
| | uaaaaus{invAb} | | | |
| D-2128 | {sGalNAc3K2AhxC6}ggucaaCfaUfcCfuagga | 903 | asAfsauguccuagGfaUfgUfugaccsusu | 904 |
| | cauus{invAb} | | | |
| D-2129 | {sGalNAc3K2AhxC6}gucaacAfuCfcUfaggac | 905 | asAfsaauguccuaGfgAfuGfuugacsusu | 906 |
| | auuus{invAb} | | | |
| D-2130 | {sGalNAc3K2AhxC6}ucaacaUfcCfUfAfGfgac | 907 | asAfsauguCfcuagGfaUfguugasusu | 908 |
| | auuuus{invAb} | | | |
| D-2131 | {sGalNAc3K2AhxC6}caacauCfcUfAfGfGfaca | 909 | asAfsaaugUfccuaGfgAfuguugsusu | 910 |
| | uuuuus{invAb} | | | |
| D-2132 | {sGalNAc3K2AhxC6}ggucaaCfaUfCfCfUfag | 911 | asAfsaUfgUfccuaggaUfgUfugaccsusu | 912 |
| | gacauus{invAb} | | | |
| D-2133 | {sGalNAc3K2AhxC6}gucaacAfuCfCfUfAfgga | 913 | asAfsaAfuGfuccuaggAfuGfuugacsusu | 914 |
| | cauuus{invAb} | | | |
| D-2134 | {sGalNAc3K2AhxC6}ggucaacaUfcCfUfAfGf | 915 | asAfsauguCfcuagGfaUfguugascsc | 916 |
| | gacauus{invAb} | | | |
| D-2135 | {sGalNAc3K2AhxC6}gucaacauCfcUfAfGfGf | 917 | asAfsaaugUfccuaGfgAfuguugsasc | 918 |
| | acauuus{invAb} | | | |
| D-2136 | {sGaINAc3K2AhxC6}ggucaacaUfCfCfUfagg | 919 | asAfsauguCfcuagGfaUfguugascsc | 920 |
| | acauus{invAb} | | | |
| D-2137 | {sGalNAc3K2AhxC6}gucaacauCfCfUfAfggac | 921 | asAfsaaugUfccuaGfgAfuguugsasc | 922 |
| | auuus{invAb} | | | |
| D-2138 | {sGalNAc3K2AhxC6}gucaacauccUfAfGfGfa | 923 | asAfsaAfuGfuccuaGfgAfuGfuugsasc | 924 |
| | cauuus{invAb} | | | |
| D-2139 | {sGalNAc3K2AhxC6}ggucaacaucCfUfAfGfg | 925 | asAfsaUfgUfccuagGfaUfgUfugascsc | 926 |
| | acauus{invAb} | | | |
| D-2140 | {sGalNAc3K2AhxC6}[invAb]ucaacaUfCfCfU | 927 | asAfsauguCfcuaggaUfgUfugasusu | 928 |
| | faggacaususu | | | |
| D-2141 | {sGalNAc3K2AhxC6}[invAb]caacauCfCfUfA | 929 | asAfsaaugUfccuaggAfuGfuugsusu | 930 |
| | fggacauususu | | | |
| D-2142 | {sGalNAc3K2AhxC6}ucaacaUfcCfUfAfGfgac | 931 | asAfsauguCfcuagGfaUfguugasusu | 932 |
| | auus{invAb} | | | |
| D-2143 | {sGalNAc3K2AhxC6}caacauCfcUfAfGfGfaca | 933 | asAfsaaugUfccuaGfgAfuguugsusu | 934 |
| | uuus{invAb} | | | |
| D-2144 | {sGalNAc3K2AhxC6}ggucaacaUfcGfAfUfGf | 935 | asAfsaugaCfcaucGfaUfguugaccsusu | 936 |
| | gucauus{invAb} | | | |
| D-2145 | {sGalNAc3K2AhxC6}gucaacauCfcAfUfCfGfa | 937 | asAfsaaucUfcgauGfgAfuguugacsusu | 938 |
| | gauuus{invAb} | | | |
| D-2146 | {sGalNAc3K2AhxC6}gucaacAfuCfCfUfAfgga | 939 | asAfsugucCfuaggAfuGfuugacsusu | 940 |
| | cauuus{invAb} | | | |
| D-2147 | {sGalNAc3K2AhxC6}cucccaCfuAfCfAfUfcaa | 941 | asGfsucuuGfauguAfgUfgggagsusu | 942 |
| | gacuus{invAb} | | | |
| D-2148 | {sGalNAc3K2AhxC6}ccacuaCfaUfCfAfAfgac | 943 | asUfsuaguCfuugaUfgUfaguggsusu | 944 |
| | uaauus{invAb} | | | |
| D-2149 | {sGalNAc3K2AhxC6}uuccuuAfuCfUfCfAfuc | 945 | usAfsagggAfugagAfuAfaggaasusu | 946 |
| | ccusus{invAb} | | | |
| D-2150 | {sGalNAc3K2AhxC6}uuccuuAfuGfAfGfAfuc | 947 | usAfsagggAfucucAfuAfaggaasusu | 948 |
| | ccusus{invAb} | | | |
| D-2151 | {sGaINAc3K2AhxC6}ucaacaUfcGfAfUfGfgu | 949 | asAfsaugaCfcaucGfaUfguugasusu | 950 |
| | cauus{invAb} | | | |
| D-2152 | {sGalNAc3K2AhxC6}uacaucAfaGfAfCfuaau | 951 | asAfscaag[GNA- | 952 |
| | cuugsusu | | A]uuagucUfuGfauguasgsu | |
| D-2153 | {sGalNAc3K2AhxC6}augcuuUfuGfCfAfugga | 953 | asGfsauag[GNA- | 954 |
| | cuauscs{invAb} | | T]ccaugcAfaAfagcaususc | |
| D-2154 | {sGalNAc3K2AhxC6}cguaugCfaGfAfAfuauu | 955 | asAfsauuGf[GNA- | 956 |
| | caaususu | | A]auauucUfgCfauacgsasu | |
| D-2155 | {sGalNAc3K2AhxC6}cguaugCfaGfAfAfuauu | 957 | asAfsaUfuGf[GNA- | 958 |
| | caaususu | | A]auauUfcUfgCfaUfaCfgsasu | |
| D-2156 | {sGalNAc3K2AhxC6}cuacauCfaAfGfAfcuaa | 959 | asCfsaaga[GNA- | 960 |
| | ucuusgsu | | T]uaguCfuUfgAfuguagsusg | |
| D-2157 | asasgccaUfgAfAfCfAfucauccuas{invAb} | 961 | asUfsaGfgAfugauguuCfaUfggcuususu | 962 |
| D-2158 | asgsccauGfaAfCfAfUfcauccuags{invAb} | 963 | usCfsuAfgGfaugauguUfcAfuggcususu | 964 |
| D-2159 | cscsaugaAfcAfUfCfAfuccuagaas{invAb} | 965 | usUfsuCfuAfggaugauGfuUfcauggsusu | 966 |
| D-2160 | usgsaacaUfcAfUfCfCfuagaaaucs{invAb} | 967 | asGfsaUfuUfcuaggauGfaUfguucasusu | 968 |
| D-2161 | gsasaguuUfuUfCfAfUfuccucagas{invAb} | 969 | asUfscUfgAfggaaugaAfaAfacuucsusu | 970 |
| D-2162 | gsgsagaaAfaUfCfUfGfuggcugggs{invAb} | 971 | asCfscCfaGfccacagaUfuUfucuccsusu | 972 |
| D-2163 | gsusggcuGfgGfGfAfGfauuguucus{invAb} | 973 | asAfsgAfaCfaaucuccCfcAfgccacsusu | 974 |
| D-2164 | gsgsaauaGfgCfAfGfGfcagacuacs{invAb} | 975 | asGfsuAfgUfcugccugCfcUfauuccsusu | 976 |
| D-2165 | gsasauagGfcAfGfGfCfagacuacus{invAb} | 977 | asAfsgUfaGfucugccuGfcCfuauucsusu | 978 |
| D-2166 | ususugcaAfaAfCfGfAfcagagcaus{invAb} | 979 | usAfsuGfcUfcugucguUfuUfgcaaasusu | 980 |
| D-2167 | gscsaaaaCfgAfCfAfGfagcauauus{invAb} | 981 | asAfsaUfaUfgcucuguCfgUfuuugcsusu | 982 |
| D-2168 | ascsgacaGfaGfCfAfUfauugguucs{invAb} | 983 | asGfsaAfcCfaauaugcUfcUfgucgususu | 984 |
| D-2169 | csgsacagAfgCfAfUfAfuugguucus{invAb} | 985 | asAfsgAfaCfcaauaugCfuCfugucgsusu | 986 |
| D-2170 | gsascagaGfcAfUfAfUfugguucugs{invAb} | 987 | asCfsaGfaAfccaauauGfcUfcugucsusu | 988 |
| D-2171 | ascsagagCfaUfAfUfUfgguucugus{invAb} | 989 | asAfscAfgAfaccaauaUfgCfucugususu | 990 |
| D-2172 | csasgagcAfuAfUfUfGfguucugugs{invAb} | 991 | asCfsaCfaGfaaccaauAfuGfcucugsusu | 992 |
| D-2173 | asgsagcaUfaUfUfGfGfuucuguggs{invAb} | 993 | asCfscAfcAfgaaccaaUfaUfgcucususu | 994 |
| D-2174 | gsasgcauAfuUfGfGfUfucugugggs{invAb} | 995 | usCfscCfaCfagaaccaAfuAfugcucsusu | 996 |
| D-2175 | csusguggGfaUfAfUfUfaauaagcgs{invAb} | 997 | asCfsgCfuUfauuaauaUfcCfcacagsusu | 998 |
| D-2176 | ususaauaAfgCfGfCfGfguguggags{invAb} | 999 | asCfsuCfcAfcaccgcgCfuUfauuaasusu | 1000 |
| D-2177 | usasauaaGfcGfCfGfGfuguggaggs{invAb} | 1001 | usCfscUfcCfacaccgcGfcUfuauuasusu | 1002 |
| D-2178 | asusaagcGfcGfGfUfGfuggaggaas{invAb} | 1003 | usUfsuCfcUfccacaccGfcGfcuuaususu | 1004 |
| D-2179 | usasagcgCfgGfUfGfUfggaggaaas{invAb} | 1005 | asUfsuUfcCfuccacacCfgCfgcuuasusu | 1006 |
| D-2180 | gsgscgucAfcUfGfCfGfcaugcguas{invAb} | 1007 | asUfsaCfgCfaugcgcaGfuGfacgccsusu | 1008 |
| D-2181 | gscsgucaCfuGfCfGfCfaugcguaus{invAb} | 1009 | asAfsuAfcGfcaugcgcAfgUfgacgcsusu | 1010 |
| D-2182 | csgsucacUfgCfGfCfAfugcguaugs{invAb} | 1011 | asCfsaUfaCfgcaugcgCfaGfugacgsusu | 1012 |
| D-2183 | asgscaacAfgAfGfAfAfgagaucuas{invAb} | 1013 | asUfsaGfaUfcucuucuCfuGfuugcususu | 1014 |
| D-2184 | csasacagAfgAfAfGfAfgaucuaucs{invAb} | 1015 | asGfsaUfaGfaucucuuCfuCfuguugsusu | 1016 |
| D-2185 | asascagaGfaAfGfAfGfaucuaucgs{invAb} | 1017 | asCfsgAfuAfgaucucuUfcUfcuguususu | 1018 |
| D-2186 | csasgagaAfgAfGfAfUfcuaucgcus{invAb} | 1019 | asAfsgCfgAfuagaucuCfuUfcucugsusu | 1020 |
| D-2187 | asgsagaaGfaGfAfUfCfuaucgcucs{invAb} | 1021 | asGfsaGfcGfauagaucUfcUfucucususu | 1022 |
| D-2188 | gsasgaagAfgAfUfCfUfaucgcucus{invAb} | 1023 | asAfsgAfgCfgauagauCfuCfuucucsusu | 1024 |
| D-2189 | asasgagaUfcUfAfUfCfgcucucuas{invAb} | 1025 | usUfsaGfaGfagcgauaGfaUfcucuususu | 1026 |
| D-2190 | gsasgaucUfaUfCfGfCfucucuaaas{invAb} | 1027 | asUfsuUfaGfagagcgaUfaGfaucucsusu | 1028 |
| D-2191 | gsasucuaUfcGfCfUfCfucuaaaucs{invAb} | 1029 | usGfsaUfuUfagagagcGfaUfagaucsusu | 1030 |
| D-2192 | asuscuauCfgCfUfCfUfcuaaaucas{invAb} | 1031 | asUfsgAfuUfuagagagCfgAfuagaususu | 1032 |
| D-2193 | csgscucuCfuAfAfAfUfcaggugaas{invAb} | 1033 | asUfsuCfaCfcugauuuAfgAfgagcgsusu | 1034 |
| D-2194 | csuscucuAfaAfUfCfAfggugaagas{invAb} | 1035 | usUfscUfuCfaccugauUfuAfgagagsusu | 1036 |
| D-2195 | asasagaaGfuGfGfGfUfgauguaacs{invAb} | 1037 | usGfsuUfaCfaucacccAfcUfucuuususu | 1038 |
| D-2196 | asasgaagUfgGfGfUfGfauguaacas{invAb} | 1039 | usUfsgUfuAfcaucaccCfaCfuucuususu | 1040 |
| D-2197 | asgsaaguGfgGfUfGfAfuguaacaas{invAb} | 1041 | asUfsuGfuUfacaucacCfcAfcuucususu | 1042 |
| D-2198 | gsasagugGfgUfGfAfUfguaacaaus{invAb} | 1043 | asAfsuUfgUfuacaucaCfcCfacuucsusu | 1044 |
| D-2199 | gsasuguaAfcAfAfUfCfguggugaas{invAb} | 1045 | asUfsuCfaCfcacgauuGfuUfacaucsusu | 1046 |
| D-2200 | asusguaaCfaAfUfCfGfuggugaaus{invAb} | 1047 | usAfsuUfcAfccacgauUfgUfuacaususu | 1048 |
| D-2201 | usgsuaacAfaUfCfGfUfggugaauas{invAb} | 1049 | usUfsaUfuCfaccacgaUfuGfuuacasusu | 1050 |
| D-2202 | gsusaacaAfuCfGfUfGfgugaauaas{invAb} | 1051 | asUfsuAfuUfcaccacgAfuUfguuacsusu | 1052 |
| D-2203 | usasacaaUfcGfUfGfGfugaauaaus{invAb} | 1053 | asAfsuUfaUfucaccacGfaUfuguuasusu | 1054 |
| D-2204 | gsgsugaaUfaAfUfGfCfugggacags{invAb} | 1055 | asCfsuGfuCfccagcauUfaUfucaccsusu | 1056 |
| D-2205 | gsusgaauAfaUfGfCfUfgggacagus{invAb} | 1057 | usAfscUfgUfcccagcaUfuAfuucacsusu | 1058 |
| D-2206 | usasaugcUfgGfGfAfCfaguauaucs{invAb} | 1059 | asGfsaUfaUfacuguccCfaGfcauuasusu | 1060 |
| D-2207 | asasugcuGfgGfAfCfAfguauauccs{invAb} | 1061 | usGfsgAfuAfuacugucCfcAfgcauususu | 1062 |
| D-2208 | asasgagaUfuAfCfCfAfagacauuus{invAb} | 1063 | asAfsaAfuGfucuugguAfaUfcucuususu | 1064 |
| D-2209 | asgsagauUfaCfCfAfAfgacauuugs{invAb} | 1065 | usCfsaAfaUfgucuuggUfaAfucucususu | 1066 |
| D-2210 | gsasgauuAfcCfAfAfGfacauuugas{invAb} | 1067 | asUfscAfaAfugucuugGfuAfaucucsusu | 1068 |
| D-2211 | usgsagguCfaAfCfAfUfccuaggacs{invAb} | 1069 | usGfsuCfcUfaggauguUfgAfccucasusu | 1070 |
| D-2212 | asgsgucaAfcAfUfCfCfuaggacaus{invAb} | 1071 | asAfsuGfuCfcuaggauGfuUfgaccususu | 1072 |
| D-2213 | gsgsucaaCfaUfCfCfUfaggacauus{invAb} | 1073 | asAfsaUfgUfccuaggaUfgUfugaccsusu | 1074 |
| D-2214 | gsuscaacAfuCfCfUfAfggacauuus{invAb} | 1075 | asAfsaAfuGfuccuaggAfuGfuugacsusu | 1076 |
| D-2215 | uscsaacaUfcCfUfAfGfgacauuuus{invAb} | 1077 | asAfsaAfaUfguccuagGfaUfguugasusu | 1078 |
| D-2216 | csasacauCfcUfAfGfGfacauuuuus{invAb} | 1079 | asAfsaAfaAfuguccuaGfgAfuguugsusu | 1080 |
| D-2217 | csasaaagCfaCfUfUfCfuuccaucgs{invAb} | 1081 | usCfsgAfuGfgaagaagUfgCfuuuugsusu | 1082 |
| D-2218 | asasaagcAfcUfUfCfUfuccaucgas{invAb} | 1083 | asUfscGfaUfggaagaaGfuGfcuuuususu | 1084 |
| D-2219 | asasagcaCfuUfCfUfUfccaucgaus{invAb} | 1085 | asAfsuCfgAfuggaagaAfgUfgcuuususu | 1086 |
| D-2220 | asasgcacUfuCfUfUfCfcaucgaugs{invAb} | 1087 | usCfsaUfcGfauggaagAfaGfugcuususu | 1088 |
| D-2221 | asgscacuUfcUfUfCfCfaucgaugas{invAb} | 1089 | asUfscAfuCfgauggaaGfaAfgugcususu | 1090 |
| D-2222 | csascuucUfuCfCfAfUfcgaugaugs{invAb} | 1091 | asCfsaUfcAfucgauggAfaGfaagugsusu | 1092 |
| D-2223 | ascsuucuUfcCfAfUfCfgaugauggs{invAb} | 1093 | usCfscAfuCfaucgaugGfaAfgaagususu | 1094 |
| D-2224 | ususccauCfgAfUfGfAfuggagagas{invAb} | 1095 | usUfscUfcUfccaucauCfgAfuggaasusu | 1096 |
| D-2225 | cscsaucgAfuGfAfUfGfgagagaaas{invAb} | 1097 | asUfsuUfcUfcuccaucAfuCfgauggsusu | 1098 |
| D-2226 | gsasuggaGfaGfAfAfAfucauggccs{invAb} | 1099 | usGfsgCfcAfugauuucUfcUfccaucsusu | 1100 |
| D-2227 | gsusggcuUfcAfGfUfGfugcggccas{invAb} | 1101 | asUfsgGfcCfgcacacuGfaAfgccacsusu | 1102 |
| D-2228 | usgsgcuuCfaGfUfGfUfgcggccacs{invAb} | 1103 | asGfsuGfgCfcgcacacUfgAfagccasusu | 1104 |
| D-2229 | gscsuucaGfuGfUfGfCfggccacgas{invAb} | 1105 | usUfscGfuGfgccgcacAfcUfgaagcsusu | 1106 |
| D-2230 | ususcaguGfuGfCfGfGfccacgaags{invAb} | 1107 | asCfsuUfcGfuggccgcAfcAfcugaasusu | 1108 |
| D-2231 | ususgugaAfuAfCfUfGfgguucaccs{invAb} | 1109 | usGfsgUfgAfacccaguAfuUfcacaasusu | 1110 |
| D-2232 | gsasauacUfgGfGfUfUfcaccaaaas{invAb} | 1111 | usUfsuUfuGfgugaaccCfaGfuauucsusu | 1112 |
| D-2233 | asusacugGfgUfUfCfAfccaaaaaus{invAb} | 1113 | asAfsuUfuUfuggugaaCfcCfaguaususu | 1114 |
| D-2234 | asgscacaAfgAfUfUfAfuggccugus{invAb} | 1115 | usAfscAfgGfccauaauCfuUfgugcususu | 1116 |
| D-2235 | csascaagAfuUfAfUfGfgccuguaus{invAb} | 1117 | asAfsuAfcAfggccauaAfuCfuugugsusu | 1118 |
| D-2236 | ascsaagaUfuAfUfGfGfccuguauus{invAb} | 1119 | asAfsaUfaCfaggccauAfaUfcuugususu | 1120 |
| D-2237 | csasagauUfaUfGfGfCfcuguauugs{invAb} | 1121 | asCfsaAfuAfcaggccaUfaAfucuugsusu | 1122 |
| D-2238 | asgsauuaUfgGfCfCfUfguauuggas{invAb} | 1123 | asUfscCfaAfuacaggcCfaUfaaucususu | 1124 |
| D-2239 | gsasuuauGfgCfCfUfGfuauuggags{invAb} | 1125 | usCfsuCfcAfauacaggCfcAfuaaucsusu | 1126 |
| D-2240 | gsasagucUfgAfUfAfGfauggaauas{invAb} | 1127 | asUfsaUfuCfcaucuauCfaGfacuucsusu | 1128 |
| D-2241 | asasgucuGfaUfAfGfAfuggaauacs{invAb} | 1129 | asGfsuAfuUfccaucuaUfcAfgacuususu | 1130 |
| D-2242 | asgsucugAfuAfGfAfUfggaauacus{invAb} | 1131 | asAfsgUfaUfuccaucuAfuCfagacususu | 1132 |
| D-2243 | gsuscugaUfaGfAfUfGfgaauacuus{invAb} | 1133 | usAfsaGfuAfuuccaucUfaUfcagacsusu | 1134 |
| D-2244 | uscsugauAfgAfUfGfGfaauacuuas{invAb} | 1135 | asUfsaAfgUfauuccauCfuAfucagasusu | 1136 |
| D-2245 | asusagauGfgAfAfUfAfcuuaccaas{invAb} | 1137 | asUfsuGfgUfaaguauuCfcAfucuaususu | 1138 |
| D-2246 | usasgaugGfaAfUfAfCfuuaccaaus{invAb} | 1139 | usAfsuUfgGfuaaguauUfcCfaucuasusu | 1140 |
| D-2247 | asgsauggAfaUfAfCfUfuaccaauas{invAb} | 1141 | usUfsaUfuGfguaaguaUfuCfcaucususu | 1142 |
| D-2248 | gsasuggaAfuAfCfUfUfaccaauaas{invAb} | 1143 | asUfsuAfuUfgguaaguAfuUfccaucsusu | 1144 |
| D-2249 | asusggaaUfaCfUfUfAfccaauaags{invAb} | 1145 | usCfsuUfaUfugguaagUfaUfuccaususu | 1146 |
| D-2250 | usgsgaauAfcUfUfAfCfcaauaagas{invAb} | 1147 | usUfscUfuAfuugguaaGfuAfuuccasusu | 1148 |
| D-2251 | asusaucaAfuAfUfCfUfuucugagas{invAb} | 1149 | asUfscUfcAfgaaagauAfuUfgauaususu | 1150 |
| D-2252 | csusuucuGfaGfAfCfUfacagaagus{invAb} | 1151 | asAfscUfuCfuguagucUfcAfgaaagsusu | 1152 |
| D-2253 | ususucugAfgAfCfUfAfcagaaguus{invAb} | 1153 | asAfsaCfuUfcuguaguCfuCfagaaasusu | 1154 |
| D-2254 | ususcugaGfaCfUfAfCfagaaguuus{invAb} | 1155 | asAfsaAfcUfucuguagUfcUfcagaasusu | 1156 |
| D-2255 | uscsugagAfcUfAfCfAfgaaguuucs{invAb} | 1157 | asGfsaAfaCfuucuguaGfuCfucagasusu | 1158 |
| D-2256 | usgsguugGfcCfAfCfAfaaaucaaas{invAb} | 1159 | usUfsuUfgAfuuuugugGfcCfaaccasusu | 1160 |
| D-2257 | asasaugaAfaUfGfAfAfuaaauaags{invAb} | 1161 | asCfsuUfaUfuuauuca UfuUfcauuususu | 1162 |
| D-2258 | ususcacaUfuUfUfUfUfcaguccugs{invAb} | 1163 | usCfsaGfgAfcugaaaaAfaUfgugaasusu | 1164 |
| D-2259 | gsusuuggCfaCfUfAfGfcagcagucs{invAb} | 1165 | usGfsaCfuGfcugcuagUfgCfcaaacsusu | 1166 |
| D-2260 | ususuggcAfcUfAfGfCfagcagucas{invAb} | 1167 | usUfsgAfcUfgcugcuaGfuGfccaaasusu | 1168 |
| D-2261 | ususggcaCfuAfGfCfAfgcagucaas{invAb} | 1169 | usUfsuGfaCfugcugcuAfgUfgccaasusu | 1170 |
| D-2262 | usgsgcacUfaGfCfAfGfcagucaaas{invAb} | 1171 | asUfsuUfgAfcugcugcUfaGfugccasusu | 1172 |
| D-2263 | gsgscacuAfgCfAfGfCfagucaaacs{invAb} | 1173 | asGfsuUfuGfacugcugCfuAfgugccsusu | 1174 |
| D-2264 | asusuuacGfuAfGfUfUfuuucauags{invAb} | 1175 | asCfsuAfuGfaaaaacuAfcGfuaaaususu | 1176 |
| D-2265 | ususacguAfgUfUfUfUfucauaggus{invAb} | 1177 | asAfscCfuAfugaaaaaCfuAfcguaasusu | 1178 |
| D-2266 | usascguaGfuUfUfUfUfcauaggucs{invAb} | 1179 | asGfsaCfcUfaugaaaaAfcUfacguasusu | 1180 |
| D-2267 | ususacauAfaAfCfAfUfacuuaaaas{invAb} | 1181 | asUfsuUfuAfaguauguUfuAfuguaasusu | 1182 |
| D-2268 | ususaaagGfuGfGfAfCfaaaagcuas{invAb} | 1183 | asUfsaGfcUfuuuguccAfcCfuuuaasusu | 1184 |
| D-2269 | usasaaggUfgGfAfCfAfaaagcuacs{invAb} | 1185 | asGfsuAfgCfuuuugucCfaCfcuuuasusu | 1186 |
| D-2270 | asasggugGfaCfAfAfAfagcuaccus{invAb} | 1187 | asAfsgGfuAfgcuuuugUfcCfaccuususu | 1188 |
| D-2271 | gsgsuggaCfaAfAfAfGfcuaccuccs{invAb} | 1189 | asGfsgAfgGfuagcuuuUfgUfccaccsusu | 1190 |
| D-2272 | ascsagcuAfaGfAfGfAfucaaguuus{invAb} | 1191 | asAfsaAfcUfugaucucUfuAfgcugususu | 1192 |
| D-2273 | csasgcuaAfgAfGfAfUfcaaguuucs{invAb} | 1193 | usGfsaAfaCfuugaucuCfuUfagcugsusu | 1194 |
| D-2274 | asgscuaaGfaGfAfUfCfaaguuucas{invAb} | 1195 | asUfsgAfaAfcuugaucUfcUfuagcususu | 1196 |
| D-2275 | cscsuggaCfaUfAfUfUfuuaagauus{invAb} | 1197 | asAfsaUfcUfuaaaauaUfgUfccaggsusu | 1198 |
| D-2276 | csusggacAfuAfUfUfUfuaagauucs{invAb} | 1199 | usGfsaAfuCfuuaaaauAfuGfuccagsusu | 1200 |
| D-2277 | csusuccuUfuUfUfCfAfuuagcccas{invAb] | 1201 | usUfsgGfgCfuaaugaaAfaAfggaagsusu | 1202 |
| D-2278 | ususccuuUfuUfCfAfUfuagcccaas{invAb} | 1203 | usUfsuGfgGfcuaaugaAfaAfaggaasusu | 1204 |
| D-2279 | cscscucuAfuAfUfUfUfccucccuus{invAb] | 1205 | asAfsaGfgGfaggaaauAfuAfgagggsusu | 1206 |
| D-2280 | usasuuucCfuCfCfCfUfuuuuauags{invAb} | 1207 | asCfsuAfuAfaaaagggAfgGfaaauasusu | 1208 |
| D-2281 | ususccucCfcUfUfUfUfuauagucus{invAb} | 1209 | asAfsgAfcUfauaaaaaGfgGfaggaasusu | 1210 |
| D-2282 | uscscuccCfuUfUfUfUfauagucuus{invAb} | 1211 | usAfsaGfaCfuauaaaaAfgGfgaggasusu | 1212 |
| D-2283 | cscsuuuuUfaUfAfGfUfcuuauaags{invAb} | 1213 | usCfsuUfaUfaagacuaUfaAfaaaggsusu | 1214 |
| D-2284 | csusuuuuAfuAfGfUfCfuuauaagas{invAb} | 1215 | asUfscUfuAfuaagacuAfuAfaaaagsusu | 1216 |
| D-2285 | ususuuuaUfaGfUfCfUfuauaagaus{invAb} | 1217 | usAfsuCfuUfauaagacUfaUfaaaaasusu | 1218 |
| D-2286 | ususuuauAfgUfCfUfUfauaagauas{invAb} | 1219 | asUfsaUfcUfuauaagaCfuAfuaaaasusu | 1220 |
| D-2287 | ususuauaGfuCfUfUfAfuaagauacs{invAb} | 1221 | usGfsuAfuCfuuauaagAfcUfauaaasusu | 1222 |
| D-2288 | ususauagUfcUfUfAfUfaagauacas{invAb} | 1223 | asUfsgUfaUfcuuauaaGfaCfuauaasusu | 1224 |
| D-2289 | usasuaguCfuUfAfUfAfagauacaus{invAb} | 1225 | asAfsuGfuAfucuuauaAfgAfcuauasusu | 1226 |
| D-2290 | uscsuuauAfaGfAfUfAfcauuaugas{invAb} | 1227 | usUfscAfuAfauguaucUfuAfuaagasusu | 1228 |
| D-2291 | ususuuaaGfuUfCfUfAfgccccaugs{invAb} | 1229 | usCfsaUfgGfggcuagaAfcUfuaaaasusu | 1230 |
| D-2292 | ususuaagUfuCfUfAfGfccccaugas{invAb} | 1231 | asUfscAfuGfgggcuagAfaCfuuaaasusu | 1232 |
| D-2293 | usasaguuCfuAfGfCfCfccaugauas{invAb} | 1233 | usUfsaUfcAfuggggcuAfgAfacuuasusu | 1234 |
| D-2294 | asasguucUfaGfCfCfCfcaugauaas{invAb} | 1235 | asUfsuAfuCfauggggcUfaGfaacuususu | 1236 |
| D-2295 | asgsuucuAfgCfCfCfCfaugauaacs{invAb} | 1237 | asGfsuUfaUfcauggggCfuAfgaacususu | 1238 |
| D-2296 | gsusucuaGfcCfCfCfAfugauaaccs{invAb} | 1239 | asGfsgUfuAfucaugggGfcUfagaacsusu | 1240 |
| D-2297 | csusagccCfcAfUfGfAfuaaccuuus{invAb} | 1241 | asAfsaAfgGfuuaucauGfgGfgcuagsusu | 1242 |
| D-2298 | asgsccccAfuGfAfUfAfaccuuuuus{invAb] | 1243 | asAfsaAfaAfgguuaucAfuGfgggcususu | 1244 |
| D-2299 | gscscccaUfgAfUfAfAfccuuuuucs{invAb} | 1245 | asGfsaAfaAfagguuauCfaUfggggcsusu | 1246 |
| D-2300 | cscscaugAfuAfAfCfCfuuuuucuus{invAb} | 1247 | asAfsaGfaAfaaagguuAfuCfaugggsusu | 1248 |
| D-2301 | cscsaugaUfaAfCfCfUfuuuucuuus{invAb} | 1249 | asAfsaAfgAfaaaagguUfaUfcauggsusu | 1250 |
| D-2302 | csasugauAfaCfCfUfUfuuucuuugs{invAb} | 1251 | asCfsaAfaGfaaaaaggUfuAfucaugsusu | 1252 |
| D-2303 | asusaaccUfuUfUfUfCfuuuguaaus{invAb} | 1253 | asAfsuUfaCfaaagaaaAfaGfguuaususu | 1254 |
| D-2304 | ususuuucUfuUfGfUfAfauuuaugcs{invAb} | 1255 | asGfscAfuAfaauuacaAfaGfaaaaasusu | 1256 |
| D-2305 | ususuucuUfuGfUfAfAfuuuaugcus{invAb} | 1257 | asAfsgCfaUfaaauuacAfaAfgaaaasusu | 1258 |
| D-2306 | gsgscuauUfaCfAfUfAfagaaacaas{invAb} | 1259 | asUfsuGfuUfucuuaugUfaAfuagccsusu | 1260 |
| D-2307 | csusauuaCfaUfAfAfGfaaacaaugs{invAb} | 1261 | asCfsaUfuGfuuucuuaUfgUfaauagsusu | 1262 |
| D-2308 | ususacauAfaGfAfAfAfcaauggacs{invAb} | 1263 | asGfsuCfcAfuuguuucUfuAfuguaasusu | 1264 |
| D-2309 | usascauaAfgAfAfAfCfaauggaccs{invAb} | 1265 | asGfsgUfcCfauuguuuCfuUfauguasusu | 1266 |
| D-2310 | ascsauaaGfaAfAfCfAfauggacccs{invAb] | 1267 | usGfsgGfuCfcauuguuUfcUfuaugususu | 1268 |
| D-2311 | asasgaaaCfaAfUfGfGfacccaagas{invAb} | 1269 | asUfscUfuGfgguccauUfgUfuucuususu | 1270 |
| D-2312 | asgsaaacAfaUfGfGfAfcccaagags{invAb} | 1271 | usCfsuCfuUfggguccaUfuGfuuucususu | 1272 |
| D-2313 | gsasaacaAfuGfGfAfCfccaagagas{invAb} | 1273 | usUfscUfcUfuggguccAfuUfguuucsusu | 1274 |
| D-2314 | asasuagaAfaAfAfAfUfaauccgacs{invAb} | 1275 | asGfsuCfgGfauuauuuUfuUfcuauususu | 1276 |
| D-2315 | asusagaaAfaAfAfUfAfauccgacus{invAb} | 1277 | asAfsgUfcGfgauuauuUfuUfucuaususu | 1278 |
| D-2316 | asasaacaAfuUfCfAfCfuaaaaauas{invAb} | 1279 | usUfsaUfuUfuuagugaAfuUfguuuususu | 1280 |
| D-2317 | usgsuaguUfaUfAfAfAfauaaaacgs{invAb} | 1281 | asCfsgUfuUfuauuuuaUfaAfcuacasusu | 1282 |
| D-2318 | asasuaaaAfcGfUfUfUfgacuucuas{invAb} | 1283 | usUfsaGfaAfgucaaacGfuUfuuauususu | 1284 |
| D-2319 | asusaaaaCfgUfUfUfGfacuucuaas{invAb} | 1285 | usUfsuAfgAfagucaaaCfgUfuuuaususu | 1286 |
| D-2320 | usasaaacGfuUfUfGfAfcuucuaaas{invAb} | 1287 | asUfsuUfaGfaagucaaAfcGfuuuuasusu | 1288 |
| D-2321 | asasaacgUfuUfGfAfCfuucuaaacs{invAb} | 1289 | asGfsuUfuAfgaagucaAfaCfguuuususu | 1290 |
| D-2322 | asasacguUfuGfAfCfUfucuaaacus{invAb} | 1291 | asAfsgUfuUfagaagucAfaAfcguuususu | 1292 |

### Example 3: Droplet Digital PCR Assay of siRNA for HSD17B13-rs738409 and HSD17B13-rs738409-rs738408

Following the manufacturers protocol, thawed human primary hepatocyte cells (Xenotech/Sekisui donor lot #HC3-38) in OptiThaw media (Xenotech cat #K8000), cells were centrifuged and post media aspiration, resuspended in OptiPlate hepatocyte media (Xenotech cat #K8200) and plated into 96 well collagen coated plates (Greiner cat #655950). Following a 2-4 hour incubation period, media was removed and replaced with OptiCulture hepatocyte media (Xenotech cat #K8300). 2-4 hours post addition of OptiCulture media, delivered GalNAc conjugated siRNAs to cells via free uptake (no transfection reagent) at various concentrations up to 3.8 uM. Cells were incubated 24-72 hours at 37° C. and 5% CO2. Cells were then lysed with Qiagen RLT buffer (79216)+1% 2-mercaptoethanol (Sigma, M-3148), and lysates were stored at −20° C. RNA was purified using a Qiagen QIACube HT instrument (9001793) and a Qiagen RNeasy 96 QIACube HT Kit (74171) according to manufacturer's instructions. Samples were analyzed using a QIAxpert system (9002340). cDNA was synthesized from RNA samples using the Applied Biosystems High Capacity cDNA Reverse Transcription kit (4368813), reactions were assembled according to manufacturer's instructions, input RNA concentration varied by sample. Reverse transcription was carried out on a BioRad tetrad thermal cycler (model #PTC-0240G) under the following conditions: 25° C. 10 minutes, 37° C. 120 minutes, 85° C. 5 minutes followed by (an optional) 4° C. infinite hold.

Droplet digital PCR (ddPCR) was performed using BioRad's QX200 AutoDG droplet digital PCR system according to manufacturer's instructions. Reactions were assembled into an Eppendorf clear 96 well PCR plate (951020303) using BioRad ddPCR Supermix for Probes (1863010), and fluorescently labeled qPCR assays for HSD17B13 (IDT Hs.PT.58.21464637, primer to probe ratio 3.6:1 and TBP (IDT Hs.PT.53a.20105486, primer to probe ratio 3.6:1) and RNase free water (Ambion, AM9937). Final primer/probe concentration is 900 nM/250 nM respectively, input cDNA concentration varied among wells. Droplets were formed using a BioRad Auto DG droplet generator (1864101) set up with manufacturer recommended consumables (BioRad DG32 cartridges 1864108, BioRad tips 1864121, Eppendorf blue 96 well PCR plate 951020362, BioRad droplet generation oil for probes 1864110 and a BioRad droplet plate assembly). Droplets were amplified on a BioRad C1000 touch thermal cycler (1851197) using the following conditions: enzyme activation 95° C. 10 minutes, denaturation 94° C. 30 seconds followed by annealing/extension 60° C. for one minute, 40 cycles using a 2° C./second ramp rate, enzyme deactivation 98° C. 10 minutes followed by (an optional) 4° C. infinite hold. Samples were then read on a BioRad QX200 Droplet Reader measuring FAM/HEX signal that correlates to HSD17B13 or TBP concentration. Data was analyzed using BioRad's QuantaSoft software package. Samples were gated by channel (fluorescent label) to determine the concentration per sample. Each sample was then expressed as the ratio of the concentration of the gene of interest (HSD17B13)/concentration of the housekeeping gene (TBP) to control for differences in sample loading. Data is then imported into Genedata Screener, where each test siRNA is normalized to the median of the neutral control wells (buffer only). IC50 values are reported in Table 3.

TABLE 3
ddPCR assay on primary hepatocyte cells

| Duplex No. | IC50 (μM) | % HSD17B13 knockdown |
|---|---|---|
| D-2107 | 0.0112 | −88.9134 |
| D-2015 | 0.0112 | −91.9705 |
| D-2016 | 0.0296 | −87.2192 |
| D-2014 | 0.0343 | −80.4788 |

### Example 4: Screening of Chemically Modified HSD17B13 siRNA Molecules in Wildtype Rats

Sprague Dawley male rats at 9-10 weeks of age and 350-400 gms body weight were obtained from Charles River Laboratories (Charles River Laboratories, Inc, MA). After acclimation, these animals were randomized based upon body weight. 6 rats were included in each group and were subcutaneously dosed with HSD17B13 siRNA at 3 milligram per kilogram body weight. The dosing compounds were diluted in phosphate buffer solution without Calcium and Magnesium (Thermo Fischer Scientific, 14190-136). 30 days after siRNA treatment, animals were euthanized, and livers were harvested. Freshly isolated left lobe of the liver was immediately snap frozen in liquid nitrogen. 30-50 mg of liver tissue was used to isolate RNA using the QIAcube HT instrument and RNeasy 96 QIAcube HT kits according to manufacturer's protocol. 2-4 ug of RNA were treated with RQ1 RNase-Free DNase (Promega, M6101). 10 ng of DNAse digested RNA was subjected to Real Time qPCR using the TaqMan RNA to CT 1 step kit (Applied Biosystems) run on the Quant Studio Real Time PCR machine. TaqMan probes for rat HSD17B13 (Rn_01450039_m1, Invitrogen Taqman expression assays) were used to measure the expression and normalized to the housekeeping gene HMBS (Hydroxymethylbilane synthase Rn01421873_g1, Invitrogen Taqman expression assays) expression. Relative fold change was calculated when compared to the PBS cohort. Data is represented as percent knockdown in the siRNA treated group with respect to PBS. A total of 23 triggers were tested. The results are shown in Table 4. Negative values indicate an increase in HSD17B13 levels.

TABLE 4
Day 30- percent silencing in the siRNA HSD17B13 treated rats

| | | % |
|---|---|---|
| | Dose | HSD17B13 |
| Duplex # | administered | knockdown |
| D-2128 | 3mpk | 30.01 |
| D-2130 | 3mpk | 29.72 |
| D-2132 | 3mpk | −3.06 |
| D-2134 | 3mpk | 35.70 |
| D-2144 | 3mpk | −10.08 |
| D-2136 | 3mpk | 44.10 |
| D-2129 | 3mpk | 22.62 |
| D-2131 | 3mpk | 15.73 |
| D-2133 | 3mpk | 13.87 |
| D-2135 | 3mpk | 17.91 |
| D-2145 | 3mpk | 1.38 |
| D-2137 | 3mpk | 36.82 |
| D-2138 | 3mpk | 1.26 |
| D-2139 | 3mpk | 15.34 |
| D-2140 | 3mpk | 45.71 |
| D-2141 | 3mpk | 30.72 |
| D-2142 | 3mpk | 66.39 |
| D-2143 | 3mpk | 31.92 |
| D-2146 | 3mpk | 4.41 |
| D-2147 | 3mpk | 10.70 |
| D-2148 | 3mpk | 15.92 |
| D-2015 | 3mpk | 35.10 |
| D-2016 | 3mpk | 24.79 |

What is claimed is:

1. An RNAi construct comprising a sense strand and an antisense strand, wherein the antisense strand comprises a region having at least 15 contiguous nucleotides differing by no more than 3 nucleotides from an antisense sequence listed in Table 1 or 2, and wherein the RNAi construct inhibits the expression of 17β-Hydroxysteroid dehydrogenase type 13 (HSD17B13).

2. The RNAi construct of claim 1, wherein the antisense strand comprises a region that is complementary to a HSD17B13 mRNA sequence.

3. The RNAi construct of claim 1, wherein the sense strand comprises a region having at least 15 contiguous nucleotides differing by no more than 3 nucleotides from an antisense sequence listed in Table 1 or 2.

4. The RNAi construct of claim 3, wherein the sense strand comprises a sequence that is sufficiently complementary to the sequence of the antisense strand to form a duplex region of about 15 to about 30 base pairs in length.

5. The RNAi construct of claim 4, wherein the duplex region is about 17 to about 24 base pairs in length.

6. (canceled)

7. (canceled)

8. (canceled)

9. (canceled)

10. The RNAi construct of claim 4, wherein the sense strand and the antisense strand are each about 15 to about 30 nucleotides in length.

11. (canceled)

12. (canceled)

13. (canceled)

14. The RNAi construct of claim 1, wherein the RNAi construct comprises at least one blunt end.

15. The RNAi construct of claim 1, wherein the RNAi construct comprises at least one nucleotide overhang of 1 to 4 unpaired nucleotides.

16. (canceled)

17. (canceled)

18. (canceled)

19. The RNAi construct of claim 1, wherein the RNAi construct comprises at least one modified nucleotide.

20. The RNAi construct of claim 19, wherein the modified nucleotide is a 2′-modified nucleotide.

21. The RNAi construct of claim 19, wherein the modified nucleotide is a 2′-fluoro modified nucleotide, a 2′-O-methyl modified nucleotide, a 2′-O-methoxyethyl modified nucleotide, a 2′-O-allyl modified nucleotide, a bicyclic nucleic acid (BNA), a glycol nucleic acid, an inverted base or combinations thereof.

22. (canceled)

23. The RNAi construct of claim 19, wherein all of the nucleotides in the sense and antisense strands are modified nucleotides.

24. (canceled)

25. The RNAi construct of claim 1, wherein the RNAi construct comprises at least one phosphorothioate internucleotide linkage.

26. The RNAi construct of claim 25, wherein the RNAi construct comprises two consecutive phosphorothioate internucleotide linkages at the 3′ end of the antisense strand.

27. The RNAi construct of claim 25, wherein the RNAi construct comprises two consecutive phosphorothioate internucleotide linkages at both the 3′ and 5′ ends of the antisense strand and two consecutive phosphorothioate internucleotide linkages at the 5′ end of the sense strand.

28. (canceled)

29. (canceled)

30. (canceled)

31. The RNAi construct of claim 1, wherein the RNAi construct reduces the expression level of HSD17B13 in liver cells following incubation with the RNAi construct as compared to the HSD17B13 expression level in liver cells that have been incubated with a control RNAi construct.

32. (canceled)

33. The RNAi construct of claim 1, wherein the RNAi construct inhibits HSD17B13 expression in primary hepatocyte cells with an IC50 of less than about 40 nM.

34. (canceled)

35. A pharmaceutical composition comprising the RNAi construct of claim 1 and a pharmaceutically acceptable carrier, excipient, or diluent.

36. A method for reducing the expression of HSD17B13 in a patient in need thereof comprising administering to the patient the RNAi construct of claim 1.

37. The method of claim 36, wherein the expression level of HSD17B13 in hepatocytes is reduced in the patient following administration of the RNAi construct as compared to the HSD17B13 expression level in a patient not receiving the RNAi construct.


SEQUENCE LISTING (1292 sequences)

SEQ ID NO: 1 - RNA, 21 nt, Artificial Sequence
  source: /note=“Description of Artificial Sequence Synthetic oligonucleotide”
  modified_base (21)..(21): Inverted abasic nucleotide
uucugcuucugaucaccaucn

SEQ ID NO: 2 - RNA, 23 nt, Artificial Sequence
  source: /note=“Description of Artificial Sequence Synthetic oligonucleotide”
ugauggugaucagaagcagaauu

SEQ ID NO: 3 - RNA, 21 nt, Artificial Sequence
  source: /note=“Description of Artificial Sequence Synthetic oligonucleotide”
  modified_base (21)..(21): Inverted abasic nucleotide
ucugcuucugaucaccaucan

SEQ ID NO: 4 - RNA, 23 nt, Artificial Sequence
  source: /note=“Description of Artificial Sequence Synthetic oligonucleotide”
augauggugaucagaagcagauu

SEQ ID NO: 5 - RNA, 21 nt, Artificial Sequence
  source: /note=“Description of Artificial Sequence Synthetic oligonucleotide”
  modified_base (21)..(21): Inverted abasic nucleotide
cuucugaucaccaucaucuan

SEQ ID NO: 6 - RNA, 23 nt, Artificial Sequence
  source: /note=“Description of Artificial Sequence Synthetic oligonucleotide”
auagaugauggugaucagaaguu

SEQ ID NO: 7 - RNA, 21 nt, Artificial Sequence
  source: /note=“Description of Artificial Sequence Synthetic oligonucleotide”
  modified_base (21)..(21): Inverted abasic nucleotide
ucucauuacuggagcugggcn

SEQ ID NO: 8 - RNA, 23 nt, Artificial Sequence
  source: /note=“Description of Artificial Sequence Synthetic oligonucleotide”
ugcccagcuccaguaaugagauu

SEQ ID NO: 9 - RNA, 21 nt, Artificial Sequence
  source: /note=“Description of Artificial Sequence Synthetic oligonucleotide”
  modified_base (21)..(21): Inverted abasic nucleotide
gugaauaaugcugggacagun

SEQ ID NO: 10 - RNA, 23 nt, Artificial Sequence
  source: /note=“Description of Artificial Sequence Synthetic oligonucleotide”
uacugucccagcauuauucacuu

SEQ ID NO: 11 - RNA, 21 nt, Artificial Sequence
  source: /note=“Description of Artificial Sequence Synthetic oligonucleotide”
  modified_base (21)..(21): Inverted abasic nucleotide
uaaugcugggacaguauaucn

SEQ ID NO: 12 - RNA, 23 nt, Artificial Sequence
  source: /note=“Description of Artificial Sequence Synthetic oligonucleotide”
agauauacugucccagcauuauu

SEQ ID NO: 13 - RNA, 21 nt, Artificial Sequence
  source: /note=“Description of Artificial Sequence Synthetic oligonucleotide”
  modified_base (21)..(21): Inverted abasic nucleotide
aaugcugggacaguauauccn

SEQ ID NO: 14 - RNA, 23 nt, Artificial Sequence
  source: /note=“Description of Artificial Sequence Synthetic oligonucleotide”
uggauauacugucccagcauuuu

SEQ ID NO: 15 - RNA, 21 nt, Artificial Sequence
  source: /note=“Description of Artificial Sequence Synthetic oligonucleotide”
  modified_base (21)..(21): Inverted abasic nucleotide
gggacaguauauccagccgan

SEQ ID NO: 16 - RNA, 23 nt, Artificial Sequence
  source: /note=“Description of Artificial Sequence Synthetic oligonucleotide”
aucggcuggauauacugucccuu

SEQ ID NO: 17 - RNA, 21 nt, Artificial Sequence
  source: /note=“Description of Artificial Sequence Synthetic oligonucleotide”
  modified_base (21)..(21): Inverted abasic nucleotide
ggacaguauauccagccgaun

SEQ ID NO: 18 - RNA, 23 nt, Artificial Sequence
  source: /note=“Description of Artificial Sequence Synthetic oligonucleotide”
aaucggcuggauauacuguccuu

SEQ ID NO: 19 - RNA, 21 nt, Artificial Sequence
  source: /note=“Description of Artificial Sequence Synthetic oligonucleotide”
  modified_base (21)..(21): Inverted abasic nucleotide
gacaguauauccagccgaucn

SEQ ID NO: 20 - RNA, 23 nt, Artificial Sequence
  source: /note=“Description of Artificial Sequence Synthetic oligonucleotide”
agaucggcuggauauacugucuu

SEQ ID NO: 21 - RNA, 21 nt, Artificial Sequence
  source: /note=“Description of Artificial Sequence Synthetic oligonucleotide”
  modified_base (21)..(21): Inverted abasic nucleotide
acaguauauccagccgaucun

SEQ ID NO: 22 - RNA, 23 nt, Artificial Sequence
  source: /note=“Description of Artificial Sequence Synthetic oligonucleotide”
aagaucggcuggauauacuguuu

SEQ ID NO: 23 - RNA, 21 nt, Artificial Sequence
  source: /note=“Description of Artificial Sequence Synthetic oligonucleotide”
  modified_base (21)..(21): Inverted abasic nucleotide
caguauauccagccgaucuun

SEQ ID NO: 24 - RNA, 23 nt, Artificial Sequence
  source: /note=“Description of Artificial Sequence Synthetic oligonucleotide”
aaagaucggcuggauauacuguu

SEQ ID NO: 25 - RNA, 21 nt, Artificial Sequence
  source: /note=“Description of Artificial Sequence Synthetic oligonucleotide”
  modified_base (21)..(21): Inverted abasic nucleotide
gacauuugaggucaacauccn

SEQ ID NO: 26 - RNA, 23 nt, Artificial Sequence
  source: /note=“Description of Artificial Sequence Synthetic oligonucleotide”
aggauguugaccucaaaugucuu

SEQ ID NO: 27 - RNA, 21 nt, Artificial Sequence
  source: /note=“Description of Artificial Sequence Synthetic oligonucleotide”
  modified_base (21)..(21): Inverted abasic nucleotide
ugaggucaacauccuaggacn

SEQ ID NO: 28 - RNA, 23 nt, Artificial Sequence
  source: /note=“Description of Artificial Sequence Synthetic oligonucleotide”
uguccuaggauguugaccucauu

SEQ ID NO: 29 - RNA, 21 nt, Artificial Sequence
  source: /note=“Description of Artificial Sequence Synthetic oligonucleotide”
  modified_base (21)..(21): Inverted abasic nucleotide
aggucaacauccuaggacaun

SEQ ID NO: 30 - RNA, 23 nt, Artificial Sequence
  source: /note=“Description of Artificial Sequence Synthetic oligonucleotide”
aauguccuaggauguugaccuuu

SEQ ID NO: 31 - RNA, 21 nt, Artificial Sequence
  source: /note=“Description of Artificial Sequence Synthetic oligonucleotide”
  modified_base (21)..(21): Inverted abasic nucleotide
ggucaacauccuaggacauun

SEQ ID NO: 32 - RNA, 23 nt, Artificial Sequence
  source: /note=“Description of Artificial Sequence Synthetic oligonucleotide”
aaauguccuaggauguugaccuu

SEQ ID NO: 33 - RNA, 21 nt, Artificial Sequence
  source: /note=“Description of Artificial Sequence Synthetic oligonucleotide”
  modified_base (21)..(21): Inverted abasic nucleotide
gucaacauccuaggacauuun

SEQ ID NO: 34 - RNA, 23 nt, Artificial Sequence
  source: /note=“Description of Artificial Sequence Synthetic oligonucleotide”
aaaauguccuaggauguugacuu

SEQ ID NO: 35 - RNA, 21 nt, Artificial Sequence
  source: /note=“Description of Artificial Sequence Synthetic oligonucleotide”
  modified_base (21)..(21): Inverted abasic nucleotide
ucaacauccuaggacauuuun

SEQ ID NO: 36 - RNA, 23 nt, Artificial Sequence
  source: /note=“Description of Artificial Sequence Synthetic oligonucleotide”
aaaaauguccuaggauguugauu

SEQ ID NO: 37 - RNA, 21 nt, Artificial Sequence
  source: /note=“Description of Artificial Sequence Synthetic oligonucleotide”
  modified_base (21)..(21): Inverted abasic nucleotide
caacauccuaggacauuuuun

SEQ ID NO: 38 - RNA, 23 nt, Artificial Sequence
  source: /note=“Description of Artificial Sequence Synthetic oligonucleotide”
aaaaaauguccuaggauguuguu

SEQ ID NO: 39 - RNA, 21 nt, Artificial Sequence
  source: /note=“Description of Artificial Sequence Synthetic oligonucleotide”
  modified_base (21)..(21): Inverted abasic nucleotide
caaaagcacuucuuccaucgn

SEQ ID NO: 40 - RNA, 23 nt, Artificial Sequence
  source: /note=“Description of Artificial Sequence Synthetic oligonucleotide”
ucgauggaagaagugcuuuuguu

SEQ ID NO: 41 - RNA, 21 nt, Artificial Sequence
  source: /note=“Description of Artificial Sequence Synthetic oligonucleotide”
  modified_base (21)..(21): Inverted abasic nucleotide
aaaagcacuucuuccaucgan

SEQ ID NO: 42 - RNA, 23 nt, Artificial Sequence
  source: /note=“Description of Artificial Sequence Synthetic oligonucleotide”
aucgauggaagaagugcuuuuuu

SEQ ID NO: 43 - RNA, 21 nt, Artificial Sequence
  source: /note=“Description of Artificial Sequence Synthetic oligonucleotide”
  modified_base (21)..(21): Inverted abasic nucleotide
aaagcacuucuuccaucgaun

SEQ ID NO: 44 - RNA, 23 nt, Artificial Sequence
  source: /note=“Description of Artificial Sequence Synthetic oligonucleotide”
aaucgauggaagaagugcuuuuu

SEQ ID NO: 45 - RNA, 21 nt, Artificial Sequence
  source: /note=“Description of Artificial Sequence Synthetic oligonucleotide”
  modified_base (21)..(21): Inverted abasic nucleotide
aagcacuucuuccaucgaugn

SEQ ID NO: 46 - RNA, 23 nt, Artificial Sequence
  source: /note=“Description of Artificial Sequence Synthetic oligonucleotide”
ucaucgauggaagaagugcuuuu

SEQ ID NO: 47 - RNA, 21 nt, Artificial Sequence
  source: /note=“Description of Artificial Sequence Synthetic oligonucleotide”
  modified_base (21)..(21): Inverted abasic nucleotide
agcacuucuuccaucgaugan

SEQ ID NO: 48 - RNA, 23 nt, Artificial Sequence
  source: /note=“Description of Artificial Sequence Synthetic oligonucleotide”
aucaucgauggaagaagugcuuu

SEQ ID NO: 49 - RNA, 21 nt, Artificial Sequence
  source: /note=“Description of Artificial Sequence Synthetic oligonucleotide”
  modified_base (21)..(21): Inverted abasic nucleotide
uuccuuaccucaucccauaun

SEQ ID NO: 50 - RNA, 23 nt, Artificial Sequence
  source: /note=“Description of Artificial Sequence Synthetic oligonucleotide”
aauaugggaugagguaaggaauu

SEQ ID NO: 51 - RNA, 21 nt, Artificial Sequence
  source: /note=“Description of Artificial Sequence Synthetic oligonucleotide”
  modified_base (21)..(21): Inverted abasic nucleotide
ccuuaccucaucccauauugn

SEQ ID NO: 52 - RNA, 23 nt, Artificial Sequence
  source: /note=“Description of Artificial Sequence Synthetic oligonucleotide”
acaauaugggaugagguaagguu

SEQ ID NO: 53 - RNA, 21 nt, Artificial Sequence
  source: /note=“Description of Artificial Sequence Synthetic oligonucleotide”
  modified_base (21)..(21): Inverted abasic nucleotide
accucaucccauauuguuccn

SEQ ID NO: 54 - RNA, 23 nt, Artificial Sequence
  source: /note=“Description of Artificial Sequence Synthetic oligonucleotide”
uggaacaauaugggaugagguuu

SEQ ID NO: 55 - RNA, 21 nt, Artificial Sequence
  source: /note=“Description of Artificial Sequence Synthetic oligonucleotide”
  modified_base (21)..(21): Inverted abasic nucleotide
ccucaucccauauuguuccan

SEQ ID NO: 56 - RNA, 23 nt, Artificial Sequence
  source: /note=“Description of Artificial Sequence Synthetic oligonucleotide”
auggaacaauaugggaugagguu

SEQ ID NO: 57 - RNA, 21 nt, Artificial Sequence
  source: /note=“Description of Artificial Sequence Synthetic oligonucleotide”
  modified_base (21)..(21): Inverted abasic nucleotide
ucccauauuguuccagcaaan

SEQ ID NO: 58 - RNA, 23 nt, Artificial Sequence
  source: /note=“Description of Artificial Sequence Synthetic oligonucleotide”
auuugcuggaacaauaugggauu

SEQ ID NO: 59 - RNA, 21 nt, Artificial Sequence
  source: /note=“Description of Artificial Sequence Synthetic oligonucleotide”
  modified_base (21)..(21): Inverted abasic nucleotide
ggcuuucacagaggucugacn

SEQ ID NO: 60 - RNA, 23 nt, Artificial Sequence
  source: /note=“Description of Artificial Sequence Synthetic oligonucleotide”
ugucagaccucugugaaagccuu

SEQ ID NO: 61 - RNA, 21 nt, Artificial Sequence
  source: /note=“Description of Artificial Sequence Synthetic oligonucleotide”
  modified_base (21)..(21): Inverted abasic nucleotide
uuugugaauacuggguucacn

SEQ ID NO: 62 - RNA, 23 nt, Artificial Sequence
  source: /note=“Description of Artificial Sequence Synthetic oligonucleotide”
agugaacccaguauucacaaauu

SEQ ID NO: 63 - RNA, 21 nt, Artificial Sequence
  source: /note=“Description of Artificial Sequence Synthetic oligonucleotide”
  modified_base (21)..(21): Inverted abasic nucleotide
uugugaauacuggguucaccn

SEQ ID NO: 64 - RNA, 23 nt, Artificial Sequence
  source: /note=“Description of Artificial Sequence Synthetic oligonucleotide”
uggugaacccaguauucacaauu

SEQ ID NO: 65 - RNA, 21 nt, Artificial Sequence
  source: /note=“Description of Artificial Sequence Synthetic oligonucleotide”
  modified_base (21)..(21): Inverted abasic nucleotide
gaauacuggguucaccaaaan

SEQ ID NO: 66 - RNA, 23 nt, Artificial Sequence
  source: /note=“Description of Artificial Sequence Synthetic oligonucleotide”
uuuuuggugaacccaguauucuu

SEQ ID NO: 67 - RNA, 21 nt, Artificial Sequence
  source: /note=“Description of Artificial Sequence Synthetic oligonucleotide”
  modified_base (21)..(21): Inverted abasic nucleotide
auacuggguucaccaaaaaun

SEQ ID NO: 68 - RNA, 23 nt, Artificial Sequence
  source: /note=“Description of Artificial Sequence Synthetic oligonucleotide”
aauuuuuggugaacccaguauuu

SEQ ID NO: 69 - RNA, 21 nt, Artificial Sequence
  source: /note=“Description of Artificial Sequence Synthetic oligonucleotide”
  modified_base (21)..(21): Inverted abasic nucleotide
uacuggguucaccaaaaaucn

SEQ ID NO: 70 - RNA, 23 nt, Artificial Sequence
  source: /note=“Description of Artificial Sequence Synthetic oligonucleotide”
agauuuuuggugaacccaguauu

SEQ ID NO: 71 - RNA, 21 nt, Artificial Sequence
  source: /note=“Description of Artificial Sequence Synthetic oligonucleotide”
  modified_base (21)..(21): Inverted abasic nucleotide
uuuuaaaucguaugcagaaun

SEQ ID NO: 72 - RNA, 23 nt, Artificial Sequence
  source: /note=“Description of Artificial Sequence Synthetic oligonucleotide”
uauucugcauacgauuuaaaauu

SEQ ID NO: 73 - RNA, 21 nt, Artificial Sequence
  source: /note=“Description of Artificial Sequence Synthetic oligonucleotide”
  modified_base (21)..(21): Inverted abasic nucleotide
uuuaaaucguaugcagaauan

SEQ ID NO: 74 - RNA, 23 nt, Artificial Sequence
  source: /note=“Description of Artificial Sequence Synthetic oligonucleotide”
auauucugcauacgauuuaaauu

SEQ ID NO: 75 - RNA, 21 nt, Artificial Sequence
  source: /note=“Description of Artificial Sequence Synthetic oligonucleotide”
  modified_base (21)..(21): Inverted abasic nucleotide
uaaaucguaugcagaauauun

SEQ ID NO: 76 - RNA, 23 nt, Artificial Sequence
  source: /note=“Description of Artificial Sequence Synthetic oligonucleotide”
aaauauucugcauacgauuuauu

SEQ ID NO: 77 - RNA, 21 nt, Artificial Sequence
  source: /note=“Description of Artificial Sequence Synthetic oligonucleotide”
  modified_base (21)..(21): Inverted abasic nucleotide
aaaucguaugcagaauauucn

SEQ ID NO: 78 - RNA, 23 nt, Artificial Sequence
  source: /note=“Description of Artificial Sequence Synthetic oligonucleotide”
ugaauauucugcauacgauuuuu

SEQ ID NO: 79 - RNA, 21 nt, Artificial Sequence
  source: /note=“Description of Artificial Sequence Synthetic oligonucleotide”
  modified_base (21)..(21): Inverted abasic nucleotide
aaucguaugcagaauauucan

SEQ ID NO: 80 - RNA, 23 nt, Artificial Sequence
  source: /note=“Description of Artificial Sequence Synthetic oligonucleotide”
uugaauauucugcauacgauuuu

SEQ ID NO: 81 - RNA, 21 nt, Artificial Sequence
  source: /note=“Description of Artificial Sequence Synthetic oligonucleotide”
  modified_base (21)..(21): Inverted abasic nucleotide
ucguaugcagaauauucaaun

SEQ ID NO: 82 - RNA, 23 nt, Artificial Sequence
  source: /note=“Description of Artificial Sequence Synthetic oligonucleotide”
aauugaauauucugcauacgauu

SEQ ID NO: 83 - RNA, 21 nt, Artificial Sequence
  source: /note=“Description of Artificial Sequence Synthetic oligonucleotide”
  modified_base (21)..(21): Inverted abasic nucleotide
cguaugcagaauauucaauun

SEQ ID NO: 84 - RNA, 23 nt, Artificial Sequence
  source: /note=“Description of Artificial Sequence Synthetic oligonucleotide”
aaauugaauauucugcauacguu

SEQ ID NO: 85 - RNA, 21 nt, Artificial Sequence
  source: /note=“Description of Artificial Sequence Synthetic oligonucleotide”
  modified_base (21)..(21): Inverted abasic nucleotide
uaugcagaauauucaauuugn

SEQ ID NO: 86 - RNA, 23 nt, Artificial Sequence
  source: /note=“Description of Artificial Sequence Synthetic oligonucleotide”
ucaaauugaauauucugcauauu

SEQ ID NO: 87 - RNA, 21 nt, Artificial Sequence
  source: /note=“Description of Artificial Sequence Synthetic oligonucleotide”
  modified_base (21)..(21): Inverted abasic nucleotide
aauauucaauuugaagcagun

SEQ ID NO: 88 - RNA, 23 nt, Artificial Sequence
  source: /note=“Description of Artificial Sequence Synthetic oligonucleotide”
aacugcuucaaauugaauauuuu

SEQ ID NO: 89 - RNA, 21 nt, Artificial Sequence
  source: /note=“Description of Artificial Sequence Synthetic oligonucleotide”
  modified_base (21)..(21): Inverted abasic nucleotide
aaaugaaaugaauaaauaagn

SEQ ID NO: 90 - RNA, 23 nt, Artificial Sequence
  source: /note=“Description of Artificial Sequence Synthetic oligonucleotide”
acuuauuuauucauuucauuuuu

SEQ ID NO: 91 - RNA, 21 nt, Artificial Sequence
  source: /note=“Description of Artificial Sequence Synthetic oligonucleotide”
  modified_base (21)..(21): Inverted abasic nucleotide
aaucaaugcugcaaagcuuun

SEQ ID NO: 92 - RNA, 23 nt, Artificial Sequence
  source: /note=“Description of Artificial Sequence Synthetic oligonucleotide”
uaaagcuuugcagcauugauuuu

SEQ ID NO: 93 - RNA, 21 nt, Artificial Sequence
  source: /note=“Description of Artificial Sequence Synthetic oligonucleotide”
  modified_base (21)..(21): Inverted abasic nucleotide
ugcugcaaagcuuuauuucan

SEQ ID NO: 94 - RNA, 23 nt, Artificial Sequence
  source: /note=“Description of Artificial Sequence Synthetic oligonucleotide”
augaaauaaagcuuugcagcauu

SEQ ID NO: 95 - RNA, 21 nt, Artificial Sequence
  source: /note=“Description of Artificial Sequence Synthetic oligonucleotide”
  modified_base (21)..(21): Inverted abasic nucleotide
gcugcaaagcuuuauuucacn

SEQ ID NO: 96 - RNA, 23 nt, Artificial Sequence
  source: /note=“Description of Artificial Sequence Synthetic oligonucleotide”
ugugaaauaaagcuuugcagcuu

SEQ ID NO: 97 - RNA, 21 nt, Artificial Sequence
  source: /note=“Description of Artificial Sequence Synthetic oligonucleotide”
  modified_base (21)..(21): Inverted abasic nucleotide
uuaaaaacauugguuuggcan

SEQ ID NO: 98 - RNA, 23 nt, Artificial Sequence
  source: /note=“Description of Artificial Sequence Synthetic oligonucleotide”
augccaaaccaauguuuuuaauu

SEQ ID NO: 99 - RNA, 21 nt, Artificial Sequence
  source: /note=“Description of Artificial Sequence Synthetic oligonucleotide”
  modified_base (21)..(21): Inverted abasic nucleotide
aaaaacauugguuuggcacun

SEQ ID NO: 100 - RNA, 23 nt, Artificial Sequence
  source: /note=“Description of Artificial Sequence Synthetic oligonucleotide”
uagugccaaaccaauguuuuuuu

SEQ ID NO: 101 - RNA, 21 nt, Artificial Sequence
  source: /note=“Description of Artificial Sequence Synthetic oligonucleotide”
  modified_base (21)..(21): Inverted abasic nucleotide
aacaagauuaauuaccugucn

SEQ ID NO: 102 - RNA, 23 nt, Artificial Sequence
  source: /note=“Description of Artificial Sequence Synthetic oligonucleotide”
agacagguaauuaaucuuguuuu

SEQ ID NO: 103 - RNA, 21 nt, Artificial Sequence
  source: /note=“Description of Artificial Sequence Synthetic oligonucleotide”
  modified_base (21)..(21): Inverted abasic nucleotide
caagauuaauuaccugucuun

SEQ ID NO: 104 - RNA, 23 nt, Artificial Sequence
  source: /note=“Description of Artificial Sequence Synthetic oligonucleotide”
aaagacagguaauuaaucuuguu

SEQ ID NO: 105 - RNA, 21 nt, Artificial Sequence
  source: /note=“Description of Artificial Sequence Synthetic oligonucleotide”
  modified_base (21)..(21): Inverted abasic nucleotide
uaauuaccugucuuccuguun

SEQ ID NO: 106 - RNA, 23 nt, Artificial Sequence
  source: /note=“Description of Artificial Sequence Synthetic oligonucleotide”
aaacaggaagacagguaauuauu

SEQ ID NO: 107 - RNA, 21 nt, Artificial Sequence
  source: /note=“Description of Artificial Sequence Synthetic oligonucleotide”
  modified_base (21)..(21): Inverted abasic nucleotide
ccugucuuccuguuucucaan

SEQ ID NO: 108 - RNA, 23 nt, Artificial Sequence
  source: /note=“Description of Artificial Sequence Synthetic oligonucleotide”
auugagaaacaggaagacagguu

SEQ ID NO: 109 - RNA, 21 nt, Artificial Sequence
  source: /note=“Description of Artificial Sequence Synthetic oligonucleotide”
  modified_base (21)..(21): Inverted abasic nucleotide
uuuccuuucaugccucuuaan

SEQ ID NO: 110 - RNA, 23 nt, Artificial Sequence
  source: /note=“Description of Artificial Sequence Synthetic oligonucleotide”
uuuaagaggcaugaaaggaaauu

SEQ ID NO: 111 - RNA, 21 nt, Artificial Sequence
  source: /note=“Description of Artificial Sequence Synthetic oligonucleotide”
  modified_base (21)..(21): Inverted abasic nucleotide
uuccuuucaugccucuuaaan

SEQ ID NO: 112 - RNA, 23 nt, Artificial Sequence
  source: /note=“Description of Artificial Sequence Synthetic oligonucleotide”
uuuuaagaggcaugaaaggaauu

SEQ ID NO: 113 - RNA, 21 nt, Artificial Sequence
  source: /note=“Description of Artificial Sequence Synthetic oligonucleotide”
  modified_base (21)..(21): Inverted abasic nucleotide
uuuuccauuuaaagguggacn

SEQ ID NO: 114 - RNA, 23 nt, Artificial Sequence
  source: /note=“Description of Artificial Sequence Synthetic oligonucleotide”
uguccaccuuuaaauggaaaauu

SEQ ID NO: 115 - RNA, 21 nt, Artificial Sequence
  source: /note=“Description of Artificial Sequence Synthetic oligonucleotide”
  modified_base (21)..(21): Inverted abasic nucleotide
uuuccauuuaaagguggacan

SEQ ID NO: 116 - RNA, 23 nt, Artificial Sequence
  source: /note=“Description of Artificial Sequence Synthetic oligonucleotide”
uuguccaccuuuaaauggaaauu

SEQ ID NO: 117 - RNA, 21 nt, Artificial Sequence
  source: /note=“Description of Artificial Sequence Synthetic oligonucleotide”
  modified_base (21)..(21): Inverted abasic nucleotide
uuccauuuaaagguggacaan

SEQ ID NO: 118 - RNA, 23 nt, Artificial Sequence
  source: /note=“Description of Artificial Sequence Synthetic oligonucleotide”
uuuguccaccuuuaaauggaauu

SEQ ID NO: 119 - RNA, 21 nt, Artificial Sequence
  source: /note=“Description of Artificial Sequence Synthetic oligonucleotide”
  modified_base (21)..(21): Inverted abasic nucleotide
uccauuuaaagguggacaaan

SEQ ID NO: 120 - RNA, 23 nt, Artificial Sequence
  source: /note=“Description of Artificial Sequence Synthetic oligonucleotide”
uuuuguccaccuuuaaauggauu

SEQ ID NO: 121 - RNA, 21 nt, Artificial Sequence
  source: /note=“Description of Artificial Sequence Synthetic oligonucleotide”
  modified_base (21)..(21): Inverted abasic nucleotide
gaacuuauuuacacagggaan

SEQ ID NO: 122 - RNA, 23 nt, Artificial Sequence
  source: /note=“Description of Artificial Sequence Synthetic oligonucleotide”
auucccuguguaaauaaguucuu

SEQ ID NO: 123 - RNA, 21 nt, Artificial Sequence
  source: /note=“Description of Artificial Sequence Synthetic oligonucleotide”
  modified_base (21)..(21): Inverted abasic nucleotide
cuuauuuacacagggaaggun

SEQ ID NO: 124 - RNA, 23 nt, Artificial Sequence
  source: /note=“Description of Artificial Sequence Synthetic oligonucleotide”
aaccuucccuguguaaauaaguu

SEQ ID NO: 125 - RNA, 21 nt, Artificial Sequence
  source: /note=“Description of Artificial Sequence Synthetic oligonucleotide”
  modified_base (21)..(21): Inverted abasic nucleotide
auuuacacagggaagguuuan

SEQ ID NO: 126 - RNA, 23 nt, Artificial Sequence
  source: /note=“Description of Artificial Sequence Synthetic oligonucleotide”
uuaaaccuucccuguguaaauuu

SEQ ID NO: 127 - RNA, 21 nt, Artificial Sequence
  source: /note=“Description of Artificial Sequence Synthetic oligonucleotide”
  modified_base (21)..(21): Inverted abasic nucleotide
uuuacacagggaagguuuaan

SEQ ID NO: 128 - RNA, 23 nt, Artificial Sequence
  source: /note=“Description of Artificial Sequence Synthetic oligonucleotide”
auuaaaccuucccuguguaaauu

SEQ ID NO: 129 - RNA, 21 nt, Artificial Sequence
  source: /note=“Description of Artificial Sequence Synthetic oligonucleotide”
  modified_base (21)..(21): Inverted abasic nucleotide
cagggaagguuuaagacugun

SEQ ID NO: 130 - RNA, 23 nt, Artificial Sequence
  source: /note=“Description of Artificial Sequence Synthetic oligonucleotide”
aacagucuuaaaccuucccuguu

SEQ ID NO: 131 - RNA, 21 nt, Artificial Sequence
  source: /note=“Description of Artificial Sequence Synthetic oligonucleotide”
  modified_base (21)..(21): Inverted abasic nucleotide
ggaagguuuaagacuguucan

SEQ ID NO: 132 - RNA, 23 nt, Artificial Sequence
  source: /note=“Description of Artificial Sequence Synthetic oligonucleotide”
uugaacagucuuaaaccuuccuu

SEQ ID NO: 133 - RNA, 21 nt, Artificial Sequence
  source: /note=“Description of Artificial Sequence Synthetic oligonucleotide”
  modified_base (21)..(21): Inverted abasic nucleotide
agguuuaagacuguucaagun

SEQ ID NO: 134 - RNA, 23 nt, Artificial Sequence
  source: /note=“Description of Artificial Sequence Synthetic oligonucleotide”
uacuugaacagucuuaaaccuuu

SEQ ID NO: 135 - RNA, 21 nt, Artificial Sequence
  source: /note=“Description of Artificial Sequence Synthetic oligonucleotide”
  modified_base (21)..(21): Inverted abasic nucleotide
gguuuaagacuguucaaguan

SEQ ID NO: 136 - RNA, 23 nt, Artificial Sequence
  source: /note=“Description of Artificial Sequence Synthetic oligonucleotide”
auacuugaacagucuuaaaccuu

SEQ ID NO: 137 - RNA, 21 nt, Artificial Sequence
  source: /note=“Description of Artificial Sequence Synthetic oligonucleotide”
  modified_base (21)..(21): Inverted abasic nucleotide
agacuguucaaguagcauucn

SEQ ID NO: 138 - RNA, 23 nt, Artificial Sequence
  source: /note=“Description of Artificial Sequence Synthetic oligonucleotide”
agaaugcuacuugaacagucuuu

SEQ ID NO: 139 - RNA, 21 nt, Artificial Sequence
  source: /note=“Description of Artificial Sequence Synthetic oligonucleotide”
  modified_base (21)..(21): Inverted abasic nucleotide
gacuguucaaguagcauuccn

SEQ ID NO: 140 - RNA, 23 nt, Artificial Sequence
  source: /note=“Description of Artificial Sequence Synthetic oligonucleotide”
uggaaugcuacuugaacagucuu

SEQ ID NO: 141 - RNA, 21 nt, Artificial Sequence
  source: /note=“Description of Artificial Sequence Synthetic oligonucleotide”
  modified_base (21)..(21): Inverted abasic nucleotide
acuguucaaguagcauuccan

SEQ ID NO: 142 - RNA, 23 nt, Artificial Sequence
  source: /note=“Description of Artificial Sequence Synthetic oligonucleotide”
uuggaaugcuacuugaacaguuu

SEQ ID NO: 143 - RNA, 21 nt, Artificial Sequence
  source: /note=“Description of Artificial Sequence Synthetic oligonucleotide”
  modified_base (21)..(21): Inverted abasic nucleotide
cuguucaaguagcauuccaan

SEQ ID NO: 144 - RNA, 23 nt, Artificial Sequence
  source: /note=“Description of Artificial Sequence Synthetic oligonucleotide”
auuggaaugcuacuugaacaguu

SEQ ID NO: 145 - RNA, 21 nt, Artificial Sequence
  source: /note=“Description of Artificial Sequence Synthetic oligonucleotide”
  modified_base (21)..(21): Inverted abasic nucleotide
uguucaaguagcauuccaaun

SEQ ID NO: 146 - RNA, 23 nt, Artificial Sequence
  source: /note=“Description of Artificial Sequence Synthetic oligonucleotide”
aauuggaaugcuacuugaacauu

SEQ ID NO: 147 - RNA, 21 nt, Artificial Sequence
  source: /note=“Description of Artificial Sequence Synthetic oligonucleotide”
  modified_base (21)..(21): Inverted abasic nucleotide
caagaacacagaaugagugcn

SEQ ID NO: 148 - RNA, 23 nt, Artificial Sequence
  source: /note=“Description of Artificial Sequence Synthetic oligonucleotide”
ugcacucauucuguguucuuguu

SEQ ID NO: 149 - RNA, 21 nt, Artificial Sequence
  source: /note=“Description of Artificial Sequence Synthetic oligonucleotide”
  modified_base (21)..(21): Inverted abasic nucleotide
acagaaugagugcacagcuan

SEQ ID NO: 150 - RNA, 23 nt, Artificial Sequence
  source: /note=“Description of Artificial Sequence Synthetic oligonucleotide”
uuagcugugcacucauucuguuu

SEQ ID NO: 151 - RNA, 21 nt, Artificial Sequence
  source: /note=“Description of Artificial Sequence Synthetic oligonucleotide”
  modified_base (21)..(21): Inverted abasic nucleotide
aggcagcuuuaucucaaccun

SEQ ID NO: 152 - RNA, 23 nt, Artificial Sequence
  source: /note=“Description of Artificial Sequence Synthetic oligonucleotide”
aagguugagauaaagcugccuuu

SEQ ID NO: 153 - RNA, 21 nt, Artificial Sequence
  source: /note=“Description of Artificial Sequence Synthetic oligonucleotide”
  modified_base (21)..(21): Inverted abasic nucleotide
uuuuaagauucagcauuugan

SEQ ID NO: 154 - RNA, 23 nt, Artificial Sequence
  source: /note=“Description of Artificial Sequence Synthetic oligonucleotide”
uucaaaugcugaaucuuaaaauu

SEQ ID NO: 155 - RNA, 21 nt, Artificial Sequence
  source: /note=“Description of Artificial Sequence Synthetic oligonucleotide”
  modified_base (21)..(21): Inverted abasic nucleotide
agauucagcauuugaaagaun

SEQ ID NO: 156 - RNA, 23 nt, Artificial Sequence
  source: /note=“Description of Artificial Sequence Synthetic oligonucleotide”
aaucuuucaaaugcugaaucuuu

SEQ ID NO: 157 - RNA, 21 nt, Artificial Sequence
  source: /note=“Description of Artificial Sequence Synthetic oligonucleotide”
  modified_base (21)..(21): Inverted abasic nucleotide
auuugaaagauuucccuagcn

SEQ ID NO: 158 - RNA, 23 nt, Artificial Sequence
  source: /note=“Description of Artificial Sequence Synthetic oligonucleotide”
agcuagggaaaucuuucaaauuu

SEQ ID NO: 159 - RNA, 21 nt, Artificial Sequence
  source: /note=“Description of Artificial Sequence Synthetic oligonucleotide”
  modified_base (21)..(21): Inverted abasic nucleotide
uucccuagccucuuccuuuun

SEQ ID NO: 160 - RNA, 23 nt, Artificial Sequence
  source: /note=“Description of Artificial Sequence Synthetic oligonucleotide”
aaaaaggaagaggcuagggaauu

SEQ ID NO: 161 - RNA, 21 nt, Artificial Sequence
  source: /note=“Description of Artificial Sequence Synthetic oligonucleotide”
  modified_base (21)..(21): Inverted abasic nucleotide
cuauucuggacuuuauuacun

SEQ ID NO: 162 - RNA, 23 nt, Artificial Sequence
  source: /note=“Description of Artificial Sequence Synthetic oligonucleotide”
aaguaauaaaguccagaauaguu

SEQ ID NO: 163 - RNA, 21 nt, Artificial Sequence
  source: /note=“Description of Artificial Sequence Synthetic oligonucleotide”
  modified_base (21)..(21): Inverted abasic nucleotide
aguccaccaaaaguggacccn

SEQ ID NO: 164 - RNA, 23 nt, Artificial Sequence
  source: /note=“Description of Artificial Sequence Synthetic oligonucleotide”
aggguccacuuuugguggacuuu

SEQ ID NO: 165 - RNA, 21 nt, Artificial Sequence
  source: /note=“Description of Artificial Sequence Synthetic oligonucleotide”
  modified_base (21)..(21): Inverted abasic nucleotide
caccaaaaguggacccucuan

SEQ ID NO: 166 - RNA, 23 nt, Artificial Sequence
  source: /note=“Description of Artificial Sequence Synthetic oligonucleotide”
auagaggguccacuuuugguguu

SEQ ID NO: 167 - RNA, 21 nt, Artificial Sequence
  source: /note=“Description of Artificial Sequence Synthetic oligonucleotide”
  modified_base (21)..(21): Inverted abasic nucleotide
accaaaaguggacccucuaun

SEQ ID NO: 168 - RNA, 23 nt, Artificial Sequence
  source: /note=“Description of Artificial Sequence Synthetic oligonucleotide”
uauagaggguccacuuuugguuu

SEQ ID NO: 169 - RNA, 21 nt, Artificial Sequence
  source: /note=“Description of Artificial Sequence Synthetic oligonucleotide”
  modified_base (21)..(21): Inverted abasic nucleotide
caaaaguggacccucuauaun

SEQ ID NO: 170 - RNA, 23 nt, Artificial Sequence
  source: /note=“Description of Artificial Sequence Synthetic oligonucleotide”
aauauagaggguccacuuuuguu

SEQ ID NO: 171 - RNA, 21 nt, Artificial Sequence
  source: /note=“Description of Artificial Sequence Synthetic oligonucleotide”
  modified_base (21)..(21): Inverted abasic nucleotide
aaaaguggacccucuauauun

SEQ ID NO: 172 - RNA, 23 nt, Artificial Sequence
  source: /note=“Description of Artificial Sequence Synthetic oligonucleotide”
aaauauagaggguccacuuuuuu

SEQ ID NO: 173 - RNA, 21 nt, Artificial Sequence
  source: /note=“Description of Artificial Sequence Synthetic oligonucleotide”
  modified_base (21)..(21): Inverted abasic nucleotide
aaaguggacccucuauauuun

SEQ ID NO: 174 - RNA, 23 nt, Artificial Sequence
  source: /note=“Description of Artificial Sequence Synthetic oligonucleotide”
aaaauauagaggguccacuuuuu

SEQ ID NO: 175 - RNA, 21 nt, Artificial Sequence
  source: /note=“Description of Artificial Sequence Synthetic oligonucleotide”
  modified_base (21)..(21): Inverted abasic nucleotide
aaguggacccucuauauuucn

SEQ ID NO: 176 - RNA, 23 nt, Artificial Sequence
  source: /note=“Description of Artificial Sequence Synthetic oligonucleotide”
agaaauauagaggguccacuuuu

SEQ ID NO: 177 - RNA, 21 nt, Artificial Sequence
  source: /note=“Description of Artificial Sequence Synthetic oligonucleotide”
  modified_base (21)..(21): Inverted abasic nucleotide
uucauauauccuuggucccan

SEQ ID NO: 178 - RNA, 23 nt, Artificial Sequence
  source: /note=“Description of Artificial Sequence Synthetic oligonucleotide”
augggaccaaggauauaugaauu

SEQ ID NO: 179 - RNA, 21 nt, Artificial Sequence
  source: /note=“Description of Artificial Sequence Synthetic oligonucleotide”
  modified_base (21)..(21): Inverted abasic nucleotide
gauguuuagacaauuuuaggn

SEQ ID NO: 180 - RNA, 23 nt, Artificial Sequence
  source: /note=“Description of Artificial Sequence Synthetic oligonucleotide”
accuaaaauugucuaaacaucuu

SEQ ID NO: 181 - RNA, 21 nt, Artificial Sequence
  source: /note=“Description of Artificial Sequence Synthetic oligonucleotide”
  modified_base (21)..(21): Inverted abasic nucleotide
auguuuagacaauuuuaggcn

SEQ ID NO: 182 - RNA, 23 nt, Artificial Sequence
  source: /note=“Description of Artificial Sequence Synthetic oligonucleotide”
agccuaaaauugucuaaacauuu

SEQ ID NO: 183 - RNA, 21 nt, Artificial Sequence
  source: /note=“Description of Artificial Sequence Synthetic oligonucleotide”
  modified_base (21)..(21): Inverted abasic nucleotide
uguuuagacaauuuuaggcun

SEQ ID NO: 184 - RNA, 23 nt, Artificial Sequence
  source: /note=“Description of Artificial Sequence Synthetic oligonucleotide”
aagccuaaaauugucuaaacauu

SEQ ID NO: 185 - RNA, 21 nt, Artificial Sequence
  source: /note=“Description of Artificial Sequence Synthetic oligonucleotide”
  modified_base (21)..(21): Inverted abasic nucleotide
guuuagacaauuuuaggcucn

SEQ ID NO: 186 - RNA, 23 nt, Artificial Sequence
  source: /note=“Description of Artificial Sequence Synthetic oligonucleotide”
ugagccuaaaauugucuaaacuu

SEQ ID NO: 187 - RNA, 21 nt, Artificial Sequence
  source: /note=“Description of Artificial Sequence Synthetic oligonucleotide”
  modified_base (21)..(21): Inverted abasic nucleotide
uuuagacaauuuuaggcucan

SEQ ID NO: 188 - RNA, 23 nt, Artificial Sequence
  source: /note=“Description of Artificial Sequence Synthetic oligonucleotide”
uugagccuaaaauugucuaaauu

SEQ ID NO: 189 - RNA, 21 nt, Artificial Sequence
  source: /note=“Description of Artificial Sequence Synthetic oligonucleotide”
  modified_base (21)..(21): Inverted abasic nucleotide
uuagacaauuuuaggcucaan

SEQ ID NO: 190 - RNA, 23 nt, Artificial Sequence
  source: /note=“Description of Artificial Sequence Synthetic oligonucleotide”
uuugagccuaaaauugucuaauu

SEQ ID NO: 191 - RNA, 21 nt, Artificial Sequence
  source: /note=“Description of Artificial Sequence Synthetic oligonucleotide”
  modified_base (21)..(21): Inverted abasic nucleotide
agacaauuuuaggcucaaaan

SEQ ID NO: 192 - RNA, 23 nt, Artificial Sequence
  source: /note=“Description of Artificial Sequence Synthetic oligonucleotide”
uuuuugagccuaaaauugucuuu

SEQ ID NO: 193 - RNA, 21 nt, Artificial Sequence
  source: /note=“Description of Artificial Sequence Synthetic oligonucleotide”
  modified_base (21)..(21): Inverted abasic nucleotide
auuuuaggcucaaaaauuaan

SEQ ID NO: 194 - RNA, 23 nt, Artificial Sequence
  source: /note=“Description of Artificial Sequence Synthetic oligonucleotide”
uuuaauuuuugagccuaaaauuu

SEQ ID NO: 195 - RNA, 21 nt, Artificial Sequence
  source: /note=“Description of Artificial Sequence Synthetic oligonucleotide”
  modified_base (21)..(21): Inverted abasic nucleotide
uuuaggcucaaaaauuaaagn

SEQ ID NO: 196 - RNA, 23 nt, Artificial Sequence
  source: /note=“Description of Artificial Sequence Synthetic oligonucleotide”
acuuuaauuuuugagccuaaauu

SEQ ID NO: 197 - RNA, 21 nt, Artificial Sequence
  source: /note=“Description of Artificial Sequence Synthetic oligonucleotide”
  modified_base (21)..(21): Inverted abasic nucleotide
uuaggcucaaaaauuaaagcn

SEQ ID NO: 198 - RNA, 23 nt, Artificial Sequence
  source: /note=“Description of Artificial Sequence Synthetic oligonucleotide”
agcuuuaauuuuugagccuaauu

SEQ ID NO: 199 - RNA, 21 nt, Artificial Sequence
  source: /note=“Description of Artificial Sequence Synthetic oligonucleotide”
  modified_base (21)..(21): Inverted abasic nucleotide
aaaaauuaaagcuaacacagn

SEQ ID NO: 200 - RNA, 23 nt, Artificial Sequence
  source: /note=“Description of Artificial Sequence Synthetic oligonucleotide”
acuguguuagcuuuaauuuuuuu

SEQ ID NO: 201 - RNA, 21 nt, Artificial Sequence
  source: /note=“Description of Artificial Sequence Synthetic oligonucleotide”
  modified_base (21)..(21): Inverted abasic nucleotide
aaaauuaaagcuaacacaggn

SEQ ID NO: 202 - RNA, 23 nt, Artificial Sequence
  source: /note=“Description of Artificial Sequence Synthetic oligonucleotide”
uccuguguuagcuuuaauuuuuu

SEQ ID NO: 203 - RNA, 21 nt, Artificial Sequence
  source: /note=“Description of Artificial Sequence Synthetic oligonucleotide”
  modified_base (21)..(21): Inverted abasic nucleotide
aaagcuaacacaggaaaaggn

SEQ ID NO: 204 - RNA, 23 nt, Artificial Sequence
  source: /note=“Description of Artificial Sequence Synthetic oligonucleotide”
uccuuuuccuguguuagcuuuuu

SEQ ID NO: 205 - RNA, 21 nt, Artificial Sequence
  source: /note=“Description of Artificial Sequence Synthetic oligonucleotide”
  modified_base (21)..(21): Inverted abasic nucleotide
aagcuaacacaggaaaaggan

SEQ ID NO: 206 - RNA, 23 nt, Artificial Sequence
  source: /note=“Description of Artificial Sequence Synthetic oligonucleotide”
uuccuuuuccuguguuagcuuuu

SEQ ID NO: 207 - RNA, 21 nt, Artificial Sequence
  source: /note=“Description of Artificial Sequence Synthetic oligonucleotide”
  modified_base (21)..(21): Inverted abasic nucleotide
ggaaaaggaacuguacuggcn

SEQ ID NO: 208 - RNA, 23 nt, Artificial Sequence
  source: /note=“Description of Artificial Sequence Synthetic oligonucleotide”
agccaguacaguuccuuuuccuu

SEQ ID NO: 209 - RNA, 21 nt, Artificial Sequence
  source: /note=“Description of Artificial Sequence Synthetic oligonucleotide”
  modified_base (21)..(21): Inverted abasic nucleotide
aggaacuguacuggcuauuan

SEQ ID NO: 210 - RNA, 23 nt, Artificial Sequence
  source: /note=“Description of Artificial Sequence Synthetic oligonucleotide”
auaauagccaguacaguuccuuu

SEQ ID NO: 211 - RNA, 21 nt, Artificial Sequence
  source: /note=“Description of Artificial Sequence Synthetic oligonucleotide”
  modified_base (21)..(21): Inverted abasic nucleotide
ccgacucccacuacaucaagn

SEQ ID NO: 212 - RNA, 23 nt, Artificial Sequence
  source: /note=“Description of Artificial Sequence Synthetic oligonucleotide”
ucuugauguagugggagucgguu

SEQ ID NO: 213 - RNA, 21 nt, Artificial Sequence
  source: /note=“Description of Artificial Sequence Synthetic oligonucleotide”
  modified_base (21)..(21): Inverted abasic nucleotide
gacucccacuacaucaagacn

SEQ ID NO: 214 - RNA, 23 nt, Artificial Sequence
  source: /note=“Description of Artificial Sequence Synthetic oligonucleotide”
agucuugauguagugggagucuu

SEQ ID NO: 215 - RNA, 21 nt, Artificial Sequence
  source: /note=“Description of Artificial Sequence Synthetic oligonucleotide”
  modified_base (21)..(21): Inverted abasic nucleotide
ucccacuacaucaagacuaan

SEQ ID NO: 216 - RNA, 23 nt, Artificial Sequence
  source: /note=“Description of Artificial Sequence Synthetic oligonucleotide”
auuagucuugauguagugggauu

SEQ ID NO: 217 - RNA, 21 nt, Artificial Sequence
  source: /note=“Description of Artificial Sequence Synthetic oligonucleotide”
  modified_base (21)..(21): Inverted abasic nucleotide
cccacuacaucaagacuaaun

SEQ ID NO: 218 - RNA, 23 nt, Artificial Sequence
  source: /note=“Description of Artificial Sequence Synthetic oligonucleotide”
aauuagucuugauguaguggguu

SEQ ID NO: 219 - RNA, 21 nt, Artificial Sequence
  source: /note=“Description of Artificial Sequence Synthetic oligonucleotide”
  modified_base (21)..(21): Inverted abasic nucleotide
cacuacaucaagacuaaucun

SEQ ID NO: 220 - RNA, 23 nt, Artificial Sequence
  source: /note=“Description of Artificial Sequence Synthetic oligonucleotide”
aagauuagucuugauguaguguu

SEQ ID NO: 221 - RNA, 21 nt, Artificial Sequence
  source: /note=“Description of Artificial Sequence Synthetic oligonucleotide”
  modified_base (21)..(21): Inverted abasic nucleotide
acuacaucaagacuaaucuun

SEQ ID NO: 222 - RNA, 23 nt, Artificial Sequence
  source: /note=“Description of Artificial Sequence Synthetic oligonucleotide”
aaagauuagucuugauguaguuu

SEQ ID NO: 223 - RNA, 21 nt, Artificial Sequence
  source: /note=“Description of Artificial Sequence Synthetic oligonucleotide”
  modified_base (21)..(21): Inverted abasic nucleotide
cuacaucaagacuaaucuugn

SEQ ID NO: 224 - RNA, 23 nt, Artificial Sequence
  source: /note=“Description of Artificial Sequence Synthetic oligonucleotide”
acaagauuagucuugauguaguu

SEQ ID NO: 225 - RNA, 21 nt, Artificial Sequence
  source: /note=“Description of Artificial Sequence Synthetic oligonucleotide”
  modified_base (21)..(21): Inverted abasic nucleotide
guuuuucacauguauuauagn

SEQ ID NO: 226 - RNA, 23 nt, Artificial Sequence
  source: /note=“Description of Artificial Sequence Synthetic oligonucleotide”
ucuauaauacaugugaaaaacuu

SEQ ID NO: 227 - RNA, 21 nt, Artificial Sequence
  source: /note=“Description of Artificial Sequence Synthetic oligonucleotide”
  modified_base (21)..(21): Inverted abasic nucleotide
ucacauguauuauagaaugcn

SEQ ID NO: 228 - RNA, 23 nt, Artificial Sequence
  source: /note=“Description of Artificial Sequence Synthetic oligonucleotide”
agcauucuauaauacaugugauu

SEQ ID NO: 229 - RNA, 21 nt, Artificial Sequence
  source: /note=“Description of Artificial Sequence Synthetic oligonucleotide”
  modified_base (21)..(21): Inverted abasic nucleotide
acauguauuauagaaugcuun

SEQ ID NO: 230 - RNA, 23 nt, Artificial Sequence
  source: /note=“Description of Artificial Sequence Synthetic oligonucleotide”
aaagcauucuauaauacauguuu

SEQ ID NO: 231 - RNA, 21 nt, Artificial Sequence
  source: /note=“Description of Artificial Sequence Synthetic oligonucleotide”
  modified_base (21)..(21): Inverted abasic nucleotide
uguauuauagaaugcuuuugn

SEQ ID NO: 232 - RNA, 23 nt, Artificial Sequence
  source: /note=“Description of Artificial Sequence Synthetic oligonucleotide”
acaaaagcauucuauaauacauu

SEQ ID NO: 233 - RNA, 21 nt, Artificial Sequence
  source: /note=“Description of Artificial Sequence Synthetic oligonucleotide”
  modified_base (21)..(21): Inverted abasic nucleotide
gaaugcuuuugcauggacuan

SEQ ID NO: 234 - RNA, 23 nt, Artificial Sequence
  source: /note=“Description of Artificial Sequence Synthetic oligonucleotide”
auaguccaugcaaaagcauucuu

SEQ ID NO: 235 - RNA, 21 nt, Artificial Sequence
  source: /note=“Description of Artificial Sequence Synthetic oligonucleotide”
  modified_base (21)..(21): Inverted abasic nucleotide
aaugcuuuugcauggacuaun

SEQ ID NO: 236 - RNA, 23 nt, Artificial Sequence
  source: /note=“Description of Artificial Sequence Synthetic oligonucleotide”
aauaguccaugcaaaagcauuuu

SEQ ID NO: 237 - RNA, 21 nt, Artificial Sequence
  source: /note=“Description of Artificial Sequence Synthetic oligonucleotide”
  modified_base (21)..(21): Inverted abasic nucleotide
gcuuuugcauggacuauccun

SEQ ID NO: 238 - RNA, 23 nt, Artificial Sequence
  source: /note=“Description of Artificial Sequence Synthetic oligonucleotide”
aaggauaguccaugcaaaagcuu

SEQ ID NO: 239 - RNA, 21 nt, Artificial Sequence
  source: /note=“Description of Artificial Sequence Synthetic oligonucleotide”
  modified_base (21)..(21): Inverted abasic nucleotide
uuugcauggacuauccucuun

SEQ ID NO: 240 - RNA, 23 nt, Artificial Sequence
  source: /note=“Description of Artificial Sequence Synthetic oligonucleotide”
aaagaggauaguccaugcaaauu

SEQ ID NO: 241 - RNA, 21 nt, Artificial Sequence
  source: /note=“Description of Artificial Sequence Synthetic oligonucleotide”
  modified_base (21)..(21): Inverted abasic nucleotide
uugcauggacuauccucuugn

SEQ ID NO: 242 - RNA, 23 nt, Artificial Sequence
  source: /note=“Description of Artificial Sequence Synthetic oligonucleotide”
acaagaggauaguccaugcaauu

SEQ ID NO: 243 - RNA, 21 nt, Artificial Sequence
  source: /note=“Description of Artificial Sequence Synthetic oligonucleotide”
  modified_base (21)..(21): Inverted abasic nucleotide
ugcauggacuauccucuugun

SEQ ID NO: 244 - RNA, 23 nt, Artificial Sequence
  source: /note=“Description of Artificial Sequence Synthetic oligonucleotide”
aacaagaggauaguccaugcauu

SEQ ID NO: 245 - RNA, 21 nt, Artificial Sequence
  source: /note=“Description of Artificial Sequence Synthetic oligonucleotide”
  modified_base (21)..(21): Inverted abasic nucleotide
gcauggacuauccucuuguun

SEQ ID NO: 246 - RNA, 23 nt, Artificial Sequence
  source: /note=“Description of Artificial Sequence Synthetic oligonucleotide”
aaacaagaggauaguccaugcuu

SEQ ID NO: 247 - RNA, 21 nt, Artificial Sequence
  source: /note=“Description of Artificial Sequence Synthetic oligonucleotide”
  modified_base (21)..(21): Inverted abasic nucleotide
auggacuauccucuuguuuun

SEQ ID NO: 248 - RNA, 23 nt, Artificial Sequence
  source: /note=“Description of Artificial Sequence Synthetic oligonucleotide”
aaaaacaagaggauaguccauuu

SEQ ID NO: 249 - RNA, 21 nt, Artificial Sequence
  source: /note=“Description of Artificial Sequence Synthetic oligonucleotide”
  modified_base (21)..(21): Inverted abasic nucleotide
ggacuauccucuuguuuuuan

SEQ ID NO: 250 - RNA, 23 nt, Artificial Sequence
  source: /note=“Description of Artificial Sequence Synthetic oligonucleotide”
auaaaaacaagaggauaguccuu

SEQ ID NO: 251 - RNA, 21 nt, Artificial Sequence
  source: /note=“Description of Artificial Sequence Synthetic oligonucleotide”
  modified_base (21)..(21): Inverted abasic nucleotide
aauaaccucuuguaguuauan

SEQ ID NO: 252 - RNA, 23 nt, Artificial Sequence
  source: /note=“Description of Artificial Sequence Synthetic oligonucleotide”
uuauaacuacaagagguuauuuu

SEQ ID NO: 253 - RNA, 21 nt, Artificial Sequence
  source: /note=“Description of Artificial Sequence Synthetic oligonucleotide”
  modified_base (21)..(21): Inverted abasic nucleotide
auaaccucuuguaguuauaan

SEQ ID NO: 254 - RNA, 23 nt, Artificial Sequence
  source: /note=“Description of Artificial Sequence Synthetic oligonucleotide”
uuuauaacuacaagagguuauuu

SEQ ID NO: 255 - RNA, 21 nt, Artificial Sequence
  source: /note=“Description of Artificial Sequence Synthetic oligonucleotide”
  modified_base (21)..(21): Inverted abasic nucleotide
accucuuguaguuauaaaaun

SEQ ID NO: 256 - RNA, 23 nt, Artificial Sequence
  source: /note=“Description of Artificial Sequence Synthetic oligonucleotide”
uauuuuauaacuacaagagguuu

SEQ ID NO: 257 - RNA, 21 nt, Artificial Sequence
  source: /note=“Description of Artificial Sequence Synthetic oligonucleotide”
  modified_base (21)..(21): Inverted abasic nucleotide
ggucaacauccuaggacauun

SEQ ID NO: 258 - RNA, 23 nt, Artificial Sequence
  source: /note=“Description of Artificial Sequence Synthetic oligonucleotide”
aaauguccuaggauguugaccuu

SEQ ID NO: 259 - RNA, 21 nt, Artificial Sequence
  source: /note=“Description of Artificial Sequence Synthetic oligonucleotide”
  modified_base (21)..(21): Inverted abasic nucleotide
gucaacauccuaggacauuun

SEQ ID NO: 260 - RNA, 23 nt, Artificial Sequence
  source: /note=“Description of Artificial Sequence Synthetic oligonucleotide”
aaaauguccuaggauguugacuu

SEQ ID NO: 261 - RNA, 21 nt, Artificial Sequence
  source: /note=“Description of Artificial Sequence Synthetic oligonucleotide”
  modified_base (21)..(21): Inverted abasic nucleotide
ucaacauccuaggacauuuun

SEQ ID NO: 262 - RNA, 21 nt, Artificial Sequence
  source: /note=“Description of Artificial Sequence Synthetic oligonucleotide”
aaauguccuaggauguugauu

SEQ ID NO: 263 - RNA, 21 nt, Artificial Sequence
  source: /note=“Description of Artificial Sequence Synthetic oligonucleotide”
  modified_base (21)..(21): Inverted abasic nucleotide
caacauccuaggacauuuuun

SEQ ID NO: 264 - RNA, 21 nt, Artificial Sequence
  source: /note=“Description of Artificial Sequence Synthetic oligonucleotide”
aaaauguccuaggauguuguu

SEQ ID NO: 265 - RNA, 21 nt, Artificial Sequence
  source: /note=“Description of Artificial Sequence Synthetic oligonucleotide”
  modified_base (21)..(21): Inverted abasic nucleotide
ggucaacauccuaggacauun

SEQ ID NO: 266 - RNA, 23 nt, Artificial Sequence
  source: /note=“Description of Artificial Sequence Synthetic oligonucleotide”
aaauguccuaggauguugaccuu

SEQ ID NO: 267 - RNA, 21 nt, Artificial Sequence
  source: /note=“Description of Artificial Sequence Synthetic oligonucleotide”
  modified_base (21)..(21): Inverted abasic nucleotide
gucaacauccuaggacauuun

SEQ ID NO: 268 - RNA, 23 nt, Artificial Sequence
  source: /note=“Description of Artificial Sequence Synthetic oligonucleotide”
aaaauguccuaggauguugacuu

SEQ ID NO: 269 - RNA, 21 nt, Artificial Sequence
  source: /note=“Description of Artificial Sequence Synthetic oligonucleotide”
  modified_base (21)..(21): Inverted abasic nucleotide
ggucaacauccuaggacauun

SEQ ID NO: 270 - RNA, 21 nt, Artificial Sequence
  source: /note=“Description of Artificial Sequence Synthetic oligonucleotide”
aaauguccuaggauguugacc

SEQ ID NO: 271 - RNA, 21 nt, Artificial Sequence
  source: /note=“Description of Artificial Sequence Synthetic oligonucleotide”
  modified_base (21)..(21): Inverted abasic nucleotide
gucaacauccuaggacauuun

SEQ ID NO: 272 - RNA, 21 nt, Artificial Sequence
  source: /note=“Description of Artificial Sequence Synthetic oligonucleotide”
aaaauguccuaggauguugac

SEQ ID NO: 273 - RNA, 21 nt, Artificial Sequence
  source: /note=“Description of Artificial Sequence Synthetic oligonucleotide”
  modified_base (21)..(21): Inverted abasic nucleotide
ggucaacauccuaggacauun

SEQ ID NO: 274 - RNA, 21 nt, Artificial Sequence
  source: /note=“Description of Artificial Sequence Synthetic oligonucleotide”
aaauguccuaggauguugacc

SEQ ID NO: 275 - RNA, 21 nt, Artificial Sequence
  source: /note=“Description of Artificial Sequence Synthetic oligonucleotide”
  modified_base (21)..(21): Inverted abasic nucleotide
gucaacauccuaggacauuun

SEQ ID NO: 276 - RNA, 21 nt, Artificial Sequence
  source: /note=“Description of Artificial Sequence Synthetic oligonucleotide”
aaaauguccuaggauguugac

SEQ ID NO: 277 - RNA, 21 nt, Artificial Sequence
  source: /note=“Description of Artificial Sequence Synthetic oligonucleotide”
  modified_base (21)..(21): Inverted abasic nucleotide
gucaacauccuaggacauuun

SEQ ID NO: 278 - RNA, 21 nt, Artificial Sequence
  source: /note=“Description of Artificial Sequence Synthetic oligonucleotide”
aaaauguccuaggauguugac

SEQ ID NO: 279 - RNA, 21 nt, Artificial Sequence
  source: /note=“Description of Artificial Sequence Synthetic oligonucleotide”
  modified_base (21)..(21): Inverted abasic nucleotide
ggucaacauccuaggacauun

SEQ ID NO: 280 - RNA, 21 nt, Artificial Sequence
  source: /note=“Description of Artificial Sequence Synthetic oligonucleotide”
aaauguccuaggauguugacc

SEQ ID NO: 281 - RNA, 20 nt, Artificial Sequence
  source: /note=“Description of Artificial Sequence Synthetic oligonucleotide”
  modified_base (1)..(1): Inverted abasic nucleotide
nucaacauccuaggacauuu

SEQ ID NO: 282 - RNA, 21 nt, Artificial Sequence
  source: /note=“Description of Artificial Sequence Synthetic oligonucleotide”
aaauguccuaggauguugauu

SEQ ID NO: 283 - RNA, 20 nt, Artificial Sequence
  source: /note=“Description of Artificial Sequence Synthetic oligonucleotide”
  modified_base (1)..(1): Inverted abasic nucleotide
ncaacauccuaggacauuuu

SEQ ID NO: 284 - RNA, 21 nt, Artificial Sequence
  source: /note=“Description of Artificial Sequence Synthetic oligonucleotide”
aaaauguccuaggauguuguu

SEQ ID NO: 285 - RNA, 19 nt, Artificial Sequence
  source: /note=“Description of Artificial Sequence Synthetic oligonucleotide”
  modified_base (19)..(19): Inverted abasic nucleotide
ucaacauccuaggacauun

SEQ ID NO: 286 - RNA, 21 nt, Artificial Sequence
  source: /note=“Description of Artificial Sequence Synthetic oligonucleotide”
aaauguccuaggauguugauu

SEQ ID NO: 287 - RNA, 19 nt, Artificial Sequence
  source: /note=“Description of Artificial Sequence Synthetic oligonucleotide”
  modified_base (19)..(19): Inverted abasic nucleotide
caacauccuaggacauuun

SEQ ID NO: 288 - RNA, 21 nt, Artificial Sequence
  source: /note=“Description of Artificial Sequence Synthetic oligonucleotide”
aaaauguccuaggauguuguu

SEQ ID NO: 289 - RNA, 21 nt, Artificial Sequence
  source: /note=“Description of Artificial Sequence Synthetic oligonucleotide”
  modified_base (21)..(21): Inverted abasic nucleotide
ggucaacaucgauggucauun

SEQ ID NO: 290 - RNA, 23 nt, Artificial Sequence
  source: /note=“Description of Artificial Sequence Synthetic oligonucleotide”
aaaugaccaucgauguugaccuu

SEQ ID NO: 291 - RNA, 21 nt, Artificial Sequence
  source: /note=“Description of Artificial Sequence Synthetic oligonucleotide”
  modified_base (21)..(21): Inverted abasic nucleotide
gucaacauccaucgagauuun

SEQ ID NO: 292 - RNA, 23 nt, Artificial Sequence
  source: /note=“Description of Artificial Sequence Synthetic oligonucleotide”
aaaaucucgauggauguugacuu

SEQ ID NO: 293 - RNA, 21 nt, Artificial Sequence
  source: /note=“Description of Artificial Sequence Synthetic oligonucleotide”
  modified_base (21)..(21): Inverted abasic nucleotide
gucaacauccuaggacauuun

SEQ ID NO: 294 - RNA, 21 nt, Artificial Sequence
  source: /note=“Description of Artificial Sequence Synthetic oligonucleotide”
aauguccuaggauguugacuu

SEQ ID NO: 295 - RNA, 21 nt, Artificial Sequence
  source: /note=“Description of Artificial Sequence Synthetic oligonucleotide”
  modified_base (21)..(21): Inverted abasic nucleotide
cucccacuacaucaagacuun

SEQ ID NO: 296 - RNA, 21 nt, Artificial Sequence
  source: /note=“Description of Artificial Sequence Synthetic oligonucleotide”
agucuugauguagugggaguu

SEQ ID NO: 297 - RNA, 21 nt, Artificial Sequence
  source: /note=“Description of Artificial Sequence Synthetic oligonucleotide”
  modified_base (21)..(21): Inverted abasic nucleotide
ccacuacaucaagacuaauun

SEQ ID NO: 298 - RNA, 21 nt, Artificial Sequence
  source: /note=“Description of Artificial Sequence Synthetic oligonucleotide”
auuagucuugauguagugguu

SEQ ID NO: 299 - RNA, 19 nt, Artificial Sequence
  source: /note=“Description of Artificial Sequence Synthetic oligonucleotide”
  modified_base (19)..(19): Inverted abasic nucleotide
uuccuuaucucaucccuun

SEQ ID NO: 300 - RNA, 21 nt, Artificial Sequence
  source: /note=“Description of Artificial Sequence Synthetic oligonucleotide”
uaagggaugagauaaggaauu

SEQ ID NO: 301 - RNA, 19 nt, Artificial Sequence
  source: /note=“Description of Artificial Sequence Synthetic oligonucleotide”
  modified_base (19)..(19): Inverted abasic nucleotide
uuccuuaugagaucccuun

SEQ ID NO: 302 - RNA, 21 nt, Artificial Sequence
  source: /note=“Description of Artificial Sequence Synthetic oligonucleotide”
uaagggaucucauaaggaauu

SEQ ID NO: 303 - RNA, 19 nt, Artificial Sequence
  source: /note=“Description of Artificial Sequence Synthetic oligonucleotide”
  modified_base (19)..(19): Inverted abasic nucleotide
ucaacaucgauggucauun

SEQ ID NO: 304 - RNA, 21 nt, Artificial Sequence
  source: /note=“Description of Artificial Sequence Synthetic oligonucleotide”
aaaugaccaucgauguugauu

SEQ ID NO: 305 - RNA, 21 nt, Artificial Sequence
  source: /note=“Description of Artificial Sequence Synthetic oligonucleotide”
uacaucaagacuaaucuuguu

SEQ ID NO: 306 - RNA, 23 nt, Artificial Sequence
  source: /note=“Description of Artificial Sequence Synthetic oligonucleotide”
aacaagauuagucuugauguagu

SEQ ID NO: 307 - RNA, 21 nt, Artificial Sequence
  source: /note=“Description of Artificial Sequence Synthetic oligonucleotide”
  modified_base (21)..(21): Inverted abasic nucleotide
augcuuuugcauggacuaucn

SEQ ID NO: 308 - DNA, 23 bp, Artificial Sequence
  source: /note=“Description of Artificial Sequence Synthetic oligonucleotide”
  source: /note=“Description of Combined DNA/RNA Molecule Synthetic oligonucleotide”
agauagtccaugcaaaagcauuc

SEQ ID NO: 309 - RNA, 21 nt, Artificial Sequence
  source: /note=“Description of Artificial Sequence Synthetic oligonucleotide”
cguaugcagaauauucaauuu

SEQ ID NO: 310 - RNA, 23 nt, Artificial Sequence
  source: /note=“Description of Artificial Sequence Synthetic oligonucleotide”
aaauugaauauucugcauacgau

SEQ ID NO: 311 - RNA, 21 nt, Artificial Sequence
  source: /note=“Description of Artificial Sequence Synthetic oligonucleotide”
cguaugcagaauauucaauuu

SEQ ID NO: 312 - RNA, 23 nt, Artificial Sequence
  source: /note=“Description of Artificial Sequence Synthetic oligonucleotide”
aaauugaauauucugcauacgau

SEQ ID NO: 313 - RNA, 21 nt, Artificial Sequence
  source: /note=“Description of Artificial Sequence Synthetic oligonucleotide”
cuacaucaagacuaaucuugu

SEQ ID NO: 314 - DNA, 23 bp, Artificial Sequence
  source: /note=“Description of Artificial Sequence Synthetic oligonucleotide”
  source: /note=“Description of Combined DNA/RNA Molecule Synthetic oligonucleotide”
acaagatuagucuugauguagug

SEQ ID NO: 315 - RNA, 21 nt, Artificial Sequence
  source: /note=“Description of Artificial Sequence Synthetic oligonucleotide”
  modified_base (21)..(21): Inverted abasic nucleotide
aagccaugaacaucauccuan

SEQ ID NO: 316 - RNA, 23 nt, Artificial Sequence
  source: /note=“Description of Artificial Sequence Synthetic oligonucleotide”
auaggaugauguucauggcuuuu

SEQ ID NO: 317 - RNA, 21 nt, Artificial Sequence
  source: /note=“Description of Artificial Sequence Synthetic oligonucleotide”
  modified_base (21)..(21): Inverted abasic nucleotide
agccaugaacaucauccuagn

SEQ ID NO: 318 - RNA, 23 nt, Artificial Sequence
  source: /note=“Description of Artificial Sequence Synthetic oligonucleotide”
ucuaggaugauguucauggcuuu

SEQ ID NO: 319 - RNA, 21 nt, Artificial Sequence
  source: /note=“Description of Artificial Sequence Synthetic oligonucleotide”
  modified_base (21)..(21): Inverted abasic nucleotide
ccaugaacaucauccuagaan

SEQ ID NO: 320 - RNA, 23 nt, Artificial Sequence
  source: /note=“Description of Artificial Sequence Synthetic oligonucleotide”
uuucuaggaugauguucaugguu

SEQ ID NO: 321 - RNA, 21 nt, Artificial Sequence
  source: /note=“Description of Artificial Sequence Synthetic oligonucleotide”
  modified_base (21)..(21): Inverted abasic nucleotide
ugaacaucauccuagaaaucn

SEQ ID NO: 322 - RNA, 23 nt, Artificial Sequence
  source: /note=“Description of Artificial Sequence Synthetic oligonucleotide”
agauuucuaggaugauguucauu

SEQ ID NO: 323 - RNA, 21 nt, Artificial Sequence
  source: /note=“Description of Artificial Sequence Synthetic oligonucleotide”
  modified_base (21)..(21): Inverted abasic nucleotide
gaaguuuuucauuccucagan

SEQ ID NO: 324 - RNA, 23 nt, Artificial Sequence
  source: /note=“Description of Artificial Sequence Synthetic oligonucleotide”
aucugaggaaugaaaaacuucuu

SEQ ID NO: 325 - RNA, 21 nt, Artificial Sequence
  source: /note=“Description of Artificial Sequence Synthetic oligonucleotide”
  modified_base (21)..(21): Inverted abasic nucleotide
ggagaaaaucuguggcugggn

SEQ ID NO: 326 - RNA, 23 nt, Artificial Sequence
  source: /note=“Description of Artificial Sequence Synthetic oligonucleotide”
acccagccacagauuuucuccuu

SEQ ID NO: 327 - RNA, 21 nt, Artificial Sequence
  source: /note=“Description of Artificial Sequence Synthetic oligonucleotide”
  modified_base (21)..(21): Inverted abasic nucleotide
guggcuggggagauuguucun

SEQ ID NO: 328 - RNA, 23 nt, Artificial Sequence
  source: /note=“Description of Artificial Sequence Synthetic oligonucleotide”
aagaacaaucuccccagccacuu

SEQ ID NO: 329 - RNA, 21 nt, Artificial Sequence
  source: /note=“Description of Artificial Sequence Synthetic oligonucleotide”
  modified_base (21)..(21): Inverted abasic nucleotide
ggaauaggcaggcagacuacn

SEQ ID NO: 330 - RNA, 23 nt, Artificial Sequence
  source: /note=“Description of Artificial Sequence Synthetic oligonucleotide”
aguagucugccugccuauuccuu

SEQ ID NO: 331 - RNA, 21 nt, Artificial Sequence
  source: /note=“Description of Artificial Sequence Synthetic oligonucleotide”
  modified_base (21)..(21): Inverted abasic nucleotide
gaauaggcaggcagacuacun

SEQ ID NO: 332 - RNA, 23 nt, Artificial Sequence
  source: /note=“Description of Artificial Sequence Synthetic oligonucleotide”
aaguagucugccugccuauucuu

SEQ ID NO: 333 - RNA, 21 nt, Artificial Sequence
  source: /note=“Description of Artificial Sequence Synthetic oligonucleotide”
  modified_base (21)..(21): Inverted abasic nucleotide
uuugcaaaacgacagagcaun

SEQ ID NO: 334 - RNA, 23 nt, Artificial Sequence
  source: /note=“Description of Artificial Sequence Synthetic oligonucleotide”
uaugcucugucguuuugcaaauu

SEQ ID NO: 335 - RNA, 21 nt, Artificial Sequence
  source: /note=“Description of Artificial Sequence Synthetic oligonucleotide”
  modified_base (21)..(21): Inverted abasic nucleotide
gcaaaacgacagagcauauun

SEQ ID NO: 336 - RNA, 23 nt, Artificial Sequence
  source: /note=“Description of Artificial Sequence Synthetic oligonucleotide”
aaauaugcucugucguuuugcuu

SEQ ID NO: 337 - RNA, 21 nt, Artificial Sequence
  source: /note=“Description of Artificial Sequence Synthetic oligonucleotide”
  modified_base (21)..(21): Inverted abasic nucleotide
acgacagagcauauugguucn

SEQ ID NO: 338 - RNA, 23 nt, Artificial Sequence
  source: /note=“Description of Artificial Sequence Synthetic oligonucleotide”
agaaccaauaugcucugucguuu

SEQ ID NO: 339 - RNA, 21 nt, Artificial Sequence
  source: /note=“Description of Artificial Sequence Synthetic oligonucleotide”
  modified_base (21)..(21): Inverted abasic nucleotide
cgacagagcauauugguucun

SEQ ID NO: 340 - RNA, 23 nt, Artificial Sequence
  source: /note=“Description of Artificial Sequence Synthetic oligonucleotide”
aagaaccaauaugcucugucguu

SEQ ID NO: 341 - RNA, 21 nt, Artificial Sequence
  source: /note=“Description of Artificial Sequence Synthetic oligonucleotide”
  modified_base (21)..(21): Inverted abasic nucleotide
gacagagcauauugguucugn

SEQ ID NO: 342 - RNA, 23 nt, Artificial Sequence
  source: /note=“Description of Artificial Sequence Synthetic oligonucleotide”
acagaaccaauaugcucugucuu

SEQ ID NO: 343 - RNA, 21 nt, Artificial Sequence
  source: /note=“Description of Artificial Sequence Synthetic oligonucleotide”
  modified_base (21)..(21): Inverted abasic nucleotide
acagagcauauugguucugun

SEQ ID NO: 344 - RNA, 23 nt, Artificial Sequence
  source: /note=“Description of Artificial Sequence Synthetic oligonucleotide”
aacagaaccaauaugcucuguuu

SEQ ID NO: 345 - RNA, 21 nt, Artificial Sequence
  source: /note=“Description of Artificial Sequence Synthetic oligonucleotide”
  modified_base (21)..(21): Inverted abasic nucleotide
cagagcauauugguucugugn

SEQ ID NO: 346 - RNA, 23 nt, Artificial Sequence
  source: /note=“Description of Artificial Sequence Synthetic oligonucleotide”
acacagaaccaauaugcucuguu

SEQ ID NO: 347 - RNA, 21 nt, Artificial Sequence
  source: /note=“Description of Artificial Sequence Synthetic oligonucleotide”
  modified_base (21)..(21): Inverted abasic nucleotide
agagcauauugguucuguggn

SEQ ID NO: 348 - RNA, 23 nt, Artificial Sequence
  source: /note=“Description of Artificial Sequence Synthetic oligonucleotide”
accacagaaccaauaugcucuuu

SEQ ID NO: 349 - RNA, 21 nt, Artificial Sequence
  source: /note=“Description of Artificial Sequence Synthetic oligonucleotide”
  modified_base (21)..(21): Inverted abasic nucleotide
gagcauauugguucugugggn

SEQ ID NO: 350 - RNA, 23 nt, Artificial Sequence
  source: /note=“Description of Artificial Sequence Synthetic oligonucleotide”
ucccacagaaccaauaugcucuu

SEQ ID NO: 351 - RNA, 21 nt, Artificial Sequence
  source: /note=“Description of Artificial Sequence Synthetic oligonucleotide”
  modified_base (21)..(21): Inverted abasic nucleotide
cugugggauauuaauaagcgn

SEQ ID NO: 352 - RNA, 23 nt, Artificial Sequence
  source: /note=“Description of Artificial Sequence Synthetic oligonucleotide”
acgcuuauuaauaucccacaguu

SEQ ID NO: 353 - RNA, 21 nt, Artificial Sequence
  source: /note=“Description of Artificial Sequence Synthetic oligonucleotide”
  modified_base (21)..(21): Inverted abasic nucleotide
uuaauaagcgcgguguggagn

SEQ ID NO: 354 - RNA, 23 nt, Artificial Sequence
  source: /note=“Description of Artificial Sequence Synthetic oligonucleotide”
acuccacaccgcgcuuauuaauu

SEQ ID NO: 355 - RNA, 21 nt, Artificial Sequence
  source: /note=“Description of Artificial Sequence Synthetic oligonucleotide”
  modified_base (21)..(21): Inverted abasic nucleotide
uaauaagcgcgguguggaggn

SEQ ID NO: 356 - RNA, 23 nt, Artificial Sequence
  source: /note=“Description of Artificial Sequence Synthetic oligonucleotide”
uccuccacaccgcgcuuauuauu

SEQ ID NO: 357 - RNA, 21 nt, Artificial Sequence
  source: /note=“Description of Artificial Sequence Synthetic oligonucleotide”
  modified_base (21)..(21): Inverted abasic nucleotide
auaagcgcgguguggaggaan

SEQ ID NO: 358 - RNA, 23 nt, Artificial Sequence
  source: /note=“Description of Artificial Sequence Synthetic oligonucleotide”
uuuccuccacaccgcgcuuauuu

SEQ ID NO: 359 - RNA, 21 nt, Artificial Sequence
  source: /note=“Description of Artificial Sequence Synthetic oligonucleotide”
  modified_base (21)..(21): Inverted abasic nucleotide
uaagcgcgguguggaggaaan

SEQ ID NO: 360 - RNA, 23 nt, Artificial Sequence
  source: /note=“Description of Artificial Sequence Synthetic oligonucleotide”
auuuccuccacaccgcgcuuauu

SEQ ID NO: 361 - RNA, 21 nt, Artificial Sequence
  source: /note=“Description of Artificial Sequence Synthetic oligonucleotide”
  modified_base (21)..(21): Inverted abasic nucleotide
ggcgucacugcgcaugcguan

SEQ ID NO: 362 - RNA, 23 nt, Artificial Sequence
  source: /note=“Description of Artificial Sequence Synthetic oligonucleotide”
auacgcaugcgcagugacgccuu

SEQ ID NO: 363 - RNA, 21 nt, Artificial Sequence
  source: /note=“Description of Artificial Sequence Synthetic oligonucleotide”
  modified_base (21)..(21): Inverted abasic nucleotide
gcgucacugcgcaugcguaun

SEQ ID NO: 364 - RNA, 23 nt, Artificial Sequence
  source: /note=“Description of Artificial Sequence Synthetic oligonucleotide”
aauacgcaugcgcagugacgcuu

SEQ ID NO: 365 - RNA, 21 nt, Artificial Sequence
  source: /note=“Description of Artificial Sequence Synthetic oligonucleotide”
  modified_base (21)..(21): Inverted abasic nucleotide
cgucacugcgcaugcguaugn

SEQ ID NO: 366 - RNA, 23 nt, Artificial Sequence
  source: /note=“Description of Artificial Sequence Synthetic oligonucleotide”
acauacgcaugcgcagugacguu

SEQ ID NO: 367 - RNA, 21 nt, Artificial Sequence
  source: /note=“Description of Artificial Sequence Synthetic oligonucleotide”
  modified_base (21)..(21): Inverted abasic nucleotide
agcaacagagaagagaucuan

SEQ ID NO: 368 - RNA, 23 nt, Artificial Sequence
  source: /note=“Description of Artificial Sequence Synthetic oligonucleotide”
auagaucucuucucuguugcuuu

SEQ ID NO: 369 - RNA, 21 nt, Artificial Sequence
  source: /note=“Description of Artificial Sequence Synthetic oligonucleotide”
  modified_base (21)..(21): Inverted abasic nucleotide
caacagagaagagaucuaucn

SEQ ID NO: 370 - RNA, 23 nt, Artificial Sequence
  source: /note=“Description of Artificial Sequence Synthetic oligonucleotide”
agauagaucucuucucuguuguu

SEQ ID NO: 371 - RNA, 21 nt, Artificial Sequence
  source: /note=“Description of Artificial Sequence Synthetic oligonucleotide”
  modified_base (21)..(21): Inverted abasic nucleotide
aacagagaagagaucuaucgn

SEQ ID NO: 372 - RNA, 23 nt, Artificial Sequence
  source: /note=“Description of Artificial Sequence Synthetic oligonucleotide”
acgauagaucucuucucuguuuu

SEQ ID NO: 373 - RNA, 21 nt, Artificial Sequence
  source: /note=“Description of Artificial Sequence Synthetic oligonucleotide”
  modified_base (21)..(21): Inverted abasic nucleotide
cagagaagagaucuaucgcun

SEQ ID NO: 374 - RNA, 23 nt, Artificial Sequence
  source: /note=“Description of Artificial Sequence Synthetic oligonucleotide”
aagcgauagaucucuucucuguu

SEQ ID NO: 375 - RNA, 21 nt, Artificial Sequence
  source: /note=“Description of Artificial Sequence Synthetic oligonucleotide”
  modified_base (21)..(21): Inverted abasic nucleotide
agagaagagaucuaucgcucn

SEQ ID NO: 376 - RNA, 23 nt, Artificial Sequence
  source: /note=“Description of Artificial Sequence Synthetic oligonucleotide”
agagcgauagaucucuucucuuu

SEQ ID NO: 377 - RNA, 21 nt, Artificial Sequence
  source: /note=“Description of Artificial Sequence Synthetic oligonucleotide”
  modified_base (21)..(21): Inverted abasic nucleotide
gagaagagaucuaucgcucun

SEQ ID NO: 378 - RNA, 23 nt, Artificial Sequence
  source: /note=“Description of Artificial Sequence Synthetic oligonucleotide”
aagagcgauagaucucuucucuu

SEQ ID NO: 379 - RNA, 21 nt, Artificial Sequence
  source: /note=“Description of Artificial Sequence Synthetic oligonucleotide”
  modified_base (21)..(21): Inverted abasic nucleotide
aagagaucuaucgcucucuan

SEQ ID NO: 380 - RNA, 23 nt, Artificial Sequence
  source: /note=“Description of Artificial Sequence Synthetic oligonucleotide”
uuagagagcgauagaucucuuuu

SEQ ID NO: 381 - RNA, 21 nt, Artificial Sequence
  source: /note=“Description of Artificial Sequence Synthetic oligonucleotide”
  modified_base (21)..(21): Inverted abasic nucleotide
gagaucuaucgcucucuaaan

SEQ ID NO: 382 - RNA, 23 nt, Artificial Sequence
  source: /note=“Description of Artificial Sequence Synthetic oligonucleotide”
auuuagagagcgauagaucucuu

SEQ ID NO: 383 - RNA, 21 nt, Artificial Sequence
  source: /note=“Description of Artificial Sequence Synthetic oligonucleotide”
  modified_base (21)..(21): Inverted abasic nucleotide
gaucuaucgcucucuaaaucn

SEQ ID NO: 384 - RNA, 23 nt, Artificial Sequence
  source: /note=“Description of Artificial Sequence Synthetic oligonucleotide”
ugauuuagagagcgauagaucuu

SEQ ID NO: 385 - RNA, 21 nt, Artificial Sequence
  source: /note=“Description of Artificial Sequence Synthetic oligonucleotide”
  modified_base (21)..(21): Inverted abasic nucleotide
aucuaucgcucucuaaaucan

SEQ ID NO: 386 - RNA, 23 nt, Artificial Sequence
  source: /note=“Description of Artificial Sequence Synthetic oligonucleotide”
augauuuagagagcgauagauuu

SEQ ID NO: 387 - RNA, 21 nt, Artificial Sequence
  source: /note=“Description of Artificial Sequence Synthetic oligonucleotide”
  modified_base (21)..(21): Inverted abasic nucleotide
cgcucucuaaaucaggugaan

SEQ ID NO: 388 - RNA, 23 nt, Artificial Sequence
  source: /note=“Description of Artificial Sequence Synthetic oligonucleotide”
auucaccugauuuagagagcguu

SEQ ID NO: 389 - RNA, 21 nt, Artificial Sequence
  source: /note=“Description of Artificial Sequence Synthetic oligonucleotide”
  modified_base (21)..(21): Inverted abasic nucleotide
cucucuaaaucaggugaagan

SEQ ID NO: 390 - RNA, 23 nt, Artificial Sequence
  source: /note=“Description of Artificial Sequence Synthetic oligonucleotide”
uucuucaccugauuuagagaguu

SEQ ID NO: 391 - RNA, 21 nt, Artificial Sequence
  source: /note=“Description of Artificial Sequence Synthetic oligonucleotide”
  modified_base (21)..(21): Inverted abasic nucleotide
aaagaagugggugauguaacn

SEQ ID NO: 392 - RNA, 23 nt, Artificial Sequence
  source: /note=“Description of Artificial Sequence Synthetic oligonucleotide”
uguuacaucacccacuucuuuuu

SEQ ID NO: 393 - RNA, 21 nt, Artificial Sequence
  source: /note=“Description of Artificial Sequence Synthetic oligonucleotide”
  modified_base (21)..(21): Inverted abasic nucleotide
aagaagugggugauguaacan

SEQ ID NO: 394 - RNA, 23 nt, Artificial Sequence
  source: /note=“Description of Artificial Sequence Synthetic oligonucleotide”
uuguuacaucacccacuucuuuu

SEQ ID NO: 395 - RNA, 21 nt, Artificial Sequence
  source: /note=“Description of Artificial Sequence Synthetic oligonucleotide”
  modified_base (21)..(21): Inverted abasic nucleotide
agaagugggugauguaacaan

SEQ ID NO: 396 - RNA, 23 nt, Artificial Sequence
  source: /note=“Description of Artificial Sequence Synthetic oligonucleotide”
auuguuacaucacccacuucuuu

SEQ ID NO: 397 - RNA, 21 nt, Artificial Sequence
  source: /note=“Description of Artificial Sequence Synthetic oligonucleotide”
  modified_base (21)..(21): Inverted abasic nucleotide
gaagugggugauguaacaaun

SEQ ID NO: 398 - RNA, 23 nt, Artificial Sequence
  source: /note=“Description of Artificial Sequence Synthetic oligonucleotide”
aauuguuacaucacccacuucuu

SEQ ID NO: 399 - RNA, 21 nt, Artificial Sequence
  source: /note=“Description of Artificial Sequence Synthetic oligonucleotide”
  modified_base (21)..(21): Inverted abasic nucleotide
gauguaacaaucguggugaan

SEQ ID NO: 400 - RNA, 23 nt, Artificial Sequence
  source: /note=“Description of Artificial Sequence Synthetic oligonucleotide”
auucaccacgauuguuacaucuu

SEQ ID NO: 401 - RNA, 21 nt, Artificial Sequence
  source: /note=“Description of Artificial Sequence Synthetic oligonucleotide”
  modified_base (21)..(21): Inverted abasic nucleotide
auguaacaaucguggugaaun

SEQ ID NO: 402 - RNA, 23 nt, Artificial Sequence
  source: /note=“Description of Artificial Sequence Synthetic oligonucleotide”
uauucaccacgauuguuacauuu

SEQ ID NO: 403 - RNA, 21 nt, Artificial Sequence
  source: /note=“Description of Artificial Sequence Synthetic oligonucleotide”
  modified_base (21)..(21): Inverted abasic nucleotide
uguaacaaucguggugaauan

SEQ ID NO: 404 - RNA, 23 nt, Artificial Sequence
  source: /note=“Description of Artificial Sequence Synthetic oligonucleotide”
uuauucaccacgauuguuacauu

SEQ ID NO: 405 - RNA, 21 nt, Artificial Sequence
  source: /note=“Description of Artificial Sequence Synthetic oligonucleotide”
  modified_base (21)..(21): Inverted abasic nucleotide
guaacaaucguggugaauaan

SEQ ID NO: 406 - RNA, 23 nt, Artificial Sequence
  source: /note=“Description of Artificial Sequence Synthetic oligonucleotide”
auuauucaccacgauuguuacuu

SEQ ID NO: 407 - RNA, 21 nt, Artificial Sequence
  source: /note=“Description of Artificial Sequence Synthetic oligonucleotide”
  modified_base (21)..(21): Inverted abasic nucleotide
uaacaaucguggugaauaaun

SEQ ID NO: 408 - RNA, 23 nt, Artificial Sequence
  source: /note=“Description of Artificial Sequence Synthetic oligonucleotide”
aauuauucaccacgauuguuauu

SEQ ID NO: 409 - RNA, 21 nt, Artificial Sequence
  source: /note=“Description of Artificial Sequence Synthetic oligonucleotide”
  modified_base (21)..(21): Inverted abasic nucleotide
ggugaauaaugcugggacagn

SEQ ID NO: 410 - RNA, 23 nt, Artificial Sequence
  source: /note=“Description of Artificial Sequence Synthetic oligonucleotide”
acugucccagcauuauucaccuu

SEQ ID NO: 411 - RNA, 21 nt, Artificial Sequence
  source: /note=“Description of Artificial Sequence Synthetic oligonucleotide”
  modified_base (21)..(21): Inverted abasic nucleotide
gugaauaaugcugggacagun

SEQ ID NO: 412 - RNA, 23 nt, Artificial Sequence
  source: /note=“Description of Artificial Sequence Synthetic oligonucleotide”
uacugucccagcauuauucacuu

SEQ ID NO: 413 - RNA, 21 nt, Artificial Sequence
  source: /note=“Description of Artificial Sequence Synthetic oligonucleotide”
  modified_base (21)..(21): Inverted abasic nucleotide
uaaugcugggacaguauaucn

SEQ ID NO: 414 - RNA, 23 nt, Artificial Sequence
  source: /note=“Description of Artificial Sequence Synthetic oligonucleotide”
agauauacugucccagcauuauu

SEQ ID NO: 415 - RNA, 21 nt, Artificial Sequence
  source: /note=“Description of Artificial Sequence Synthetic oligonucleotide”
  modified_base (21)..(21): Inverted abasic nucleotide
aaugcugggacaguauauccn

SEQ ID NO: 416 - RNA, 23 nt, Artificial Sequence
  source: /note=“Description of Artificial Sequence Synthetic oligonucleotide”
uggauauacugucccagcauuuu

SEQ ID NO: 417 - RNA, 21 nt, Artificial Sequence
  source: /note=“Description of Artificial Sequence Synthetic oligonucleotide”
  modified_base (21)..(21): Inverted abasic nucleotide
aagagauuaccaagacauuun

SEQ ID NO: 418 - RNA, 23 nt, Artificial Sequence
  source: /note=“Description of Artificial Sequence Synthetic oligonucleotide”
aaaaugucuugguaaucucuuuu

SEQ ID NO: 419 - RNA, 21 nt, Artificial Sequence
  source: /note=“Description of Artificial Sequence Synthetic oligonucleotide”
  modified_base (21)..(21): Inverted abasic nucleotide
agagauuaccaagacauuugn

SEQ ID NO: 420 - RNA, 23 nt, Artificial Sequence
  source: /note=“Description of Artificial Sequence Synthetic oligonucleotide”
ucaaaugucuugguaaucucuuu

SEQ ID NO: 421 - RNA, 21 nt, Artificial Sequence
  source: /note=“Description of Artificial Sequence Synthetic oligonucleotide”
  modified_base (21)..(21): Inverted abasic nucleotide
gagauuaccaagacauuugan

SEQ ID NO: 422 - RNA, 23 nt, Artificial Sequence
  source: /note=“Description of Artificial Sequence Synthetic oligonucleotide”
aucaaaugucuugguaaucucuu

SEQ ID NO: 423 - RNA, 21 nt, Artificial Sequence
  source: /note=“Description of Artificial Sequence Synthetic oligonucleotide”
  modified_base (21)..(21): Inverted abasic nucleotide
ugaggucaacauccuaggacn

SEQ ID NO: 424 - RNA, 23 nt, Artificial Sequence
  source: /note=“Description of Artificial Sequence Synthetic oligonucleotide”
uguccuaggauguugaccucauu

SEQ ID NO: 425 - RNA, 21 nt, Artificial Sequence
  source: /note=“Description of Artificial Sequence Synthetic oligonucleotide”
  modified_base (21)..(21): Inverted abasic nucleotide
aggucaacauccuaggacaun

SEQ ID NO: 426 - RNA, 23 nt, Artificial Sequence
  source: /note=“Description of Artificial Sequence Synthetic oligonucleotide”
aauguccuaggauguugaccuuu

SEQ ID NO: 427 - RNA, 21 nt, Artificial Sequence
  source: /note=“Description of Artificial Sequence Synthetic oligonucleotide”
  modified_base (21)..(21): Inverted abasic nucleotide
ggucaacauccuaggacauun

SEQ ID NO: 428 - RNA, 23 nt, Artificial Sequence
  source: /note=“Description of Artificial Sequence Synthetic oligonucleotide”
aaauguccuaggauguugaccuu

SEQ ID NO: 429 - RNA, 21 nt, Artificial Sequence
  source: /note=“Description of Artificial Sequence Synthetic oligonucleotide”
  modified_base (21)..(21): Inverted abasic nucleotide
gucaacauccuaggacauuun

SEQ ID NO: 430 - RNA, 23 nt, Artificial Sequence
  source: /note=“Description of Artificial Sequence Synthetic oligonucleotide”
aaaauguccuaggauguugacuu

SEQ ID NO: 431 - RNA, 21 nt, Artificial Sequence
  source: /note=“Description of Artificial Sequence Synthetic oligonucleotide”
  modified_base (21)..(21): Inverted abasic nucleotide
ucaacauccuaggacauuuun

SEQ ID NO: 432 - RNA, 23 nt, Artificial Sequence
  source: /note=“Description of Artificial Sequence Synthetic oligonucleotide”
aaaaauguccuaggauguugauu

SEQ ID NO: 433 - RNA, 21 nt, Artificial Sequence
  source: /note=“Description of Artificial Sequence Synthetic oligonucleotide”
  modified_base (21)..(21): Inverted abasic nucleotide
caacauccuaggacauuuuun

SEQ ID NO: 434 - RNA, 23 nt, Artificial Sequence
  source: /note=“Description of Artificial Sequence Synthetic oligonucleotide”
aaaaaauguccuaggauguuguu

SEQ ID NO: 435 - RNA, 21 nt, Artificial Sequence
  source: /note=“Description of Artificial Sequence Synthetic oligonucleotide”
  modified_base (21)..(21): Inverted abasic nucleotide
caaaagcacuucuuccaucgn

SEQ ID NO: 436 - RNA, 23 nt, Artificial Sequence
  source: /note=“Description of Artificial Sequence Synthetic oligonucleotide”
ucgauggaagaagugcuuuuguu

SEQ ID NO: 437 - RNA, 21 nt, Artificial Sequence
  source: /note=“Description of Artificial Sequence Synthetic oligonucleotide”
  modified_base (21)..(21): Inverted abasic nucleotide
aaaagcacuucuuccaucgan

SEQ ID NO: 438 - RNA, 23 nt, Artificial Sequence
  source: /note=“Description of Artificial Sequence Synthetic oligonucleotide”
aucgauggaagaagugcuuuuuu

SEQ ID NO: 439 - RNA, 21 nt, Artificial Sequence
  source: /note=“Description of Artificial Sequence Synthetic oligonucleotide”
  modified_base (21)..(21): Inverted abasic nucleotide
aaagcacuucuuccaucgaun

SEQ ID NO: 440 - RNA, 23 nt, Artificial Sequence
  source: /note=“Description of Artificial Sequence Synthetic oligonucleotide”
aaucgauggaagaagugcuuuuu

SEQ ID NO: 441 - RNA, 21 nt, Artificial Sequence
  source: /note=“Description of Artificial Sequence Synthetic oligonucleotide”
  modified_base (21)..(21): Inverted abasic nucleotide
aagcacuucuuccaucgaugn

SEQ ID NO: 442 - RNA, 23 nt, Artificial Sequence
  source: /note=“Description of Artificial Sequence Synthetic oligonucleotide”
ucaucgauggaagaagugcuuuu

SEQ ID NO: 443 - RNA, 21 nt, Artificial Sequence
  source: /note=“Description of Artificial Sequence Synthetic oligonucleotide”
  modified_base (21)..(21): Inverted abasic nucleotide
agcacuucuuccaucgaugan

SEQ ID NO: 444 - RNA, 23 nt, Artificial Sequence
  source: /note=“Description of Artificial Sequence Synthetic oligonucleotide”
aucaucgauggaagaagugcuuu

SEQ ID NO: 445 - RNA, 21 nt, Artificial Sequence
  source: /note=“Description of Artificial Sequence Synthetic oligonucleotide”
  modified_base (21)..(21): Inverted abasic nucleotide
cacuucuuccaucgaugaugn

SEQ ID NO: 446 - RNA, 23 nt, Artificial Sequence
  source: /note=“Description of Artificial Sequence Synthetic oligonucleotide”
acaucaucgauggaagaaguguu

SEQ ID NO: 447 - RNA, 21 nt, Artificial Sequence
  source: /note=“Description of Artificial Sequence Synthetic oligonucleotide”
  modified_base (21)..(21): Inverted abasic nucleotide
acuucuuccaucgaugauggn

SEQ ID NO: 448 - RNA, 23 nt, Artificial Sequence
  source: /note=“Description of Artificial Sequence Synthetic oligonucleotide”
uccaucaucgauggaagaaguuu

SEQ ID NO: 449 - RNA, 21 nt, Artificial Sequence
  source: /note=“Description of Artificial Sequence Synthetic oligonucleotide”
  modified_base (21)..(21): Inverted abasic nucleotide
uuccaucgaugauggagagan

SEQ ID NO: 450 - RNA, 23 nt, Artificial Sequence
  source: /note=“Description of Artificial Sequence Synthetic oligonucleotide”
uucucuccaucaucgauggaauu

SEQ ID NO: 451 - RNA, 21 nt, Artificial Sequence
  source: /note=“Description of Artificial Sequence Synthetic oligonucleotide”
  modified_base (21)..(21): Inverted abasic nucleotide
ccaucgaugauggagagaaan

SEQ ID NO: 452 - RNA, 23 nt, Artificial Sequence
  source: /note=“Description of Artificial Sequence Synthetic oligonucleotide”
auuucucuccaucaucgaugguu

SEQ ID NO: 453 - RNA, 21 nt, Artificial Sequence
  source: /note=“Description of Artificial Sequence Synthetic oligonucleotide”
  modified_base (21)..(21): Inverted abasic nucleotide
gauggagagaaaucauggccn

SEQ ID NO: 454 - RNA, 23 nt, Artificial Sequence
  source: /note=“Description of Artificial Sequence Synthetic oligonucleotide”
uggccaugauuucucuccaucuu

SEQ ID NO: 455 - RNA, 21 nt, Artificial Sequence
  source: /note=“Description of Artificial Sequence Synthetic oligonucleotide”
  modified_base (21)..(21): Inverted abasic nucleotide
guggcuucagugugcggccan

SEQ ID NO: 456 - RNA, 23 nt, Artificial Sequence
  source: /note=“Description of Artificial Sequence Synthetic oligonucleotide”
auggccgcacacugaagccacuu

SEQ ID NO: 457 - RNA, 21 nt, Artificial Sequence
  source: /note=“Description of Artificial Sequence Synthetic oligonucleotide”
  modified_base (21)..(21): Inverted abasic nucleotide
uggcuucagugugcggccacn

SEQ ID NO: 458 - RNA, 23 nt, Artificial Sequence
  source: /note=“Description of Artificial Sequence Synthetic oligonucleotide”
aguggccgcacacugaagccauu

SEQ ID NO: 459 - RNA, 21 nt, Artificial Sequence
  source: /note=“Description of Artificial Sequence Synthetic oligonucleotide”
  modified_base (21)..(21): Inverted abasic nucleotide
gcuucagugugcggccacgan

SEQ ID NO: 460 - RNA, 23 nt, Artificial Sequence
  source: /note=“Description of Artificial Sequence Synthetic oligonucleotide”
uucguggccgcacacugaagcuu

SEQ ID NO: 461 - RNA, 21 nt, Artificial Sequence
  source: /note=“Description of Artificial Sequence Synthetic oligonucleotide”
  modified_base (21)..(21): Inverted abasic nucleotide
uucagugugcggccacgaagn

SEQ ID NO: 462 - RNA, 23 nt, Artificial Sequence
  source: /note=“Description of Artificial Sequence Synthetic oligonucleotide”
acuucguggccgcacacugaauu

SEQ ID NO: 463 - RNA, 21 nt, Artificial Sequence
  source: /note=“Description of Artificial Sequence Synthetic oligonucleotide”
  modified_base (21)..(21): Inverted abasic nucleotide
uugugaauacuggguucaccn

SEQ ID NO: 464 - RNA, 23 nt, Artificial Sequence
  source: /note=“Description of Artificial Sequence Synthetic oligonucleotide”
uggugaacccaguauucacaauu

SEQ ID NO: 465 - RNA, 21 nt, Artificial Sequence
  source: /note=“Description of Artificial Sequence Synthetic oligonucleotide”
  modified_base (21)..(21): Inverted abasic nucleotide
gaauacuggguucaccaaaan

SEQ ID NO: 466 - RNA, 23 nt, Artificial Sequence
  source: /note=“Description of Artificial Sequence Synthetic oligonucleotide”
uuuuuggugaacccaguauucuu

SEQ ID NO: 467 - RNA, 21 nt, Artificial Sequence
  source: /note=“Description of Artificial Sequence Synthetic oligonucleotide”
  modified_base (21)..(21): Inverted abasic nucleotide
auacuggguucaccaaaaaun

SEQ ID NO: 468 - RNA, 23 nt, Artificial Sequence
  source: /note=“Description of Artificial Sequence Synthetic oligonucleotide”
aauuuuuggugaacccaguauuu

SEQ ID NO: 469 - RNA, 21 nt, Artificial Sequence
  source: /note=“Description of Artificial Sequence Synthetic oligonucleotide”
  modified_base (21)..(21): Inverted abasic nucleotide
agcacaagauuauggccugun

SEQ ID NO: 470 - RNA, 23 nt, Artificial Sequence
  source: /note=“Description of Artificial Sequence Synthetic oligonucleotide”
uacaggccauaaucuugugcuuu

SEQ ID NO: 471 - RNA, 21 nt, Artificial Sequence
  source: /note=“Description of Artificial Sequence Synthetic oligonucleotide”
  modified_base (21)..(21): Inverted abasic nucleotide
cacaagauuauggccuguaun

SEQ ID NO: 472 - RNA, 23 nt, Artificial Sequence
  source: /note=“Description of Artificial Sequence Synthetic oligonucleotide”
aauacaggccauaaucuuguguu

SEQ ID NO: 473 - RNA, 21 nt, Artificial Sequence
  source: /note=“Description of Artificial Sequence Synthetic oligonucleotide”
  modified_base (21)..(21): Inverted abasic nucleotide
acaagauuauggccuguauun

SEQ ID NO: 474 - RNA, 23 nt, Artificial Sequence
  source: /note=“Description of Artificial Sequence Synthetic oligonucleotide”
aaauacaggccauaaucuuguuu

SEQ ID NO: 475 - RNA, 21 nt, Artificial Sequence
  source: /note=“Description of Artificial Sequence Synthetic oligonucleotide”
  modified_base (21)..(21): Inverted abasic nucleotide
caagauuauggccuguauugn

SEQ ID NO: 476 - RNA, 23 nt, Artificial Sequence
  source: /note=“Description of Artificial Sequence Synthetic oligonucleotide”
acaauacaggccauaaucuuguu

SEQ ID NO: 477 - RNA, 21 nt, Artificial Sequence
  source: /note=“Description of Artificial Sequence Synthetic oligonucleotide”
  modified_base (21)..(21): Inverted abasic nucleotide
agauuauggccuguauuggan

SEQ ID NO: 478 - RNA, 23 nt, Artificial Sequence
  source: /note=“Description of Artificial Sequence Synthetic oligonucleotide”
auccaauacaggccauaaucuuu

SEQ ID NO: 479 - RNA, 21 nt, Artificial Sequence
  source: /note=“Description of Artificial Sequence Synthetic oligonucleotide”
  modified_base (21)..(21): Inverted abasic nucleotide
gauuauggccuguauuggagn

SEQ ID NO: 480 - RNA, 23 nt, Artificial Sequence
  source: /note=“Description of Artificial Sequence Synthetic oligonucleotide”
ucuccaauacaggccauaaucuu

SEQ ID NO: 481 - RNA, 21 nt, Artificial Sequence
  source: /note=“Description of Artificial Sequence Synthetic oligonucleotide”
  modified_base (21)..(21): Inverted abasic nucleotide
gaagucugauagauggaauan

SEQ ID NO: 482 - RNA, 23 nt, Artificial Sequence
  source: /note=“Description of Artificial Sequence Synthetic oligonucleotide”
auauuccaucuaucagacuucuu

SEQ ID NO: 483 - RNA, 21 nt, Artificial Sequence
  source: /note=“Description of Artificial Sequence Synthetic oligonucleotide”
  modified_base (21)..(21): Inverted abasic nucleotide
aagucugauagauggaauacn

SEQ ID NO: 484 - RNA, 23 nt, Artificial Sequence
  source: /note=“Description of Artificial Sequence Synthetic oligonucleotide”
aguauuccaucuaucagacuuuu

SEQ ID NO: 485 - RNA, 21 nt, Artificial Sequence
  source: /note=“Description of Artificial Sequence Synthetic oligonucleotide”
  modified_base (21)..(21): Inverted abasic nucleotide
agucugauagauggaauacun

SEQ ID NO: 486 - RNA, 23 nt, Artificial Sequence
  source: /note=“Description of Artificial Sequence Synthetic oligonucleotide”
aaguauuccaucuaucagacuuu

SEQ ID NO: 487 - RNA, 21 nt, Artificial Sequence
  source: /note=“Description of Artificial Sequence Synthetic oligonucleotide”
  modified_base (21)..(21): Inverted abasic nucleotide
gucugauagauggaauacuun

SEQ ID NO: 488 - RNA, 23 nt, Artificial Sequence
  source: /note=“Description of Artificial Sequence Synthetic oligonucleotide”
uaaguauuccaucuaucagacuu

SEQ ID NO: 489 - RNA, 21 nt, Artificial Sequence
  source: /note=“Description of Artificial Sequence Synthetic oligonucleotide”
  modified_base (21)..(21): Inverted abasic nucleotide
ucugauagauggaauacuuan

SEQ ID NO: 490 - RNA, 23 nt, Artificial Sequence
  source: /note=“Description of Artificial Sequence Synthetic oligonucleotide”
auaaguauuccaucuaucagauu

SEQ ID NO: 491 - RNA, 21 nt, Artificial Sequence
  source: /note=“Description of Artificial Sequence Synthetic oligonucleotide”
  modified_base (21)..(21): Inverted abasic nucleotide
auagauggaauacuuaccaan

SEQ ID NO: 492 - RNA, 23 nt, Artificial Sequence
  source: /note=“Description of Artificial Sequence Synthetic oligonucleotide”
auugguaaguauuccaucuauuu

SEQ ID NO: 493 - RNA, 21 nt, Artificial Sequence
  source: /note=“Description of Artificial Sequence Synthetic oligonucleotide”
  modified_base (21)..(21): Inverted abasic nucleotide
uagauggaauacuuaccaaun

SEQ ID NO: 494 - RNA, 23 nt, Artificial Sequence
  source: /note=“Description of Artificial Sequence Synthetic oligonucleotide”
uauugguaaguauuccaucuauu

SEQ ID NO: 495 - RNA, 21 nt, Artificial Sequence
  source: /note=“Description of Artificial Sequence Synthetic oligonucleotide”
  modified_base (21)..(21): Inverted abasic nucleotide
agauggaauacuuaccaauan

SEQ ID NO: 496 - RNA, 23 nt, Artificial Sequence
  source: /note=“Description of Artificial Sequence Synthetic oligonucleotide”
uuauugguaaguauuccaucuuu

SEQ ID NO: 497 - RNA, 21 nt, Artificial Sequence
  source: /note=“Description of Artificial Sequence Synthetic oligonucleotide”
  modified_base (21)..(21): Inverted abasic nucleotide
gauggaauacuuaccaauaan

SEQ ID NO: 498 - RNA, 23 nt, Artificial Sequence
  source: /note=“Description of Artificial Sequence Synthetic oligonucleotide”
auuauugguaaguauuccaucuu

SEQ ID NO: 499 - RNA, 21 nt, Artificial Sequence
  source: /note=“Description of Artificial Sequence Synthetic oligonucleotide”
  modified_base (21)..(21): Inverted abasic nucleotide
auggaauacuuaccaauaagn

SEQ ID NO: 500 - RNA, 23 nt, Artificial Sequence
  source: /note=“Description of Artificial Sequence Synthetic oligonucleotide”
ucuuauugguaaguauuccauuu

SEQ ID NO: 501 - RNA, 21 nt, Artificial Sequence
  source: /note=“Description of Artificial Sequence Synthetic oligonucleotide”
  modified_base (21)..(21): Inverted abasic nucleotide
uggaauacuuaccaauaagan

SEQ ID NO: 502 - RNA, 23 nt, Artificial Sequence
  source: /note=“Description of Artificial Sequence Synthetic oligonucleotide”
uucuuauugguaaguauuccauu

SEQ ID NO: 503 - RNA, 21 nt, Artificial Sequence
  source: /note=“Description of Artificial Sequence Synthetic oligonucleotide”
  modified_base (21)..(21): Inverted abasic nucleotide
auaucaauaucuuucugagan

SEQ ID NO: 504 - RNA, 23 nt, Artificial Sequence
  source: /note=“Description of Artificial Sequence Synthetic oligonucleotide”
aucucagaaagauauugauauuu

SEQ ID NO: 505 - RNA, 21 nt, Artificial Sequence
  source: /note=“Description of Artificial Sequence Synthetic oligonucleotide”
  modified_base (21)..(21): Inverted abasic nucleotide
cuuucugagacuacagaagun

SEQ ID NO: 506 - RNA, 23 nt, Artificial Sequence
  source: /note=“Description of Artificial Sequence Synthetic oligonucleotide”
aacuucuguagucucagaaaguu

SEQ ID NO: 507 - RNA, 21 nt, Artificial Sequence
  source: /note=“Description of Artificial Sequence Synthetic oligonucleotide”
  modified_base (21)..(21): Inverted abasic nucleotide
uuucugagacuacagaaguun

SEQ ID NO: 508 - RNA, 23 nt, Artificial Sequence
  source: /note=“Description of Artificial Sequence Synthetic oligonucleotide”
aaacuucuguagucucagaaauu

SEQ ID NO: 509 - RNA, 21 nt, Artificial Sequence
  source: /note=“Description of Artificial Sequence Synthetic oligonucleotide”
  modified_base (21)..(21): Inverted abasic nucleotide
uucugagacuacagaaguuun

SEQ ID NO: 510 - RNA, 23 nt, Artificial Sequence
  source: /note=“Description of Artificial Sequence Synthetic oligonucleotide”
aaaacuucuguagucucagaauu

SEQ ID NO: 511 - RNA, 21 nt, Artificial Sequence
  source: /note=“Description of Artificial Sequence Synthetic oligonucleotide”
  modified_base (21)..(21): Inverted abasic nucleotide
ucugagacuacagaaguuucn

SEQ ID NO: 512 - RNA, 23 nt, Artificial Sequence
  source: /note=“Description of Artificial Sequence Synthetic oligonucleotide”
agaaacuucuguagucucagauu

SEQ ID NO: 513 - RNA, 21 nt, Artificial Sequence
  source: /note=“Description of Artificial Sequence Synthetic oligonucleotide”
  modified_base (21)..(21): Inverted abasic nucleotide
ugguuggccacaaaaucaaan

SEQ ID NO: 514 - RNA, 23 nt, Artificial Sequence
  source: /note=“Description of Artificial Sequence Synthetic oligonucleotide”
uuuugauuuuguggccaaccauu

SEQ ID NO: 515 - RNA, 21 nt, Artificial Sequence
  source: /note=“Description of Artificial Sequence Synthetic oligonucleotide”
  modified_base (21)..(21): Inverted abasic nucleotide
aaaugaaaugaauaaauaagn

SEQ ID NO: 516 - RNA, 23 nt, Artificial Sequence
  source: /note=“Description of Artificial Sequence Synthetic oligonucleotide”
acuuauuuauucauuucauuuuu

SEQ ID NO: 517 - RNA, 21 nt, Artificial Sequence
  source: /note=“Description of Artificial Sequence Synthetic oligonucleotide”
  modified_base (21)..(21): Inverted abasic nucleotide
uucacauuuuuucaguccugn

SEQ ID NO: 518 - RNA, 23 nt, Artificial Sequence
  source: /note=“Description of Artificial Sequence Synthetic oligonucleotide”
ucaggacugaaaaaaugugaauu

SEQ ID NO: 519 - RNA, 21 nt, Artificial Sequence
  source: /note=“Description of Artificial Sequence Synthetic oligonucleotide”
  modified_base (21)..(21): Inverted abasic nucleotide
guuuggcacuagcagcagucn

SEQ ID NO: 520 - RNA, 23 nt, Artificial Sequence
  source: /note=“Description of Artificial Sequence Synthetic oligonucleotide”
ugacugcugcuagugccaaacuu

SEQ ID NO: 521 - RNA, 21 nt, Artificial Sequence
  source: /note=“Description of Artificial Sequence Synthetic oligonucleotide”
  modified_base (21)..(21): Inverted abasic nucleotide
uuuggcacuagcagcagucan

SEQ ID NO: 522 - RNA, 23 nt, Artificial Sequence
  source: /note=“Description of Artificial Sequence Synthetic oligonucleotide”
uugacugcugcuagugccaaauu

SEQ ID NO: 523 - RNA, 21 nt, Artificial Sequence
  source: /note=“Description of Artificial Sequence Synthetic oligonucleotide”
  modified_base (21)..(21): Inverted abasic nucleotide
uuggcacuagcagcagucaan

SEQ ID NO: 524 - RNA, 23 nt, Artificial Sequence
  source: /note=“Description of Artificial Sequence Synthetic oligonucleotide”
uuugacugcugcuagugccaauu

SEQ ID NO: 525 - RNA, 21 nt, Artificial Sequence
  source: /note=“Description of Artificial Sequence Synthetic oligonucleotide”
  modified_base (21)..(21): Inverted abasic nucleotide
uggcacuagcagcagucaaan

SEQ ID NO: 526 - RNA, 23 nt, Artificial Sequence
  source: /note=“Description of Artificial Sequence Synthetic oligonucleotide”
auuugacugcugcuagugccauu

SEQ ID NO: 527 - RNA, 21 nt, Artificial Sequence
  source: /note=“Description of Artificial Sequence Synthetic oligonucleotide”
  modified_base (21)..(21): Inverted abasic nucleotide
ggcacuagcagcagucaaacn

SEQ ID NO: 528 - RNA, 23 nt, Artificial Sequence
  source: /note=“Description of Artificial Sequence Synthetic oligonucleotide”
aguuugacugcugcuagugccuu

SEQ ID NO: 529 - RNA, 21 nt, Artificial Sequence
  source: /note=“Description of Artificial Sequence Synthetic oligonucleotide”
  modified_base (21)..(21): Inverted abasic nucleotide
auuuacguaguuuuucauagn

SEQ ID NO: 530 - RNA, 23 nt, Artificial Sequence
  source: /note=“Description of Artificial Sequence Synthetic oligonucleotide”
acuaugaaaaacuacguaaauuu

SEQ ID NO: 531 - RNA, 21 nt, Artificial Sequence
  source: /note=“Description of Artificial Sequence Synthetic oligonucleotide”
  modified_base (21)..(21): Inverted abasic nucleotide
uuacguaguuuuucauaggun

SEQ ID NO: 532 - RNA, 23 nt, Artificial Sequence
  source: /note=“Description of Artificial Sequence Synthetic oligonucleotide”
aaccuaugaaaaacuacguaauu

SEQ ID NO: 533 - RNA, 21 nt, Artificial Sequence
  source: /note=“Description of Artificial Sequence Synthetic oligonucleotide”
  modified_base (21)..(21): Inverted abasic nucleotide
uacguaguuuuucauaggucn

SEQ ID NO: 534 - RNA, 23 nt, Artificial Sequence
  source: /note=“Description of Artificial Sequence Synthetic oligonucleotide”
agaccuaugaaaaacuacguauu

SEQ ID NO: 535 - RNA, 21 nt, Artificial Sequence
  source: /note=“Description of Artificial Sequence Synthetic oligonucleotide”
  modified_base (21)..(21): Inverted abasic nucleotide
uuacauaaacauacuuaaaan

SEQ ID NO: 536 - RNA, 23 nt, Artificial Sequence
  source: /note=“Description of Artificial Sequence Synthetic oligonucleotide”
auuuuaaguauguuuauguaauu

SEQ ID NO: 537 - RNA, 21 nt, Artificial Sequence
  source: /note=“Description of Artificial Sequence Synthetic oligonucleotide”
  modified_base (21)..(21): Inverted abasic nucleotide
uuaaagguggacaaaagcuan

SEQ ID NO: 538 - RNA, 23 nt, Artificial Sequence
  source: /note=“Description of Artificial Sequence Synthetic oligonucleotide”
auagcuuuuguccaccuuuaauu

SEQ ID NO: 539 - RNA, 21 nt, Artificial Sequence
  source: /note=“Description of Artificial Sequence Synthetic oligonucleotide”
  modified_base (21)..(21): Inverted abasic nucleotide
uaaagguggacaaaagcuacn

SEQ ID NO: 540 - RNA, 23 nt, Artificial Sequence
  source: /note=“Description of Artificial Sequence Synthetic oligonucleotide”
aguagcuuuuguccaccuuuauu

SEQ ID NO: 541 - RNA, 21 nt, Artificial Sequence
  source: /note=“Description of Artificial Sequence Synthetic oligonucleotide”
  modified_base (21)..(21): Inverted abasic nucleotide
aagguggacaaaagcuaccun

SEQ ID NO: 542 - RNA, 23 nt, Artificial Sequence
  source: /note=“Description of Artificial Sequence Synthetic oligonucleotide”
aagguagcuuuuguccaccuuuu

SEQ ID NO: 543 - RNA, 21 nt, Artificial Sequence
  source: /note=“Description of Artificial Sequence Synthetic oligonucleotide”
  modified_base (21)..(21): Inverted abasic nucleotide
gguggacaaaagcuaccuccn

SEQ ID NO: 544 - RNA, 23 nt, Artificial Sequence
  source: /note=“Description of Artificial Sequence Synthetic oligonucleotide”
aggagguagcuuuuguccaccuu

SEQ ID NO: 545 - RNA, 21 nt, Artificial Sequence
  source: /note=“Description of Artificial Sequence Synthetic oligonucleotide”
  modified_base (21)..(21): Inverted abasic nucleotide
acagcuaagagaucaaguuun

SEQ ID NO: 546 - RNA, 23 nt, Artificial Sequence
  source: /note=“Description of Artificial Sequence Synthetic oligonucleotide”
aaaacuugaucucuuagcuguuu

SEQ ID NO: 547 - RNA, 21 nt, Artificial Sequence
  source: /note=“Description of Artificial Sequence Synthetic oligonucleotide”
  modified_base (21)..(21): Inverted abasic nucleotide
cagcuaagagaucaaguuucn

SEQ ID NO: 548 - RNA, 23 nt, Artificial Sequence
  source: /note=“Description of Artificial Sequence Synthetic oligonucleotide”
ugaaacuugaucucuuagcuguu

SEQ ID NO: 549 - RNA, 21 nt, Artificial Sequence
  source: /note=“Description of Artificial Sequence Synthetic oligonucleotide”
  modified_base (21)..(21): Inverted abasic nucleotide
agcuaagagaucaaguuucan

SEQ ID NO: 550 - RNA, 23 nt, Artificial Sequence
  source: /note=“Description of Artificial Sequence Synthetic oligonucleotide”
augaaacuugaucucuuagcuuu

SEQ ID NO: 551 - RNA, 21 nt, Artificial Sequence
  source: /note=“Description of Artificial Sequence Synthetic oligonucleotide”
  modified_base (21)..(21): Inverted abasic nucleotide
ccuggacauauuuuaagauun

SEQ ID NO: 552 - RNA, 23 nt, Artificial Sequence
  source: /note=“Description of Artificial Sequence Synthetic oligonucleotide”
aaaucuuaaaauauguccagguu

SEQ ID NO: 553 - RNA, 21 nt, Artificial Sequence
  source: /note=“Description of Artificial Sequence Synthetic oligonucleotide”
  modified_base (21)..(21): Inverted abasic nucleotide
cuggacauauuuuaagauucn

SEQ ID NO: 554 - RNA, 23 nt, Artificial Sequence
  source: /note=“Description of Artificial Sequence Synthetic oligonucleotide”
ugaaucuuaaaauauguccaguu

SEQ ID NO: 555 - RNA, 21 nt, Artificial Sequence
  source: /note=“Description of Artificial Sequence Synthetic oligonucleotide”
  modified_base (21)..(21): Inverted abasic nucleotide
cuuccuuuuucauuagcccan

SEQ ID NO: 556 - RNA, 23 nt, Artificial Sequence
  source: /note=“Description of Artificial Sequence Synthetic oligonucleotide”
uugggcuaaugaaaaaggaaguu

SEQ ID NO: 557 - RNA, 21 nt, Artificial Sequence
  source: /note=“Description of Artificial Sequence Synthetic oligonucleotide”
  modified_base (21)..(21): Inverted abasic nucleotide
uuccuuuuucauuagcccaan

SEQ ID NO: 558 - RNA, 23 nt, Artificial Sequence
  source: /note=“Description of Artificial Sequence Synthetic oligonucleotide”
uuugggcuaaugaaaaaggaauu

SEQ ID NO: 559 - RNA, 21 nt, Artificial Sequence
  source: /note=“Description of Artificial Sequence Synthetic oligonucleotide”
  modified_base (21)..(21): Inverted abasic nucleotide
cccucuauauuuccucccuun

SEQ ID NO: 560 - RNA, 23 nt, Artificial Sequence
  source: /note=“Description of Artificial Sequence Synthetic oligonucleotide”
aaagggaggaaauauagaggguu

SEQ ID NO: 561 - RNA, 21 nt, Artificial Sequence
  source: /note=“Description of Artificial Sequence Synthetic oligonucleotide”
  modified_base (21)..(21): Inverted abasic nucleotide
uauuuccucccuuuuuauagn

SEQ ID NO: 562 - RNA, 23 nt, Artificial Sequence
  source: /note=“Description of Artificial Sequence Synthetic oligonucleotide”
acuauaaaaagggaggaaauauu

SEQ ID NO: 563 - RNA, 21 nt, Artificial Sequence
  source: /note=“Description of Artificial Sequence Synthetic oligonucleotide”
  modified_base (21)..(21): Inverted abasic nucleotide
uuccucccuuuuuauagucun

SEQ ID NO: 564 - RNA, 23 nt, Artificial Sequence
  source: /note=“Description of Artificial Sequence Synthetic oligonucleotide”
aagacuauaaaaagggaggaauu

SEQ ID NO: 565 - RNA, 21 nt, Artificial Sequence
  source: /note=“Description of Artificial Sequence Synthetic oligonucleotide”
  modified_base (21)..(21): Inverted abasic nucleotide
uccucccuuuuuauagucuun

SEQ ID NO: 566 - RNA, 23 nt, Artificial Sequence
  source: /note=“Description of Artificial Sequence Synthetic oligonucleotide”
uaagacuauaaaaagggaggauu

SEQ ID NO: 567 - RNA, 21 nt, Artificial Sequence
  source: /note=“Description of Artificial Sequence Synthetic oligonucleotide”
  modified_base (21)..(21): Inverted abasic nucleotide
ccuuuuuauagucuuauaagn

SEQ ID NO: 568 - RNA, 23 nt, Artificial Sequence
  source: /note=“Description of Artificial Sequence Synthetic oligonucleotide”
ucuuauaagacuauaaaaagguu

SEQ ID NO: 569 - RNA, 21 nt, Artificial Sequence
  source: /note=“Description of Artificial Sequence Synthetic oligonucleotide”
  modified_base (21)..(21): Inverted abasic nucleotide
cuuuuuauagucuuauaagan

SEQ ID NO: 570 - RNA, 23 nt, Artificial Sequence
  source: /note=“Description of Artificial Sequence Synthetic oligonucleotide”
aucuuauaagacuauaaaaaguu

SEQ ID NO: 571 - RNA, 21 nt, Artificial Sequence
  source: /note=“Description of Artificial Sequence Synthetic oligonucleotide”
  modified_base (21)..(21): Inverted abasic nucleotide
uuuuuauagucuuauaagaun

SEQ ID NO: 572 - RNA, 23 nt, Artificial Sequence
  source: /note=“Description of Artificial Sequence Synthetic oligonucleotide”
uaucuuauaagacuauaaaaauu

SEQ ID NO: 573 - RNA, 21 nt, Artificial Sequence
  source: /note=“Description of Artificial Sequence Synthetic oligonucleotide”
  modified_base (21)..(21): Inverted abasic nucleotide
uuuuauagucuuauaagauan

SEQ ID NO: 574 - RNA, 23 nt, Artificial Sequence
  source: /note=“Description of Artificial Sequence Synthetic oligonucleotide”
auaucuuauaagacuauaaaauu

SEQ ID NO: 575 - RNA, 21 nt, Artificial Sequence
  source: /note=“Description of Artificial Sequence Synthetic oligonucleotide”
  modified_base (21)..(21): Inverted abasic nucleotide
uuuauagucuuauaagauacn

SEQ ID NO: 576 - RNA, 23 nt, Artificial Sequence
  source: /note=“Description of Artificial Sequence Synthetic oligonucleotide”
uguaucuuauaagacuauaaauu

SEQ ID NO: 577 - RNA, 21 nt, Artificial Sequence
  source: /note=“Description of Artificial Sequence Synthetic oligonucleotide”
  modified_base (21)..(21): Inverted abasic nucleotide
uuauagucuuauaagauacan

SEQ ID NO: 578 - RNA, 23 nt, Artificial Sequence
  source: /note=“Description of Artificial Sequence Synthetic oligonucleotide”
auguaucuuauaagacuauaauu

SEQ ID NO: 579 - RNA, 21 nt, Artificial Sequence
  source: /note=“Description of Artificial Sequence Synthetic oligonucleotide”
  modified_base (21)..(21): Inverted abasic nucleotide
uauagucuuauaagauacaun

SEQ ID NO: 580 - RNA, 23 nt, Artificial Sequence
  source: /note=“Description of Artificial Sequence Synthetic oligonucleotide”
aauguaucuuauaagacuauauu

SEQ ID NO: 581 - RNA, 21 nt, Artificial Sequence
  source: /note=“Description of Artificial Sequence Synthetic oligonucleotide”
  modified_base (21)..(21): Inverted abasic nucleotide
ucuuauaagauacauuaugan

SEQ ID NO: 582 - RNA, 23 nt, Artificial Sequence
  source: /note=“Description of Artificial Sequence Synthetic oligonucleotide”
uucauaauguaucuuauaagauu

SEQ ID NO: 583 - RNA, 21 nt, Artificial Sequence
  source: /note=“Description of Artificial Sequence Synthetic oligonucleotide”
  modified_base (21)..(21): Inverted abasic nucleotide
uuuuaaguucuagccccaugn

SEQ ID NO: 584 - RNA, 23 nt, Artificial Sequence
  source: /note=“Description of Artificial Sequence Synthetic oligonucleotide”
ucauggggcuagaacuuaaaauu

SEQ ID NO: 585 - RNA, 21 nt, Artificial Sequence
  source: /note=“Description of Artificial Sequence Synthetic oligonucleotide”
  modified_base (21)..(21): Inverted abasic nucleotide
uuuaaguucuagccccaugan

SEQ ID NO: 586 - RNA, 23 nt, Artificial Sequence
  source: /note=“Description of Artificial Sequence Synthetic oligonucleotide”
aucauggggcuagaacuuaaauu

SEQ ID NO: 587 - RNA, 21 nt, Artificial Sequence
  source: /note=“Description of Artificial Sequence Synthetic oligonucleotide”
  modified_base (21)..(21): Inverted abasic nucleotide
uaaguucuagccccaugauan

SEQ ID NO: 588 - RNA, 23 nt, Artificial Sequence
  source: /note=“Description of Artificial Sequence Synthetic oligonucleotide”
uuaucauggggcuagaacuuauu

SEQ ID NO: 589 - RNA, 21 nt, Artificial Sequence
  source: /note=“Description of Artificial Sequence Synthetic oligonucleotide”
  modified_base (21)..(21): Inverted abasic nucleotide
aaguucuagccccaugauaan

SEQ ID NO: 590 - RNA, 23 nt, Artificial Sequence
  source: /note=“Description of Artificial Sequence Synthetic oligonucleotide”
auuaucauggggcuagaacuuuu

SEQ ID NO: 591 - RNA, 21 nt, Artificial Sequence
  source: /note=“Description of Artificial Sequence Synthetic oligonucleotide”
  modified_base (21)..(21): Inverted abasic nucleotide
aguucuagccccaugauaacn

SEQ ID NO: 592 - RNA, 23 nt, Artificial Sequence
  source: /note=“Description of Artificial Sequence Synthetic oligonucleotide”
aguuaucauggggcuagaacuuu

SEQ ID NO: 593 - RNA, 21 nt, Artificial Sequence
  source: /note=“Description of Artificial Sequence Synthetic oligonucleotide”
  modified_base (21)..(21): Inverted abasic nucleotide
guucuagccccaugauaaccn

SEQ ID NO: 594 - RNA, 23 nt, Artificial Sequence
  source: /note=“Description of Artificial Sequence Synthetic oligonucleotide”
agguuaucauggggcuagaacuu

SEQ ID NO: 595 - RNA, 21 nt, Artificial Sequence
  source: /note=“Description of Artificial Sequence Synthetic oligonucleotide”
  modified_base (21)..(21): Inverted abasic nucleotide
cuagccccaugauaaccuuun

SEQ ID NO: 596 - RNA, 23 nt, Artificial Sequence
  source: /note=“Description of Artificial Sequence Synthetic oligonucleotide”
aaaagguuaucauggggcuaguu

SEQ ID NO: 597 - RNA, 21 nt, Artificial Sequence
  source: /note=“Description of Artificial Sequence Synthetic oligonucleotide”
  modified_base (21)..(21): Inverted abasic nucleotide
agccccaugauaaccuuuuun

SEQ ID NO: 598 - RNA, 23 nt, Artificial Sequence
  source: /note=“Description of Artificial Sequence Synthetic oligonucleotide”
aaaaaagguuaucauggggcuuu

SEQ ID NO: 599 - RNA, 21 nt, Artificial Sequence
  source: /note=“Description of Artificial Sequence Synthetic oligonucleotide”
  modified_base (21)..(21): Inverted abasic nucleotide
gccccaugauaaccuuuuucn

SEQ ID NO: 600 - RNA, 23 nt, Artificial Sequence
  source: /note=“Description of Artificial Sequence Synthetic oligonucleotide”
agaaaaagguuaucauggggcuu

SEQ ID NO: 601 - RNA, 21 nt, Artificial Sequence
  source: /note=“Description of Artificial Sequence Synthetic oligonucleotide”
  modified_base (21)..(21): Inverted abasic nucleotide
cccaugauaaccuuuuucuun

SEQ ID NO: 602 - RNA, 23 nt, Artificial Sequence
  source: /note=“Description of Artificial Sequence Synthetic oligonucleotide”
aaagaaaaagguuaucauggguu

SEQ ID NO: 603 - RNA, 21 nt, Artificial Sequence
  source: /note=“Description of Artificial Sequence Synthetic oligonucleotide”
  modified_base (21)..(21): Inverted abasic nucleotide
ccaugauaaccuuuuucuuun

SEQ ID NO: 604 - RNA, 23 nt, Artificial Sequence
  source: /note=“Description of Artificial Sequence Synthetic oligonucleotide”
aaaagaaaaagguuaucaugguu

SEQ ID NO: 605 - RNA, 21 nt, Artificial Sequence
  source: /note=“Description of Artificial Sequence Synthetic oligonucleotide”
  modified_base (21)..(21): Inverted abasic nucleotide
caugauaaccuuuuucuuugn

SEQ ID NO: 606 - RNA, 23 nt, Artificial Sequence
  source: /note=“Description of Artificial Sequence Synthetic oligonucleotide”
acaaagaaaaagguuaucauguu

SEQ ID NO: 607 - RNA, 21 nt, Artificial Sequence
  source: /note=“Description of Artificial Sequence Synthetic oligonucleotide”
  modified_base (21)..(21): Inverted abasic nucleotide
auaaccuuuuucuuuguaaun

SEQ ID NO: 608 - RNA, 23 nt, Artificial Sequence
  source: /note=“Description of Artificial Sequence Synthetic oligonucleotide”
aauuacaaagaaaaagguuauuu

SEQ ID NO: 609 - RNA, 21 nt, Artificial Sequence
  source: /note=“Description of Artificial Sequence Synthetic oligonucleotide”
  modified_base (21)..(21): Inverted abasic nucleotide
uuuuucuuuguaauuuaugcn

SEQ ID NO: 610 - RNA, 23 nt, Artificial Sequence
  source: /note=“Description of Artificial Sequence Synthetic oligonucleotide”
agcauaaauuacaaagaaaaauu

SEQ ID NO: 611 - RNA, 21 nt, Artificial Sequence
  source: /note=“Description of Artificial Sequence Synthetic oligonucleotide”
  modified_base (21)..(21): Inverted abasic nucleotide
uuuucuuuguaauuuaugcun

SEQ ID NO: 612 - RNA, 23 nt, Artificial Sequence
  source: /note=“Description of Artificial Sequence Synthetic oligonucleotide”
aagcauaaauuacaaagaaaauu

SEQ ID NO: 613 - RNA, 21 nt, Artificial Sequence
  source: /note=“Description of Artificial Sequence Synthetic oligonucleotide”
  modified_base (21)..(21): Inverted abasic nucleotide
ggcuauuacauaagaaacaan

SEQ ID NO: 614 - RNA, 23 nt, Artificial Sequence
  source: /note=“Description of Artificial Sequence Synthetic oligonucleotide”
auuguuucuuauguaauagccuu

SEQ ID NO: 615 - RNA, 21 nt, Artificial Sequence
  source: /note=“Description of Artificial Sequence Synthetic oligonucleotide”
  modified_base (21)..(21): Inverted abasic nucleotide
cuauuacauaagaaacaaugn

SEQ ID NO: 616 - RNA, 23 nt, Artificial Sequence
  source: /note=“Description of Artificial Sequence Synthetic oligonucleotide”
acauuguuucuuauguaauaguu

SEQ ID NO: 617 - RNA, 21 nt, Artificial Sequence
  source: /note=“Description of Artificial Sequence Synthetic oligonucleotide”
  modified_base (21)..(21): Inverted abasic nucleotide
uuacauaagaaacaauggacn

SEQ ID NO: 618 - RNA, 23 nt, Artificial Sequence
  source: /note=“Description of Artificial Sequence Synthetic oligonucleotide”
aguccauuguuucuuauguaauu

SEQ ID NO: 619 - RNA, 21 nt, Artificial Sequence
  source: /note=“Description of Artificial Sequence Synthetic oligonucleotide”
  modified_base (21)..(21): Inverted abasic nucleotide
uacauaagaaacaauggaccn

SEQ ID NO: 620 - RNA, 23 nt, Artificial Sequence
  source: /note=“Description of Artificial Sequence Synthetic oligonucleotide”
agguccauuguuucuuauguauu

SEQ ID NO: 621 - RNA, 21 nt, Artificial Sequence
  source: /note=“Description of Artificial Sequence Synthetic oligonucleotide”
  modified_base (21)..(21): Inverted abasic nucleotide
acauaagaaacaauggacccn

SEQ ID NO: 622 - RNA, 23 nt, Artificial Sequence
  source: /note=“Description of Artificial Sequence Synthetic oligonucleotide”
uggguccauuguuucuuauguuu

SEQ ID NO: 623 - RNA, 21 nt, Artificial Sequence
  source: /note=“Description of Artificial Sequence Synthetic oligonucleotide”
  modified_base (21)..(21): Inverted abasic nucleotide
aagaaacaauggacccaagan

SEQ ID NO: 624 - RNA, 23 nt, Artificial Sequence
  source: /note=“Description of Artificial Sequence Synthetic oligonucleotide”
aucuuggguccauuguuucuuuu

SEQ ID NO: 625 - RNA, 21 nt, Artificial Sequence
  source: /note=“Description of Artificial Sequence Synthetic oligonucleotide”
  modified_base (21)..(21): Inverted abasic nucleotide
agaaacaauggacccaagagn

SEQ ID NO: 626 - RNA, 23 nt, Artificial Sequence
  source: /note=“Description of Artificial Sequence Synthetic oligonucleotide”
ucucuuggguccauuguuucuuu

SEQ ID NO: 627 - RNA, 21 nt, Artificial Sequence
  source: /note=“Description of Artificial Sequence Synthetic oligonucleotide”
  modified_base (21)..(21): Inverted abasic nucleotide
gaaacaauggacccaagagan

SEQ ID NO: 628 - RNA, 23 nt, Artificial Sequence
  source: /note=“Description of Artificial Sequence Synthetic oligonucleotide”
uucucuuggguccauuguuucuu

SEQ ID NO: 629 - RNA, 21 nt, Artificial Sequence
  source: /note=“Description of Artificial Sequence Synthetic oligonucleotide”
  modified_base (21)..(21): Inverted abasic nucleotide
aauagaaaaaauaauccgacn

SEQ ID NO: 630 - RNA, 23 nt, Artificial Sequence
  source: /note=“Description of Artificial Sequence Synthetic oligonucleotide”
agucggauuauuuuuucuauuuu

SEQ ID NO: 631 - RNA, 21 nt, Artificial Sequence
  source: /note=“Description of Artificial Sequence Synthetic oligonucleotide”
  modified_base (21)..(21): Inverted abasic nucleotide
auagaaaaaauaauccgacun

SEQ ID NO: 632 - RNA, 23 nt, Artificial Sequence
  source: /note=“Description of Artificial Sequence Synthetic oligonucleotide”
aagucggauuauuuuuucuauuu

SEQ ID NO: 633 - RNA, 21 nt, Artificial Sequence
  source: /note=“Description of Artificial Sequence Synthetic oligonucleotide”
  modified_base (21)..(21): Inverted abasic nucleotide
aaaacaauucacuaaaaauan

SEQ ID NO: 634 - RNA, 23 nt, Artificial Sequence
  source: /note=“Description of Artificial Sequence Synthetic oligonucleotide”
uuauuuuuagugaauuguuuuuu

SEQ ID NO: 635 - RNA, 21 nt, Artificial Sequence
  source: /note=“Description of Artificial Sequence Synthetic oligonucleotide”
  modified_base (21)..(21): Inverted abasic nucleotide
uguaguuauaaaauaaaacgn

SEQ ID NO: 636 - RNA, 23 nt, Artificial Sequence
  source: /note=“Description of Artificial Sequence Synthetic oligonucleotide”
acguuuuauuuuauaacuacauu

SEQ ID NO: 637 - RNA, 21 nt, Artificial Sequence
  source: /note=“Description of Artificial Sequence Synthetic oligonucleotide”
  modified_base (21)..(21): Inverted abasic nucleotide
aauaaaacguuugacuucuan

SEQ ID NO: 638 - RNA, 23 nt, Artificial Sequence
  source: /note=“Description of Artificial Sequence Synthetic oligonucleotide”
uuagaagucaaacguuuuauuuu

SEQ ID NO: 639 - RNA, 21 nt, Artificial Sequence
  source: /note=“Description of Artificial Sequence Synthetic oligonucleotide”
  modified_base (21)..(21): Inverted abasic nucleotide
auaaaacguuugacuucuaan

SEQ ID NO: 640 - RNA, 23 nt, Artificial Sequence
  source: /note=“Description of Artificial Sequence Synthetic oligonucleotide”
uuuagaagucaaacguuuuauuu

SEQ ID NO: 641 - RNA, 21 nt, Artificial Sequence
  source: /note=“Description of Artificial Sequence Synthetic oligonucleotide”
  modified_base (21)..(21): Inverted abasic nucleotide
uaaaacguuugacuucuaaan

SEQ ID NO: 642 - RNA, 23 nt, Artificial Sequence
  source: /note=“Description of Artificial Sequence Synthetic oligonucleotide”
auuuagaagucaaacguuuuauu

SEQ ID NO: 643 - RNA, 21 nt, Artificial Sequence
  source: /note=“Description of Artificial Sequence Synthetic oligonucleotide”
  modified_base (21)..(21): Inverted abasic nucleotide
aaaacguuugacuucuaaacn

SEQ ID NO: 644 - RNA, 23 nt, Artificial Sequence
  source: /note=“Description of Artificial Sequence Synthetic oligonucleotide”
aguuuagaagucaaacguuuuuu

SEQ ID NO: 645 - RNA, 21 nt, Artificial Sequence
  source: /note=“Description of Artificial Sequence Synthetic oligonucleotide”
  modified_base (21)..(21): Inverted abasic nucleotide
aaacguuugacuucuaaacun

SEQ ID NO: 646 - RNA, 23 nt, Artificial Sequence
  source: /note=“Description of Artificial Sequence Synthetic oligonucleotide”
aaguuuagaagucaaacguuuuu

SEQ ID NO: 647 - RNA, 21 nt, Artificial Sequence
  source: /note=“Description of Artificial Sequence Synthetic oligonucleotide”
  modified_base (21)..(21): Inverted abasic nucleotide
uucugcuucugaucaccaucn

SEQ ID NO: 648 - RNA, 23 nt, Artificial Sequence
  source: /note=“Description of Artificial Sequence Synthetic oligonucleotide”
ugauggugaucagaagcagaauu

SEQ ID NO: 649 - RNA, 21 nt, Artificial Sequence
  source: /note=“Description of Artificial Sequence Synthetic oligonucleotide”
  modified_base (21)..(21): Inverted abasic nucleotide
ucugcuucugaucaccaucan

SEQ ID NO: 650 - RNA, 23 nt, Artificial Sequence
  source: /note=“Description of Artificial Sequence Synthetic oligonucleotide”
augauggugaucagaagcagauu

SEQ ID NO: 651 - RNA, 21 nt, Artificial Sequence
  source: /note=“Description of Artificial Sequence Synthetic oligonucleotide”
  modified_base (21)..(21): Inverted abasic nucleotide
cuucugaucaccaucaucuan

SEQ ID NO: 652 - RNA, 23 nt, Artificial Sequence
  source: /note=“Description of Artificial Sequence Synthetic oligonucleotide”
auagaugauggugaucagaaguu

SEQ ID NO: 653 - RNA, 21 nt, Artificial Sequence
  source: /note=“Description of Artificial Sequence Synthetic oligonucleotide”
  modified_base (21)..(21): Inverted abasic nucleotide
ucucauuacuggagcugggcn

SEQ ID NO: 654 - RNA, 23 nt, Artificial Sequence
  source: /note=“Description of Artificial Sequence Synthetic oligonucleotide”
ugcccagcuccaguaaugagauu

SEQ ID NO: 655 - RNA, 21 nt, Artificial Sequence
  source: /note=“Description of Artificial Sequence Synthetic oligonucleotide”
  modified_base (21)..(21): Inverted abasic nucleotide
gugaauaaugcugggacagun

SEQ ID NO: 656 - RNA, 23 nt, Artificial Sequence
  source: /note=“Description of Artificial Sequence Synthetic oligonucleotide”
uacugucccagcauuauucacuu

SEQ ID NO: 657 - RNA, 21 nt, Artificial Sequence
  source: /note=“Description of Artificial Sequence Synthetic oligonucleotide”
  modified_base (21)..(21): Inverted abasic nucleotide
uaaugcugggacaguauaucn

SEQ ID NO: 658 - RNA, 23 nt, Artificial Sequence
  source: /note=“Description of Artificial Sequence Synthetic oligonucleotide”
agauauacugucccagcauuauu

SEQ ID NO: 659 - RNA, 21 nt, Artificial Sequence
  source: /note=“Description of Artificial Sequence Synthetic oligonucleotide”
  modified_base (21)..(21): Inverted abasic nucleotide
aaugcugggacaguauauccn

SEQ ID NO: 660 - RNA, 23 nt, Artificial Sequence
  source: /note=“Description of Artificial Sequence Synthetic oligonucleotide”
uggauauacugucccagcauuuu

SEQ ID NO: 661 - RNA, 21 nt, Artificial Sequence
  source: /note=“Description of Artificial Sequence Synthetic oligonucleotide”
  modified_base (21)..(21): Inverted abasic nucleotide
gggacaguauauccagccgan

SEQ ID NO: 662 - RNA, 23 nt, Artificial Sequence
  source: /note=“Description of Artificial Sequence Synthetic oligonucleotide”
aucggcuggauauacugucccuu

SEQ ID NO: 663 - RNA, 21 nt, Artificial Sequence
  source: /note=“Description of Artificial Sequence Synthetic oligonucleotide”
  modified_base (21)..(21): Inverted abasic nucleotide
ggacaguauauccagccgaun

SEQ ID NO: 664 - RNA, 23 nt, Artificial Sequence
  source: /note=“Description of Artificial Sequence Synthetic oligonucleotide”
aaucggcuggauauacuguccuu

SEQ ID NO: 665 - RNA, 21 nt, Artificial Sequence
  source: /note=“Description of Artificial Sequence Synthetic oligonucleotide”
  modified_base (21)..(21): Inverted abasic nucleotide
gacaguauauccagccgaucn

SEQ ID NO: 666 - RNA, 23 nt, Artificial Sequence
  source: /note=“Description of Artificial Sequence Synthetic oligonucleotide”
agaucggcuggauauacugucuu

SEQ ID NO: 667 - RNA, 21 nt, Artificial Sequence
  source: /note=“Description of Artificial Sequence Synthetic oligonucleotide”
  modified_base (21)..(21): Inverted abasic nucleotide
acaguauauccagccgaucun

SEQ ID NO: 668 - RNA, 23 nt, Artificial Sequence
  source: /note=“Description of Artificial Sequence Synthetic oligonucleotide”
aagaucggcuggauauacuguuu

SEQ ID NO: 669 - RNA, 21 nt, Artificial Sequence
  source: /note=“Description of Artificial Sequence Synthetic oligonucleotide”
  modified_base (21)..(21): Inverted abasic nucleotide
caguauauccagccgaucuun

SEQ ID NO: 670 - RNA, 23 nt, Artificial Sequence
  source: /note=“Description of Artificial Sequence Synthetic oligonucleotide”
aaagaucggcuggauauacuguu

SEQ ID NO: 671 - RNA, 21 nt, Artificial Sequence
  source: /note=“Description of Artificial Sequence Synthetic oligonucleotide”
  modified_base (21)..(21): Inverted abasic nucleotide
gacauuugaggucaacauccn

SEQ ID NO: 672 - RNA, 23 nt, Artificial Sequence
  source: /note=“Description of Artificial Sequence Synthetic oligonucleotide”
aggauguugaccucaaaugucuu

SEQ ID NO: 673 - RNA, 21 nt, Artificial Sequence
  source: /note=“Description of Artificial Sequence Synthetic oligonucleotide”
  modified_base (21)..(21): Inverted abasic nucleotide
ugaggucaacauccuaggacn

SEQ ID NO: 674 - RNA, 23 nt, Artificial Sequence
  source: /note=“Description of Artificial Sequence Synthetic oligonucleotide”
uguccuaggauguugaccucauu

SEQ ID NO: 675 - RNA, 21 nt, Artificial Sequence
  source: /note=“Description of Artificial Sequence Synthetic oligonucleotide”
  modified_base (21)..(21): Inverted abasic nucleotide
aggucaacauccuaggacaun

SEQ ID NO: 676 - RNA, 23 nt, Artificial Sequence
  source: /note=“Description of Artificial Sequence Synthetic oligonucleotide”
aauguccuaggauguugaccuuu

SEQ ID NO: 677 - RNA, 21 nt, Artificial Sequence
  source: /note=“Description of Artificial Sequence Synthetic oligonucleotide”
  modified_base (21)..(21): Inverted abasic nucleotide
ggucaacauccuaggacauun

SEQ ID NO: 678 - RNA, 23 nt, Artificial Sequence
  source: /note=“Description of Artificial Sequence Synthetic oligonucleotide”
aaauguccuaggauguugaccuu

SEQ ID NO: 679 - RNA, 21 nt, Artificial Sequence
  source: /note=“Description of Artificial Sequence Synthetic oligonucleotide”
  modified_base (21)..(21): Inverted abasic nucleotide
gucaacauccuaggacauuun

SEQ ID NO: 680 - RNA, 23 nt, Artificial Sequence
  source: /note=“Description of Artificial Sequence Synthetic oligonucleotide”
aaaauguccuaggauguugacuu

SEQ ID NO: 681 - RNA, 21 nt, Artificial Sequence
  source: /note=“Description of Artificial Sequence Synthetic oligonucleotide”
  modified_base (21)..(21): Inverted abasic nucleotide
ucaacauccuaggacauuuun

SEQ ID NO: 682 - RNA, 23 nt, Artificial Sequence
  source: /note=“Description of Artificial Sequence Synthetic oligonucleotide”
aaaaauguccuaggauguugauu

SEQ ID NO: 683 - RNA, 21 nt, Artificial Sequence
  source: /note=“Description of Artificial Sequence Synthetic oligonucleotide”
  modified_base (21)..(21): Inverted abasic nucleotide
caacauccuaggacauuuuun

SEQ ID NO: 684 - RNA, 23 nt, Artificial Sequence
  source: /note=“Description of Artificial Sequence Synthetic oligonucleotide”
aaaaaauguccuaggauguuguu

SEQ ID NO: 685 - RNA, 21 nt, Artificial Sequence
  source: /note=“Description of Artificial Sequence Synthetic oligonucleotide”
  modified_base (21)..(21): Inverted abasic nucleotide
caaaagcacuucuuccaucgn

SEQ ID NO: 686 - RNA, 23 nt, Artificial Sequence
  source: /note=“Description of Artificial Sequence Synthetic oligonucleotide”
ucgauggaagaagugcuuuuguu

SEQ ID NO: 687 - RNA, 21 nt, Artificial Sequence
  source: /note=“Description of Artificial Sequence Synthetic oligonucleotide”
  modified_base (21)..(21): Inverted abasic nucleotide
aaaagcacuucuuccaucgan

SEQ ID NO: 688 - RNA, 23 nt, Artificial Sequence
  source: /note=“Description of Artificial Sequence Synthetic oligonucleotide”
aucgauggaagaagugcuuuuuu

SEQ ID NO: 689 - RNA, 21 nt, Artificial Sequence
  source: /note=“Description of Artificial Sequence Synthetic oligonucleotide”
  modified_base (21)..(21): Inverted abasic nucleotide
aaagcacuucuuccaucgaun

SEQ ID NO: 690 - RNA, 23 nt, Artificial Sequence
  source: /note=“Description of Artificial Sequence Synthetic oligonucleotide”
aaucgauggaagaagugcuuuuu

SEQ ID NO: 691 - RNA, 21 nt, Artificial Sequence
  source: /note=“Description of Artificial Sequence Synthetic oligonucleotide”
  modified_base (21)..(21): Inverted abasic nucleotide
aagcacuucuuccaucgaugn

SEQ ID NO: 692 - RNA, 23 nt, Artificial Sequence
  source: /note=“Description of Artificial Sequence Synthetic oligonucleotide”
ucaucgauggaagaagugcuuuu

SEQ ID NO: 693 - RNA, 21 nt, Artificial Sequence
  source: /note=“Description of Artificial Sequence Synthetic oligonucleotide”
  modified_base (21)..(21): Inverted abasic nucleotide
agcacuucuuccaucgaugan

SEQ ID NO: 694 - RNA, 23 nt, Artificial Sequence
  source: /note=“Description of Artificial Sequence Synthetic oligonucleotide”
aucaucgauggaagaagugcuuu

SEQ ID NO: 695 - RNA, 21 nt, Artificial Sequence
  source: /note=“Description of Artificial Sequence Synthetic oligonucleotide”
  modified_base (21)..(21): Inverted abasic nucleotide
uuccuuaccucaucccauaun

SEQ ID NO: 696 - RNA, 23 nt, Artificial Sequence
  source: /note=“Description of Artificial Sequence Synthetic oligonucleotide”
aauaugggaugagguaaggaauu

SEQ ID NO: 697 - RNA, 21 nt, Artificial Sequence
  source: /note=“Description of Artificial Sequence Synthetic oligonucleotide”
  modified_base (21)..(21): Inverted abasic nucleotide
ccuuaccucaucccauauugn

SEQ ID NO: 698 - RNA, 23 nt, Artificial Sequence
  source: /note=“Description of Artificial Sequence Synthetic oligonucleotide”
acaauaugggaugagguaagguu

SEQ ID NO: 699 - RNA, 21 nt, Artificial Sequence
  source: /note=“Description of Artificial Sequence Synthetic oligonucleotide”
  modified_base (21)..(21): Inverted abasic nucleotide
accucaucccauauuguuccn

SEQ ID NO: 700 - RNA, 23 nt, Artificial Sequence
  source: /note=“Description of Artificial Sequence Synthetic oligonucleotide”
uggaacaauaugggaugagguuu

SEQ ID NO: 701 - RNA, 21 nt, Artificial Sequence
  source: /note=“Description of Artificial Sequence Synthetic oligonucleotide”
  modified_base (21)..(21): Inverted abasic nucleotide
ccucaucccauauuguuccan

SEQ ID NO: 702 - RNA, 23 nt, Artificial Sequence
  source: /note=“Description of Artificial Sequence Synthetic oligonucleotide”
auggaacaauaugggaugagguu

SEQ ID NO: 703 - RNA, 21 nt, Artificial Sequence
  source: /note=“Description of Artificial Sequence Synthetic oligonucleotide”
  modified_base (21)..(21): Inverted abasic nucleotide
ucccauauuguuccagcaaan

SEQ ID NO: 704 - RNA, 23 nt, Artificial Sequence
  source: /note=“Description of Artificial Sequence Synthetic oligonucleotide”
auuugcuggaacaauaugggauu

SEQ ID NO: 705 - RNA, 21 nt, Artificial Sequence
  source: /note=“Description of Artificial Sequence Synthetic oligonucleotide”
  modified_base (21)..(21): Inverted abasic nucleotide
ggcuuucacagaggucugacn

SEQ ID NO: 706 - RNA, 23 nt, Artificial Sequence
  source: /note=“Description of Artificial Sequence Synthetic oligonucleotide”
ugucagaccucugugaaagccuu

SEQ ID NO: 707 - RNA, 21 nt, Artificial Sequence
  source: /note=“Description of Artificial Sequence Synthetic oligonucleotide”
  modified_base (21)..(21): Inverted abasic nucleotide
uuugugaauacuggguucacn

SEQ ID NO: 708 - RNA, 23 nt, Artificial Sequence
  source: /note=“Description of Artificial Sequence Synthetic oligonucleotide”
agugaacccaguauucacaaauu

SEQ ID NO: 709 - RNA, 21 nt, Artificial Sequence
  source: /note=“Description of Artificial Sequence Synthetic oligonucleotide”
  modified_base (21)..(21): Inverted abasic nucleotide
uugugaauacuggguucaccn

SEQ ID NO: 710 - RNA, 23 nt, Artificial Sequence
  source: /note=“Description of Artificial Sequence Synthetic oligonucleotide”
uggugaacccaguauucacaauu

SEQ ID NO: 711 - RNA, 21 nt, Artificial Sequence
  source: /note=“Description of Artificial Sequence Synthetic oligonucleotide”
  modified_base (21)..(21): Inverted abasic nucleotide
gaauacuggguucaccaaaan

SEQ ID NO: 712 - RNA, 23 nt, Artificial Sequence
  source: /note=“Description of Artificial Sequence Synthetic oligonucleotide”
uuuuuggugaacccaguauucuu

SEQ ID NO: 713 - RNA, 21 nt, Artificial Sequence
  source: /note=“Description of Artificial Sequence Synthetic oligonucleotide”
  modified_base (21)..(21): Inverted abasic nucleotide
auacuggguucaccaaaaaun

SEQ ID NO: 714 - RNA, 23 nt, Artificial Sequence
  source: /note=“Description of Artificial Sequence Synthetic oligonucleotide”
aauuuuuggugaacccaguauuu

SEQ ID NO: 715 - RNA, 21 nt, Artificial Sequence
  source: /note=“Description of Artificial Sequence Synthetic oligonucleotide”
  modified_base (21)..(21): Inverted abasic nucleotide
uacuggguucaccaaaaaucn

SEQ ID NO: 716 - RNA, 23 nt, Artificial Sequence
  source: /note=“Description of Artificial Sequence Synthetic oligonucleotide”
agauuuuuggugaacccaguauu

SEQ ID NO: 717 - RNA, 21 nt, Artificial Sequence
  source: /note=“Description of Artificial Sequence Synthetic oligonucleotide”
  modified_base (21)..(21): Inverted abasic nucleotide
uuuuaaaucguaugcagaaun

SEQ ID NO: 718 - RNA, 23 nt, Artificial Sequence
  source: /note=“Description of Artificial Sequence Synthetic oligonucleotide”
uauucugcauacgauuuaaaauu

SEQ ID NO: 719 - RNA, 21 nt, Artificial Sequence
  source: /note=“Description of Artificial Sequence Synthetic oligonucleotide”
  modified_base (21)..(21): Inverted abasic nucleotide
uuuaaaucguaugcagaauan

SEQ ID NO: 720 - RNA, 23 nt, Artificial Sequence
  source: /note=“Description of Artificial Sequence Synthetic oligonucleotide”
auauucugcauacgauuuaaauu

SEQ ID NO: 721 - RNA, 21 nt, Artificial Sequence
  source: /note=“Description of Artificial Sequence Synthetic oligonucleotide”
  modified_base (21)..(21): Inverted abasic nucleotide
uaaaucguaugcagaauauun

SEQ ID NO: 722 - RNA, 23 nt, Artificial Sequence
  source: /note=“Description of Artificial Sequence Synthetic oligonucleotide”
aaauauucugcauacgauuuauu

SEQ ID NO: 723 - RNA, 21 nt, Artificial Sequence
  source: /note=“Description of Artificial Sequence Synthetic oligonucleotide”
  modified_base (21)..(21): Inverted abasic nucleotide
aaaucguaugcagaauauucn

SEQ ID NO: 724 - RNA, 23 nt, Artificial Sequence
  source: /note=“Description of Artificial Sequence Synthetic oligonucleotide”
ugaauauucugcauacgauuuuu

SEQ ID NO: 725 - RNA, 21 nt, Artificial Sequence
  source: /note=“Description of Artificial Sequence Synthetic oligonucleotide”
  modified_base (21)..(21): Inverted abasic nucleotide
aaucguaugcagaauauucan

SEQ ID NO: 726 - RNA, 23 nt, Artificial Sequence
  source: /note=“Description of Artificial Sequence Synthetic oligonucleotide”
uugaauauucugcauacgauuuu

SEQ ID NO: 727 - RNA, 21 nt, Artificial Sequence
  source: /note=“Description of Artificial Sequence Synthetic oligonucleotide”
  modified_base (21)..(21): Inverted abasic nucleotide
ucguaugcagaauauucaaun

SEQ ID NO: 728 - RNA, 23 nt, Artificial Sequence
  source: /note=“Description of Artificial Sequence Synthetic oligonucleotide”
aauugaauauucugcauacgauu

SEQ ID NO: 729 - RNA, 21 nt, Artificial Sequence
  source: /note=“Description of Artificial Sequence Synthetic oligonucleotide”
  modified_base (21)..(21): Inverted abasic nucleotide
cguaugcagaauauucaauun

SEQ ID NO: 730 - RNA, 23 nt, Artificial Sequence
  source: /note=“Description of Artificial Sequence Synthetic oligonucleotide”
aaauugaauauucugcauacguu

SEQ ID NO: 731 - RNA, 21 nt, Artificial Sequence
  source: /note=“Description of Artificial Sequence Synthetic oligonucleotide”
  modified_base (21)..(21): Inverted abasic nucleotide
uaugcagaauauucaauuugn

SEQ ID NO: 732 - RNA, 23 nt, Artificial Sequence
  source: /note=“Description of Artificial Sequence Synthetic oligonucleotide”
ucaaauugaauauucugcauauu

SEQ ID NO: 733 - RNA, 21 nt, Artificial Sequence
  source: /note=“Description of Artificial Sequence Synthetic oligonucleotide”
  modified_base (21)..(21): Inverted abasic nucleotide
aauauucaauuugaagcagun

SEQ ID NO: 734 - RNA, 23 nt, Artificial Sequence
  source: /note=“Description of Artificial Sequence Synthetic oligonucleotide”
aacugcuucaaauugaauauuuu

SEQ ID NO: 735 - RNA, 21 nt, Artificial Sequence
  source: /note=“Description of Artificial Sequence Synthetic oligonucleotide”
  modified_base (21)..(21): Inverted abasic nucleotide
aaaugaaaugaauaaauaagn

SEQ ID NO: 736 - RNA, 23 nt, Artificial Sequence
  source: /note=“Description of Artificial Sequence Synthetic oligonucleotide”
acuuauuuauucauuucauuuuu

SEQ ID NO: 737 - RNA, 21 nt, Artificial Sequence
  source: /note=“Description of Artificial Sequence Synthetic oligonucleotide”
  modified_base (21)..(21): Inverted abasic nucleotide
aaucaaugcugcaaagcuuun

SEQ ID NO: 738 - RNA, 23 nt, Artificial Sequence
  source: /note=“Description of Artificial Sequence Synthetic oligonucleotide”
uaaagcuuugcagcauugauuuu

SEQ ID NO: 739 - RNA, 21 nt, Artificial Sequence
  source: /note=“Description of Artificial Sequence Synthetic oligonucleotide”
  modified_base (21)..(21): Inverted abasic nucleotide
ugcugcaaagcuuuauuucan

SEQ ID NO: 740 - RNA, 23 nt, Artificial Sequence
  source: /note=“Description of Artificial Sequence Synthetic oligonucleotide”
augaaauaaagcuuugcagcauu

SEQ ID NO: 741 - RNA, 21 nt, Artificial Sequence
  source: /note=“Description of Artificial Sequence Synthetic oligonucleotide”
  modified_base (21)..(21): Inverted abasic nucleotide
gcugcaaagcuuuauuucacn

SEQ ID NO: 742 - RNA, 23 nt, Artificial Sequence
  source: /note=“Description of Artificial Sequence Synthetic oligonucleotide”
ugugaaauaaagcuuugcagcuu

SEQ ID NO: 743 - RNA, 21 nt, Artificial Sequence
  source: /note=“Description of Artificial Sequence Synthetic oligonucleotide”
  modified_base (21)..(21): Inverted abasic nucleotide
uuaaaaacauugguuuggcan

SEQ ID NO: 744 - RNA, 23 nt, Artificial Sequence
  source: /note=“Description of Artificial Sequence Synthetic oligonucleotide”
augccaaaccaauguuuuuaauu

SEQ ID NO: 745 - RNA, 21 nt, Artificial Sequence
  source: /note=“Description of Artificial Sequence Synthetic oligonucleotide”
  modified_base (21)..(21): Inverted abasic nucleotide
aaaaacauugguuuggcacun

SEQ ID NO: 746 - RNA, 23 nt, Artificial Sequence
  source: /note=“Description of Artificial Sequence Synthetic oligonucleotide”
uagugccaaaccaauguuuuuuu

SEQ ID NO: 747 - RNA, 21 nt, Artificial Sequence
  source: /note=“Description of Artificial Sequence Synthetic oligonucleotide”
  modified_base (21)..(21): Inverted abasic nucleotide
aacaagauuaauuaccugucn

SEQ ID NO: 748 - RNA, 23 nt, Artificial Sequence
  source: /note=“Description of Artificial Sequence Synthetic oligonucleotide”
agacagguaauuaaucuuguuuu

SEQ ID NO: 749 - RNA, 21 nt, Artificial Sequence
  source: /note=“Description of Artificial Sequence Synthetic oligonucleotide”
  modified_base (21)..(21): Inverted abasic nucleotide
caagauuaauuaccugucuun

SEQ ID NO: 750 - RNA, 23 nt, Artificial Sequence
  source: /note=“Description of Artificial Sequence Synthetic oligonucleotide”
aaagacagguaauuaaucuuguu

SEQ ID NO: 751 - RNA, 21 nt, Artificial Sequence
  source: /note=“Description of Artificial Sequence Synthetic oligonucleotide”
  modified_base (21)..(21): Inverted abasic nucleotide
uaauuaccugucuuccuguun

SEQ ID NO: 752 - RNA, 23 nt, Artificial Sequence
  source: /note=“Description of Artificial Sequence Synthetic oligonucleotide”
aaacaggaagacagguaauuauu

SEQ ID NO: 753 - RNA, 21 nt, Artificial Sequence
  source: /note=“Description of Artificial Sequence Synthetic oligonucleotide”
  modified_base (21)..(21): Inverted abasic nucleotide
ccugucuuccuguuucucaan

SEQ ID NO: 754 - RNA, 23 nt, Artificial Sequence
  source: /note=“Description of Artificial Sequence Synthetic oligonucleotide”
auugagaaacaggaagacagguu

SEQ ID NO: 755 - RNA, 21 nt, Artificial Sequence
  source: /note=“Description of Artificial Sequence Synthetic oligonucleotide”
  modified_base (21)..(21): Inverted abasic nucleotide
uuuccuuucaugccucuuaan

SEQ ID NO: 756 - RNA, 23 nt, Artificial Sequence
  source: /note=“Description of Artificial Sequence Synthetic oligonucleotide”
uuuaagaggcaugaaaggaaauu

SEQ ID NO: 757 - RNA, 21 nt, Artificial Sequence
  source: /note=“Description of Artificial Sequence Synthetic oligonucleotide”
  modified_base (21)..(21): Inverted abasic nucleotide
uuccuuucaugccucuuaaan

SEQ ID NO: 758 - RNA, 23 nt, Artificial Sequence
  source: /note=“Description of Artificial Sequence Synthetic oligonucleotide”
uuuuaagaggcaugaaaggaauu

SEQ ID NO: 759 - RNA, 21 nt, Artificial Sequence
  source: /note=“Description of Artificial Sequence Synthetic oligonucleotide”
  modified_base (21)..(21): Inverted abasic nucleotide
uuuuccauuuaaagguggacn

SEQ ID NO: 760 - RNA, 23 nt, Artificial Sequence
  source: /note=“Description of Artificial Sequence Synthetic oligonucleotide”
uguccaccuuuaaauggaaaauu

SEQ ID NO: 761 - RNA, 21 nt, Artificial Sequence
  source: /note=“Description of Artificial Sequence Synthetic oligonucleotide”
  modified_base (21)..(21): Inverted abasic nucleotide
uuuccauuuaaagguggacan

SEQ ID NO: 762 - RNA, 23 nt, Artificial Sequence
  source: /note=“Description of Artificial Sequence Synthetic oligonucleotide”
uuguccaccuuuaaauggaaauu

SEQ ID NO: 763 - RNA, 21 nt, Artificial Sequence
  source: /note=“Description of Artificial Sequence Synthetic oligonucleotide”
  modified_base (21)..(21): Inverted abasic nucleotide
uuccauuuaaagguggacaan

SEQ ID NO: 764 - RNA, 23 nt, Artificial Sequence
  source: /note=“Description of Artificial Sequence Synthetic oligonucleotide”
uuuguccaccuuuaaauggaauu

SEQ ID NO: 765 - RNA, 21 nt, Artificial Sequence
  source: /note=“Description of Artificial Sequence Synthetic oligonucleotide”
  modified_base (21)..(21): Inverted abasic nucleotide
uccauuuaaagguggacaaan

SEQ ID NO: 766 - RNA, 23 nt, Artificial Sequence
  source: /note=“Description of Artificial Sequence Synthetic oligonucleotide”
uuuuguccaccuuuaaauggauu

SEQ ID NO: 767 - RNA, 21 nt, Artificial Sequence
  source: /note=“Description of Artificial Sequence Synthetic oligonucleotide”
  modified_base (21)..(21): Inverted abasic nucleotide
gaacuuauuuacacagggaan

SEQ ID NO: 768 - RNA, 23 nt, Artificial Sequence
  source: /note=“Description of Artificial Sequence Synthetic oligonucleotide”
auucccuguguaaauaaguucuu

SEQ ID NO: 769 - RNA, 21 nt, Artificial Sequence
  source: /note=“Description of Artificial Sequence Synthetic oligonucleotide”
  modified_base (21)..(21): Inverted abasic nucleotide
cuuauuuacacagggaaggun

SEQ ID NO: 770 - RNA, 23 nt, Artificial Sequence
  source: /note=“Description of Artificial Sequence Synthetic oligonucleotide”
aaccuucccuguguaaauaaguu

SEQ ID NO: 771 - RNA, 21 nt, Artificial Sequence
  source: /note=“Description of Artificial Sequence Synthetic oligonucleotide”
  modified_base (21)..(21): Inverted abasic nucleotide
auuuacacagggaagguuuan

SEQ ID NO: 772 - RNA, 23 nt, Artificial Sequence
  source: /note=“Description of Artificial Sequence Synthetic oligonucleotide”
uuaaaccuucccuguguaaauuu

SEQ ID NO: 773 - RNA, 21 nt, Artificial Sequence
  source: /note=“Description of Artificial Sequence Synthetic oligonucleotide”
  modified_base (21)..(21): Inverted abasic nucleotide
uuuacacagggaagguuuaan

SEQ ID NO: 774 - RNA, 23 nt, Artificial Sequence
  source: /note=“Description of Artificial Sequence Synthetic oligonucleotide”
auuaaaccuucccuguguaaauu

SEQ ID NO: 775 - RNA, 21 nt, Artificial Sequence
  source: /note=“Description of Artificial Sequence Synthetic oligonucleotide”
  modified_base (21)..(21): Inverted abasic nucleotide
cagggaagguuuaagacugun

SEQ ID NO: 776 - RNA, 23 nt, Artificial Sequence
  source: /note=“Description of Artificial Sequence Synthetic oligonucleotide”
aacagucuuaaaccuucccuguu

SEQ ID NO: 777 - RNA, 21 nt, Artificial Sequence
  source: /note=“Description of Artificial Sequence Synthetic oligonucleotide”
  modified_base (21)..(21): Inverted abasic nucleotide
ggaagguuuaagacuguucan

SEQ ID NO: 778 - RNA, 23 nt, Artificial Sequence
  source: /note=“Description of Artificial Sequence Synthetic oligonucleotide”
uugaacagucuuaaaccuuccuu

SEQ ID NO: 779 - RNA, 21 nt, Artificial Sequence
  source: /note=“Description of Artificial Sequence Synthetic oligonucleotide”
  modified_base (21)..(21): Inverted abasic nucleotide
agguuuaagacuguucaagun

SEQ ID NO: 780 - RNA, 23 nt, Artificial Sequence
  source: /note=“Description of Artificial Sequence Synthetic oligonucleotide”
uacuugaacagucuuaaaccuuu

SEQ ID NO: 781 - RNA, 21 nt, Artificial Sequence
  source: /note=“Description of Artificial Sequence Synthetic oligonucleotide”
  modified_base (21)..(21): Inverted abasic nucleotide
gguuuaagacuguucaaguan

SEQ ID NO: 782 - RNA, 23 nt, Artificial Sequence
  source: /note=“Description of Artificial Sequence Synthetic oligonucleotide”
auacuugaacagucuuaaaccuu

SEQ ID NO: 783 - RNA, 21 nt, Artificial Sequence
  source: /note=“Description of Artificial Sequence Synthetic oligonucleotide”
  modified_base (21)..(21): Inverted abasic nucleotide
agacuguucaaguagcauucn

SEQ ID NO: 784 - RNA, 23 nt, Artificial Sequence
  source: /note=“Description of Artificial Sequence Synthetic oligonucleotide”
agaaugcuacuugaacagucuuu

SEQ ID NO: 785 - RNA, 21 nt, Artificial Sequence
  source: /note=“Description of Artificial Sequence Synthetic oligonucleotide”
  modified_base (21)..(21): Inverted abasic nucleotide
gacuguucaaguagcauuccn

SEQ ID NO: 786 - RNA, 23 nt, Artificial Sequence
  source: /note=“Description of Artificial Sequence Synthetic oligonucleotide”
uggaaugcuacuugaacagucuu

SEQ ID NO: 787 - RNA, 21 nt, Artificial Sequence
  source: /note=“Description of Artificial Sequence Synthetic oligonucleotide”
  modified_base (21)..(21): Inverted abasic nucleotide
acuguucaaguagcauuccan

SEQ ID NO: 788 - RNA, 23 nt, Artificial Sequence
  source: /note=“Description of Artificial Sequence Synthetic oligonucleotide”
uuggaaugcuacuugaacaguuu

SEQ ID NO: 789 - RNA, 21 nt, Artificial Sequence
  source: /note=“Description of Artificial Sequence Synthetic oligonucleotide”
  modified_base (21)..(21): Inverted abasic nucleotide
cuguucaaguagcauuccaan

SEQ ID NO: 790 - RNA, 23 nt, Artificial Sequence
  source: /note=“Description of Artificial Sequence Synthetic oligonucleotide”
auuggaaugcuacuugaacaguu

SEQ ID NO: 791 - RNA, 21 nt, Artificial Sequence
  source: /note=“Description of Artificial Sequence Synthetic oligonucleotide”
  modified_base (21)..(21): Inverted abasic nucleotide
uguucaaguagcauuccaaun

SEQ ID NO: 792 - RNA, 23 nt, Artificial Sequence
  source: /note=“Description of Artificial Sequence Synthetic oligonucleotide”
aauuggaaugcuacuugaacauu

SEQ ID NO: 793 - RNA, 21 nt, Artificial Sequence
  source: /note=“Description of Artificial Sequence Synthetic oligonucleotide”
  modified_base (21)..(21): Inverted abasic nucleotide
caagaacacagaaugagugcn

SEQ ID NO: 794 - RNA, 23 nt, Artificial Sequence
  source: /note=“Description of Artificial Sequence Synthetic oligonucleotide”
ugcacucauucuguguucuuguu

SEQ ID NO: 795 - RNA, 21 nt, Artificial Sequence
  source: /note=“Description of Artificial Sequence Synthetic oligonucleotide”
  modified_base (21)..(21): Inverted abasic nucleotide
acagaaugagugcacagcuan

SEQ ID NO: 796 - RNA, 23 nt, Artificial Sequence
  source: /note=“Description of Artificial Sequence Synthetic oligonucleotide”
uuagcugugcacucauucuguuu

SEQ ID NO: 797 - RNA, 21 nt, Artificial Sequence
  source: /note=“Description of Artificial Sequence Synthetic oligonucleotide”
  modified_base (21)..(21): Inverted abasic nucleotide
aggcagcuuuaucucaaccun

SEQ ID NO: 798 - RNA, 23 nt, Artificial Sequence
  source: /note=“Description of Artificial Sequence Synthetic oligonucleotide”
aagguugagauaaagcugccuuu

SEQ ID NO: 799 - RNA, 21 nt, Artificial Sequence
  source: /note=“Description of Artificial Sequence Synthetic oligonucleotide”
  modified_base (21)..(21): Inverted abasic nucleotide
uuuuaagauucagcauuugan

SEQ ID NO: 800 - RNA, 23 nt, Artificial Sequence
  source: /note=“Description of Artificial Sequence Synthetic oligonucleotide”
uucaaaugcugaaucuuaaaauu

SEQ ID NO: 801 - RNA, 21 nt, Artificial Sequence
  source: /note=“Description of Artificial Sequence Synthetic oligonucleotide”
  modified_base (21)..(21): Inverted abasic nucleotide
agauucagcauuugaaagaun

SEQ ID NO: 802 - RNA, 23 nt, Artificial Sequence
  source: /note=“Description of Artificial Sequence Synthetic oligonucleotide”
aaucuuucaaaugcugaaucuuu

SEQ ID NO: 803 - RNA, 21 nt, Artificial Sequence
  source: /note=“Description of Artificial Sequence Synthetic oligonucleotide”
  modified_base (21)..(21): Inverted abasic nucleotide
auuugaaagauuucccuagcn

SEQ ID NO: 804 - RNA, 23 nt, Artificial Sequence
  source: /note=“Description of Artificial Sequence Synthetic oligonucleotide”
agcuagggaaaucuuucaaauuu

SEQ ID NO: 805 - RNA, 21 nt, Artificial Sequence
  source: /note=“Description of Artificial Sequence Synthetic oligonucleotide”
  modified_base (21)..(21): Inverted abasic nucleotide
uucccuagccucuuccuuuun

SEQ ID NO: 806 - RNA, 23 nt, Artificial Sequence
  source: /note=“Description of Artificial Sequence Synthetic oligonucleotide”
aaaaaggaagaggcuagggaauu

SEQ ID NO: 807 - RNA, 21 nt, Artificial Sequence
  source: /note=“Description of Artificial Sequence Synthetic oligonucleotide”
  modified_base (21)..(21): Inverted abasic nucleotide
cuauucuggacuuuauuacun

SEQ ID NO: 808 - RNA, 23 nt, Artificial Sequence
  source: /note=“Description of Artificial Sequence Synthetic oligonucleotide”
aaguaauaaaguccagaauaguu

SEQ ID NO: 809 - RNA, 21 nt, Artificial Sequence
  source: /note=“Description of Artificial Sequence Synthetic oligonucleotide”
  modified_base (21)..(21): Inverted abasic nucleotide
aguccaccaaaaguggacccn

SEQ ID NO: 810 - RNA, 23 nt, Artificial Sequence
  source: /note=“Description of Artificial Sequence Synthetic oligonucleotide”
aggguccacuuuugguggacuuu

SEQ ID NO: 811 - RNA, 21 nt, Artificial Sequence
  source: /note=“Description of Artificial Sequence Synthetic oligonucleotide”
  modified_base (21)..(21): Inverted abasic nucleotide
caccaaaaguggacccucuan

SEQ ID NO: 812 - RNA, 23 nt, Artificial Sequence
  source: /note=“Description of Artificial Sequence Synthetic oligonucleotide”
auagaggguccacuuuugguguu

SEQ ID NO: 813 - RNA, 21 nt, Artificial Sequence
  source: /note=“Description of Artificial Sequence Synthetic oligonucleotide”
  modified_base (21)..(21): Inverted abasic nucleotide
accaaaaguggacccucuaun

SEQ ID NO: 814 - RNA, 23 nt, Artificial Sequence
  source: /note=“Description of Artificial Sequence Synthetic oligonucleotide”
uauagaggguccacuuuugguuu

SEQ ID NO: 815 - RNA, 21 nt, Artificial Sequence
  source: /note=“Description of Artificial Sequence Synthetic oligonucleotide”
  modified_base (21)..(21): Inverted abasic nucleotide
caaaaguggacccucuauaun

SEQ ID NO: 816 - RNA, 23 nt, Artificial Sequence
  source: /note=“Description of Artificial Sequence Synthetic oligonucleotide”
aauauagaggguccacuuuuguu

SEQ ID NO: 817 - RNA, 21 nt, Artificial Sequence
  source: /note=“Description of Artificial Sequence Synthetic oligonucleotide”
  modified_base (21)..(21): Inverted abasic nucleotide
aaaaguggacccucuauauun

SEQ ID NO: 818 - RNA, 23 nt, Artificial Sequence
  source: /note=“Description of Artificial Sequence Synthetic oligonucleotide”
aaauauagaggguccacuuuuuu

SEQ ID NO: 819 - RNA, 21 nt, Artificial Sequence
  source: /note=“Description of Artificial Sequence Synthetic oligonucleotide”
  modified_base (21)..(21): Inverted abasic nucleotide
aaaguggacccucuauauuun

SEQ ID NO: 820 - RNA, 23 nt, Artificial Sequence
  source: /note=“Description of Artificial Sequence Synthetic oligonucleotide”
aaaauauagaggguccacuuuuu

SEQ ID NO: 821 - RNA, 21 nt, Artificial Sequence
  source: /note=“Description of Artificial Sequence Synthetic oligonucleotide”
  modified_base (21)..(21): Inverted abasic nucleotide
aaguggacccucuauauuucn

SEQ ID NO: 822 - RNA, 23 nt, Artificial Sequence
  source: /note=“Description of Artificial Sequence Synthetic oligonucleotide”
agaaauauagaggguccacuuuu

SEQ ID NO: 823 - RNA, 21 nt, Artificial Sequence
  source: /note=“Description of Artificial Sequence Synthetic oligonucleotide”
  modified_base (21)..(21): Inverted abasic nucleotide
uucauauauccuuggucccan

SEQ ID NO: 824 - RNA, 23 nt, Artificial Sequence
  source: /note=“Description of Artificial Sequence Synthetic oligonucleotide”
augggaccaaggauauaugaauu

SEQ ID NO: 825 - RNA, 21 nt, Artificial Sequence
  source: /note=“Description of Artificial Sequence Synthetic oligonucleotide”
  modified_base (21)..(21): Inverted abasic nucleotide
gauguuuagacaauuuuaggn

SEQ ID NO: 826 - RNA, 23 nt, Artificial Sequence
  source: /note=“Description of Artificial Sequence Synthetic oligonucleotide”
accuaaaauugucuaaacaucuu

SEQ ID NO: 827 - RNA, 21 nt, Artificial Sequence
  source: /note=“Description of Artificial Sequence Synthetic oligonucleotide”
  modified_base (21)..(21): Inverted abasic nucleotide
auguuuagacaauuuuaggcn

SEQ ID NO: 828 - RNA, 23 nt, Artificial Sequence
  source: /note=“Description of Artificial Sequence Synthetic oligonucleotide”
agccuaaaauugucuaaacauuu

SEQ ID NO: 829 - RNA, 21 nt, Artificial Sequence
  source: /note=“Description of Artificial Sequence Synthetic oligonucleotide”
  modified_base (21)..(21): Inverted abasic nucleotide
uguuuagacaauuuuaggcun

SEQ ID NO: 830 - RNA, 23 nt, Artificial Sequence
  source: /note=“Description of Artificial Sequence Synthetic oligonucleotide”
aagccuaaaauugucuaaacauu

SEQ ID NO: 831 - RNA, 21 nt, Artificial Sequence
  source: /note=“Description of Artificial Sequence Synthetic oligonucleotide”
  modified_base (21)..(21): Inverted abasic nucleotide
guuuagacaauuuuaggcucn

SEQ ID NO: 832 - RNA, 23 nt, Artificial Sequence
  source: /note=“Description of Artificial Sequence Synthetic oligonucleotide”
ugagccuaaaauugucuaaacuu

SEQ ID NO: 833 - RNA, 21 nt, Artificial Sequence
  source: /note=“Description of Artificial Sequence Synthetic oligonucleotide”
  modified_base (21)..(21): Inverted abasic nucleotide
uuuagacaauuuuaggcucan

SEQ ID NO: 834 - RNA, 23 nt, Artificial Sequence
  source: /note=“Description of Artificial Sequence Synthetic oligonucleotide”
uugagccuaaaauugucuaaauu

SEQ ID NO: 835 - RNA, 21 nt, Artificial Sequence
  source: /note=“Description of Artificial Sequence Synthetic oligonucleotide”
  modified_base (21)..(21): Inverted abasic nucleotide
uuagacaauuuuaggcucaan

SEQ ID NO: 836 - RNA, 23 nt, Artificial Sequence
  source: /note=“Description of Artificial Sequence Synthetic oligonucleotide”
uuugagccuaaaauugucuaauu

SEQ ID NO: 837 - RNA, 21 nt, Artificial Sequence
  source: /note=“Description of Artificial Sequence Synthetic oligonucleotide”
  modified_base (21)..(21): Inverted abasic nucleotide
agacaauuuuaggcucaaaan

SEQ ID NO: 838 - RNA, 23 nt, Artificial Sequence
  source: /note=“Description of Artificial Sequence Synthetic oligonucleotide”
uuuuugagccuaaaauugucuuu

SEQ ID NO: 839 - RNA, 21 nt, Artificial Sequence
  source: /note=“Description of Artificial Sequence Synthetic oligonucleotide”
  modified_base (21)..(21): Inverted abasic nucleotide
auuuuaggcucaaaaauuaan

SEQ ID NO: 840 - RNA, 23 nt, Artificial Sequence
  source: /note=“Description of Artificial Sequence Synthetic oligonucleotide”
uuuaauuuuugagccuaaaauuu

SEQ ID NO: 841 - RNA, 21 nt, Artificial Sequence
  source: /note=“Description of Artificial Sequence Synthetic oligonucleotide”
  modified_base (21)..(21): Inverted abasic nucleotide
uuuaggcucaaaaauuaaagn

SEQ ID NO: 842 - RNA, 23 nt, Artificial Sequence
  source: /note=“Description of Artificial Sequence Synthetic oligonucleotide”
acuuuaauuuuugagccuaaauu

SEQ ID NO: 843 - RNA, 21 nt, Artificial Sequence
  source: /note=“Description of Artificial Sequence Synthetic oligonucleotide”
  modified_base (21)..(21): Inverted abasic nucleotide
uuaggcucaaaaauuaaagcn

SEQ ID NO: 844 - RNA, 23 nt, Artificial Sequence
  source: /note=“Description of Artificial Sequence Synthetic oligonucleotide”
agcuuuaauuuuugagccuaauu

SEQ ID NO: 845 - RNA, 21 nt, Artificial Sequence
  source: /note=“Description of Artificial Sequence Synthetic oligonucleotide”
  modified_base (21)..(21): Inverted abasic nucleotide
aaaaauuaaagcuaacacagn

SEQ ID NO: 846 - RNA, 23 nt, Artificial Sequence
  source: /note=“Description of Artificial Sequence Synthetic oligonucleotide”
acuguguuagcuuuaauuuuuuu

SEQ ID NO: 847 - RNA, 21 nt, Artificial Sequence
  source: /note=“Description of Artificial Sequence Synthetic oligonucleotide”
  modified_base (21)..(21): Inverted abasic nucleotide
aaaauuaaagcuaacacaggn

SEQ ID NO: 848 - RNA, 23 nt, Artificial Sequence
  source: /note=“Description of Artificial Sequence Synthetic oligonucleotide”
uccuguguuagcuuuaauuuuuu

SEQ ID NO: 849 - RNA, 21 nt, Artificial Sequence
  source: /note=“Description of Artificial Sequence Synthetic oligonucleotide”
  modified_base (21)..(21): Inverted abasic nucleotide
aaagcuaacacaggaaaaggn

SEQ ID NO: 850 - RNA, 23 nt, Artificial Sequence
  source: /note=“Description of Artificial Sequence Synthetic oligonucleotide”
uccuuuuccuguguuagcuuuuu

SEQ ID NO: 851 - RNA, 21 nt, Artificial Sequence
  source: /note=“Description of Artificial Sequence Synthetic oligonucleotide”
  modified_base (21)..(21): Inverted abasic nucleotide
aagcuaacacaggaaaaggan

SEQ ID NO: 852 - RNA, 23 nt, Artificial Sequence
  source: /note=“Description of Artificial Sequence Synthetic oligonucleotide”
uuccuuuuccuguguuagcuuuu

SEQ ID NO: 853 - RNA, 21 nt, Artificial Sequence
  source: /note=“Description of Artificial Sequence Synthetic oligonucleotide”
  modified_base (21)..(21): Inverted abasic nucleotide
ggaaaaggaacuguacuggcn

SEQ ID NO: 854 - RNA, 23 nt, Artificial Sequence
  source: /note=“Description of Artificial Sequence Synthetic oligonucleotide”
agccaguacaguuccuuuuccuu

SEQ ID NO: 855 - RNA, 21 nt, Artificial Sequence
  source: /note=“Description of Artificial Sequence Synthetic oligonucleotide”
  modified_base (21)..(21): Inverted abasic nucleotide
aggaacuguacuggcuauuan

SEQ ID NO: 856 - RNA, 23 nt, Artificial Sequence
  source: /note=“Description of Artificial Sequence Synthetic oligonucleotide”
auaauagccaguacaguuccuuu

SEQ ID NO: 857 - RNA, 21 nt, Artificial Sequence
  source: /note=“Description of Artificial Sequence Synthetic oligonucleotide”
  modified_base (21)..(21): Inverted abasic nucleotide
ccgacucccacuacaucaagn

SEQ ID NO: 858 - RNA, 23 nt, Artificial Sequence
  source: /note=“Description of Artificial Sequence Synthetic oligonucleotide”
ucuugauguagugggagucgguu

SEQ ID NO: 859 - RNA, 21 nt, Artificial Sequence
  source: /note=“Description of Artificial Sequence Synthetic oligonucleotide”
  modified_base (21)..(21): Inverted abasic nucleotide
gacucccacuacaucaagacn

SEQ ID NO: 860 - RNA, 23 nt, Artificial Sequence
  source: /note=“Description of Artificial Sequence Synthetic oligonucleotide”
agucuugauguagugggagucuu

SEQ ID NO: 861 - RNA, 21 nt, Artificial Sequence
  source: /note=“Description of Artificial Sequence Synthetic oligonucleotide”
  modified_base (21)..(21): Inverted abasic nucleotide
ucccacuacaucaagacuaan

SEQ ID NO: 862 - RNA, 23 nt, Artificial Sequence
  source: /note=“Description of Artificial Sequence Synthetic oligonucleotide”
auuagucuugauguagugggauu

SEQ ID NO: 863 - RNA, 21 nt, Artificial Sequence
  source: /note=“Description of Artificial Sequence Synthetic oligonucleotide”
  modified_base (21)..(21): Inverted abasic nucleotide
cccacuacaucaagacuaaun

SEQ ID NO: 864 - RNA, 23 nt, Artificial Sequence
  source: /note=“Description of Artificial Sequence Synthetic oligonucleotide”
aauuagucuugauguaguggguu

SEQ ID NO: 865 - RNA, 21 nt, Artificial Sequence
  source: /note=“Description of Artificial Sequence Synthetic oligonucleotide”
  modified_base (21)..(21): Inverted abasic nucleotide
cacuacaucaagacuaaucun

SEQ ID NO: 866 - RNA, 23 nt, Artificial Sequence
  source: /note=“Description of Artificial Sequence Synthetic oligonucleotide”
aagauuagucuugauguaguguu

SEQ ID NO: 867 - RNA, 21 nt, Artificial Sequence
  source: /note=“Description of Artificial Sequence Synthetic oligonucleotide”
  modified_base (21)..(21): Inverted abasic nucleotide
acuacaucaagacuaaucuun

SEQ ID NO: 868 - RNA, 23 nt, Artificial Sequence
  source: /note=“Description of Artificial Sequence Synthetic oligonucleotide”
aaagauuagucuugauguaguuu

SEQ ID NO: 869 - RNA, 21 nt, Artificial Sequence
  source: /note=“Description of Artificial Sequence Synthetic oligonucleotide”
  modified_base (21)..(21): Inverted abasic nucleotide
cuacaucaagacuaaucuugn

SEQ ID NO: 870 - RNA, 23 nt, Artificial Sequence
  source: /note=“Description of Artificial Sequence Synthetic oligonucleotide”
acaagauuagucuugauguaguu

SEQ ID NO: 871 - RNA, 21 nt, Artificial Sequence
  source: /note=“Description of Artificial Sequence Synthetic oligonucleotide”
  modified_base (21)..(21): Inverted abasic nucleotide
guuuuucacauguauuauagn

SEQ ID NO: 872 - RNA, 23 nt, Artificial Sequence
  source: /note=“Description of Artificial Sequence Synthetic oligonucleotide”
ucuauaauacaugugaaaaacuu

SEQ ID NO: 873 - RNA, 21 nt, Artificial Sequence
  source: /note=“Description of Artificial Sequence Synthetic oligonucleotide”
  modified_base (21)..(21): Inverted abasic nucleotide
ucacauguauuauagaaugcn

SEQ ID NO: 874 - RNA, 23 nt, Artificial Sequence
  source: /note=“Description of Artificial Sequence Synthetic oligonucleotide”
agcauucuauaauacaugugauu

SEQ ID NO: 875 - RNA, 21 nt, Artificial Sequence
  source: /note=“Description of Artificial Sequence Synthetic oligonucleotide”
  modified_base (21)..(21): Inverted abasic nucleotide
acauguauuauagaaugcuun

SEQ ID NO: 876 - RNA, 23 nt, Artificial Sequence
  source: /note=“Description of Artificial Sequence Synthetic oligonucleotide”
aaagcauucuauaauacauguuu

SEQ ID NO: 877 - RNA, 21 nt, Artificial Sequence
  source: /note=“Description of Artificial Sequence Synthetic oligonucleotide”
  modified_base (21)..(21): Inverted abasic nucleotide
uguauuauagaaugcuuuugn

SEQ ID NO: 878 - RNA, 23 nt, Artificial Sequence
  source: /note=“Description of Artificial Sequence Synthetic oligonucleotide”
acaaaagcauucuauaauacauu

SEQ ID NO: 879 - RNA, 21 nt, Artificial Sequence
  source: /note=“Description of Artificial Sequence Synthetic oligonucleotide”
  modified_base (21)..(21): Inverted abasic nucleotide
gaaugcuuuugcauggacuan

SEQ ID NO: 880 - RNA, 23 nt, Artificial Sequence
  source: /note=“Description of Artificial Sequence Synthetic oligonucleotide”
auaguccaugcaaaagcauucuu

SEQ ID NO: 881 - RNA, 21 nt, Artificial Sequence
  source: /note=“Description of Artificial Sequence Synthetic oligonucleotide”
  modified_base (21)..(21): Inverted abasic nucleotide
aaugcuuuugcauggacuaun

SEQ ID NO: 882 - RNA, 23 nt, Artificial Sequence
  source: /note=“Description of Artificial Sequence Synthetic oligonucleotide”
aauaguccaugcaaaagcauuuu

SEQ ID NO: 883 - RNA, 21 nt, Artificial Sequence
  source: /note=“Description of Artificial Sequence Synthetic oligonucleotide”
  modified_base (21)..(21): Inverted abasic nucleotide
gcuuuugcauggacuauccun

SEQ ID NO: 884 - RNA, 23 nt, Artificial Sequence
  source: /note=“Description of Artificial Sequence Synthetic oligonucleotide”
aaggauaguccaugcaaaagcuu

SEQ ID NO: 885 - RNA, 21 nt, Artificial Sequence
  source: /note=“Description of Artificial Sequence Synthetic oligonucleotide”
  modified_base (21)..(21): Inverted abasic nucleotide
uuugcauggacuauccucuun

SEQ ID NO: 886 - RNA, 23 nt, Artificial Sequence
  source: /note=“Description of Artificial Sequence Synthetic oligonucleotide”
aaagaggauaguccaugcaaauu

SEQ ID NO: 887 - RNA, 21 nt, Artificial Sequence
  source: /note=“Description of Artificial Sequence Synthetic oligonucleotide”
  modified_base (21)..(21): Inverted abasic nucleotide
uugcauggacuauccucuugn

SEQ ID NO: 888 - RNA, 23 nt, Artificial Sequence
  source: /note=“Description of Artificial Sequence Synthetic oligonucleotide”
acaagaggauaguccaugcaauu

SEQ ID NO: 889 - RNA, 21 nt, Artificial Sequence
  source: /note=“Description of Artificial Sequence Synthetic oligonucleotide”
  modified_base (21)..(21): Inverted abasic nucleotide
ugcauggacuauccucuugun

SEQ ID NO: 890 - RNA, 23 nt, Artificial Sequence
  source: /note=“Description of Artificial Sequence Synthetic oligonucleotide”
aacaagaggauaguccaugcauu

SEQ ID NO: 891 - RNA, 21 nt, Artificial Sequence
  source: /note=“Description of Artificial Sequence Synthetic oligonucleotide”
  modified_base (21)..(21): Inverted abasic nucleotide
gcauggacuauccucuuguun

SEQ ID NO: 892 - RNA, 23 nt, Artificial Sequence
  source: /note=“Description of Artificial Sequence Synthetic oligonucleotide”
aaacaagaggauaguccaugcuu

SEQ ID NO: 893 - RNA, 21 nt, Artificial Sequence
  source: /note=“Description of Artificial Sequence Synthetic oligonucleotide”
  modified_base (21)..(21): Inverted abasic nucleotide
auggacuauccucuuguuuun

SEQ ID NO: 894 - RNA, 23 nt, Artificial Sequence
  source: /note=“Description of Artificial Sequence Synthetic oligonucleotide”
aaaaacaagaggauaguccauuu

SEQ ID NO: 895 - RNA, 21 nt, Artificial Sequence
  source: /note=“Description of Artificial Sequence Synthetic oligonucleotide”
  modified_base (21)..(21): Inverted abasic nucleotide
ggacuauccucuuguuuuuan

SEQ ID NO: 896 - RNA, 23 nt, Artificial Sequence
  source: /note=“Description of Artificial Sequence Synthetic oligonucleotide”
auaaaaacaagaggauaguccuu

SEQ ID NO: 897 - RNA, 21 nt, Artificial Sequence
  source: /note=“Description of Artificial Sequence Synthetic oligonucleotide”
  modified_base (21)..(21): Inverted abasic nucleotide
aauaaccucuuguaguuauan

SEQ ID NO: 898 - RNA, 23 nt, Artificial Sequence
  source: /note=“Description of Artificial Sequence Synthetic oligonucleotide”
uuauaacuacaagagguuauuuu

SEQ ID NO: 899 - RNA, 21 nt, Artificial Sequence
  source: /note=“Description of Artificial Sequence Synthetic oligonucleotide”
  modified_base (21)..(21): Inverted abasic nucleotide
auaaccucuuguaguuauaan

SEQ ID NO: 900 - RNA, 23 nt, Artificial Sequence
  source: /note=“Description of Artificial Sequence Synthetic oligonucleotide”
uuuauaacuacaagagguuauuu

SEQ ID NO: 901 - RNA, 21 nt, Artificial Sequence
  source: /note=“Description of Artificial Sequence Synthetic oligonucleotide”
  modified_base (21)..(21): Inverted abasic nucleotide
accucuuguaguuauaaaaun

SEQ ID NO: 902 - RNA, 23 nt, Artificial Sequence
  source: /note=“Description of Artificial Sequence Synthetic oligonucleotide”
uauuuuauaacuacaagagguuu

SEQ ID NO: 903 - RNA, 21 nt, Artificial Sequence
  source: /note=“Description of Artificial Sequence Synthetic oligonucleotide”
  modified_base (21)..(21): Inverted abasic nucleotide
ggucaacauccuaggacauun

SEQ ID NO: 904 - RNA, 23 nt, Artificial Sequence
  source: /note=“Description of Artificial Sequence Synthetic oligonucleotide”
aaauguccuaggauguugaccuu

SEQ ID NO: 905 - RNA, 21 nt, Artificial Sequence
  source: /note=“Description of Artificial Sequence Synthetic oligonucleotide”
  modified_base (21)..(21): Inverted abasic nucleotide
gucaacauccuaggacauuun

SEQ ID NO: 906 - RNA, 23 nt, Artificial Sequence
  source: /note=“Description of Artificial Sequence Synthetic oligonucleotide”
aaaauguccuaggauguugacuu

SEQ ID NO: 907 - RNA, 21 nt, Artificial Sequence
  source: /note=“Description of Artificial Sequence Synthetic oligonucleotide”
  modified_base (21)..(21): Inverted abasic nucleotide
ucaacauccuaggacauuuun

SEQ ID NO: 908 - RNA, 21 nt, Artificial Sequence
  source: /note=“Description of Artificial Sequence Synthetic oligonucleotide”
aaauguccuaggauguugauu

SEQ ID NO: 909 - RNA, 21 nt, Artificial Sequence
  source: /note=“Description of Artificial Sequence Synthetic oligonucleotide”
  modified_base (21)..(21): Inverted abasic nucleotide
caacauccuaggacauuuuun

SEQ ID NO: 910 - RNA, 21 nt, Artificial Sequence
  source: /note=“Description of Artificial Sequence Synthetic oligonucleotide”
aaaauguccuaggauguuguu

SEQ ID NO: 911 - RNA, 21 nt, Artificial Sequence
  source: /note=“Description of Artificial Sequence Synthetic oligonucleotide”
  modified_base (21)..(21): Inverted abasic nucleotide
ggucaacauccuaggacauun

SEQ ID NO: 912 - RNA, 23 nt, Artificial Sequence
  source: /note=“Description of Artificial Sequence Synthetic oligonucleotide”
aaauguccuaggauguugaccuu

SEQ ID NO: 913 - RNA, 21 nt, Artificial Sequence
  source: /note=“Description of Artificial Sequence Synthetic oligonucleotide”
  modified_base (21)..(21): Inverted abasic nucleotide
gucaacauccuaggacauuun

SEQ ID NO: 914 - RNA, 23 nt, Artificial Sequence
  source: /note=“Description of Artificial Sequence Synthetic oligonucleotide”
aaaauguccuaggauguugacuu

SEQ ID NO: 915 - RNA, 21 nt, Artificial Sequence
  source: /note=“Description of Artificial Sequence Synthetic oligonucleotide”
  modified_base (21)..(21): Inverted abasic nucleotide
ggucaacauccuaggacauun

SEQ ID NO: 916 - RNA, 21 nt, Artificial Sequence
  source: /note=“Description of Artificial Sequence Synthetic oligonucleotide”
aaauguccuaggauguugacc

SEQ ID NO: 917 - RNA, 21 nt, Artificial Sequence
  source: /note=“Description of Artificial Sequence Synthetic oligonucleotide”
  modified_base (21)..(21): Inverted abasic nucleotide
gucaacauccuaggacauuun

SEQ ID NO: 918 - RNA, 21 nt, Artificial Sequence
  source: /note=“Description of Artificial Sequence Synthetic oligonucleotide”
aaaauguccuaggauguugac

SEQ ID NO: 919 - RNA, 21 nt, Artificial Sequence
  source: /note=“Description of Artificial Sequence Synthetic oligonucleotide”
  modified_base (21)..(21): Inverted abasic nucleotide
ggucaacauccuaggacauun

SEQ ID NO: 920 - RNA, 21 nt, Artificial Sequence
  source: /note=“Description of Artificial Sequence Synthetic oligonucleotide”
aaauguccuaggauguugacc

SEQ ID NO: 921 - RNA, 21 nt, Artificial Sequence
  source: /note=“Description of Artificial Sequence Synthetic oligonucleotide”
  modified_base (21)..(21): Inverted abasic nucleotide
gucaacauccuaggacauuun

SEQ ID NO: 922 - RNA, 21 nt, Artificial Sequence
  source: /note=“Description of Artificial Sequence Synthetic oligonucleotide”
aaaauguccuaggauguugac

SEQ ID NO: 923 - RNA, 21 nt, Artificial Sequence
  source: /note=“Description of Artificial Sequence Synthetic oligonucleotide”
  modified_base (21)..(21): Inverted abasic nucleotide
gucaacauccuaggacauuun

SEQ ID NO: 924 - RNA, 21 nt, Artificial Sequence
  source: /note=“Description of Artificial Sequence Synthetic oligonucleotide”
aaaauguccuaggauguugac

SEQ ID NO: 925 - RNA, 21 nt, Artificial Sequence
  source: /note=“Description of Artificial Sequence Synthetic oligonucleotide”
  modified_base (21)..(21): Inverted abasic nucleotide
ggucaacauccuaggacauun

SEQ ID NO: 926 - RNA, 21 nt, Artificial Sequence
  source: /note=“Description of Artificial Sequence Synthetic oligonucleotide”
aaauguccuaggauguugacc

SEQ ID NO: 927 - RNA, 20 nt, Artificial Sequence
  source: /note=“Description of Artificial Sequence Synthetic oligonucleotide”
  modified_base (1)..(1): Inverted abasic nucleotide
nucaacauccuaggacauuu

SEQ ID NO: 928 - RNA, 21 nt, Artificial Sequence
  source: /note=“Description of Artificial Sequence Synthetic oligonucleotide”
aaauguccuaggauguugauu

SEQ ID NO: 929 - RNA, 20 nt, Artificial Sequence
  source: /note=“Description of Artificial Sequence Synthetic oligonucleotide”
  modified_base (1)..(1): Inverted abasic nucleotide
ncaacauccuaggacauuuu

SEQ ID NO: 930 - RNA, 21 nt, Artificial Sequence
  source: /note=“Description of Artificial Sequence Synthetic oligonucleotide”
aaaauguccuaggauguuguu

SEQ ID NO: 931 - RNA, 19 nt, Artificial Sequence
  source: /note=“Description of Artificial Sequence Synthetic oligonucleotide”
  modified_base (19)..(19): Inverted abasic nucleotide
ucaacauccuaggacauun

SEQ ID NO: 932 - RNA, 21 nt, Artificial Sequence
  source: /note=“Description of Artificial Sequence Synthetic oligonucleotide”
aaauguccuaggauguugauu

SEQ ID NO: 933 - RNA, 19 nt, Artificial Sequence
  source: /note=“Description of Artificial Sequence Synthetic oligonucleotide”
  modified_base (19)..(19): Inverted abasic nucleotide
caacauccuaggacauuun

SEQ ID NO: 934 - RNA, 21 nt, Artificial Sequence
  source: /note=“Description of Artificial Sequence Synthetic oligonucleotide”
aaaauguccuaggauguuguu

SEQ ID NO: 935 - RNA, 21 nt, Artificial Sequence
  source: /note=“Description of Artificial Sequence Synthetic oligonucleotide”
  modified_base (21)..(21): Inverted abasic nucleotide
ggucaacaucgauggucauun

SEQ ID NO: 936 - RNA, 23 nt, Artificial Sequence
  source: /note=“Description of Artificial Sequence Synthetic oligonucleotide”
aaaugaccaucgauguugaccuu

SEQ ID NO: 937 - RNA, 21 nt, Artificial Sequence
  source: /note=“Description of Artificial Sequence Synthetic oligonucleotide”
  modified_base (21)..(21): Inverted abasic nucleotide
gucaacauccaucgagauuun

SEQ ID NO: 938 - RNA, 23 nt, Artificial Sequence
  source: /note=“Description of Artificial Sequence Synthetic oligonucleotide”
aaaaucucgauggauguugacuu

SEQ ID NO: 939 - RNA, 21 nt, Artificial Sequence
  source: /note=“Description of Artificial Sequence Synthetic oligonucleotide”
  modified_base (21)..(21): Inverted abasic nucleotide
gucaacauccuaggacauuun

SEQ ID NO: 940 - RNA, 21 nt, Artificial Sequence
  source: /note=“Description of Artificial Sequence Synthetic oligonucleotide”
aauguccuaggauguugacuu

SEQ ID NO: 941 - RNA, 21 nt, Artificial Sequence
  source: /note=“Description of Artificial Sequence Synthetic oligonucleotide”
  modified_base (21)..(21): Inverted abasic nucleotide
cucccacuacaucaagacuun

SEQ ID NO: 942 - RNA, 21 nt, Artificial Sequence
  source: /note=“Description of Artificial Sequence Synthetic oligonucleotide”
agucuugauguagugggaguu

SEQ ID NO: 943 - RNA, 21 nt, Artificial Sequence
  source: /note=“Description of Artificial Sequence Synthetic oligonucleotide”
  modified_base (21)..(21): Inverted abasic nucleotide
ccacuacaucaagacuaauun

SEQ ID NO: 944 - RNA, 21 nt, Artificial Sequence
  source: /note=“Description of Artificial Sequence Synthetic oligonucleotide”
auuagucuugauguagugguu

SEQ ID NO: 945 - RNA, 19 nt, Artificial Sequence
  source: /note=“Description of Artificial Sequence Synthetic oligonucleotide”
  modified_base (19)..(19): Inverted abasic nucleotide
uuccuuaucucaucccuun

SEQ ID NO: 946 - RNA, 21 nt, Artificial Sequence
  source: /note=“Description of Artificial Sequence Synthetic oligonucleotide”
uaagggaugagauaaggaauu

SEQ ID NO: 947 - RNA, 19 nt, Artificial Sequence
  source: /note=“Description of Artificial Sequence Synthetic oligonucleotide”
  modified_base (19)..(19): Inverted abasic nucleotide
uuccuuaugagaucccuun

SEQ ID NO: 948 - RNA, 21 nt, Artificial Sequence
  source: /note=“Description of Artificial Sequence Synthetic oligonucleotide”
uaagggaucucauaaggaauu

SEQ ID NO: 949 - RNA, 19 nt, Artificial Sequence
  source: /note=“Description of Artificial Sequence Synthetic oligonucleotide”
  modified_base (19)..(19): Inverted abasic nucleotide
ucaacaucgauggucauun

SEQ ID NO: 950 - RNA, 21 nt, Artificial Sequence
  source: /note=“Description of Artificial Sequence Synthetic oligonucleotide”
aaaugaccaucgauguugauu

SEQ ID NO: 951 - RNA, 21 nt, Artificial Sequence
  source: /note=“Description of Artificial Sequence Synthetic oligonucleotide”
uacaucaagacuaaucuuguu

SEQ ID NO: 952 - RNA, 23 nt, Artificial Sequence
  source: /note=“Description of Artificial Sequence Synthetic oligonucleotide”
aacaagauuagucuugauguagu

SEQ ID NO: 953 - RNA, 21 nt, Artificial Sequence
  source: /note=“Description of Artificial Sequence Synthetic oligonucleotide”
  modified_base (21)..(21): Inverted abasic nucleotide
augcuuuugcauggacuaucn

SEQ ID NO: 954 - DNA, 23 bp, Artificial Sequence
  source: /note=“Description of Artificial Sequence Synthetic oligonucleotide”
  source: /note=“Description of Combined DNA/RNA Molecule Synthetic oligonucleotide”
agauagtccaugcaaaagcauuc

SEQ ID NO: 955 - RNA, 21 nt, Artificial Sequence
  source: /note=“Description of Artificial Sequence Synthetic oligonucleotide”
cguaugcagaauauucaauuu

SEQ ID NO: 956 - RNA, 23 nt, Artificial Sequence
  source: /note=“Description of Artificial Sequence Synthetic oligonucleotide”
aaauugaauauucugcauacgau

SEQ ID NO: 957 - RNA, 21 nt, Artificial Sequence
  source: /note=“Description of Artificial Sequence Synthetic oligonucleotide”
cguaugcagaauauucaauuu

SEQ ID NO: 958 - RNA, 23 nt, Artificial Sequence
  source: /note=“Description of Artificial Sequence Synthetic oligonucleotide”
aaauugaauauucugcauacgau

SEQ ID NO: 959 - RNA, 21 nt, Artificial Sequence
  source: /note=“Description of Artificial Sequence Synthetic oligonucleotide”
cuacaucaagacuaaucuugu

SEQ ID NO: 960 - DNA, 23 bp, Artificial Sequence
  source: /note=“Description of Artificial Sequence Synthetic oligonucleotide”
  source: /note=“Description of Combined DNA/RNA Molecule Synthetic oligonucleotide”
acaagatuagucuugauguagug

SEQ ID NO: 961 - RNA, 21 nt, Artificial Sequence
  source: /note=“Description of Artificial Sequence Synthetic oligonucleotide”
  modified_base (21)..(21): Inverted abasic nucleotide
aagccaugaacaucauccuan

SEQ ID NO: 962 - RNA, 23 nt, Artificial Sequence
  source: /note=“Description of Artificial Sequence Synthetic oligonucleotide”
auaggaugauguucauggcuuuu

SEQ ID NO: 963 - RNA, 21 nt, Artificial Sequence
  source: /note=“Description of Artificial Sequence Synthetic oligonucleotide”
  modified_base (21)..(21): Inverted abasic nucleotide
agccaugaacaucauccuagn

SEQ ID NO: 964 - RNA, 23 nt, Artificial Sequence
  source: /note=“Description of Artificial Sequence Synthetic oligonucleotide”
ucuaggaugauguucauggcuuu

SEQ ID NO: 965 - RNA, 21 nt, Artificial Sequence
  source: /note=“Description of Artificial Sequence Synthetic oligonucleotide”
  modified_base (21)..(21): Inverted abasic nucleotide
ccaugaacaucauccuagaan

SEQ ID NO: 966 - RNA, 23 nt, Artificial Sequence
  source: /note=“Description of Artificial Sequence Synthetic oligonucleotide”
uuucuaggaugauguucaugguu

SEQ ID NO: 967 - RNA, 21 nt, Artificial Sequence
  source: /note=“Description of Artificial Sequence Synthetic oligonucleotide”
  modified_base (21)..(21): Inverted abasic nucleotide
ugaacaucauccuagaaaucn

SEQ ID NO: 968 - RNA, 23 nt, Artificial Sequence
  source: /note=“Description of Artificial Sequence Synthetic oligonucleotide”
agauuucuaggaugauguucauu

SEQ ID NO: 969 - RNA, 21 nt, Artificial Sequence
  source: /note=“Description of Artificial Sequence Synthetic oligonucleotide”
  modified_base (21)..(21): Inverted abasic nucleotide
gaaguuuuucauuccucagan

SEQ ID NO: 970 - RNA, 23 nt, Artificial Sequence
  source: /note=“Description of Artificial Sequence Synthetic oligonucleotide”
aucugaggaaugaaaaacuucuu

SEQ ID NO: 971 - RNA, 21 nt, Artificial Sequence
  source: /note=“Description of Artificial Sequence Synthetic oligonucleotide”
  modified_base (21)..(21): Inverted abasic nucleotide
ggagaaaaucuguggcugggn

SEQ ID NO: 972 - RNA, 23 nt, Artificial Sequence
  source: /note=“Description of Artificial Sequence Synthetic oligonucleotide”
acccagccacagauuuucuccuu

SEQ ID NO: 973 - RNA, 21 nt, Artificial Sequence
  source: /note=“Description of Artificial Sequence Synthetic oligonucleotide”
  modified_base (21)..(21): Inverted abasic nucleotide
guggcuggggagauuguucun

SEQ ID NO: 974 - RNA, 23 nt, Artificial Sequence
  source: /note=“Description of Artificial Sequence Synthetic oligonucleotide”
aagaacaaucuccccagccacuu

SEQ ID NO: 975 - RNA, 21 nt, Artificial Sequence
  source: /note=“Description of Artificial Sequence Synthetic oligonucleotide”
  modified_base (21)..(21): Inverted abasic nucleotide
ggaauaggcaggcagacuacn

SEQ ID NO: 976 - RNA, 23 nt, Artificial Sequence
  source: /note=“Description of Artificial Sequence Synthetic oligonucleotide”
aguagucugccugccuauuccuu

SEQ ID NO: 977 - RNA, 21 nt, Artificial Sequence
  source: /note=“Description of Artificial Sequence Synthetic oligonucleotide”
  modified_base (21)..(21): Inverted abasic nucleotide
gaauaggcaggcagacuacun

SEQ ID NO: 978 - RNA, 23 nt, Artificial Sequence
  source: /note=“Description of Artificial Sequence Synthetic oligonucleotide”
aaguagucugccugccuauucuu

SEQ ID NO: 979 - RNA, 21 nt, Artificial Sequence
  source: /note=“Description of Artificial Sequence Synthetic oligonucleotide”
  modified_base (21)..(21): Inverted abasic nucleotide
uuugcaaaacgacagagcaun

SEQ ID NO: 980 - RNA, 23 nt, Artificial Sequence
  source: /note=“Description of Artificial Sequence Synthetic oligonucleotide”
uaugcucugucguuuugcaaauu

SEQ ID NO: 981 - RNA, 21 nt, Artificial Sequence
  source: /note=“Description of Artificial Sequence Synthetic oligonucleotide”
  modified_base (21)..(21): Inverted abasic nucleotide
gcaaaacgacagagcauauun

SEQ ID NO: 982 - RNA, 23 nt, Artificial Sequence
  source: /note=“Description of Artificial Sequence Synthetic oligonucleotide”
aaauaugcucugucguuuugcuu

SEQ ID NO: 983 - RNA, 21 nt, Artificial Sequence
  source: /note=“Description of Artificial Sequence Synthetic oligonucleotide”
  modified_base (21)..(21): Inverted abasic nucleotide
acgacagagcauauugguucn

SEQ ID NO: 984 - RNA, 23 nt, Artificial Sequence
  source: /note=“Description of Artificial Sequence Synthetic oligonucleotide”
agaaccaauaugcucugucguuu

SEQ ID NO: 985 - RNA, 21 nt, Artificial Sequence
  source: /note=“Description of Artificial Sequence Synthetic oligonucleotide”
  modified_base (21)..(21): Inverted abasic nucleotide
cgacagagcauauugguucun

SEQ ID NO: 986 - RNA, 23 nt, Artificial Sequence
  source: /note=“Description of Artificial Sequence Synthetic oligonucleotide”
aagaaccaauaugcucugucguu

SEQ ID NO: 987 - RNA, 21 nt, Artificial Sequence
  source: /note=“Description of Artificial Sequence Synthetic oligonucleotide”
  modified_base (21)..(21): Inverted abasic nucleotide
gacagagcauauugguucugn

SEQ ID NO: 988 - RNA, 23 nt, Artificial Sequence
  source: /note=“Description of Artificial Sequence Synthetic oligonucleotide”
acagaaccaauaugcucugucuu

SEQ ID NO: 989 - RNA, 21 nt, Artificial Sequence
  source: /note=“Description of Artificial Sequence Synthetic oligonucleotide”
  modified_base (21)..(21): Inverted abasic nucleotide
acagagcauauugguucugun

SEQ ID NO: 990 - RNA, 23 nt, Artificial Sequence
  source: /note=“Description of Artificial Sequence Synthetic oligonucleotide”
aacagaaccaauaugcucuguuu

SEQ ID NO: 991 - RNA, 21 nt, Artificial Sequence
  source: /note=“Description of Artificial Sequence Synthetic oligonucleotide”
  modified_base (21)..(21): Inverted abasic nucleotide
cagagcauauugguucugugn

SEQ ID NO: 992 - RNA, 23 nt, Artificial Sequence
  source: /note=“Description of Artificial Sequence Synthetic oligonucleotide”
acacagaaccaauaugcucuguu

SEQ ID NO: 993 - RNA, 21 nt, Artificial Sequence
  source: /note=“Description of Artificial Sequence Synthetic oligonucleotide”
  modified_base (21)..(21): Inverted abasic nucleotide
agagcauauugguucuguggn

SEQ ID NO: 994 - RNA, 23 nt, Artificial Sequence
  source: /note=“Description of Artificial Sequence Synthetic oligonucleotide”
accacagaaccaauaugcucuuu

SEQ ID NO: 995 - RNA, 21 nt, Artificial Sequence
  source: /note=“Description of Artificial Sequence Synthetic oligonucleotide”
  modified_base (21)..(21): Inverted abasic nucleotide
gagcauauugguucugugggn

SEQ ID NO: 996 - RNA, 23 nt, Artificial Sequence
  source: /note=“Description of Artificial Sequence Synthetic oligonucleotide”
ucccacagaaccaauaugcucuu

SEQ ID NO: 997 - RNA, 21 nt, Artificial Sequence
  source: /note=“Description of Artificial Sequence Synthetic oligonucleotide”
  modified_base (21)..(21): Inverted abasic nucleotide
cugugggauauuaauaagcgn

SEQ ID NO: 998 - RNA, 23 nt, Artificial Sequence
  source: /note=“Description of Artificial Sequence Synthetic oligonucleotide”
acgcuuauuaauaucccacaguu

SEQ ID NO: 999 - RNA, 21 nt, Artificial Sequence
  source: /note=“Description of Artificial Sequence Synthetic oligonucleotide”
  modified_base (21)..(21): Inverted abasic nucleotide
uuaauaagcgcgguguggagn

SEQ ID NO: 1000 - RNA, 23 nt, Artificial Sequence
  source: /note=“Description of Artificial Sequence Synthetic oligonucleotide”
acuccacaccgcgcuuauuaauu

SEQ ID NO: 1001 - RNA, 21 nt, Artificial Sequence
  source: /note=“Description of Artificial Sequence Synthetic oligonucleotide”
  modified_base (21)..(21): Inverted abasic nucleotide
uaauaagcgcgguguggaggn

SEQ ID NO: 1002 - RNA, 23 nt, Artificial Sequence
  source: /note=“Description of Artificial Sequence Synthetic oligonucleotide”
uccuccacaccgcgcuuauuauu

SEQ ID NO: 1003 - RNA, 21 nt, Artificial Sequence
  source: /note=“Description of Artificial Sequence Synthetic oligonucleotide”
  modified_base (21)..(21): Inverted abasic nucleotide
auaagcgcgguguggaggaan

SEQ ID NO: 1004 - RNA, 23 nt, Artificial Sequence
  source: /note=“Description of Artificial Sequence Synthetic oligonucleotide”
uuuccuccacaccgcgcuuauuu

SEQ ID NO: 1005 - RNA, 21 nt, Artificial Sequence
  source: /note=“Description of Artificial Sequence Synthetic oligonucleotide”
  modified_base (21)..(21): Inverted abasic nucleotide
uaagcgcgguguggaggaaan

SEQ ID NO: 1006 - RNA, 23 nt, Artificial Sequence
  source: /note=“Description of Artificial Sequence Synthetic oligonucleotide”
auuuccuccacaccgcgcuuauu

SEQ ID NO: 1007 - RNA, 21 nt, Artificial Sequence
  source: /note=“Description of Artificial Sequence Synthetic oligonucleotide”
  modified_base (21)..(21): Inverted abasic nucleotide
ggcgucacugcgcaugcguan

SEQ ID NO: 1008 - RNA, 23 nt, Artificial Sequence
  source: /note=“Description of Artificial Sequence Synthetic oligonucleotide”
auacgcaugcgcagugacgccuu

SEQ ID NO: 1009 - RNA, 21 nt, Artificial Sequence
  source: /note=“Description of Artificial Sequence Synthetic oligonucleotide”
  modified_base (21)..(21): Inverted abasic nucleotide
gcgucacugcgcaugcguaun

SEQ ID NO: 1010 - RNA, 23 nt, Artificial Sequence
  source: /note=“Description of Artificial Sequence Synthetic oligonucleotide”
aauacgcaugcgcagugacgcuu

SEQ ID NO: 1011 - RNA, 21 nt, Artificial Sequence
  source: /note=“Description of Artificial Sequence Synthetic oligonucleotide”
  modified_base (21)..(21): Inverted abasic nucleotide
cgucacugcgcaugcguaugn

SEQ ID NO: 1012 - RNA, 23 nt, Artificial Sequence
  source: /note=“Description of Artificial Sequence Synthetic oligonucleotide”
acauacgcaugcgcagugacguu

SEQ ID NO: 1013 - RNA, 21 nt, Artificial Sequence
  source: /note=“Description of Artificial Sequence Synthetic oligonucleotide”
  modified_base (21)..(21): Inverted abasic nucleotide
agcaacagagaagagaucuan

SEQ ID NO: 1014 - RNA, 23 nt, Artificial Sequence
  source: /note=“Description of Artificial Sequence Synthetic oligonucleotide”
auagaucucuucucuguugcuuu

SEQ ID NO: 1015 - RNA, 21 nt, Artificial Sequence
  source: /note=“Description of Artificial Sequence Synthetic oligonucleotide”
  modified_base (21)..(21): Inverted abasic nucleotide
caacagagaagagaucuaucn

SEQ ID NO: 1016 - RNA, 23 nt, Artificial Sequence
  source: /note=“Description of Artificial Sequence Synthetic oligonucleotide”
agauagaucucuucucuguuguu

SEQ ID NO: 1017 - RNA, 21 nt, Artificial Sequence
  source: /note=“Description of Artificial Sequence Synthetic oligonucleotide”
  modified_base (21)..(21): Inverted abasic nucleotide
aacagagaagagaucuaucgn

SEQ ID NO: 1018 - RNA, 23 nt, Artificial Sequence
  source: /note=“Description of Artificial Sequence Synthetic oligonucleotide”
acgauagaucucuucucuguuuu

SEQ ID NO: 1019 - RNA, 21 nt, Artificial Sequence
  source: /note=“Description of Artificial Sequence Synthetic oligonucleotide”
  modified_base (21)..(21): Inverted abasic nucleotide
cagagaagagaucuaucgcun

SEQ ID NO: 1020 - RNA, 23 nt, Artificial Sequence
  source: /note=“Description of Artificial Sequence Synthetic oligonucleotide”
aagcgauagaucucuucucuguu

SEQ ID NO: 1021 - RNA, 21 nt, Artificial Sequence
  source: /note=“Description of Artificial Sequence Synthetic oligonucleotide”
  modified_base (21)..(21): Inverted abasic nucleotide
agagaagagaucuaucgcucn

SEQ ID NO: 1022 - RNA, 23 nt, Artificial Sequence
  source: /note=“Description of Artificial Sequence Synthetic oligonucleotide”
agagcgauagaucucuucucuuu

SEQ ID NO: 1023 - RNA, 21 nt, Artificial Sequence
  source: /note=“Description of Artificial Sequence Synthetic oligonucleotide”
  modified_base (21)..(21): Inverted abasic nucleotide
gagaagagaucuaucgcucun

SEQ ID NO: 1024 - RNA, 23 nt, Artificial Sequence
  source: /note=“Description of Artificial Sequence Synthetic oligonucleotide”
aagagcgauagaucucuucucuu

SEQ ID NO: 1025 - RNA, 21 nt, Artificial Sequence
  source: /note=“Description of Artificial Sequence Synthetic oligonucleotide”
  modified_base (21)..(21): Inverted abasic nucleotide
aagagaucuaucgcucucuan

SEQ ID NO: 1026 - RNA, 23 nt, Artificial Sequence
  source: /note=“Description of Artificial Sequence Synthetic oligonucleotide”
uuagagagcgauagaucucuuuu

SEQ ID NO: 1027 - RNA, 21 nt, Artificial Sequence
  source: /note=“Description of Artificial Sequence Synthetic oligonucleotide”
  modified_base (21)..(21): Inverted abasic nucleotide
gagaucuaucgcucucuaaan

SEQ ID NO: 1028 - RNA, 23 nt, Artificial Sequence
  source: /note=“Description of Artificial Sequence Synthetic oligonucleotide”
auuuagagagcgauagaucucuu

SEQ ID NO: 1029 - RNA, 21 nt, Artificial Sequence
  source: /note=“Description of Artificial Sequence Synthetic oligonucleotide”
  modified_base (21)..(21): Inverted abasic nucleotide
gaucuaucgcucucuaaaucn

SEQ ID NO: 1030 - RNA, 23 nt, Artificial Sequence
  source: /note=“Description of Artificial Sequence Synthetic oligonucleotide”
ugauuuagagagcgauagaucuu

SEQ ID NO: 1031 - RNA, 21 nt, Artificial Sequence
  source: /note=“Description of Artificial Sequence Synthetic oligonucleotide”
  modified_base (21)..(21): Inverted abasic nucleotide
aucuaucgcucucuaaaucan

SEQ ID NO: 1032 - RNA, 23 nt, Artificial Sequence
  source: /note=“Description of Artificial Sequence Synthetic oligonucleotide”
augauuuagagagcgauagauuu

SEQ ID NO: 1033 - RNA, 21 nt, Artificial Sequence
  source: /note=“Description of Artificial Sequence Synthetic oligonucleotide”
  modified_base (21)..(21): Inverted abasic nucleotide
cgcucucuaaaucaggugaan

SEQ ID NO: 1034 - RNA, 23 nt, Artificial Sequence
  source: /note=“Description of Artificial Sequence Synthetic oligonucleotide”
auucaccugauuuagagagcguu

SEQ ID NO: 1035 - RNA, 21 nt, Artificial Sequence
  source: /note=“Description of Artificial Sequence Synthetic oligonucleotide”
  modified_base (21)..(21): Inverted abasic nucleotide
cucucuaaaucaggugaagan

SEQ ID NO: 1036 - RNA, 23 nt, Artificial Sequence
  source: /note=“Description of Artificial Sequence Synthetic oligonucleotide”
uucuucaccugauuuagagaguu

SEQ ID NO: 1037 - RNA, 21 nt, Artificial Sequence
  source: /note=“Description of Artificial Sequence Synthetic oligonucleotide”
  modified_base (21)..(21): Inverted abasic nucleotide
aaagaagugggugauguaacn

SEQ ID NO: 1038 - RNA, 23 nt, Artificial Sequence
  source: /note=“Description of Artificial Sequence Synthetic oligonucleotide”
uguuacaucacccacuucuuuuu

SEQ ID NO: 1039 - RNA, 21 nt, Artificial Sequence
  source: /note=“Description of Artificial Sequence Synthetic oligonucleotide”
  modified_base (21)..(21): Inverted abasic nucleotide
aagaagugggugauguaacan

SEQ ID NO: 1040 - RNA, 23 nt, Artificial Sequence
  source: /note=“Description of Artificial Sequence Synthetic oligonucleotide”
uuguuacaucacccacuucuuuu

SEQ ID NO: 1041 - RNA, 21 nt, Artificial Sequence
  source: /note=“Description of Artificial Sequence Synthetic oligonucleotide”
  modified_base (21)..(21): Inverted abasic nucleotide
agaagugggugauguaacaan

SEQ ID NO: 1042 - RNA, 23 nt, Artificial Sequence
  source: /note=“Description of Artificial Sequence Synthetic oligonucleotide”
auuguuacaucacccacuucuuu

SEQ ID NO: 1043 - RNA, 21 nt, Artificial Sequence
  source: /note=“Description of Artificial Sequence Synthetic oligonucleotide”
  modified_base (21)..(21): Inverted abasic nucleotide
gaagugggugauguaacaaun

SEQ ID NO: 1044 - RNA, 23 nt, Artificial Sequence
  source: /note=“Description of Artificial Sequence Synthetic oligonucleotide”
aauuguuacaucacccacuucuu

SEQ ID NO: 1045 - RNA, 21 nt, Artificial Sequence
  source: /note=“Description of Artificial Sequence Synthetic oligonucleotide”
  modified_base (21)..(21): Inverted abasic nucleotide
gauguaacaaucguggugaan

SEQ ID NO: 1046 - RNA, 23 nt, Artificial Sequence
  source: /note=“Description of Artificial Sequence Synthetic oligonucleotide”
auucaccacgauuguuacaucuu

SEQ ID NO: 1047 - RNA, 21 nt, Artificial Sequence
  source: /note=“Description of Artificial Sequence Synthetic oligonucleotide”
  modified_base (21)..(21): Inverted abasic nucleotide
auguaacaaucguggugaaun

SEQ ID NO: 1048 - RNA, 23 nt, Artificial Sequence
  source: /note=“Description of Artificial Sequence Synthetic oligonucleotide”
uauucaccacgauuguuacauuu

SEQ ID NO: 1049 - RNA, 21 nt, Artificial Sequence
  source: /note=“Description of Artificial Sequence Synthetic oligonucleotide”
  modified_base (21)..(21): Inverted abasic nucleotide
uguaacaaucguggugaauan

SEQ ID NO: 1050 - RNA, 23 nt, Artificial Sequence
  source: /note=“Description of Artificial Sequence Synthetic oligonucleotide”
uuauucaccacgauuguuacauu

SEQ ID NO: 1051 - RNA, 21 nt, Artificial Sequence
  source: /note=“Description of Artificial Sequence Synthetic oligonucleotide”
  modified_base (21)..(21): Inverted abasic nucleotide
guaacaaucguggugaauaan

SEQ ID NO: 1052 - RNA, 23 nt, Artificial Sequence
  source: /note=“Description of Artificial Sequence Synthetic oligonucleotide”
auuauucaccacgauuguuacuu

SEQ ID NO: 1053 - RNA, 21 nt, Artificial Sequence
  source: /note=“Description of Artificial Sequence Synthetic oligonucleotide”
  modified_base (21)..(21): Inverted abasic nucleotide
uaacaaucguggugaauaaun

SEQ ID NO: 1054 - RNA, 23 nt, Artificial Sequence
  source: /note=“Description of Artificial Sequence Synthetic oligonucleotide”
aauuauucaccacgauuguuauu

SEQ ID NO: 1055 - RNA, 21 nt, Artificial Sequence
  source: /note=“Description of Artificial Sequence Synthetic oligonucleotide”
  modified_base (21)..(21): Inverted abasic nucleotide
ggugaauaaugcugggacagn

SEQ ID NO: 1056 - RNA, 23 nt, Artificial Sequence
  source: /note=“Description of Artificial Sequence Synthetic oligonucleotide”
acugucccagcauuauucaccuu

SEQ ID NO: 1057 - RNA, 21 nt, Artificial Sequence
  source: /note=“Description of Artificial Sequence Synthetic oligonucleotide”
  modified_base (21)..(21): Inverted abasic nucleotide
gugaauaaugcugggacagun

SEQ ID NO: 1058 - RNA, 23 nt, Artificial Sequence
  source: /note=“Description of Artificial Sequence Synthetic oligonucleotide”
uacugucccagcauuauucacuu

SEQ ID NO: 1059 - RNA, 21 nt, Artificial Sequence
  source: /note=“Description of Artificial Sequence Synthetic oligonucleotide”
  modified_base (21)..(21): Inverted abasic nucleotide
uaaugcugggacaguauaucn

SEQ ID NO: 1060 - RNA, 23 nt, Artificial Sequence
  source: /note=“Description of Artificial Sequence Synthetic oligonucleotide”
agauauacugucccagcauuauu

SEQ ID NO: 1061 - RNA, 21 nt, Artificial Sequence
  source: /note=“Description of Artificial Sequence Synthetic oligonucleotide”
  modified_base (21)..(21): Inverted abasic nucleotide
aaugcugggacaguauauccn

SEQ ID NO: 1062 - RNA, 23 nt, Artificial Sequence
  source: /note=“Description of Artificial Sequence Synthetic oligonucleotide”
uggauauacugucccagcauuuu

SEQ ID NO: 1063 - RNA, 21 nt, Artificial Sequence
  source: /note=“Description of Artificial Sequence Synthetic oligonucleotide”
  modified_base (21)..(21): Inverted abasic nucleotide
aagagauuaccaagacauuun

SEQ ID NO: 1064 - RNA, 23 nt, Artificial Sequence
  source: /note=“Description of Artificial Sequence Synthetic oligonucleotide”
aaaaugucuugguaaucucuuuu

SEQ ID NO: 1065 - RNA, 21 nt, Artificial Sequence
  source: /note=“Description of Artificial Sequence Synthetic oligonucleotide”
  modified_base (21)..(21): Inverted abasic nucleotide
agagauuaccaagacauuugn

SEQ ID NO: 1066 - RNA, 23 nt, Artificial Sequence
  source: /note=“Description of Artificial Sequence Synthetic oligonucleotide”
ucaaaugucuugguaaucucuuu

SEQ ID NO: 1067 - RNA, 21 nt, Artificial Sequence
  source: /note=“Description of Artificial Sequence Synthetic oligonucleotide”
  modified_base (21)..(21): Inverted abasic nucleotide
gagauuaccaagacauuugan

SEQ ID NO: 1068 - RNA, 23 nt, Artificial Sequence
  source: /note=“Description of Artificial Sequence Synthetic oligonucleotide”
aucaaaugucuugguaaucucuu

SEQ ID NO: 1069 - RNA, 21 nt, Artificial Sequence
  source: /note=“Description of Artificial Sequence Synthetic oligonucleotide”
  modified_base (21)..(21): Inverted abasic nucleotide
ugaggucaacauccuaggacn

SEQ ID NO: 1070 - RNA, 23 nt, Artificial Sequence
  source: /note=“Description of Artificial Sequence Synthetic oligonucleotide”
uguccuaggauguugaccucauu

SEQ ID NO: 1071 - RNA, 21 nt, Artificial Sequence
  source: /note=“Description of Artificial Sequence Synthetic oligonucleotide”
  modified_base (21)..(21): Inverted abasic nucleotide
aggucaacauccuaggacaun

SEQ ID NO: 1072 - RNA, 23 nt, Artificial Sequence
  source: /note=“Description of Artificial Sequence Synthetic oligonucleotide”
aauguccuaggauguugaccuuu

SEQ ID NO: 1073 - RNA, 21 nt, Artificial Sequence
  source: /note=“Description of Artificial Sequence Synthetic oligonucleotide”
  modified_base (21)..(21): Inverted abasic nucleotide
ggucaacauccuaggacauun

SEQ ID NO: 1074 - RNA, 23 nt, Artificial Sequence
  source: /note=“Description of Artificial Sequence Synthetic oligonucleotide”
aaauguccuaggauguugaccuu

SEQ ID NO: 1075 - RNA, 21 nt, Artificial Sequence
  source: /note=“Description of Artificial Sequence Synthetic oligonucleotide”
  modified_base (21)..(21): Inverted abasic nucleotide
gucaacauccuaggacauuun

SEQ ID NO: 1076 - RNA, 23 nt, Artificial Sequence
  source: /note=“Description of Artificial Sequence Synthetic oligonucleotide”
aaaauguccuaggauguugacuu

SEQ ID NO: 1077 - RNA, 21 nt, Artificial Sequence
  source: /note=“Description of Artificial Sequence Synthetic oligonucleotide”
  modified_base (21)..(21): Inverted abasic nucleotide
ucaacauccuaggacauuuun

SEQ ID NO: 1078 - RNA, 23 nt, Artificial Sequence
  source: /note=“Description of Artificial Sequence Synthetic oligonucleotide”
aaaaauguccuaggauguugauu

SEQ ID NO: 1079 - RNA, 21 nt, Artificial Sequence
  source: /note=“Description of Artificial Sequence Synthetic oligonucleotide”
  modified_base (21)..(21): Inverted abasic nucleotide
caacauccuaggacauuuuun

SEQ ID NO: 1080 - RNA, 23 nt, Artificial Sequence
  source: /note=“Description of Artificial Sequence Synthetic oligonucleotide”
aaaaaauguccuaggauguuguu

SEQ ID NO: 1081 - RNA, 21 nt, Artificial Sequence
  source: /note=“Description of Artificial Sequence Synthetic oligonucleotide”
  modified_base (21)..(21): Inverted abasic nucleotide
caaaagcacuucuuccaucgn

SEQ ID NO: 1082 - RNA, 23 nt, Artificial Sequence
  source: /note=“Description of Artificial Sequence Synthetic oligonucleotide”
ucgauggaagaagugcuuuuguu

SEQ ID NO: 1083 - RNA, 21 nt, Artificial Sequence
  source: /note=“Description of Artificial Sequence Synthetic oligonucleotide”
  modified_base (21)..(21): Inverted abasic nucleotide
aaaagcacuucuuccaucgan

SEQ ID NO: 1084 - RNA, 23 nt, Artificial Sequence
  source: /note=“Description of Artificial Sequence Synthetic oligonucleotide”
aucgauggaagaagugcuuuuuu

SEQ ID NO: 1085 - RNA, 21 nt, Artificial Sequence
  source: /note=“Description of Artificial Sequence Synthetic oligonucleotide”
  modified_base (21)..(21): Inverted abasic nucleotide
aaagcacuucuuccaucgaun

SEQ ID NO: 1086 - RNA, 23 nt, Artificial Sequence
  source: /note=“Description of Artificial Sequence Synthetic oligonucleotide”
aaucgauggaagaagugcuuuuu

SEQ ID NO: 1087 - RNA, 21 nt, Artificial Sequence
  source: /note=“Description of Artificial Sequence Synthetic oligonucleotide”
  modified_base (21)..(21): Inverted abasic nucleotide
aagcacuucuuccaucgaugn

SEQ ID NO: 1088 - RNA, 23 nt, Artificial Sequence
  source: /note=“Description of Artificial Sequence Synthetic oligonucleotide”
ucaucgauggaagaagugcuuuu

SEQ ID NO: 1089 - RNA, 21 nt, Artificial Sequence
  source: /note=“Description of Artificial Sequence Synthetic oligonucleotide”
  modified_base (21)..(21): Inverted abasic nucleotide
agcacuucuuccaucgaugan

SEQ ID NO: 1090 - RNA, 23 nt, Artificial Sequence
  source: /note=“Description of Artificial Sequence Synthetic oligonucleotide”
aucaucgauggaagaagugcuuu

SEQ ID NO: 1091 - RNA, 21 nt, Artificial Sequence
  source: /note=“Description of Artificial Sequence Synthetic oligonucleotide”
  modified_base (21)..(21): Inverted abasic nucleotide
cacuucuuccaucgaugaugn

SEQ ID NO: 1092 - RNA, 23 nt, Artificial Sequence
  source: /note=“Description of Artificial Sequence Synthetic oligonucleotide”
acaucaucgauggaagaaguguu

SEQ ID NO: 1093 - RNA, 21 nt, Artificial Sequence
  source: /note=“Description of Artificial Sequence Synthetic oligonucleotide”
  modified_base (21)..(21): Inverted abasic nucleotide
acuucuuccaucgaugauggn

SEQ ID NO: 1094 - RNA, 23 nt, Artificial Sequence
  source: /note=“Description of Artificial Sequence Synthetic oligonucleotide”
uccaucaucgauggaagaaguuu

SEQ ID NO: 1095 - RNA, 21 nt, Artificial Sequence
  source: /note=“Description of Artificial Sequence Synthetic oligonucleotide”
  modified_base (21)..(21): Inverted abasic nucleotide
uuccaucgaugauggagagan

SEQ ID NO: 1096 - RNA, 23 nt, Artificial Sequence
  source: /note=“Description of Artificial Sequence Synthetic oligonucleotide”
uucucuccaucaucgauggaauu

SEQ ID NO: 1097 - RNA, 21 nt, Artificial Sequence
  source: /note=“Description of Artificial Sequence Synthetic oligonucleotide”
  modified_base (21)..(21): Inverted abasic nucleotide
ccaucgaugauggagagaaan

SEQ ID NO: 1098 - RNA, 23 nt, Artificial Sequence
  source: /note=“Description of Artificial Sequence Synthetic oligonucleotide”
auuucucuccaucaucgaugguu

SEQ ID NO: 1099 - RNA, 21 nt, Artificial Sequence
  source: /note=“Description of Artificial Sequence Synthetic oligonucleotide”
  modified_base (21)..(21): Inverted abasic nucleotide
gauggagagaaaucauggccn

SEQ ID NO: 1100 - RNA, 23 nt, Artificial Sequence
  source: /note=“Description of Artificial Sequence Synthetic oligonucleotide”
uggccaugauuucucuccaucuu

SEQ ID NO: 1101 - RNA, 21 nt, Artificial Sequence
  source: /note=“Description of Artificial Sequence Synthetic oligonucleotide”
  modified_base (21)..(21): Inverted abasic nucleotide
guggcuucagugugcggccan

SEQ ID NO: 1102 - RNA, 23 nt, Artificial Sequence
  source: /note=“Description of Artificial Sequence Synthetic oligonucleotide”
auggccgcacacugaagccacuu

SEQ ID NO: 1103 - RNA, 21 nt, Artificial Sequence
  source: /note=“Description of Artificial Sequence Synthetic oligonucleotide”
  modified_base (21)..(21): Inverted abasic nucleotide
uggcuucagugugcggccacn

SEQ ID NO: 1104 - RNA, 23 nt, Artificial Sequence
  source: /note=“Description of Artificial Sequence Synthetic oligonucleotide”
aguggccgcacacugaagccauu

SEQ ID NO: 1105 - RNA, 21 nt, Artificial Sequence
  source: /note=“Description of Artificial Sequence Synthetic oligonucleotide”
  modified_base (21)..(21): Inverted abasic nucleotide
gcuucagugugcggccacgan

SEQ ID NO: 1106 - RNA, 23 nt, Artificial Sequence
  source: /note=“Description of Artificial Sequence Synthetic oligonucleotide”
uucguggccgcacacugaagcuu

SEQ ID NO: 1107 - RNA, 21 nt, Artificial Sequence
  source: /note=“Description of Artificial Sequence Synthetic oligonucleotide”
  modified_base (21)..(21): Inverted abasic nucleotide
uucagugugcggccacgaagn

SEQ ID NO: 1108 - RNA, 23 nt, Artificial Sequence
  source: /note=“Description of Artificial Sequence Synthetic oligonucleotide”
acuucguggccgcacacugaauu

SEQ ID NO: 1109 - RNA, 21 nt, Artificial Sequence
  source: /note=“Description of Artificial Sequence Synthetic oligonucleotide”
  modified_base (21)..(21): Inverted abasic nucleotide
uugugaauacuggguucaccn

SEQ ID NO: 1110 - RNA, 23 nt, Artificial Sequence
  source: /note=“Description of Artificial Sequence Synthetic oligonucleotide”
uggugaacccaguauucacaauu

SEQ ID NO: 1111 - RNA, 21 nt, Artificial Sequence
  source: /note=“Description of Artificial Sequence Synthetic oligonucleotide”
  modified_base (21)..(21): Inverted abasic nucleotide
gaauacuggguucaccaaaan

SEQ ID NO: 1112 - RNA, 23 nt, Artificial Sequence
  source: /note=“Description of Artificial Sequence Synthetic oligonucleotide”
uuuuuggugaacccaguauucuu

SEQ ID NO: 1113 - RNA, 21 nt, Artificial Sequence
  source: /note=“Description of Artificial Sequence Synthetic oligonucleotide”
  modified_base (21)..(21): Inverted abasic nucleotide
auacuggguucaccaaaaaun

SEQ ID NO: 1114 - RNA, 23 nt, Artificial Sequence
  source: /note=“Description of Artificial Sequence Synthetic oligonucleotide”
aauuuuuggugaacccaguauuu

SEQ ID NO: 1115 - RNA, 21 nt, Artificial Sequence
  source: /note=“Description of Artificial Sequence Synthetic oligonucleotide”
  modified_base (21)..(21): Inverted abasic nucleotide
agcacaagauuauggccugun

SEQ ID NO: 1116 - RNA, 23 nt, Artificial Sequence
  source: /note=“Description of Artificial Sequence Synthetic oligonucleotide”
uacaggccauaaucuugugcuuu

SEQ ID NO: 1117 - RNA, 21 nt, Artificial Sequence
  source: /note=“Description of Artificial Sequence Synthetic oligonucleotide”
  modified_base (21)..(21): Inverted abasic nucleotide
cacaagauuauggccuguaun

SEQ ID NO: 1118 - RNA, 23 nt, Artificial Sequence
  source: /note=“Description of Artificial Sequence Synthetic oligonucleotide”
aauacaggccauaaucuuguguu

SEQ ID NO: 1119 - RNA, 21 nt, Artificial Sequence
  source: /note=“Description of Artificial Sequence Synthetic oligonucleotide”
  modified_base (21)..(21): Inverted abasic nucleotide
acaagauuauggccuguauun

SEQ ID NO: 1120 - RNA, 23 nt, Artificial Sequence
  source: /note=“Description of Artificial Sequence Synthetic oligonucleotide”
aaauacaggccauaaucuuguuu

SEQ ID NO: 1121 - RNA, 21 nt, Artificial Sequence
  source: /note=“Description of Artificial Sequence Synthetic oligonucleotide”
  modified_base (21)..(21): Inverted abasic nucleotide
caagauuauggccuguauugn

SEQ ID NO: 1122 - RNA, 23 nt, Artificial Sequence
  source: /note=“Description of Artificial Sequence Synthetic oligonucleotide”
acaauacaggccauaaucuuguu

SEQ ID NO: 1123 - RNA, 21 nt, Artificial Sequence
  source: /note=“Description of Artificial Sequence Synthetic oligonucleotide”
  modified_base (21)..(21): Inverted abasic nucleotide
agauuauggccuguauuggan

SEQ ID NO: 1124 - RNA, 23 nt, Artificial Sequence
  source: /note=“Description of Artificial Sequence Synthetic oligonucleotide”
auccaauacaggccauaaucuuu

SEQ ID NO: 1125 - RNA, 21 nt, Artificial Sequence
  source: /note=“Description of Artificial Sequence Synthetic oligonucleotide”
  modified_base (21)..(21): Inverted abasic nucleotide
gauuauggccuguauuggagn

SEQ ID NO: 1126 - RNA, 23 nt, Artificial Sequence
  source: /note=“Description of Artificial Sequence Synthetic oligonucleotide”
ucuccaauacaggccauaaucuu

SEQ ID NO: 1127 - RNA, 21 nt, Artificial Sequence
  source: /note=“Description of Artificial Sequence Synthetic oligonucleotide”
  modified_base (21)..(21): Inverted abasic nucleotide
gaagucugauagauggaauan

SEQ ID NO: 1128 - RNA, 23 nt, Artificial Sequence
  source: /note=“Description of Artificial Sequence Synthetic oligonucleotide”
auauuccaucuaucagacuucuu

SEQ ID NO: 1129 - RNA, 21 nt, Artificial Sequence
  source: /note=“Description of Artificial Sequence Synthetic oligonucleotide”
  modified_base (21)..(21): Inverted abasic nucleotide
aagucugauagauggaauacn

SEQ ID NO: 1130 - RNA, 23 nt, Artificial Sequence
  source: /note=“Description of Artificial Sequence Synthetic oligonucleotide”
aguauuccaucuaucagacuuuu

SEQ ID NO: 1131 - RNA, 21 nt, Artificial Sequence
  source: /note=“Description of Artificial Sequence Synthetic oligonucleotide”
  modified_base (21)..(21): Inverted abasic nucleotide
agucugauagauggaauacun

SEQ ID NO: 1132 - RNA, 23 nt, Artificial Sequence
  source: /note=“Description of Artificial Sequence Synthetic oligonucleotide”
aaguauuccaucuaucagacuuu

SEQ ID NO: 1133 - RNA, 21 nt, Artificial Sequence
  source: /note=“Description of Artificial Sequence Synthetic oligonucleotide”
  modified_base (21)..(21): Inverted abasic nucleotide
gucugauagauggaauacuun

SEQ ID NO: 1134 - RNA, 23 nt, Artificial Sequence
  source: /note=“Description of Artificial Sequence Synthetic oligonucleotide”
uaaguauuccaucuaucagacuu

SEQ ID NO: 1135 - RNA, 21 nt, Artificial Sequence
  source: /note=“Description of Artificial Sequence Synthetic oligonucleotide”
  modified_base (21)..(21): Inverted abasic nucleotide
ucugauagauggaauacuuan

SEQ ID NO: 1136 - RNA, 23 nt, Artificial Sequence
  source: /note=“Description of Artificial Sequence Synthetic oligonucleotide”
auaaguauuccaucuaucagauu

SEQ ID NO: 1137 - RNA, 21 nt, Artificial Sequence
  source: /note=“Description of Artificial Sequence Synthetic oligonucleotide”
  modified_base (21)..(21): Inverted abasic nucleotide
auagauggaauacuuaccaan

SEQ ID NO: 1138 - RNA, 23 nt, Artificial Sequence
  source: /note=“Description of Artificial Sequence Synthetic oligonucleotide”
auugguaaguauuccaucuauuu

SEQ ID NO: 1139 - RNA, 21 nt, Artificial Sequence
  source: /note=“Description of Artificial Sequence Synthetic oligonucleotide”
  modified_base (21)..(21): Inverted abasic nucleotide
uagauggaauacuuaccaaun

SEQ ID NO: 1140 - RNA, 23 nt, Artificial Sequence
  source: /note=“Description of Artificial Sequence Synthetic oligonucleotide”
uauugguaaguauuccaucuauu

SEQ ID NO: 1141 - RNA, 21 nt, Artificial Sequence
  source: /note=“Description of Artificial Sequence Synthetic oligonucleotide”
  modified_base (21)..(21): Inverted abasic nucleotide
agauggaauacuuaccaauan

SEQ ID NO: 1142 - RNA, 23 nt, Artificial Sequence
  source: /note=“Description of Artificial Sequence Synthetic oligonucleotide”
uuauugguaaguauuccaucuuu

SEQ ID NO: 1143 - RNA, 21 nt, Artificial Sequence
  source: /note=“Description of Artificial Sequence Synthetic oligonucleotide”
  modified_base (21)..(21): Inverted abasic nucleotide
gauggaauacuuaccaauaan

SEQ ID NO: 1144 - RNA, 23 nt, Artificial Sequence
  source: /note=“Description of Artificial Sequence Synthetic oligonucleotide”
auuauugguaaguauuccaucuu

SEQ ID NO: 1145 - RNA, 21 nt, Artificial Sequence
  source: /note=“Description of Artificial Sequence Synthetic oligonucleotide”
  modified_base (21)..(21): Inverted abasic nucleotide
auggaauacuuaccaauaagn

SEQ ID NO: 1146 - RNA, 23 nt, Artificial Sequence
  source: /note=“Description of Artificial Sequence Synthetic oligonucleotide”
ucuuauugguaaguauuccauuu

SEQ ID NO: 1147 - RNA, 21 nt, Artificial Sequence
  source: /note=“Description of Artificial Sequence Synthetic oligonucleotide”
  modified_base (21)..(21): Inverted abasic nucleotide
uggaauacuuaccaauaagan

SEQ ID NO: 1148 - RNA, 23 nt, Artificial Sequence
  source: /note=“Description of Artificial Sequence Synthetic oligonucleotide”
uucuuauugguaaguauuccauu

SEQ ID NO: 1149 - RNA, 21 nt, Artificial Sequence
  source: /note=“Description of Artificial Sequence Synthetic oligonucleotide”
  modified_base (21)..(21): Inverted abasic nucleotide
auaucaauaucuuucugagan

SEQ ID NO: 1150 - RNA, 23 nt, Artificial Sequence
  source: /note=“Description of Artificial Sequence Synthetic oligonucleotide”
aucucagaaagauauugauauuu

SEQ ID NO: 1151 - RNA, 21 nt, Artificial Sequence
  source: /note=“Description of Artificial Sequence Synthetic oligonucleotide”
  modified_base (21)..(21): Inverted abasic nucleotide
cuuucugagacuacagaagun

SEQ ID NO: 1152 - RNA, 23 nt, Artificial Sequence
  source: /note=“Description of Artificial Sequence Synthetic oligonucleotide”
aacuucuguagucucagaaaguu

SEQ ID NO: 1153 - RNA, 21 nt, Artificial Sequence
  source: /note=“Description of Artificial Sequence Synthetic oligonucleotide”
  modified_base (21)..(21): Inverted abasic nucleotide
uuucugagacuacagaaguun

SEQ ID NO: 1154 - RNA, 23 nt, Artificial Sequence
  source: /note=“Description of Artificial Sequence Synthetic oligonucleotide”
aaacuucuguagucucagaaauu

SEQ ID NO: 1155 - RNA, 21 nt, Artificial Sequence
  source: /note=“Description of Artificial Sequence Synthetic oligonucleotide”
  modified_base (21)..(21): Inverted abasic nucleotide
uucugagacuacagaaguuun

SEQ ID NO: 1156 - RNA, 23 nt, Artificial Sequence
  source: /note=“Description of Artificial Sequence Synthetic oligonucleotide”
aaaacuucuguagucucagaauu

SEQ ID NO: 1157 - RNA, 21 nt, Artificial Sequence
  source: /note=“Description of Artificial Sequence Synthetic oligonucleotide”
  modified_base (21)..(21): Inverted abasic nucleotide
ucugagacuacagaaguuucn

SEQ ID NO: 1158 - RNA, 23 nt, Artificial Sequence
  source: /note=“Description of Artificial Sequence Synthetic oligonucleotide”
agaaacuucuguagucucagauu

SEQ ID NO: 1159 - RNA, 21 nt, Artificial Sequence
  source: /note=“Description of Artificial Sequence Synthetic oligonucleotide”
  modified_base (21)..(21): Inverted abasic nucleotide
ugguuggccacaaaaucaaan

SEQ ID NO: 1160 - RNA, 23 nt, Artificial Sequence
  source: /note=“Description of Artificial Sequence Synthetic oligonucleotide”
uuuugauuuuguggccaaccauu

SEQ ID NO: 1161 - RNA, 21 nt, Artificial Sequence
  source: /note=“Description of Artificial Sequence Synthetic oligonucleotide”
  modified_base (21)..(21): Inverted abasic nucleotide
aaaugaaaugaauaaauaagn

SEQ ID NO: 1162 - RNA, 23 nt, Artificial Sequence
  source: /note=“Description of Artificial Sequence Synthetic oligonucleotide”
acuuauuuauucauuucauuuuu

SEQ ID NO: 1163 - RNA, 21 nt, Artificial Sequence
  source: /note=“Description of Artificial Sequence Synthetic oligonucleotide”
  modified_base (21)..(21): Inverted abasic nucleotide
uucacauuuuuucaguccugn

SEQ ID NO: 1164 - RNA, 23 nt, Artificial Sequence
  source: /note=“Description of Artificial Sequence Synthetic oligonucleotide”
ucaggacugaaaaaaugugaauu

SEQ ID NO: 1165 - RNA, 21 nt, Artificial Sequence
  source: /note=“Description of Artificial Sequence Synthetic oligonucleotide”
  modified_base (21)..(21): Inverted abasic nucleotide
guuuggcacuagcagcagucn

SEQ ID NO: 1166 - RNA, 23 nt, Artificial Sequence
  source: /note=“Description of Artificial Sequence Synthetic oligonucleotide”
ugacugcugcuagugccaaacuu

SEQ ID NO: 1167 - RNA, 21 nt, Artificial Sequence
  source: /note=“Description of Artificial Sequence Synthetic oligonucleotide”
  modified_base (21)..(21): Inverted abasic nucleotide
uuuggcacuagcagcagucan

SEQ ID NO: 1168 - RNA, 23 nt, Artificial Sequence
  source: /note=“Description of Artificial Sequence Synthetic oligonucleotide”
uugacugcugcuagugccaaauu

SEQ ID NO: 1169 - RNA, 21 nt, Artificial Sequence
  source: /note=“Description of Artificial Sequence Synthetic oligonucleotide”
  modified_base (21)..(21): Inverted abasic nucleotide
uuggcacuagcagcagucaan

SEQ ID NO: 1170 - RNA, 23 nt, Artificial Sequence
  source: /note=“Description of Artificial Sequence Synthetic oligonucleotide”
uuugacugcugcuagugccaauu

SEQ ID NO: 1171 - RNA, 21 nt, Artificial Sequence
  source: /note=“Description of Artificial Sequence Synthetic oligonucleotide”
  modified_base (21)..(21): Inverted abasic nucleotide
uggcacuagcagcagucaaan

SEQ ID NO: 1172 - RNA, 23 nt, Artificial Sequence
  source: /note=“Description of Artificial Sequence Synthetic oligonucleotide”
auuugacugcugcuagugccauu

SEQ ID NO: 1173 - RNA, 21 nt, Artificial Sequence
  source: /note=“Description of Artificial Sequence Synthetic oligonucleotide”
  modified_base (21)..(21): Inverted abasic nucleotide
ggcacuagcagcagucaaacn

SEQ ID NO: 1174 - RNA, 23 nt, Artificial Sequence
  source: /note=“Description of Artificial Sequence Synthetic oligonucleotide”
aguuugacugcugcuagugccuu

SEQ ID NO: 1175 - RNA, 21 nt, Artificial Sequence
  source: /note=“Description of Artificial Sequence Synthetic oligonucleotide”
  modified_base (21)..(21): Inverted abasic nucleotide
auuuacguaguuuuucauagn

SEQ ID NO: 1176 - RNA, 23 nt, Artificial Sequence
  source: /note=“Description of Artificial Sequence Synthetic oligonucleotide”
acuaugaaaaacuacguaaauuu

SEQ ID NO: 1177 - RNA, 21 nt, Artificial Sequence
  source: /note=“Description of Artificial Sequence Synthetic oligonucleotide”
  modified_base (21)..(21): Inverted abasic nucleotide
uuacguaguuuuucauaggun

SEQ ID NO: 1178 - RNA, 23 nt, Artificial Sequence
  source: /note=“Description of Artificial Sequence Synthetic oligonucleotide”
aaccuaugaaaaacuacguaauu

SEQ ID NO: 1179 - RNA, 21 nt, Artificial Sequence
  source: /note=“Description of Artificial Sequence Synthetic oligonucleotide”
  modified_base (21)..(21): Inverted abasic nucleotide
uacguaguuuuucauaggucn

SEQ ID NO: 1180 - RNA, 23 nt, Artificial Sequence
  source: /note=“Description of Artificial Sequence Synthetic oligonucleotide”
agaccuaugaaaaacuacguauu

SEQ ID NO: 1181 - RNA, 21 nt, Artificial Sequence
  source: /note=“Description of Artificial Sequence Synthetic oligonucleotide”
  modified_base (21)..(21): Inverted abasic nucleotide
uuacauaaacauacuuaaaan

SEQ ID NO: 1182 - RNA, 23 nt, Artificial Sequence
  source: /note=“Description of Artificial Sequence Synthetic oligonucleotide”
auuuuaaguauguuuauguaauu

SEQ ID NO: 1183 - RNA, 21 nt, Artificial Sequence
  source: /note=“Description of Artificial Sequence Synthetic oligonucleotide”
  modified_base (21)..(21): Inverted abasic nucleotide
uuaaagguggacaaaagcuan

SEQ ID NO: 1184 - RNA, 23 nt, Artificial Sequence
  source: /note=“Description of Artificial Sequence Synthetic oligonucleotide”
auagcuuuuguccaccuuuaauu

SEQ ID NO: 1185 - RNA, 21 nt, Artificial Sequence
  source: /note=“Description of Artificial Sequence Synthetic oligonucleotide”
  modified_base (21)..(21): Inverted abasic nucleotide
uaaagguggacaaaagcuacn

SEQ ID NO: 1186 - RNA, 23 nt, Artificial Sequence
  source: /note=“Description of Artificial Sequence Synthetic oligonucleotide”
aguagcuuuuguccaccuuuauu

SEQ ID NO: 1187 - RNA, 21 nt, Artificial Sequence
  source: /note=“Description of Artificial Sequence Synthetic oligonucleotide”
  modified_base (21)..(21): Inverted abasic nucleotide
aagguggacaaaagcuaccun

SEQ ID NO: 1188 - RNA, 23 nt, Artificial Sequence
  source: /note=“Description of Artificial Sequence Synthetic oligonucleotide”
aagguagcuuuuguccaccuuuu

SEQ ID NO: 1189 - RNA, 21 nt, Artificial Sequence
  source: /note=“Description of Artificial Sequence Synthetic oligonucleotide”
  modified_base (21)..(21): Inverted abasic nucleotide
gguggacaaaagcuaccuccn

SEQ ID NO: 1190 - RNA, 23 nt, Artificial Sequence
  source: /note=“Description of Artificial Sequence Synthetic oligonucleotide”
aggagguagcuuuuguccaccuu

SEQ ID NO: 1191 - RNA, 21 nt, Artificial Sequence
  source: /note=“Description of Artificial Sequence Synthetic oligonucleotide”
  modified_base (21)..(21): Inverted abasic nucleotide
acagcuaagagaucaaguuun

SEQ ID NO: 1192 - RNA, 23 nt, Artificial Sequence
  source: /note=“Description of Artificial Sequence Synthetic oligonucleotide”
aaaacuugaucucuuagcuguuu

SEQ ID NO: 1193 - RNA, 21 nt, Artificial Sequence
  source: /note=“Description of Artificial Sequence Synthetic oligonucleotide”
  modified_base (21)..(21): Inverted abasic nucleotide
cagcuaagagaucaaguuucn

SEQ ID NO: 1194 - RNA, 23 nt, Artificial Sequence
  source: /note=“Description of Artificial Sequence Synthetic oligonucleotide”
ugaaacuugaucucuuagcuguu

SEQ ID NO: 1195 - RNA, 21 nt, Artificial Sequence
  source: /note=“Description of Artificial Sequence Synthetic oligonucleotide”
  modified_base (21)..(21): Inverted abasic nucleotide
agcuaagagaucaaguuucan

SEQ ID NO: 1196 - RNA, 23 nt, Artificial Sequence
  source: /note=“Description of Artificial Sequence Synthetic oligonucleotide”
augaaacuugaucucuuagcuuu

SEQ ID NO: 1197 - RNA, 21 nt, Artificial Sequence
  source: /note=“Description of Artificial Sequence Synthetic oligonucleotide”
  modified_base (21)..(21): Inverted abasic nucleotide
ccuggacauauuuuaagauun

SEQ ID NO: 1198 - RNA, 23 nt, Artificial Sequence
  source: /note=“Description of Artificial Sequence Synthetic oligonucleotide”
aaaucuuaaaauauguccagguu

SEQ ID NO: 1199 - RNA, 21 nt, Artificial Sequence
  source: /note=“Description of Artificial Sequence Synthetic oligonucleotide”
  modified_base (21)..(21): Inverted abasic nucleotide
cuggacauauuuuaagauucn

SEQ ID NO: 1200 - RNA, 23 nt, Artificial Sequence
  source: /note=“Description of Artificial Sequence Synthetic oligonucleotide”
ugaaucuuaaaauauguccaguu

SEQ ID NO: 1201 - RNA, 21 nt, Artificial Sequence
  source: /note=“Description of Artificial Sequence Synthetic oligonucleotide”
  modified_base (21)..(21): Inverted abasic nucleotide
cuuccuuuuucauuagcccan

SEQ ID NO: 1202 - RNA, 23 nt, Artificial Sequence
  source: /note=“Description of Artificial Sequence Synthetic oligonucleotide”
uugggcuaaugaaaaaggaaguu

SEQ ID NO: 1203 - RNA, 21 nt, Artificial Sequence
  source: /note=“Description of Artificial Sequence Synthetic oligonucleotide”
  modified_base (21)..(21): Inverted abasic nucleotide
uuccuuuuucauuagcccaan

SEQ ID NO: 1204 - RNA, 23 nt, Artificial Sequence
  source: /note=“Description of Artificial Sequence Synthetic oligonucleotide”
uuugggcuaaugaaaaaggaauu

SEQ ID NO: 1205 - RNA, 21 nt, Artificial Sequence
  source: /note=“Description of Artificial Sequence Synthetic oligonucleotide”
  modified_base (21)..(21): Inverted abasic nucleotide
cccucuauauuuccucccuun

SEQ ID NO: 1206 - RNA, 23 nt, Artificial Sequence
  source: /note=“Description of Artificial Sequence Synthetic oligonucleotide”
aaagggaggaaauauagaggguu

SEQ ID NO: 1207 - RNA, 21 nt, Artificial Sequence
  source: /note=“Description of Artificial Sequence Synthetic oligonucleotide”
  modified_base (21)..(21): Inverted abasic nucleotide
uauuuccucccuuuuuauagn

SEQ ID NO: 1208 - RNA, 23 nt, Artificial Sequence
  source: /note=“Description of Artificial Sequence Synthetic oligonucleotide”
acuauaaaaagggaggaaauauu

SEQ ID NO: 1209 - RNA, 21 nt, Artificial Sequence
  source: /note=“Description of Artificial Sequence Synthetic oligonucleotide”
  modified_base (21)..(21): Inverted abasic nucleotide
uuccucccuuuuuauagucun

SEQ ID NO: 1210 - RNA, 23 nt, Artificial Sequence
  source: /note=“Description of Artificial Sequence Synthetic oligonucleotide”
aagacuauaaaaagggaggaauu

SEQ ID NO: 1211 - RNA, 21 nt, Artificial Sequence
  source: /note=“Description of Artificial Sequence Synthetic oligonucleotide”
  modified_base (21)..(21): Inverted abasic nucleotide
uccucccuuuuuauagucuun

SEQ ID NO: 1212 - RNA, 23 nt, Artificial Sequence
  source: /note=“Description of Artificial Sequence Synthetic oligonucleotide”
uaagacuauaaaaagggaggauu

SEQ ID NO: 1213 - RNA, 21 nt, Artificial Sequence
  source: /note=“Description of Artificial Sequence Synthetic oligonucleotide”
  modified_base (21)..(21): Inverted abasic nucleotide
ccuuuuuauagucuuauaagn

SEQ ID NO: 1214 - RNA, 23 nt, Artificial Sequence
  source: /note=“Description of Artificial Sequence Synthetic oligonucleotide”
ucuuauaagacuauaaaaagguu

SEQ ID NO: 1215 - RNA, 21 nt, Artificial Sequence
  source: /note=“Description of Artificial Sequence Synthetic oligonucleotide”
  modified_base (21)..(21): Inverted abasic nucleotide
cuuuuuauagucuuauaagan

SEQ ID NO: 1216 - RNA, 23 nt, Artificial Sequence
  source: /note=“Description of Artificial Sequence Synthetic oligonucleotide”
aucuuauaagacuauaaaaaguu

SEQ ID NO: 1217 - RNA, 21 nt, Artificial Sequence
  source: /note=“Description of Artificial Sequence Synthetic oligonucleotide”
  modified_base (21)..(21): Inverted abasic nucleotide
uuuuuauagucuuauaagaun

SEQ ID NO: 1218 - RNA, 23 nt, Artificial Sequence
  source: /note=“Description of Artificial Sequence Synthetic oligonucleotide”
uaucuuauaagacuauaaaaauu

SEQ ID NO: 1219 - RNA, 21 nt, Artificial Sequence
  source: /note=“Description of Artificial Sequence Synthetic oligonucleotide”
  modified_base (21)..(21): Inverted abasic nucleotide
uuuuauagucuuauaagauan

SEQ ID NO: 1220 - RNA, 23 nt, Artificial Sequence
  source: /note=“Description of Artificial Sequence Synthetic oligonucleotide”
auaucuuauaagacuauaaaauu

SEQ ID NO: 1221 - RNA, 21 nt, Artificial Sequence
  source: /note=“Description of Artificial Sequence Synthetic oligonucleotide”
  modified_base (21)..(21): Inverted abasic nucleotide
uuuauagucuuauaagauacn

SEQ ID NO: 1222 - RNA, 23 nt, Artificial Sequence
  source: /note=“Description of Artificial Sequence Synthetic oligonucleotide”
uguaucuuauaagacuauaaauu

SEQ ID NO: 1223 - RNA, 21 nt, Artificial Sequence
  source: /note=“Description of Artificial Sequence Synthetic oligonucleotide”
  modified_base (21)..(21): Inverted abasic nucleotide
uuauagucuuauaagauacan

SEQ ID NO: 1224 - RNA, 23 nt, Artificial Sequence
  source: /note=“Description of Artificial Sequence Synthetic oligonucleotide”
auguaucuuauaagacuauaauu

SEQ ID NO: 1225 - RNA, 21 nt, Artificial Sequence
  source: /note=“Description of Artificial Sequence Synthetic oligonucleotide”
  modified_base (21)..(21): Inverted abasic nucleotide
uauagucuuauaagauacaun

SEQ ID NO: 1226 - RNA, 23 nt, Artificial Sequence
  source: /note=“Description of Artificial Sequence Synthetic oligonucleotide”
aauguaucuuauaagacuauauu

SEQ ID NO: 1227 - RNA, 21 nt, Artificial Sequence
  source: /note=“Description of Artificial Sequence Synthetic oligonucleotide”
  modified_base (21)..(21): Inverted abasic nucleotide
ucuuauaagauacauuaugan

SEQ ID NO: 1228 - RNA, 23 nt, Artificial Sequence
  source: /note=“Description of Artificial Sequence Synthetic oligonucleotide”
uucauaauguaucuuauaagauu

SEQ ID NO: 1229 - RNA, 21 nt, Artificial Sequence
  source: /note=“Description of Artificial Sequence Synthetic oligonucleotide”
  modified_base (21)..(21): Inverted abasic nucleotide
uuuuaaguucuagccccaugn

SEQ ID NO: 1230 - RNA, 23 nt, Artificial Sequence
  source: /note=“Description of Artificial Sequence Synthetic oligonucleotide”
ucauggggcuagaacuuaaaauu

SEQ ID NO: 1231 - RNA, 21 nt, Artificial Sequence
  source: /note=“Description of Artificial Sequence Synthetic oligonucleotide”
  modified_base (21)..(21): Inverted abasic nucleotide
uuuaaguucuagccccaugan

SEQ ID NO: 1232 - RNA, 23 nt, Artificial Sequence
  source: /note=“Description of Artificial Sequence Synthetic oligonucleotide”
aucauggggcuagaacuuaaauu

SEQ ID NO: 1233 - RNA, 21 nt, Artificial Sequence
  source: /note=“Description of Artificial Sequence Synthetic oligonucleotide”
  modified_base (21)..(21): Inverted abasic nucleotide
uaaguucuagccccaugauan

SEQ ID NO: 1234 - RNA, 23 nt, Artificial Sequence
  source: /note=“Description of Artificial Sequence Synthetic oligonucleotide”
uuaucauggggcuagaacuuauu

SEQ ID NO: 1235 - RNA, 21 nt, Artificial Sequence
  source: /note=“Description of Artificial Sequence Synthetic oligonucleotide”
  modified_base (21)..(21): Inverted abasic nucleotide
aaguucuagccccaugauaan

SEQ ID NO: 1236 - RNA, 23 nt, Artificial Sequence
  source: /note=“Description of Artificial Sequence Synthetic oligonucleotide”
auuaucauggggcuagaacuuuu

SEQ ID NO: 1237 - RNA, 21 nt, Artificial Sequence
  source: /note=“Description of Artificial Sequence Synthetic oligonucleotide”
  modified_base (21)..(21): Inverted abasic nucleotide
aguucuagccccaugauaacn

SEQ ID NO: 1238 - RNA, 23 nt, Artificial Sequence
  source: /note=“Description of Artificial Sequence Synthetic oligonucleotide”
aguuaucauggggcuagaacuuu

SEQ ID NO: 1239 - RNA, 21 nt, Artificial Sequence
  source: /note=“Description of Artificial Sequence Synthetic oligonucleotide”
  modified_base (21)..(21): Inverted abasic nucleotide
guucuagccccaugauaaccn

SEQ ID NO: 1240 - RNA, 23 nt, Artificial Sequence
  source: /note=“Description of Artificial Sequence Synthetic oligonucleotide”
agguuaucauggggcuagaacuu

SEQ ID NO: 1241 - RNA, 21 nt, Artificial Sequence
  source: /note=“Description of Artificial Sequence Synthetic oligonucleotide”
  modified_base (21)..(21): Inverted abasic nucleotide
cuagccccaugauaaccuuun

SEQ ID NO: 1242 - RNA, 23 nt, Artificial Sequence
  source: /note=“Description of Artificial Sequence Synthetic oligonucleotide”
aaaagguuaucauggggcuaguu

SEQ ID NO: 1243 - RNA, 21 nt, Artificial Sequence
  source: /note=“Description of Artificial Sequence Synthetic oligonucleotide”
  modified_base (21)..(21): Inverted abasic nucleotide
agccccaugauaaccuuuuun

SEQ ID NO: 1244 - RNA, 23 nt, Artificial Sequence
  source: /note=“Description of Artificial Sequence Synthetic oligonucleotide”
aaaaaagguuaucauggggcuuu

SEQ ID NO: 1245 - RNA, 21 nt, Artificial Sequence
  source: /note=“Description of Artificial Sequence Synthetic oligonucleotide”
  modified_base (21)..(21): Inverted abasic nucleotide
gccccaugauaaccuuuuucn

SEQ ID NO: 1246 - RNA, 23 nt, Artificial Sequence
  source: /note=“Description of Artificial Sequence Synthetic oligonucleotide”
agaaaaagguuaucauggggcuu

SEQ ID NO: 1247 - RNA, 21 nt, Artificial Sequence
  source: /note=“Description of Artificial Sequence Synthetic oligonucleotide”
  modified_base (21)..(21): Inverted abasic nucleotide
cccaugauaaccuuuuucuun

SEQ ID NO: 1248 - RNA, 23 nt, Artificial Sequence
  source: /note=“Description of Artificial Sequence Synthetic oligonucleotide”
aaagaaaaagguuaucauggguu

SEQ ID NO: 1249 - RNA, 21 nt, Artificial Sequence
  source: /note=“Description of Artificial Sequence Synthetic oligonucleotide”
  modified_base (21)..(21): Inverted abasic nucleotide
ccaugauaaccuuuuucuuun

SEQ ID NO: 1250 - RNA, 23 nt, Artificial Sequence
  source: /note=“Description of Artificial Sequence Synthetic oligonucleotide”
aaaagaaaaagguuaucaugguu

SEQ ID NO: 1251 - RNA, 21 nt, Artificial Sequence
  source: /note=“Description of Artificial Sequence Synthetic oligonucleotide”
  modified_base (21)..(21): Inverted abasic nucleotide
caugauaaccuuuuucuuugn

SEQ ID NO: 1252 - RNA, 23 nt, Artificial Sequence
  source: /note=“Description of Artificial Sequence Synthetic oligonucleotide”
acaaagaaaaagguuaucauguu

SEQ ID NO: 1253 - RNA, 21 nt, Artificial Sequence
  source: /note=“Description of Artificial Sequence Synthetic oligonucleotide”
  modified_base (21)..(21): Inverted abasic nucleotide
auaaccuuuuucuuuguaaun

SEQ ID NO: 1254 - RNA, 23 nt, Artificial Sequence
  source: /note=“Description of Artificial Sequence Synthetic oligonucleotide”
aauuacaaagaaaaagguuauuu

SEQ ID NO: 1255 - RNA, 21 nt, Artificial Sequence
  source: /note=“Description of Artificial Sequence Synthetic oligonucleotide”
  modified_base (21)..(21): Inverted abasic nucleotide
uuuuucuuuguaauuuaugcn

SEQ ID NO: 1256 - RNA, 23 nt, Artificial Sequence
  source: /note=“Description of Artificial Sequence Synthetic oligonucleotide”
agcauaaauuacaaagaaaaauu

SEQ ID NO: 1257 - RNA, 21 nt, Artificial Sequence
  source: /note=“Description of Artificial Sequence Synthetic oligonucleotide”
  modified_base (21)..(21): Inverted abasic nucleotide
uuuucuuuguaauuuaugcun

SEQ ID NO: 1258 - RNA, 23 nt, Artificial Sequence
  source: /note=“Description of Artificial Sequence Synthetic oligonucleotide”
aagcauaaauuacaaagaaaauu

SEQ ID NO: 1259 - RNA, 21 nt, Artificial Sequence
  source: /note=“Description of Artificial Sequence Synthetic oligonucleotide”
  modified_base (21)..(21): Inverted abasic nucleotide
ggcuauuacauaagaaacaan

SEQ ID NO: 1260 - RNA, 23 nt, Artificial Sequence
  source: /note=“Description of Artificial Sequence Synthetic oligonucleotide”
auuguuucuuauguaauagccuu

SEQ ID NO: 1261 - RNA, 21 nt, Artificial Sequence
  source: /note=“Description of Artificial Sequence Synthetic oligonucleotide”
  modified_base (21)..(21): Inverted abasic nucleotide
cuauuacauaagaaacaaugn

SEQ ID NO: 1262 - RNA, 23 nt, Artificial Sequence
  source: /note=“Description of Artificial Sequence Synthetic oligonucleotide”
acauuguuucuuauguaauaguu

SEQ ID NO: 1263 - RNA, 21 nt, Artificial Sequence
  source: /note=“Description of Artificial Sequence Synthetic oligonucleotide”
  modified_base (21)..(21): Inverted abasic nucleotide
uuacauaagaaacaauggacn

SEQ ID NO: 1264 - RNA, 23 nt, Artificial Sequence
  source: /note=“Description of Artificial Sequence Synthetic oligonucleotide”
aguccauuguuucuuauguaauu

SEQ ID NO: 1265 - RNA, 21 nt, Artificial Sequence
  source: /note=“Description of Artificial Sequence Synthetic oligonucleotide”
  modified_base (21)..(21): Inverted abasic nucleotide
uacauaagaaacaauggaccn

SEQ ID NO: 1266 - RNA, 23 nt, Artificial Sequence
  source: /note=“Description of Artificial Sequence Synthetic oligonucleotide”
agguccauuguuucuuauguauu

SEQ ID NO: 1267 - RNA, 21 nt, Artificial Sequence
  source: /note=“Description of Artificial Sequence Synthetic oligonucleotide”
  modified_base (21)..(21): Inverted abasic nucleotide
acauaagaaacaauggacccn

SEQ ID NO: 1268 - RNA, 23 nt, Artificial Sequence
  source: /note=“Description of Artificial Sequence Synthetic oligonucleotide”
uggguccauuguuucuuauguuu

SEQ ID NO: 1269 - RNA, 21 nt, Artificial Sequence
  source: /note=“Description of Artificial Sequence Synthetic oligonucleotide”
  modified_base (21)..(21): Inverted abasic nucleotide
aagaaacaauggacccaagan

SEQ ID NO: 1270 - RNA, 23 nt, Artificial Sequence
  source: /note=“Description of Artificial Sequence Synthetic oligonucleotide”
aucuuggguccauuguuucuuuu

SEQ ID NO: 1271 - RNA, 21 nt, Artificial Sequence
  source: /note=“Description of Artificial Sequence Synthetic oligonucleotide”
  modified_base (21)..(21): Inverted abasic nucleotide
agaaacaauggacccaagagn

SEQ ID NO: 1272 - RNA, 23 nt, Artificial Sequence
  source: /note=“Description of Artificial Sequence Synthetic oligonucleotide”
ucucuuggguccauuguuucuuu

SEQ ID NO: 1273 - RNA, 21 nt, Artificial Sequence
  source: /note=“Description of Artificial Sequence Synthetic oligonucleotide”
  modified_base (21)..(21): Inverted abasic nucleotide
gaaacaauggacccaagagan

SEQ ID NO: 1274 - RNA, 23 nt, Artificial Sequence
  source: /note=“Description of Artificial Sequence Synthetic oligonucleotide”
uucucuuggguccauuguuucuu

SEQ ID NO: 1275 - RNA, 21 nt, Artificial Sequence
  source: /note=“Description of Artificial Sequence Synthetic oligonucleotide”
  modified_base (21)..(21): Inverted abasic nucleotide
aauagaaaaaauaauccgacn

SEQ ID NO: 1276 - RNA, 23 nt, Artificial Sequence
  source: /note=“Description of Artificial Sequence Synthetic oligonucleotide”
agucggauuauuuuuucuauuuu

SEQ ID NO: 1277 - RNA, 21 nt, Artificial Sequence
  source: /note=“Description of Artificial Sequence Synthetic oligonucleotide”
  modified_base (21)..(21): Inverted abasic nucleotide
auagaaaaaauaauccgacun

SEQ ID NO: 1278 - RNA, 23 nt, Artificial Sequence
  source: /note=“Description of Artificial Sequence Synthetic oligonucleotide”
aagucggauuauuuuuucuauuu

SEQ ID NO: 1279 - RNA, 21 nt, Artificial Sequence
  source: /note=“Description of Artificial Sequence Synthetic oligonucleotide”
  modified_base (21)..(21): Inverted abasic nucleotide
aaaacaauucacuaaaaauan

SEQ ID NO: 1280 - RNA, 23 nt, Artificial Sequence
  source: /note=“Description of Artificial Sequence Synthetic oligonucleotide”
uuauuuuuagugaauuguuuuuu

SEQ ID NO: 1281 - RNA, 21 nt, Artificial Sequence
  source: /note=“Description of Artificial Sequence Synthetic oligonucleotide”
  modified_base (21)..(21): Inverted abasic nucleotide
uguaguuauaaaauaaaacgn

SEQ ID NO: 1282 - RNA, 23 nt, Artificial Sequence
  source: /note=“Description of Artificial Sequence Synthetic oligonucleotide”
acguuuuauuuuauaacuacauu

SEQ ID NO: 1283 - RNA, 21 nt, Artificial Sequence
  source: /note=“Description of Artificial Sequence Synthetic oligonucleotide”
  modified_base (21)..(21): Inverted abasic nucleotide
aauaaaacguuugacuucuan

SEQ ID NO: 1284 - RNA, 23 nt, Artificial Sequence
  source: /note=“Description of Artificial Sequence Synthetic oligonucleotide”
uuagaagucaaacguuuuauuuu

SEQ ID NO: 1285 - RNA, 21 nt, Artificial Sequence
  source: /note=“Description of Artificial Sequence Synthetic oligonucleotide”
  modified_base (21)..(21): Inverted abasic nucleotide
auaaaacguuugacuucuaan

SEQ ID NO: 1286 - RNA, 23 nt, Artificial Sequence
  source: /note=“Description of Artificial Sequence Synthetic oligonucleotide”
uuuagaagucaaacguuuuauuu

SEQ ID NO: 1287 - RNA, 21 nt, Artificial Sequence
  source: /note=“Description of Artificial Sequence Synthetic oligonucleotide”
  modified_base (21)..(21): Inverted abasic nucleotide
uaaaacguuugacuucuaaan

SEQ ID NO: 1288 - RNA, 23 nt, Artificial Sequence
  source: /note=“Description of Artificial Sequence Synthetic oligonucleotide”
auuuagaagucaaacguuuuauu

SEQ ID NO: 1289 - RNA, 21 nt, Artificial Sequence
  source: /note=“Description of Artificial Sequence Synthetic oligonucleotide”
  modified_base (21)..(21): Inverted abasic nucleotide
aaaacguuugacuucuaaacn

SEQ ID NO: 1290 - RNA, 23 nt, Artificial Sequence
  source: /note=“Description of Artificial Sequence Synthetic oligonucleotide”
aguuuagaagucaaacguuuuuu

SEQ ID NO: 1291 - RNA, 21 nt, Artificial Sequence
  source: /note=“Description of Artificial Sequence Synthetic oligonucleotide”
  modified_base (21)..(21): Inverted abasic nucleotide
aaacguuugacuucuaaacun

SEQ ID NO: 1292 - RNA, 23 nt, Artificial Sequence
  source: /note=“Description of Artificial Sequence Synthetic oligonucleotide”
aaguuuagaagucaaacguuuuu